(19) United States
(12) Patent Application Publication
(10) Pub. No.: US 2026/0365758 A1
(43) Pub. Date: Nov. 19, 2026

(54) ANTI-STEAP1 ANTIGEN-BINDING MOLECULES AND USES THEREOF

(71) Applicant: Genentech, Inc., South San Francisco, CA (US)

(72) Inventors: Meredith Carroll HAZEN, Belmont, CA (US); Isidro HOTZEL, Brisbane, CA (US); Julien LAFRANCE-VANASSE, Oakland, CA (US); Gabriele Maria SCHAEFER, San Mateo, CA (US); Dhaya SESHASAYEE, Cupertino, CA (US)

(21) Appl. No.: 18/997,242

(22) PCT Filed: Jul. 21, 2023
(86) PCT No.: PCT/US2023/070740

§ 371 Date: Jan. 21, 2025

Related U.S. Application Data
(60) Provisional application No. 63/391,568, filed on Jul. 22, 2022.

(51) Int. Cl. C07K 16/40; A61K 9/00; A61K 39/00; A61K 45/06; A61P 35/00; C07K 16/28; C07K 16/30

(52) U.S. Cl. CPC C07K 16/40; A61K 9/0019; A61K 45/06; A61P 35/00; C07K 16/2809; C07K 16/30; C07K 16/3069; A61K 2039/505; A61K 2039/54; C07K 2317/24; C07K 2317/31; C07K 2317/33; C07K 2317/522; C07K 2317/524; C07K 2317/526; C07K 2317/565; C07K 2317/567; C07K 2317/73; C07K 2317/92

(57) ABSTRACT

The present invention provides monospecific and multispecific antigen-binding molecules and compositions thereof. The invention also features methods of treating a disease or disorder, such as cancer, methods of inhibiting tumor growth, kits for use with the one or more methods.

### SEQUENCE LISTING

The instant application contains a Sequence Listing which has been submitted electronically in XML format and is hereby incorporated by reference in its entirety. Said XML copy, created on Jul. 18, 2023, is named “50474-301W02_Sequence_Listing” and is 170,087 bytes in size.

### TECHNICAL FIELD

The present invention relates to antigen-binding molecules that bind to STEAP1, including monospecific and multispecific antibodies, compositions thereof, and methods for treating diseases such as cancer.

### BACKGROUND

Cancer is a heterogeneous disease which evolves at the genetic, phenotypic, and pathological levels. Current therapies include chemotherapy, radiation therapy, surgery, hormone therapy, targeted therapy, immunotherapy, and stem cell transplant. Targeted therapy and immunotherapy have shown promise in mediating potent killing of tumor cells. However, the presence of intratumoral heterogeneity, cancer subtypes, and mutations leading to acquired resistance present challenges in target selection. There remains a need for developing therapeutic agents that recognize one or more targets shared among the different cancer subtypes and those that acquired resistance driving by mutations.

### SUMMARY

At least one aspect of the invention described herein relates to a multispecific antigen-binding molecule comprising: (A) a first antigen-binding domain that binds to STEAP1 and comprises an anti-STEAP1 heavy chain variable region (VH) comprising CDR-H1, CDR-H2, and CDR-H3 of a VH sequence selected from SEQ ID NOs: 7, 17-25, 30-34, 38 and 68; and an anti-STEAP1 light chain variable region (VL) comprising CDR-L1, CDR-L2, and CDR-L3 of a VL sequence selected from SEQ ID NOs: 8, 26-29, 39 and 69; and (B) a second antigen-binding domain that binds to a T cell receptor.

At least another aspect of the invention described herein relates to a multispecific antigen-binding molecule comprising: (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3; and (B) a second antigen-binding domain that binds to a T cell receptor; wherein:
CDR-H1 comprises Xaa¹Xaa²YMA (SEQ ID NO: 35); wherein Xaa¹ is Asp (D) or Asn (N); and Xaa² is His (H), Tyr (Y), or Phe (F);
CDR-H2 comprises YIXaa³YDGXaa⁴Xaa⁵TXaa⁶YGDSVKG (SEQ ID NO: 36); wherein Xaa³ is Asp (D) or Ser (S); Xaa⁴ is Gly (G), Asp (D), or Leu (L); Xaa⁵ is Ser (S), Asp (D), or Asn (N); and Xaa⁶ is Ser (S) or Tyr (Y);
CDR-H3 comprises RSGXaa⁷YHVGYAMXaa⁸Xaa⁹ (SEQ ID NO: 37); wherein Xaa⁷ is Phe (F) or Tyr (Y); Xaa⁸ is Asn (N) or Asp (D); and Xaa⁹ is Ala (A) or Gly (G);
CDR-L1 comprises the amino acid sequence of SEQ ID NO: 4; CDR-L2 comprises the amino acid sequence of SEQ ID NO: 5; and CDR-L3 comprises the amino acid sequence of SEQ ID NO: 6.

In some embodiments, the CDR-H1 comprises the amino acid sequence of SEQ ID NO: 10, 1, or 9.

In some embodiments, the CDR-H2 comprises the amino acid sequence of SEQ ID NO: 2, 11, 12, or 13.

In some embodiments, the CDR-H3 comprises the amino acid sequence of SEQ ID NO: 16, 3, 14, or 15.

In some embodiments, the first antigen-binding domain comprises: CDR-H1 comprising the amino acid sequence of SEQ ID NO: 10; CDR-H2 comprising the amino acid sequence of SEQ ID NO: 2; CDR-H3 comprising the amino acid sequence of SEQ ID NO: 16; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the first antigen-binding domain comprises: CDR-H1 comprising the amino acid sequence of SEQ ID NO: 1; CDR-H2 comprising the amino acid sequence of SEQ ID NO: 2; CDR-H3 comprising the amino acid sequence of SEQ ID NO: 3; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the first antigen-binding domain comprises: CDR-H1 comprising the amino acid sequence of SEQ ID NO: 9; CDR-H2 comprising the amino acid sequence of SEQ ID NO: 11; CDR-H3 comprising the amino acid sequence of SEQ ID NO: 3; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the first antigen-binding domain comprises: CDR-H1 comprising the amino acid sequence of SEQ ID NO: 9; CDR-H2 comprising the amino acid sequence of SEQ ID NO: 12; CDR-H3 comprising the amino acid sequence of SEQ ID NO: 3; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the first antigen-binding domain comprises: CDR-H1 comprising the amino acid sequence of SEQ ID NO: 9; CDR-H2 comprising the amino acid sequence of SEQ ID NO: 12; CDR-H3 comprising the amino acid sequence of SEQ ID NO: 14; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the first antigen-binding domain comprises: CDR-H1 comprising the amino acid sequence of SEQ ID NO: 10; CDR-H2 comprising the amino acid sequence of SEQ ID NO: 13; CDR-H3 comprising the amino acid sequence of SEQ ID NO: 15; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the multispecific antigen-binding molecule is a humanized antibody.

In some embodiments, the first antigen-binding domain and the second antigen-binding domain each independently comprises an IgG framework region, optionally an IgG1 or IgG4 framework region.

In some embodiments, the first antigen-binding domain comprises an anti-STEAP1 heavy chain variable region comprising a VH sequence having at least 80%, 85%, 90%, 95%, or 100% sequence identity to SEQ ID NO: 7, 17-25, 30-34, 38, or 68.

In some embodiments, the first antigen-binding domain comprises an anti-STEAP1 light chain variable region comprising a VL sequence having at least 80%, 85%, 90%, 95%, or 100% sequence identity to SEQ ID NO: 8, 26-29, 39, or 69.

In some embodiments, the first antigen-binding domain comprises an anti-STEAP1 heavy chain variable region comprising a VH sequence having at least 80%, 85%, 90%, 95%, or 100% sequence identity to SEQ ID NO: 68, and an anti-STEAP1 light chain variable region comprising a VL sequence having at least 80%, 85%, 90%, 95%, or 100% sequence identity to SEQ ID NO: 69.

In some embodiments, the first antigen-binding domain comprises an anti-STEAP1 heavy chain variable region comprising a VH sequence having at least 80%, 85%, 90%, 95%, or 100% sequence identity to SEQ ID NO: 34, and an anti-STEAP1 light chain variable region comprising a VL sequence having at least 80%, 85%, 90%, 95%, or 100% sequence identity to SEQ ID NO: 27.

In some embodiments, the first antigen-binding domain comprises an anti-STEAP1 heavy chain variable region comprising a VH sequence having at least 80%, 85%, 90%, 95%, or 100% sequence identity to SEQ ID NO: 30, and an anti-STEAP1 light chain variable region comprising a VL sequence having at least 80%, 85%, 90%, 95%, or 100% sequence identity to SEQ ID NO: 27.

In some embodiments, the first antigen-binding domain comprises an anti-STEAP1 heavy chain variable region comprising a VH sequence having at least 80%, 85%, 90%, 95%, or 100% sequence identity to SEQ ID NO: 31, and an anti-STEAP1 light chain variable region comprising a VL sequence having at least 80%, 85%, 90%, 95%, or 100% sequence identity to SEQ ID NO: 27.

In some embodiments, the first antigen-binding domain comprises an anti-STEAP1 heavy chain variable region comprising a VH sequence having at least 80%, 85%, 90%, 95%, or 100% sequence identity to SEQ ID NO: 32, and an anti-STEAP1 light chain variable region comprising a VL sequence having at least 80%, 85%, 90%, 95%, or 100% sequence identity to SEQ ID NO: 27.

In some embodiments, the first antigen-binding domain comprises an anti-STEAP1 heavy chain variable region comprising a VH sequence having at least 80%, 85%, 90%, 95%, or 100% sequence identity to SEQ ID NO: 33, and an anti-STEAP1 light chain variable region comprising a VL sequence having at least 80%, 85%, 90%, 95%, or 100% sequence identity to SEQ ID NO: 27.

In some embodiments, the T cell receptor is cluster of differentiation 3 (CD3).

In some embodiments, the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the sequences of SEQ ID NOs: 48-53, respectively.

In some embodiments, the second antigen-binding domain that binds to CD3 comprises an anti-CD3 heavy chain variable region comprising a VH sequence having at least 80%, 85%, 90%, 95%, or 100% sequence identity to SEQ ID NO: 54, and a VL sequence having at least 80%, 85%, 90%, 95%, or 100% sequence identity to SEQ ID NO: 55.

In some embodiments, the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the sequences of SEQ ID NO: 40-45, respectively.

In some embodiments, the second antigen-binding domain that binds to CD3 comprises an anti-CD3 heavy chain variable region comprising a VH sequence having at least 80%, 85%, 90%, 95%, or 100% sequence identity to SEQ ID NO: 46, and an anti-CD3 light chain variable region comprising a VL sequence having at least 80%, 85%, 90%, 95%, or 100% sequence identity to SEQ ID NO: 47.

In some embodiments, the multispecific antigen binding molecule comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, a second CH2 (CH2₂) domain, and a second CH3 (CH3₂) domain.

In some embodiments, at least one of the one or more heavy chain constant domains is paired with another heavy chain constant domain.

In some embodiments, the CH3₁ and CH3₂ domains each comprise a protuberance or cavity, and wherein the protuberance or cavity in the CH3₁ domain is positionable in the cavity or protuberance, respectively, in the CH3₂ domain.

In some embodiments, the CH3₁ and CH3₂ domains meet at an interface between said protuberance and cavity.

In some embodiments, the CH2₁ and CH2₂ domains each comprise a protuberance or cavity, and wherein the protuberance or cavity in the CH2₁ domain is positionable in the cavity or protuberance, respectively, in the CH2₂ domain.

In some embodiments, the CH2₁ and CH2₂ domains meet at an interface between said protuberance and cavity.

In some embodiments, the first antigen-binding domain comprises an anti-STEAP1 heavy chain constant region sequence having at least 80%, 85%, 90%, 95%, or 100% sequence identity to SEQ ID NO: 83 and an anti-STEAP1 light chain constant region sequence having at least 80%, 85%, 90%, 95%, or 100% sequence identity to SEQ ID NO: 82; and the second antigen-binding domain comprises an anti-CD heavy chain constant region sequence having at least 80%, 85%, 90%, 95%, or 100% sequence identity to SEQ ID NO: 85 and an anti-CD light chain constant region sequence having at least 80%, 85%, 90%, 95%, or 100% sequence identity to SEQ ID NO: 84.

In some embodiments, the antigen-binding molecule comprises an anti-STEAP1 heavy chain constant region sequence having at least 80%, 85%, 90%, 95%, or 100% sequence identity to SEQ ID NO: 85 and an anti-STEAP1 light chain constant region sequence having at least 80%, 85%, 90%, 95%, or 100% sequence identity to SEQ ID NO: 84; and the second antigen-binding domain comprises an anti-CD3 heavy chain constant region sequence having at least 80%, 85%, 90%, 95%, or 100% sequence identity to SEQ ID NO: 83 and an anti-CD3 light chain constant region sequence having at least 80%, 85%, 90%, 95%, or 100% sequence identity to SEQ ID NO: 82.

In some embodiments, the multispecific antigen-binding molecule comprises an anti-STEAP1 heavy chain comprising SEQ ID NO: 73, an anti-STEAP1 light chain comprising SEQ ID NO: 72, an anti-CD3 heavy chain comprising SEQ ID NO: 79, and an anti-CD3 light chain comprising SEQ ID NO: 78.

In some embodiments, the multispecific antigen-binding molecule comprises an anti-STEAP1 heavy chain comprising SEQ ID NO: 73, an anti-STEAP1 light chain comprising SEQ ID NO: 72, an anti-CD3 heavy chain comprising SEQ ID NO: 81, and an anti-CD3 light chain comprising SEQ ID NO: 80.

In some embodiments, the multispecific antigen-binding molecule comprises an anti-STEAP1 heavy chain comprising SEQ ID NO: 71, an anti-STEAP1 light chain comprising SEQ ID NO: 70, an anti-CD3 heavy chain comprising SEQ ID NO: 79, and an anti-CD3 light chain comprising SEQ ID NO: 78.

In some embodiments, the multispecific antigen-binding molecule comprises an anti-STEAP1 heavy chain comprising SEQ ID NO: 71, an anti-STEAP1 light chain comprising SEQ ID NO: 70, an anti-CD3 heavy chain comprising SEQ ID NO: 81, and an anti-CD3 light chain comprising SEQ ID NO: 80.

In some embodiments, the antigen-binding molecule is a single-chain Fv (scFv), trispecific (Fab3), bispecific (Fab2), diabody ((VL-VH)2 or (VH-VL)2), triabody (trivalent), tetrabody (tetravalent), minibody ((scFV-CH)2), bispecific single-chain Fv (Bis-scFv), IgGdeltaCH2, scFv-Fc, or (scFv)2-Fc.

In some embodiments, the multispecific antigen-binding molecule is a multispecific antibody, preferably a bispecific or trispecific antibody.

In some embodiments, the multispecific antigen-binding molecule further comprises a third antigen-binding domain that binds to a tumor-associated antigen. In some embodiments, the tumor-associated antigen is a receptor expressed on a prostate cancer cell or Ewing sarcoma. In some embodiments, the tumor-associated antigen is prostate-specific membrane antigen (PSMA), STEAP2, prostate stem cell antigen (PSCA), epithelial cell adhesion molecule (EpCAM), prostate-specific antigen (PSA), prostatic acid phosphatase (PAP), or HBA-71.

In some embodiments, the multispecific antigen-binding molecule binds to human STEAP1, cynomolgus monkey STEAP1, or a combination thereof, preferably wherein the multispecific antigen-binding molecule binds to the human STEAP1 of SEQ ID NO: 65.

In some embodiments, the first antigen-binding domain binds to at least one, at least two, at least three, at least four, or at least five residue selected from Ser101, His102, Gln103, Trp195, Gln198, Gln202, and Lys281 of STEAP1, wherein the residue positions 101, 102, 103, 195, 198, 202, and 281 correspond to positions 101, 102, 103, 195, 198, 202, and 281 set forth in SEQ ID NO: 65. In some embodiments, the first antigen-binding domain binds to Ser101, His102, Gln103, Trp195, Gln198, Gln202, and Lys281 of STEAP1. In some embodiments, the heavy chain variable region of the first antigen-binding domain forms a hydrogen bond with at least one, at least two, at least three, at least four, or at least five residue selected from Ser101, His102, Gln103, Trp195, Gln198, Gln202, and Lys281 of STEAP1. In some embodiments, at least one, at least two, at least three, at least four, or at least five residue selected from Leu56, Ser73, Asn74, Gly101, Tyr103, and Tyr107 of the heavy chain variable region of the first antigen-binding domain forms a hydrogen bond with at least one, at least two, at least three, at least four, or at least five residue selected from Ser101, His102, Gln103, Trp195, Gln198, Gln202, and Lys281 of STEAP1, wherein the residue positions of the heavy chain variable region of the first antigen-binding domain correspond to positions 56, 73, 74, 101, 103, and 107 set forth in SEQ ID NO: 18.

In some embodiments, the first antigen-binding domain binds to at least one, at least two, or at least three residue selected from Gln201, Gln202, Asn203 and Lys204 of STEAP1, wherein the residue positions 201, 202, 203 and 204 correspond to positions 201, 202, 203 and 204 set forth in SEQ ID NO: 65. In some embodiments, the first antigen-binding domain binds to Gln201, Gln202, Asn203 and Lys204 of STEAP1. In some embodiments, the light chain variable region of the first antigen-binding domain forms a hydrogen bond with at least one residue selected from Gln201 and Gln202 of STEAP1. In some embodiments, at least one residue selected from Tyr53 and Tyr54 of the light chain variable region of the first antigen-binding domain forms a hydrogen bond with at least one residue selected from Gln201 and Gln202, wherein the residue positions of the light chain variable region of the first antigen-binding domain correspond to positions 53 and 54 set forth in SEQ ID NO: 18. In some embodiments, a residue of the light chain variable region of the first antigen-binding domain forms Van der Waals interactions with at least one residue selected from Asn203 and Lys204 of STEAP1.

In some embodiments, the multispecific antigen-binding molecule has a Cₘₐₓ of about 11, 11.5, 12, 12.6, 13, 13.5, 15, 18, 20, 23.4, 25, 27.9, 29.1, or 30 μg/mL.

In some embodiments, the multispecific antigen-binding molecule has a Cₘₐₓ of about 11 μg/mL to about 30 μg/mL.

In some embodiments, the multispecific antigen-binding molecule has a Cₘₐₓ of about 20 μg/mL to about 28 μg/mL.

In some embodiments, the multispecific antigen-binding molecule has a Cₘₐₓ of about 24 μg/mL.

In some embodiments, the multispecific antigen-binding molecule has an EC₅₀ of about 0.6, 0.56, 0.5, 0.45, 0.4, 0.35, 0.3, 0.25, 0.2, 0.15, 0.1, 0.09, 0.05, or lower.

In some embodiments, the multispecific antigen-binding molecule has an EC₅₀ of about 0.05 to about 0.8.

In some embodiments, the EC₅₀ is determined in a cell killing assay at 72 hours with human CD8+ T cells and STEAP1-expressing LNCaP-X1.2 cells, and the EC₅₀ is about 0.05 to about 0.4.

In some embodiments, the EC₅₀ is about 0.08 or about 0.3.

In some embodiments, the EC₅₀ is determined in a cell killing assay at 72 hours with human CD8+ T cells and STEAP1-expressing LNCaPX1.2KO3-13 cells, and the EC₅₀ is about 0.1 to about 0.8.

In some embodiments, the EC₅₀ is about 0.1 or about 0.7.

In some embodiments, the multispecific antigen-binding molecule binds to STEAP1 monovalently.

At least another aspect of the invention described herein relates to an antigen-binding molecule that binds to STEAP1, comprising an anti-STEAP1 heavy chain variable region (VH) comprising CDR-H1, CDR-H2, and CDR-H3 of a VH sequence selected from SEQ ID NOs: 7, 17-25, 30-34, 38, and 68; and an anti-STEAP1 light chain variable region (VL) comprising CDR-L1, CDR-L2, and CDR-L3 of a VL sequence selected from SEQ ID NOs: 8, 26-29, 39, and 69.

At least another aspect of the invention described herein relates to an antigen-binding molecule that binds to STEAP1, comprising: an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3; wherein CDR-H1 comprises Xaa¹Xaa²YMA (SEQ ID NO: 35); wherein Xaa¹ is Asp (D) or Asn (N); and Xaa² is His (H), Tyr (Y), or Phe (F);
CDR-H2 comprises YIXaa³YDGXaa⁴Xaa⁵TXaa⁶YGDSVKG (SEQ ID NO: 36); wherein Xaa³ is Asp (D) or Ser (S); Xaa⁴ is Gly (G), Asp (D), or Leu (L); Xaa⁵ is Ser (S), Asp (D), or Asn (N); and Xaa⁶ is Ser (S) or Tyr (Y);
CDR-H3 comprises RSGXaa⁷YHVGYAMXaa⁸Xaa⁹ (SEQ ID NO: 37); wherein Xaa⁷ is Phe (F) or Tyr (Y); Xaa⁸ is Asn (N) or Asp (D); and Xaa⁹ is Ala (A) or Gly (G);
CDR-L1 comprises the amino acid sequence of SEQ ID NO: 4; CDR-L2 comprises the amino acid sequence of SEQ ID NO: 5; and CDR-L3 comprises the amino acid sequence of SEQ ID NO: 6.

In some embodiments, the CDR-H1 comprises the amino acid sequence of SEQ ID NO: 10, 1, or 9. In some embodiments, the CDR-H2 comprises the amino acid sequence of SEQ ID NO: 2, 11, 12, or 13. In some embodiments, the CDR-H3 comprises the amino acid sequence of SEQ ID NO: 16, 3, 14, or 15.

In some embodiments, the antigen-binding molecule comprises: CDR-H1 comprising the amino acid sequence of SEQ ID NO: 10; CDR-H2 comprising the amino acid sequence of SEQ ID NO: 2; CDR-H3 comprising the amino acid sequence of SEQ ID NO: 16; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the antigen-binding molecule comprises: CDR-H1 comprising the amino acid sequence of SEQ ID NO: 1; CDR-H2 comprising the amino acid sequence of SEQ ID NO: 2; CDR-H3 comprising the amino acid sequence of SEQ ID NO: 3; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the antigen-binding molecule comprises: CDR-H1 comprising the amino acid sequence of SEQ ID NO: 9; CDR-H2 comprising the amino acid sequence of SEQ ID NO: 11; CDR-H3 comprising the amino acid sequence of SEQ ID NO: 3; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the antigen-binding molecule comprises: CDR-H1 comprising the amino acid sequence of SEQ ID NO: 9; CDR-H2 comprising the amino acid sequence of SEQ ID NO: 12; CDR-H3 comprising the amino acid sequence of SEQ ID NO: 3; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the antigen-binding molecule comprises: CDR-H1 comprising the amino acid sequence of SEQ ID NO: 9; CDR-H2 comprising the amino acid sequence of SEQ ID NO: 12; CDR-H3 comprising the amino acid sequence of SEQ ID NO: 14; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the antigen-binding molecule comprises: CDR-H1 comprising the amino acid sequence of SEQ ID NO: 10; CDR-H2 comprising the amino acid sequence of SEQ ID NO: 13; CDR-H3 comprising the amino acid sequence of SEQ ID NO: 15; CDR-L1 comprising the amino acid sequence of SEQ ID NO: 4; CDR-L2 comprising the amino acid sequence of SEQ ID NO: 5; and CDR-L3 comprising the amino acid sequence of SEQ ID NO: 6.

In some embodiments, the antigen-binding molecule is a humanized antibody.

In some embodiments, the antigen-binding molecule comprises an IgG framework region, optionally an IgG1 framework region.

In some embodiments, the antigen-binding molecule comprises an anti-STEAP1 heavy chain variable region comprising a VH sequence having at least 80%, 85%, 90%, 95%, or 100% sequence identity to SEQ ID NO: 7, 17-25, 30-34, 38, or 68.

In some embodiments, the antigen-binding molecule comprises an anti-STEAP1 light chain variable region comprising a VL sequence having at least 80%, 85%, 90%, 95%, or 100% sequence identity to SEQ ID NO: 8, 26-29, 39, or 69.

In some embodiments, the antigen-binding molecule comprises an anti-STEAP1 heavy chain variable region comprising a VH sequence having at least 80%, 85%, 90%, 95%, or 100% sequence identity to SEQ ID NO: 68, and an anti-STEAP1 light chain variable region comprising a VL sequence having at least 80%, 85%, 90%, 95%, or 100% sequence identity to SEQ ID NO: 69.

In some embodiments, the antigen-binding molecule comprises an anti-STEAP1 heavy chain variable region comprising a VH sequence having at least 80%, 85%, 90%, 95%, or 100% sequence identity to SEQ ID NO: 34, and an anti-STEAP1 light chain variable region comprising a VL sequence having at least 80%, 85%, 90%, 95%, or 100% sequence identity to SEQ ID NO: 27.

In some embodiments, the antigen-binding molecule comprises an anti-STEAP1 heavy chain variable region comprising a VH sequence having at least 80%, 85%, 90%, 95%, or 100% sequence identity to SEQ ID NO: 30, and an anti-STEAP1 light chain variable region comprising a VL sequence having at least 80%, 85%, 90%, 95%, or 100% sequence identity to SEQ ID NO: 27.

In some embodiments, the antigen-binding molecule comprises an anti-STEAP1 heavy chain variable region comprising a VH sequence having at least 80%, 85%, 90%, 95%, or 100% sequence identity to SEQ ID NO: 31, and an anti-STEAP1 light chain variable region comprising a VL sequence having at least 80%, 85%, 90%, 95%, or 100% sequence identity to SEQ ID NO: 27.

In some embodiments, the antigen-binding molecule comprises an anti-STEAP1 heavy chain variable region comprising a VH sequence having at least 80%, 85%, 90%, 95%, or 100% sequence identity to SEQ ID NO: 32, and an anti-STEAP1 light chain variable region comprising a VL sequence having at least 80%, 85%, 90%, 95%, or 100% sequence identity to SEQ ID NO: 27.

In some embodiments, the antigen-binding molecule comprises an anti-STEAP1 heavy chain variable region comprising a VH sequence having at least 80%, 85%, 90%, 95%, or 100% sequence identity to SEQ ID NO: 33, and an anti-STEAP1 light chain variable region comprising a VL sequence having at least 80%, 85%, 90%, 95%, or 100% sequence identity to SEQ ID NO: 27.

In some embodiments, the antigen-binding molecule comprises an anti-STEAP1 heavy chain constant region sequence having at least 80%, 85%, 90%, 95%, or 100% sequence identity to SEQ ID NO: 83 and an anti-STEAP1 light chain constant region sequence having at least 80%, 85%, 90%, 95%, or 100% sequence identity to SEQ ID NO: 82.

In some embodiments, the antigen-binding molecule comprises an anti-STEAP1 heavy chain constant region sequence having at least 80%, 85%, 90%, 95%, or 100% sequence identity to SEQ ID NO: 85 and an anti-STEAP1 light chain constant region sequence having at least 80%, 85%, 90%, 95%, or 100% sequence identity to SEQ ID NO: 84.

In some embodiments, the antigen-binding molecule comprises an anti-STEAP1 heavy chain comprising SEQ ID NO: 73 and an anti-STEAP1 light chain comprising SEQ ID NO: 72. In some embodiments, the antigen-binding molecule comprises an anti-STEAP1 heavy chain comprising SEQ ID NO: 71 and an anti-STEAP1 light chain comprising SEQ ID NO: 70.

In some embodiments, the antigen-binding molecule is a full-length antibody or a fragment thereof.

In some embodiments, the antigen-binding molecule is a Fab, Fab′, F(ab′)2, Fv, Fd, single-chain Fv (scFv), trispecific (Fab3), bispecific (Fab2), diabody ((VL-VH)2 or (VH-VL)2), triabody (trivalent), tetrabody (tetravalent), minibody ((scFV-CH)2), bispecific single-chain Fv (Bis-scFv), IgGdeltaCH2, scFv-Fc, or (scFv)2-Fc.

In some embodiments, the antigen-binding molecule is a multispecific antibody, preferably wherein the antigen-binding molecule is a bispecific antibody or a trispecific antibody.

In some embodiments, the antigen-binding molecule further comprises an antigen-binding domain that binds to a T cell receptor. In some embodiments, the T cell receptor is cluster of differentiation 3 (CD3).

In some embodiments, the antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein CDR-H1, CDR-H2, CDR-H3, CDR-L1, CDR-L2, and CDR-L3 comprise the amino acid sequences of SEQ ID NOs: 48-53, respectively.

In some embodiments, the antigen-binding domain that binds to CD3 comprises an anti-CD3 heavy chain variable region comprising a VH sequence having at least 80%, 85%, 90%, 95%, or 100% sequence identity to SEQ ID NO: 54, and an anti-CD3 light chain variable region comprising a VL sequence having at least 80%, 85%, 90%, 95%, or 100% sequence identity to SEQ ID NO: 55.

In some embodiments, the antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein CDR-H1, CDR-H2, CDR-H3, CDR-L1, CDR-L2, and CDR-L3 comprise the amino acid sequences of SEQ ID NO: 40-45, respectively.

In some embodiments, the antigen-binding domain that binds to CD3 comprises an anti-CD3 heavy chain variable region comprising a VH sequence having at least 80%, 85%, 90%, 95%, or 100% sequence identity to SEQ ID NO: 46, and an anti-CD3 light chain variable region comprising a VL sequence having at least 80%, 85%, 90%, 95%, or 100% sequence identity to SEQ ID NO: 47.

In some embodiments, the antigen-binding molecule further comprises an additional antigen-binding domain that binds to a tumor-associated antigen. In some embodiments, the tumor-associated antigen is a receptor expressed on a prostate cancer cell or Ewing sarcoma. In some embodiments, the tumor-associated antigen is prostate-specific membrane antigen (PSMA), STEAP2, prostate stem cell antigen (PSCA), epithelial cell adhesion molecule (EpCAM), prostate-specific antigen (PSA), prostatic acid phosphatase (PAP), or HBA-71.

In some embodiments, the antigen-binding molecule binds to human STEAP1, cynomolgus monkey STEAP1, or a combination thereof, preferably wherein the antigen-binding molecule binds to the human STEAP1 of SEQ ID NO: 65.

In some embodiments, the antigen-binding molecule binds to at least one, at least two, at least three, at least four, or at least five residue selected from Ser101, His102, Gln103, Trp195, Gln198, Gln202, and Lys281 of STEAP1, wherein the residue positions 101, 102, 103, 195, 198, 202, and 281 correspond to positions 101, 102, 103, 195, 198, 202, and 281 set forth in SEQ ID NO: 65.

In some embodiments, the antigen-binding molecule binds to Ser101, His102, Gln103, Trp195, Gln198, Gln202, and Lys281 of STEAP1. In some embodiments, the heavy chain variable region of the antigen-binding molecule forms a hydrogen bond with at least one, at least two, at least three, at least four, or at least five residues selected from Ser101, His102, Gln103, Trp195, Gln198, Gln202, and Lys281 of STEAP1. In some embodiments, at least one, at least two, at least three, at least four, or at least five residues selected from Leu56, Ser73, Asn74, Gly101, Tyr103, and Tyr107 of the heavy chain variable region of the antigen-binding molecule forms a hydrogen bond with at least one, at least two, at least three, at least four, or at least five residues selected from Ser101, His102, Gln103, Trp195, Gln198, Gln202, and Lys281 of STEAP1, wherein the residue positions of the heavy chain variable region of the antigen-binding molecule correspond to positions 56, 73, 74, 101, 103, and 107 set forth in SEQ ID NO: 18.

In some embodiments, the antigen-binding molecule binds to at least one, at least two, at or at least three residue selected from Gln201, Gln202, Asn203 and Lys204 of STEAP1, wherein the residue positions 201, 202, 203 and 204 correspond to positions 201, 202, 203 and 204 set forth in SEQ ID NO: 65. In some embodiments, the antigen-binding molecule binds to Gln201, Gln202, Asn203 and Lys204 of STEAP1. In some embodiments, the light chain variable region of the antigen-binding molecule forms a hydrogen bond with at least one residue selected from Gln201 and Gln202 of STEAP1. In some embodiments, at least one residue selected from Tyr53 and Tyr54 of the light chain variable region of the antigen-binding molecule forms a hydrogen bond with at least one residue selected from Gln201 and Gln202, wherein the residue positions of the light chain variable region of the antigen-binding molecule correspond to positions 53 and 54 set forth in SEQ ID NO: 18. In some embodiments, a residue of the light chain variable region of the antigen-binding molecule forms Van der Waals interactions with at least one residue selected from Asn203 and Lys204 of STEAP1.

In some embodiments, the antigen-binding molecule binds to STEAP1 monovalently.

At least another aspect of the invention described herein relates to an antibody comprising a first antigen-binding domain that binds to human STEAP1 at one or more residues selected from Ser101, His102, Gln103, Trp195, Gln198, Gln202, and Lys281, wherein the residue positions 101, 102, 103, 195, 198, 202, and 281 correspond to positions 101, 102, 103, 195, 198, 202, and 281 set forth in SEQ ID NO: 65.

In some embodiments, the first antigen-binding domain binds to at least one, at least two, at least three, at least four, or at least five residue selected from Ser101, His102, Gln103, Trp195, Gln198, Gln202, and Lys281 of SEQ ID NO: 65. In some embodiments, the first antigen-binding domain binds to Ser101, His102, Gln103, Trp195, Gln198, Gln202, and Lys281 of SEQ ID NO: 65. In some embodiments, the heavy chain variable region of the first antigen-binding domain forms a hydrogen bond with at least one, at least two, at least three, at least four, or at least five residues selected from Ser101, His102, Gln103, Trp195, Gln198, Gln202, and Lys281 of STEAP1. In some embodiments, at least one, at least two, at least three, at least four, or at least five residues selected from Leu56, Ser73, Asn74, Gly101, Tyr103, and Tyr107 of the heavy chain variable region of the first antigen-binding domain forms a hydrogen bond with at least one, at least two, at least three, at least four, or at least five residues selected from Ser101, His102, Gln103, Trp195, Gln198, Gln202, and Lys281 of STEAP1, wherein the residue positions of the heavy chain variable region of the first antigen-binding domain correspond to positions 56, 73, 74, 101, 103, and 107 set forth in SEQ ID NO: 18.

In some embodiments, the first antigen-binding domain comprises an anti-STEAP1 heavy chain variable region (VH) comprising CDR-H1, CDR-H2, and CDR-H3 of a VH sequence selected from SEQ ID NO: 7, 17-25, 30-34, 38, and 68; and three CDRs of an anti-STEAP1 light chain variable region (VL) comprising CDR-L1, CDR-L2, and CDR-L3 of a VL sequence selected from SEQ ID NO: 8, 26-29, 39, and 69.

In some embodiments, the first antigen-binding domain comprises an anti-STEAP1 heavy chain variable region comprising a VH sequence comprising at least 80%, 85%, 90%, 95%, or 100% sequence identity to SEQ ID NO: 7, 17-25, 30-34, 38, or 68.

In some embodiments, the first antigen-binding domain comprises an anti-STEAP1 light chain variable region comprising a VL sequence comprising at least 80%, 85%, 90%, 95%, or 100% sequence identity to SEQ ID NO: 8, 26-29, 39, or 69.

In some embodiments, the antibody further comprises a second antigen-binding domain that binds to a T cell receptor. In some embodiments, the T cell receptor is cluster of differentiation 3 (CD3).

In some embodiments, the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the amino acid sequences of SEQ ID NO: 48-53, respectively.

In some embodiments, the second antigen-binding domain that binds to CD3 comprises an anti-CD3 heavy chain variable region comprising a VH sequence having at least 80%, 85%, 90%, 95%, or 100% sequence identity to SEQ ID NO: 54 and an anti-CD3 light chain variable region comprising a VL sequence having at least 80%, 85%, 90%, 95%, or 100% sequence identity to SEQ ID NO: 55.

In some embodiments, the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the amino acid sequences of SEQ ID NO: 40-45, respectively.

In some embodiments, the second antigen-binding domain that binds to CD3 comprises an anti-CD3 heavy chain variable region comprising a VH sequence having at least 80%, 85%, 90%, 95%, or 100% sequence identity to SEQ ID NO: 46 and an anti-CD3 light chain variable region comprising a VL sequence having at least 80%, 85%, 90%, 95%, or 100% sequence identity to SEQ ID NO: 47.

In some embodiments, the multispecific antigen-binding molecule further comprises a third antigen-binding domain that binds to a tumor-associated antigen. In some embodiments, the tumor-associated antigen is a receptor expressed on a prostate cancer cell. In some embodiments, the tumor-associated antigen is prostate-specific membrane antigen (PSMA), STEAP2, prostate stem cell antigen (PSCA), epithelial cell adhesion molecule (EpCAM), prostate-specific antigen (PSA), or prostatic acid phosphatase (PAP).

At least another aspect of the invention described herein relates to one or more isolated nucleic acids individually or together encoding the multispecific antigen-binding molecule described herein, the antigen-binding molecule described herein, or the antibody described herein.

At least another aspect of the invention described herein relates to one or more vectors individually or together comprising the isolated nucleic acid(s) described herein.

At least another aspect of the invention described herein relates to one or more host cells individually or together comprising the isolated nucleic acid(s) described herein or the vector(s) described herein. In some embodiments, the host cell is a mammalian cell. In some embodiments, the mammalian cell is a Chinese hamster ovary (CHO) cell. In some embodiments, the host cell is an insect cell. In some embodiments, the host cell is a prokaryotic cell.

At least another aspect of the invention described herein relates to a method of producing the multispecific antigen-binding molecule described herein, the antigen-binding molecule described herein, or the antibody described herein, comprising culturing the host cell(s) described herein in a culture medium. In some embodiments, the method further comprises harvesting the multispecific antigen-binding molecule described herein, the antigen-binding molecule described herein, or the antibody described herein from the host cell(s) or the culture medium.

At least another aspect of the invention described herein relates to a pharmaceutical composition comprising the multispecific antigen-binding molecule described herein, the antigen-binding molecule described herein, or the antibody described herein. In some embodiments, the pharmaceutical composition further comprises a pharmaceutically acceptable carrier, excipient, or diluent.

In some embodiments, the pharmaceutical composition is for use as a medicament. In some embodiments, the pharmaceutical composition is for use in treating or delaying progression of a STEAP1 expressing cancer. In some embodiments, the pharmaceutical composition is for use in treating or delaying progression of prostate cancer or Ewing sarcoma.

At least another aspect of the invention described herein relates to use of the multispecific antigen-binding molecule described herein, the antigen-binding molecule described herein, or the antibody described herein for use in treating or delaying progression of a STEAP1-expressing cancer in a subject in need thereof.

At least another aspect of the invention described herein relates to use of the multispecific antigen-binding molecule described herein, the antigen-binding molecule described herein, or the antibody described herein for use in inhibiting or reducing the proliferation of a STEAP1 expressing cancer cell.

In some embodiments, the STEAP1-expressing cancer is a solid tumor.

In some embodiments, the STEAP1-expressing cancer is prostate cancer or Ewing sarcoma.

In some embodiments, the multispecific antigen-binding molecule described herein, the antigen-binding molecule described herein, or the antibody described herein is to be used in combination with an additional therapeutic agent or an additional therapeutic regimen.

In some embodiments, the additional therapeutic agent comprises a chemotherapeutic agent, an immunotherapeutic agent, a targeted therapy, radiation therapy, or a combination thereof.

In some embodiments, the additional therapeutic agent comprises a first-line, second-line, or third-line therapy.

In some embodiments, the additional therapeutic regimen comprises surgery.

In some embodiments, the multispecific antigen-binding molecule described herein, the antigen-binding molecule described herein, or the antibody described herein and the additional therapeutic agent are to be administered simultaneously.

In some embodiments, the multispecific antigen-binding molecule described herein, the antigen-binding molecule described herein, or the antibody described herein and the additional therapeutic agent are to be administered sequentially.

In some embodiments, the multispecific antigen-binding molecule described herein, the antigen-binding molecule described herein, or the antibody described herein is to be administered first prior to administering the additional therapeutic agent.

In some embodiments, the multispecific antigen-binding molecule described herein, the antigen-binding molecule described herein, or the antibody described herein is to be administered after administering the additional therapeutic agent.

In some embodiments, the multispecific antigen-binding molecule described herein, the antigen-binding molecule described herein, or the antibody described herein is to be administered systemically.

In some embodiments, the multispecific antigen-binding molecule described herein, the antigen-binding molecule described herein, or the antibody described herein is to be administered locally.

In some embodiments, the multispecific antigen-binding molecule described herein, the antigen-binding molecule described herein, or the antibody described herein is to be administered by parenteral administration.

In some embodiments, the multispecific antigen-binding molecule described herein, the antigen-binding molecule described herein, or the antibody described herein is to be administered intravenously or subcutaneously.

In some embodiments, the subject is a human.

At least another aspect of the invention described herein relates to use of the multispecific antigen-binding molecule described herein, the antigen-binding molecule described herein, or the antibody described herein in the manufacture of a medicament for treating or delaying progression of a STEAP1 expressing cancer, optionally for treating or delaying progression of prostate cancer or Ewing sarcoma.

At least another aspect of the invention described herein relates to use of the multispecific antigen-binding molecule described herein, the antigen-binding molecule described herein, or the antibody described herein in the manufacture of a medicament for inhibiting or reducing the proliferation of a STEAP1 expressing cancer cell, optionally a prostate cancer cell or Ewing sarcoma cell.

At least another aspect of the invention described herein relates to a method for treating or delaying progression of a STEAP1-expressing cancer in a subject in need thereof, comprising administering to the subject an effective amount of the multispecific antigen-binding molecule described herein, the antigen-binding molecule described herein, or the antibody described herein.

In some embodiments, the STEAP1 expressing cancer is a solid tumor. In some embodiments, the STEAP1 expressing cancer is prostate cancer or Ewing sarcoma.

In some embodiments, the method further comprises administering to the subject an additional therapeutic agent or an additional therapeutic regimen. In some embodiments, the additional therapeutic agent comprises a chemotherapeutic agent, an immunotherapeutic agent, a targeted therapy, radiation therapy, or a combination thereof. In some embodiments, the additional therapeutic agent comprises a first-line, second-line, or third-line therapy. In some embodiments, the additional therapeutic regimen comprises surgery.

In some embodiments, the additional therapeutic agent are administered simultaneously with the multispecific antigen-binding molecule described herein, the antigen-binding molecule described herein, or the antibody described herein.

In some embodiments, the additional therapeutic agent are administered sequentially with the multispecific antigen-binding molecule described herein, the antigen-binding molecule described herein, or the antibody described herein.

In some embodiments, the multispecific antigen-binding molecule described herein, the antigen-binding molecule described herein, or the antibody described herein is administered first prior to administering the additional therapeutic agent.

In some embodiments, the multispecific antigen-binding molecule described herein, the antigen-binding molecule described herein, or the antibody described herein is administered after administering the additional therapeutic agent.

In some embodiments, the multispecific antigen-binding molecule described herein, the antigen-binding molecule described herein, or the antibody described herein is administered systemically. In some embodiments, the multispecific antigen-binding molecule described herein, the antigen-binding molecule described herein, or the antibody described herein is administered locally.

In some embodiments, the multispecific antigen-binding molecule described herein, the antigen-binding molecule described herein, or the antibody described herein is administered by parenteral administration. In some embodiments, the multispecific antigen-binding molecule described herein, the antigen-binding molecule described herein, or the antibody described herein is administered intravenously or subcutaneously.

In some embodiments, the subject is a human.

At least another aspect of the invention described herein relates to a method of inhibiting or reducing the proliferation of a STEAP1 expressing cell, comprising contacting the cell with, the multispecific antigen-binding molecule described herein, the antigen-binding molecule described herein, or the antibody described herein for a time sufficient to inhibit proliferation of the cell.

In some embodiments, the cell is a prostate cancer cell or Ewing sarcoma cell. In some embodiments the method is an in vivo method. In some embodiments the method is an in vitro or an ex vivo method.

At least another aspect of the invention described herein relates to a kit comprising the multispecific antigen-binding molecule described herein, the antigen-binding molecule described herein, the antibody described herein, the isolated nucleic acid(s) described herein, the vector(s) described herein, the host cell(s) described herein, or the pharmaceutical composition described herein, optionally comprising a set of instructions.

### BRIEF DESCRIPTION OF THE DRAWINGS

FIG. 1A illustrates screening of select rat monoclonal antibodies for specific binding to STEAP1-expressing cells. The rat monoclonal antibodies are STEAP1-44, STEAP1-45, STEAP1-81, STEAP1-75, STEAP1-103, STEAP1-10, STEAP1-23, STEAP1-67, STEAP1-34, STEAP1-19, and STEAP1-92.

FIG. 1B illustrates screening of select rat monoclonal antibodies for specific binding to STEAP1-expressing cells. The rat monoclonal antibodies are STEAP1-48, STEAP1-15, STEAP1-28, STEAP1-95, STEAP1-54, STEAP1-69, STEAP1-2, STEAP1-59, STEAP1-49, and STEAP1-62. Ab120 is the control antibody.

FIG. 1C shows binding of antibody STEAP1-44 in a titration assay. Arrows indicate the minimal concentration in which binding of STEAP1-44 to 293-hSTEAP1, PC3-hSTEAP1, LNCaP X1.2, and 22Rv1 cells was observed, which was 0.39 nM, 6.25 nM, 3.125 nM, and 12.5 nM, respectively.

FIG. 2 illustrates variable region sequences of antibody STEAP1-44 and STEAP1-44 clonal variants obtained by deep sequencing aligned to closest rat germline segment sequences (top sequences). For the clonal variants obtained by deep sequencing (“NGS”) only heavy chains are available. These were combined with the STEAP1-44 light chain to produce full-length antibodies and thus share light chains. Presumed somatic mutations are shown in black background. CDR boundaries according to the Kabat and Chothia systems shown above the alignments, with Kabat CDRs boundaries underlined. Residue numbering is according to the Kabat system. Dots represent sequence gaps introduced by aligning software. Germline segment names are shown in the alignments. The SEQ ID NOs for the Kabat CDRs and the VH and VL sequences are shown in Table 1. The SEQ ID NOs for the Chothia CDRs are shown in Table 2.

FIG. 3 illustrates scoring of somatic mutations in the STEAP1-44 clonotype group in the deep sequencing dataset. Sequence read counts with each of the amino acid mutations are shown for each position. Black boxes indicate germline segment wild-type residue. Residues highlighted in grey show the most prevalent somatic mutations in CDR regions.

FIG. 4A shows testing of STEAP1-44 clonal variants binding to STEAP1 on surface of cells.

FIG. 4B shows testing of STEAP1-44 clonal variants binding to soluble recombinant STEAP1.

FIG. 5A shows light chain variable region sequences of STEAP1-44 humanization variants aligned to humanization frameworks. Differences in variants with humanization variants are shown in black background. CDR boundaries according to the Kabat and Chothia systems shown above the alignments, with Kabat CDRs boundaries underlined. Residue numbering is according to the Kabat system. Dots represent sequence gaps introduced by aligning software. The SEQ ID NOs for the Kabat CDRs and the VL sequences are illustrated in Table 1. The SEQ ID NOs for the Chothia CDRs are illustrated in Table 2.

FIG. 5B shows heavy chain variable region sequences of STEAP1-44 humanization variants aligned to humanization frameworks. Differences in variants with humanization frameworks are shown in black background. CDR boundaries according to the Kabat and Chothia systems shown above the alignments, with Kabat CDRs boundaries underlined. Residue numbering is according to the Kabat system. Dots represent sequence gaps introduced by aligning software. The SEQ ID NOs for the Kabat CDRs and the VH sequences are illustrated in Table 1. The SEQ ID NOs for the Chothia CDRs are illustrated in Table 2.

FIG. 6A shows median fluorescence units (MFI) for antibody humanization variants tested at two different concentrations. Variants are ranked from strongest to weakest binding from left to right, in the same order in both concentrations tested.

FIG. 6B shows humanized variant huAb44.v6 with the somatic mutations of 5 NGS-based variants were tested for binding to LNCaP-X1.2 cells expressing STEAP1. huAb44.v6 is the parental humanized clone, chAb44 is a human IgG1 with the rat variable domains of Ab44 and huAb44.v6.01 to 05 are the sequence variants of huAb44.v6 with somatic mutations identified by repertoire NGS.

FIG. 6C shows sequence of humanized antibody variant huAb44.v6.05 aligned to humanized antibody huAb44.v6. Differences in huAb44.v6.05 relative huAb44.v6 are shown in black background. CDR boundaries according to the Kabat and Chothia systems shown above the alignments, with Kabat CDRs boundaries underlined. Residue numbering is according to the Kabat system. The SEQ ID NOs for the Kabat CDRs and VH and VL region sequences are illustrated in Table 1. The SEQ ID NOs for the Chothia CDRs are illustrated in Table 2.

FIG. 7A shows bispecific anti-STEAP1/anti-CD3 antibodies tested in cell killing assays with human CD8+ T cells and STEAP1-expressing LNCaP-X1.2 cells. The anti-CD3 arm of the TDB molecules is indicated as MD1.

FIG. 7B shows bispecific anti-STEAP1/anti-CD3 antibodies tested in cell killing assays with human CD8+ T cells and STEAP1-expressing LNCaP-X1.2 cells. The anti-CD3 arm of the TDB molecules is indicated as either MD1 or 40G5c.

FIG. 8A shows STEAP1 levels in gene editing-modified LNCaPX1.2 sublines (LNCaP-X1.2-KO-3-13, LNCaP-X1.KO-2-11) measured by fluorescence activated cell sorting (FACS) analysis or Western blot. LNCaP-X1.2-KO-2-8 lacks STEAP1 expression.

FIG. 8B shows huAb44v6.05 TDBs were tested in cell killing assays with human CD8+ T cells and indicated cell lines.

FIG. 8C shows target-dependent T cell activation (24 h) for huAb44.v6.05/40G5c.

FIG. 8D shows target-dependent cytokine secretion (24 h) for huAb44.v6.05/40G5c.

FIG. 9 illustrates STEAP1-TDBs suppressing growth of established LNCaP-X1.2, LNCaP-X1.2-KO-3-13, and LNCaP-X1.KO-2-11 tumors in NSG mice supplemented with human PBMCs. Animals received a single IV dose at day 0 of indicated STEAP1-TDB. Doses varied from 0.1 mg/kg to 0.5 mg/kg. Individual tumor volumes were plotted for every treatment group. Dotted lines indicate the fitted tumor volume for the control group (vehicle), solid lines indicate individual tumors.

FIG. 10A illustrates concentration-time profiles of anti-STEAP1 antibodies following a single intravenous dose of 1 mg/kg in female SCID mouse.

FIG. 10B shows concentration-time profiles of anti-STEAP1/CD3 TDBs and anti-gD following a single intravenous dose of 1 mg/kg in female SCID mouse.

FIG. 10C shows concentration-time profiles of huAb44.v6.05/40G5c and huAb44.v6.05/MD1 TDBs following intravenous administration in cynomolgus monkeys.

FIG. 11A shows cryo-EM reconstruction of STEAP1 in complex with Ab44 at a resolution of ~3 Å. An isosurface rendering of the STEAP1 homotrimer (arrows 1, 2, and 3 indicate protomers) in complex with three Ab44 Fabs (arrow 4 indicates heavy chains, arrow 5 indicates light chains, arrow 6 indicates constant regions) is shown. STEAP1 extracellular loops ECL1, ECL2 and ECL3 are indicated by arrows 7, 8 and 9, respectively.

FIG. 11B shows ribbon rendering of the structure. A side view (along the plane of the membrane, equivalent to panel A) and a top view are shown. For clarity, heme from only one subunit is shown (indicated by arrow 10). Numbering of the remaining arrows is the same as in FIG. 11A.

FIG. 12A shows interactions between Ab44 Fab and STEAP1 subunit A (two orthogonal side views rotated by 90 degrees are shown). STEAP1 residues located within 4 Å of Ab44 Fab are shown in spheres. Numbering of the arrows is the same as in FIG. 11B. STEAP1 subunits B and C have been omitted for clarity.

FIG. 12B shows interactions between Ab44 Fab and STEAP1 neighboring subunit B. Views are identical to panel A. STEAP1 subunits A and C have been omitted for clarity. Numbering of the arrows is the same as in FIG. 11B.

FIG. 13A shows surface representation of the STEAP1:Fab44 complex. The surfaces buried by complex formation are indicated by arrow 1 (Fab A to STEAP1 subunit A) and arrow 2 (Fab A to STEAP1 subunit B); surfaces buried representing homotypic interactions are indicated by arrow 3 (Fab A to Fab B) or arrow 4 (Fab A to Fab C). STEAP1 monomers are indicated by letters A, B, and C. Light chains are indicated by arrow 5 and heavy chains are indicated by arrows 6, 7, and 8.

FIG. 13B shows open-book representation of the same surfaces with surface area measurements for each buried surface indicated.

FIG. 14 shows organization of Ab44 and Vandortuzumab upon binding to STEAP1. Multiple polar homotypic Fab interactions (shown in spheres) are observed along the three-fold axis of the STEAP1:Ab44 structure and are mostly driven by LC-LC contacts (left panel, viewed from the extracellular side). In contrast, limited interactions are observed between Vandortuzumab Fabs (right panel). Light chains are indicated by arrow 1 and heavy chains are indicated by arrows 2, 3, and 4. For clarity, STEAP1 homotrimer is not shown (except for the residues located within 4 Å of Fab A, represented in spheres).

FIG. 15 shows sequence alignment of human, Macaca fascicularis and Pongo abelili. Expected transmembrane helices are highlighted in yellow and extracellular loops 1, 2 and 3 are highlighted in green, red and orange boxes, respectively.

FIG. 16 shows sequence alignment of variable regions of humanized antibody variant huAb44.v6, huAb44.v6.01, huAb44.v6.02, huAb44.v6.03, huAb44.v6.04, and huAb44.v6.05. CDR sequences according to Kabat definition are underlined. The SEQ ID NOs for the Kabat CDRs and the VH and VL sequences are illustrated in Table 1. The SEQ ID NOs for the Chothia CDRs are illustrated in Table 2.

FIG. 17 shows sequence alignment of STEAP1-specific variable regions of bispecific antibodies. CDR sequences according to Kabat definition are underlined. The SEQ ID NOs for the Kabat CDRs and the VH and VL sequences are illustrated in Table 1. The SEQ ID NOs for the Chothia CDRs are illustrated in Table 2.

FIG. 18 shows sequence alignment of CD3-specific variable regions of bispecific antibodies. CDR sequences according to Kabat definition are underlined. The SEQ ID NOs for the Kabat CDRs and the VH and VL sequences are illustrated in Table 1. The SEQ ID NOs for the Chothia CDRs are illustrated in Table 2.

FIG. 19 shows sequence alignment of constant regions of STEAP1-specific chains.

FIG. 20 shows sequence alignment of constant regions of CD3-specific chains.

FIG. 21 shows sequence alignment of variable regions of rabbit anti-human STEAP1 antibodies. CDR boundaries according to the Kabat and Chothia systems shown above the alignments, with Kabat CDRs boundaries underlined. Residue numbering is according to the Kabat system. Dots represent sequence gaps introduced by aligning software. The SEQ ID NOs for the Kabat CDRs and the VH and VL sequences are shown in Table 1. The SEQ ID NOs for the Chothia CDRs are shown in Table 2.

FIG. 22 shows a comparison of binding of huAb44v6.05 and vandortuzumab to LNCaP-X1.2 cells expressing STEAP1 as assessed by FACS. MFI for antibody variants was tested at the indicated concentrations. The EC50 for vandortuzumab was 3.5 nM, and the EC50 for huAb44.v6.05 was 1.9 nM.

FIG. 23A shows bispecific anti-STEAP1/anti-CD3 antibodies tested in cell killing assays with human CD8+ T cells and STEAP1-expressing LNCaP-X1.2 cells. Vandortuzumab- and huAb44v6.05-containing TDBs were compared. The anti-CD3 arm of the TDB molecules was either MD1 or 40G5c, as indicated.

FIG. 23B shows bispecific anti-STEAP1/anti-CD3 antibodies tested in cell killing assays with human CD8+ T cells and STEAP1-expressing LNCaPX1.2KO3-13 cells. Vandortuzumab- and huAb44v6.05-containing TDBs were compared. The anti-CD3 arm of the TDB molecules was either MD1 or 40G5c, as indicated.

FIGS. 24A-24D are a series of graphs showing a comparison of huAb44.v6.05/40G5c and vandortuzumab/40G5c TDBs in a T-cell activation assay. FIG. 24A shows results of CD4 T cell activation using LNCaP-X12 cells. FIG. 24B shows results of CD8 T cell activation using LNCaP-X12 cells. FIG. 24C shows results of CD4 T cell activation using LNCaP-X1.2-KO-3-13 cells. FIG. 24D shows results of CD8 T cell activation using LNCaP-X1.2-KO-3-13 cells. For each of these experiments, the ratio of PBMCs to target was 10:1, and the incubation time was 24 h.

FIGS. 25A-25J are a series of graphs showing a comparison of huAb44.v6.05/40G5c and vandortuzumab/40G5c in a cytokine secretion assay at 24 h. FIGS. 25A-25E show results with LNCaP-X1.2 cells, and FIGS. 25F-25J show results with LNCaP-X1.2-KO-3-13 cells. FIGS. 25A and 25F show interferon (IFN) gamma secretion. FIGS. 25B and 25G show tumor necrosis factor (TNF) alpha secretion. FIGS. 25C and 25H show IL-2 secretion. FIGS. 25D and 251 show IL-6 secretion. FIGS. 25E and 25J show granzyme B secretion.

### DETAILED DESCRIPTION

### I. Definitions

The term “about” as used herein refers to the usual error range for the respective value readily known to the skilled person in this technical field. Reference to “about” a value or parameter herein includes (and describes) embodiments that are directed to that value or parameter per se.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}). Affinity can be measured by common methods known in the art, including those described herein. Specific illustrative and exemplary embodiments for measuring binding affinity are described in the following.

As used herein, the term “binds to” refers to measurable and reproducible interactions such as binding between a target and an antigen-binding molecule (e.g., an antibody), which is determinative of the presence of the target in the presence of a heterogeneous population of molecules including biological molecules. For example, an antigen-binding molecule that specifically binds to a target (which can be an epitope) is an antigen-binding molecule that binds this target with greater affinity, avidity, more readily, and/or with greater duration than it binds to other targets. In one embodiment, the extent of binding of an antigen-binding molecule to an unrelated target is less than about 10% of the binding of the antigen-binding molecule to the target as measured, e.g., by surface plasmon resonance (SPR), radioimmunoassay (RIA), or Kinetic Exclusion Assay (KinExA®). In certain embodiments, an antigen-binding molecule that specifically binds to a target has an equilibrium dissociation constant (K_{D}) of ≤1 μM, ≤100 nM, ≤10 nM, ≤1 nM, or ≤0.1 nM. In certain embodiments, an antigen-binding molecule specifically binds to an epitope on a protein that is conserved among the protein from different species. In another embodiment, specific binding can include, but does not require exclusive binding.

As used herein, the term “antigen-binding molecule” refers to a molecule that specifically binds to a target epitope, antigen, ligand, or receptor. Antigen-binding molecules include, but are not limited to, antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner. An “affinity matured” antibody refers to an antibody with one or more alterations in one or more complementarity determining regions (CDRs) or hypervariable regions (HVRs), compared to a parent antibody which does not possess such alterations, such alterations resulting in an improvement in the affinity of the antibody for antigen.

The term “antigen-binding domain” refers to a part of a compound or a molecule that specifically binds to a target epitope, antigen, ligand, or receptor. Molecules featuring antigen-binding domains include, but are not limited to, antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “antibody” herein is used in the broadest sense and encompasses various antibody structures, including but not limited to monoclonal antibodies, polyclonal antibodies, multispecific antibodies (e.g., bispecific or trispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nat. Biotech. 23:1126-1136 (2005).

A “single-chain variable fragment” or “scFv” is a fusion protein of the variable domains of the heavy (VH) and light chains (VL) of an antibody, connected by a linker. In particular, the linker is typically a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

The term “antigen” denotes a proteinaceous or non-proteinaceous molecule to which an antigen-binding molecule (e.g., an antibody) binds. The antigen can include protein, protein fragment, or a hapten.

The term “epitope” denotes the site on an antigen, either proteinaceous or non-proteinaceous, to which an anti-STEAP1 antigen-binding molecule (e.g., an anti-STEAP1 antibody) binds. Epitopes can be formed both from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an anti-STEAP1 antigen-binding molecule after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antigen-binding molecules (e.g., antibodies) binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies,” Harlow and Lane (Cold Spring Harbor Press, Cold Spring Harbor, NY).

Antigen Structure-based Antibody Profiling (ASAP), also known as Modification-Assisted Profiling (MAP), allows binning of a multitude of monoclonal antibodies specifically binding to STEAP1 based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Additionally, competitive binding can be used to easily determine whether an antigen-binding molecule (e.g., an antibody) binds to the same epitope of STEAP1 as, or competes for binding with, a reference anti-STEAP1 antibody. For example, an “antigen-binding molecule that binds to the same epitope” as a reference anti-STEAP1 antibody refers to an antigen-binding molecule that blocks binding of the reference anti-STEAP1 antibody to its antigen in a competition assay by 50% or more, and conversely, the reference antibody blocks binding of the antigen-binding molecule to its antigen in a competition assay by 50% or more. Also for example, to determine if an antigen-binding molecule binds to the same epitope as a reference anti-STEAP1 antibody, the reference antibody is allowed to bind to STEAP1 under saturating conditions. After removal of the excess of the reference anti-STEAP1 antibody, the ability of an anti-STEAP1 antigen-binding molecule in question to bind to STEAP1 is assessed. If the anti-STEAP1 antigen-binding molecule is able to bind to STEAP1 after saturation binding of the reference anti-STEAP1 antibody, it can be concluded that the anti-STEAP1 antigen-binding molecule in question binds to a different epitope than the reference anti-STEAP1 antibody. But, if the anti-STEAP1 antigen-binding molecule in question is not able to bind to STEAP1 after saturation binding of the reference anti-STEAP1 antibody, then the anti-STEAP1 antigen-binding molecule in question may bind to the same epitope as the epitope bound by the reference anti-STEAP1 antibody. To confirm whether the antigen-binding molecule in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay may be carried out in two set-ups, i.e., with both of the molecules being the saturating antibody. If, in both set-ups, only the first (saturating) antibody is capable of binding to STEAP1, then it can be concluded that the anti-STEAP1 antigen-binding molecule in question and the reference anti-STEAP1 antibody compete for binding to STEAP1.

The term “STEAP1” refers to any STEAP1 from any vertebrate source, including mammals such as primates (e.g., humans or non-human primates) and rodents (e.g., mice and rats). The term encompasses “full-length” STEAP1 and naturally occurring variants of STEAP1, including, for example, splice variants or allelic variants. STEAP1 is a member of the six-transmembrane epithelial antigen of the prostate (STEAP) protein family. The STEAP protein family comprises five members, STEAP1, STEAP2, STEAP3, STEAP4, and STEAP4. STEAP1, STEAP2, and STEAP4 have been observed to be over-expressed in different cancer cells while minimally expressed in normal tissues. STEAP1 includes, e.g., human STEAP1 (UniProtKB Reference Number: Q9UHE8-1; SEQ ID NO: 65), cynomolgus monkey STEAP1 (UniProtKB Reference Number: A0A2K5X1J3; SEQ ID NO: 66), and orangutan STEAP1 (UniProtKB Reference Number: H2PMZ0; SEQ ID NO: 67).

Below is an exemplary amino acid sequence for human STEAP1:

(SEQ ID NO: 65)
MESRKDITNQEELWKMKPRRNLEEDDYLHKDTGETSMLKRPVLLH
LHQTAHADEFDCPSELQHTQELFPQWHLPIKIAAIIASLTFLYTL
LREVIHPLATSHQQYFYKIPILVINKVLPMVSITLLALVYLPGVI
AAIVQLHNGTKYKKFPHWLDKWMLTRKQFGLLSFFFAVLHAIYSL
SYPMRRSYRYKLLNWAYQQVQQNKEDAWIEHDVWRMEIYVSLGIV
GLAILALLAVTSIPSVSDSLTWREFHYIQSKLGIVSLLLGTIHAL
IFAWNKWIDIKQFVWYTPPTFMIAVFLPIVVLIFKSILFLPCLRK
KILKIRHGWEDVTKINKTEICSQL.

Below is an exemplary amino acid sequence for cynomolgus monkey (Macaca fascicularis) STEAP1:

(SEQ ID NO: 66)
MESRKDITNEEELWKMKPRRNLEEDDYLHKDTGETSMLKRPVLLH
LHQTAHADEFDCPSELQHTQELFPQWHLPIKIAAIIASLTFLYTL
LREVIHPLATSHQQYFYKIPILVINKVLPMVSITLLALVYLPGVI
AAIVQLHNGTKYKKFPHWLDKWMLTRKQFGLLSFFFAVLHAIYSL
SYPMRRSYRYKLLNWAYQQVQQNKEDAWIEHDVWRMEIYVSLGIV
GLAILALLAVTSIPSVSDSLTWREFHYIQSKLGIVSLLLATIHAL
IFAWNKWIDIKQFVWYTPPTFMIAVFLPVVVLIFKSILFLPCLRK
KILKIRHGWEDVTKINKMEISSQL.

Below is an exemplary amino acid sequence for orangutan (Pongo abelii) STEAP1:

(SEQ ID NO: 67)
MESRKDITNQEELWKMKPRRNLEEDDYLHKDTGETSMLKRPVLLH
LHQTAHADEFDCPSELQQTRELFPQWHLPIKIAAIIASLTFLYTL
LREVIHPLATSHQQYFYKIPILVINKVLPMVSITLLALVYLPGVI
AAIVQLHNGTKYKKFPHWLDKWMLTRKQFGLLSFFFAVLHAIYSL
SYPMRRSYRYKLLNWAYQQVQQNKEDAWIEHDVWRMEIYVSLGIV
GLAILALLAVTSIPSVSDSLTWREFHYIQSKLGIVSLLLGTIHAL
IFAWNKWIDIKQFVWYTPPTFMIAVILPIVVLIFKSILFLPCLRK
KILKIRHGWEDVTKINKTEISSQL.

The term “anti-STEAP1 antigen-binding molecule” or “antigen-binding molecule that binds STEAP1” refer to any molecule that is capable of binding to STEAP1 with sufficient affinity such that the molecule is useful as a diagnostic and/or therapeutic agent in targeting STEAP1. In one embodiment, the extent of binding of an anti-STEAP1 antigen-binding molecule to an unrelated, non-STEAP1 protein is less than about 10% of the binding of the antigen-binding molecule to STEAP1 as measured, e.g., by surface plasmon resonance (SPR), radioimmunoassay (RIA), or Kinetic Exclusion Assay (KinExA®). In certain aspects, an anti-STEAP1 antigen-binding molecule has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antigen-binding molecule is said to “specifically bind” to STEAP1 when the antigen-binding molecule has a K_{D} of 1 μM or less. In certain aspects, an anti-STEAP1 antigen-binding molecule binds to an epitope of STEAP1 that is conserved among STEAP from different species.

The term “anti-STEAP1 antibody” or “an antibody that binds to STEAP1” refers to an antibody that is capable of binding to STEAP1 with sufficient affinity such that the molecule is useful as a diagnostic and/or therapeutic agent in targeting STEAP1. In one embodiment, the extent of binding of an anti-STEAP1 antibody to an unrelated, non-STEAP1 protein is less than about 10% of the binding of the antibody to STEAP1 as measured, e.g., by surface plasmon resonance (SPR), radioimmunoassay (RIA), or Kinetic Exclusion Assay (KinExA®). In certain aspects, an anti-STEAP1 antibody has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to STEAP1 when the antibody has a K_{D} of 1 μM or less. In certain aspects, an anti-STEAP1 antibody binds to an epitope of STEAP1 that is conserved among STEAP from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3F or CD3γ), as well as any form of CD3 that results from processing in the cell. The term also encompasses naturally occurring variants of CD3, including, for example, splice variants or allelic variants. CD3 includes, for example, human CD3F protein (NCBI RefSeq No. NP_000724), which is 207 amino acids in length, and human CD3γ protein (NCBI RefSeq No. NP_000064), which is 182 amino acids in length.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by surface plasmon resonance (SPR), radioimmunoassay (RIA), or Kinetic Exclusion Assay (KinExA®). In certain embodiments, an antibody that binds to CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species. In some embodiments, the anti-CD3 antibody is described in International Patent Application Publication No. WO 2015/095392, which is incorporated by reference herein in its entirety. In some embodiments, the anti-CD3 antibody is described in U.S. Pat. No. 10,174,124, which is incorporated by reference herein in its entirety. In other embodiments, the anti-CD3 antibody is described in International Patent Application No. PCT/US2020/064635, which is incorporated by reference herein in its entirety.

In some embodiments, the anti-CD3 antibody is 40G5c. In some embodiments, the anti-CD3 antibody is 38E4V1.MD1. The SEQ ID NOs for the CDRs and the VH and VL sequences of 40G5c and 38E4V1.MD1 are listed in Tables 1 and 2.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG1 isotype. In certain aspects, the antibody is of the IgG₁ isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The term “hypervariable region” or “HVR” as used herein refers to each of the regions of an antibody variable domain which are hypervariable in sequence and which determine antigen binding specificity, for example, “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs) or hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “constant region derived from human origin” or “human constant region” denotes a constant heavy chain region of a human antibody of the subclass IgG1, IgG2, IgG3, or IgG4 and/or a constant light chain kappa or lambda region. Such constant regions are well known in the state of the art and e.g., described by Kabat, E. A., et al., Sequences of Proteins of Immunological Interest, 5th ed., Public Health Service, National Institutes of Health, Bethesda, MD (1991) (see also e.g., Johnson and Wu, Nucleic Acids Res. 28 (2000) 214-218; Kabat et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat et al., Sequences of Proteins of Immunological Interest, 5th ed., Public Health Service, National Institutes of Health, Bethesda, MD (1991), NIH Publication 91-3242.

The terms “Fc region” or “Fc domain” are herein used interchangeably to refer to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. The term encompasses truncated Fc regions, such as those having a C-terminal truncation (e.g., a ΔGK truncation, e.g., as described in Hu et al., Biotechnol. Prog. 2017, 33: 786-794 and Jiang et al., J. Pharm. Sci. 2016, 105: 2066-2072. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. A “subunit” of an Fc domain as used herein refers to one of the two polypeptides forming the dimeric Fc domain, i.e., a polypeptide comprising C-terminal constant regions of an immunoglobulin heavy chain, capable of stable self-association. In one embodiment, a subunit of an IgG Fc domain comprises an IgG CH2 and an IgG CH3 constant domain.

“Framework” or “FR” refers to variable domain residues other than complementary determining regions (CDRs) or hypervariable region (HVR) residues. The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006), regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human CDRs or HVRs and amino acid residues from human FRs. In certain embodiments, a humanized antibody will comprise substantially all of at least one, and typically two, variable domains, in which all or substantially all of the CDRs or HVRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, the term “monospecific” refers to an antigen-binding molecule that binds to a single antigen, e.g., STEAP1. As used herein, the term “multispecific” refers to an antigen-binding molecule that binds to multiple different antigens, e.g., STEAP1 and CD3. For example, “multispecific antibodies” are monoclonal antibodies that have binding specificities for at least two different sites, i.e., different epitopes on different antigens or different epitopes on the same antigen.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (Δ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by Pearson and Lipman, Proc. Natl. Acad. Sci. USA 85:2444-2448 (1988); Pearson Meth. Enzymol. 266:227-258 (1996); and Pearson et al., Genomics 46:24-36 (1997) and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al., Nat. Med. 23(7):815-817, 2017 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-STEAP1 antibody” refers to one or more nucleic acid molecules encoding anti-STEAP1 antibody heavy and light chains (or fragments thereof), including such nucleic acid molecule(s) in a single vector or separate vectors, and such nucleic acid molecule(s) present at one or more locations in a host cell.

“Isolated nucleic acid encoding an anti-CD3 antibody” refers to one or more nucleic acid molecules encoding anti-CD3 antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “administering” is meant a method of giving a dosage of a compound (e.g., an antigen-binding molecule of the invention or a nucleic acid encoding an antigen-binding molecule of the invention) or a composition (e.g., a pharmaceutical composition, e.g., a pharmaceutical composition including an antigen-binding molecule of the invention) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

By “reduce” or “inhibit” is meant the ability to cause an overall decrease, for example, of 20% or greater, of 50% or greater, or of 75%, 85%, 90%, 95%, or greater. In certain embodiments, reduce or inhibit can refer to the effector function of an antigen-binding molecule that is mediated by the Fc region, such effector functions specifically including complement-dependent cytotoxicity (CDC), antibody-dependent cellular cytotoxicity (ADCC), and antibody-dependent cellular phagocytosis (ADCP).

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, 1, or 2 cancer. Examples of cancer include, but are not limited to, solid tumors such as brain cancer, breast cancer, colorectal cancer, endometrial cancer, kidney cancer, liver cancer, lung cancer, melanoma, pancreatic cancer, prostate cancer, stomach cancer, or thyroid cancer; and hematologic malignancies such as Burkitt's lymphoma (BL), multiple myeloma, diffuse large B cell lymphoma (DLBCL), follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), or Waldenström macroglobulinemia (WM).

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “STEAP1-expressing cancer” refers to a cancer those cancer cells are characterized with an over-expression of STEAP1 compared to the expression level of STEAP1 in equivalent non-cancerous cells. Examples of STEAP1-expressing cancer include, but are not limited to, breast cancer, bladder cancer, cervical cancer, colorectal cancer, Ewing sarcoma, lung cancer, ovarian cancer, and prostate cancer.

Prostate cancer is one of the most common cancer in men. Prostate cancer types can include adenocarcinomas of the prostate, sarcoma of the prostate, transitional cell carcinoma of the prostate, small cell carcinomas of the prostate, neuroendocrine tumors of the prostate, and castrate-resistant prostate cancer (CRPC). Prostate cancer can also be classified based on molecular signature. For example, prostate cancers can be classified into those with rearrangements in ETS family transcription factors (e.g., ERG, ETV1, ETV4, and FLI1) and those negative for ETS factors. ETS positive prostate cancer can also include alterations in PI3K and p53 signaling. ETS negative prostate cancers can show recurrent mutations in SPOP, FOXA1, and IDH1; deletions of CHD1; and overexpression of SPINK1. In some instances, the prostate cancer is a metastatic prostate cancer (e.g., metastatic castrate-resistant prostate cancer). In other instances, the prostate cancer is a relapsed or refractory prostate cancer.

Ewing sarcoma (also referred to as Ewing's sarcoma or Ewing's tumor) is a cancer of the bones and soft tissue (e.g., cartilage or nerves). There are several types of Ewing sarcoma, such as Ewing sarcoma of bone, extraosseous Ewing sarcoma, peripheral primitive neuroectodermal tumor (pPNET), and Askin tumor. There is no known cause of Ewing sarcoma. Symptoms include pain and swelling at the site of the tumor.

The term “tumor antigen,” as used herein, may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are tumor antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. Exemplary tumor antigens include, but are not limited to, prostate-specific membrane antigen (PSMA), prostate stem cell antigen (PSCA), epithelial cell adhesion molecule (EpCAM), prostate-specific antigen (PSA), prostatic acid phosphatase (PAP), STEAP2, and HBA-71. In some cases, the TAAs for prostate cancer include prostate-specific membrane antigen (PSMA), prostate stem cell antigen (PSCA), epithelial cell adhesion molecule (EpCAM), prostate-specific antigen (PSA), and prostatic acid phosphatase (PAP). In some cases, the TAA for Ewing sarcoma include HBA-71, an antigen localized at the cell-surface glycocalyx of tumor cells.

The term “T cell receptor” or TCR refers to a receptor expressed on a T cell. Exemplary T cell receptors include, but are not limited to, CD3.

An “effective amount” of a compound, for example, a bispecific antigen-binding molecule of the invention or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers.

In some cases, the additional therapeutic agent comprises a second-line therapy, a third-line therapy, a fourth-line therapy, or a fifth-line therapy. As used herein, a second-line therapy encompasses treatments that are utilized after the primary or first-line treatment stops. A third-line therapy, a fourth-line therapy, or a fifth-line therapy encompass subsequent treatments. As indicated by the naming convention, a third-line therapy encompass a treatment course upon which a primary and second-line therapy have stopped.

As used herein, the terms “individual,” “patient,” or “subject” are used interchangeably, and each refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual, patient, or subject is a human.

### II. Compositions

In one aspect, the invention is based, in part, on antigen-binding molecules (e.g., mono-specific and/or multispecific antigen-binding molecules). In certain embodiments, the antigen-binding molecules bind to STEAP1. In certain embodiments, the antigen-binding molecules are multispecific antigen-binding molecules that bind to STEAP1 and to one or more additional antigens of interest. In certain embodiments, the multispecific antigen-binding molecules bind to STEAP1 monovalently. Antigen-binding molecules of the invention are useful, e.g., for treating or delaying the progression of a cancer (e.g., STEAP1-expressing cancer).

Exemplary Antigen-Binding Molecules that Bind to STEAP1
Antigen-Binding Molecules that Bind to STEAP1

In some embodiments, the invention provides isolated antigen-binding molecules that bind to STEAP1. In some embodiments, an antigen-binding molecule of the present invention comprises at least one, at least two, at least three, at least four, at least five, or all six CDRs as illustrated in Table 1 (Kabat) or Table 2 (Chothia). In some instances, the antigen-binding molecule comprises a VH and/or a VL as illustrated in Table 1.

TABLE 1

| | CDR (Kabat) | | | | | | V | |
|---|---|---|---|---|---|---|---|---|
| Listing of SEQ. ID. NOs. | H1 | H2 | H3 | L1 | L2 | L3 | VH | VL |
| STEAP1-44 | 1 | 2 | 3 | 4 | 5 | 6 | 7 | 8 |
| STEAP1-44.NGS.HC1 | 9 | 11 | 3 | 4 | 5 | 6 | 17 | 8 |
| STEAP1-44.NGS.HC2 | 9 | 12 | 3 | 4 | 5 | 6 | 18 | 8 |
| STEAP1-44.NGS.HC3 | 9 | 12 | 14 | 4 | 5 | 6 | 19 | 8 |
| STEAP1-44.NGS.HC4 | 10 | 13 | 15 | 4 | 5 | 6 | 20 | 8 |
| STEAP1-44.NGS.HC5 | 10 | 2 | 16 | 4 | 5 | 6 | 21 | 8 |
| huAb44.v1 | 1 | 2 | 3 | 4 | 5 | 6 | 22 | 26 |
| huAb44.v2 | 1 | 2 | 3 | 4 | 5 | 6 | 22 | 27 |
| huAb44.v3 | 1 | 2 | 3 | 4 | 5 | 6 | 22 | 28 |
| huAb44.v4 | 1 | 2 | 3 | 4 | 5 | 6 | 22 | 29 |
| huAb44.v5 | 1 | 2 | 3 | 4 | 5 | 6 | 23 | 26 |
| huAb44.v6 | 1 | 2 | 3 | 4 | 5 | 6 | 23 | 27 |
| huAb44.v7 | 1 | 2 | 3 | 4 | 5 | 6 | 23 | 28 |
| huAb44.v8 | 1 | 2 | 3 | 4 | 5 | 6 | 23 | 29 |
| huAb44.v9 | 1 | 2 | 3 | 4 | 5 | 6 | 24 | 26 |
| huAb44.v10 | 1 | 2 | 3 | 4 | 5 | 6 | 24 | 27 |
| huAb44.v11 | 1 | 2 | 3 | 4 | 5 | 6 | 24 | 28 |
| huAb44.v12 | 1 | 2 | 3 | 4 | 5 | 6 | 24 | 29 |
| huAb44.v13 | 1 | 2 | 3 | 4 | 5 | 6 | 25 | 26 |
| huAb44.v14 | 1 | 2 | 3 | 4 | 5 | 6 | 25 | 27 |
| huAb44.v15 | 1 | 2 | 3 | 4 | 5 | 6 | 25 | 28 |
| huAb44.v16 | 1 | 2 | 3 | 4 | 5 | 6 | 25 | 29 |
| huAb44.v6.01 | 9 | 11 | 3 | 4 | 5 | 6 | 30 | 27 |
| huAb44.v6.02 | 9 | 12 | 3 | 4 | 5 | 6 | 31 | 27 |
| huAb44.v6.03 | 9 | 12 | 14 | 4 | 5 | 6 | 32 | 27 |
| huAb44.v6.04 | 10 | 13 | 15 | 4 | 5 | 6 | 33 | 27 |
| huAb44.v6.05 | 10 | 2 | 16 | 4 | 5 | 6 | 34 | 27 |
| huAb44.v6.05 EKKE | 10 | 2 | 16 | 4 | 5 | 6 | 68 | 69 |
| huAb44 Consensus | 35 | 36 | 37 | 4 | 5 | 6 | 38 | 39 |
| 38E4v1.MD1 | 40 | 41 | 42 | 43 | 44 | 45 | 46 | 47 |
| 40G5c | 48 | 49 | 50 | 51 | 52 | 53 | 54 | 55 |
| rbAb3404 | 90 | 91 | 92 | 93 | 94 | 95 | 96 | 97 |
| rbAb3349 | 98 | 99 | 100 | 101 | 102 | 103 | 104 | 105 |

TABLE 2

| | CDR (Chothia) | | | | | |
|---|---|---|---|---|---|---|
| Listing of SEQ. ID. NOs. | H1 | H2 | H3 | L1 | L2 | L3 |
| STEAP1-44 | 56 | 57 | 3 | 4 | 5 | 6 |
| STEAP1-44.NGS.HC1 | 58 | 60 | 3 | 4 | 5 | 6 |
| STEAP1-44.NGS.HC2 | 58 | 61 | 3 | 4 | 5 | 6 |
| STEAP1-44.NGS.HC3 | 58 | 61 | 14 | 4 | 5 | 6 |
| STEAP1-44.NGS.HC4 | 59 | 62 | 15 | 4 | 5 | 6 |
| STEAP1-44.NGS.HC5 | 59 | 57 | 16 | 4 | 5 | 6 |
| huAb44.v1 | 56 | 57 | 3 | 4 | 5 | 6 |
| huAb44.v2 | 56 | 57 | 3 | 4 | 5 | 6 |
| huAb44.v3 | 56 | 57 | 3 | 4 | 5 | 6 |
| huAb44.v4 | 56 | 57 | 3 | 4 | 5 | 6 |
| huAb44.v5 | 56 | 57 | 3 | 4 | 5 | 6 |
| huAb44.v6 | 56 | 57 | 3 | 4 | 5 | 6 |
| huAb44.v7 | 56 | 57 | 3 | 4 | 5 | 6 |
| huAb44.v8 | 56 | 57 | 3 | 4 | 5 | 6 |
| huAb44.v9 | 56 | 57 | 3 | 4 | 5 | 6 |
| huAb44.v10 | 56 | 57 | 3 | 4 | 5 | 6 |
| huAb44.v11 | 56 | 57 | 3 | 4 | 5 | 6 |
| huAb44.v12 | 56 | 57 | 3 | 4 | 5 | 6 |
| huAb44.v13 | 56 | 57 | 3 | 4 | 5 | 6 |
| huAb44.v14 | 56 | 57 | 3 | 4 | 5 | 6 |
| huAb44.v15 | 56 | 57 | 3 | 4 | 5 | 6 |
| huAb44.v16 | 56 | 57 | 3 | 4 | 5 | 6 |
| huAb44.v6.01 | 58 | 60 | 3 | 4 | 5 | 6 |
| huAb44.v6.02 | 58 | 61 | 3 | 4 | 5 | 6 |
| huAb44.v6.03 | 58 | 61 | 14 | 4 | 5 | 6 |
| huAb44.v6.04 | 59 | 62 | 15 | 4 | 5 | 6 |
| huAb44.v6.05 | 59 | 57 | 16 | 4 | 5 | 6 |
| huAb44 Consensus | 63 | 64 | 37 | 4 | 5 | 6 |
| 38E4v1.MD1 | 86 | 87 | 42 | 43 | 44 | 45 |
| 40G5c | 88 | 89 | 50 | 51 | 52 | 53 |
| rbAb3404 | 106 | 107 | 92 | 93 | 94 | 95 |
| rbAb3349 | 108 | 109 | 100 | 101 | 102 | 103 |

In some embodiments, the antigen-binding molecule that binds to STEAP1 comprises of an anti-STEAP1 heavy chain variable region (VH) comprising, consisting essentially of, or consisting of CDR-H1, CDR-H2, and/or CDR-H3 of a VH sequence selected from SEQ ID NO: 7, 17-25, 30-34, and 38; and/or an anti-STEAP1 light chain variable region (VL) comprising, consisting essentially of, or consisting of CDR-L1, CDR-L2, and/or CDR-L3 of a VL sequence selected from SEQ ID NO: 8, 26-29, 39, and 69. In some instances, at least one, at least two, at least three, at least four, at least five, or all six CDRs are selected from the CDR(s) of the VH sequence selected from SEQ ID NOs: 7, 17-25, 30-34, 38, and 68 and the VL sequence selected from SEQ ID NO: 8, 26-29, 39, and 69. In some cases, the six CDRs are defined according to the Kabat numbering (see Table 1). In other cases, the six CDRs are defined according to the Chothia numbering (see Table 2). In additional cases, the six CDRs are defined according to the EU numbering.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein:
CDR-H1 comprises Xaa¹Xaa²YMA (SEQ ID NO: 35);
wherein
   Xaa¹ is Asp (D) or Asn (N); and
   Xaa² is His (H), Tyr (Y), or Phe (F);
CDR-H2 comprises YIXaa³YDGXaa⁴Xaa⁵TXaa⁶YGDSVKG (SEQ ID NO: 36);
wherein
   Xaa³ is Asp (D) or Ser (S);
   Xaa⁴ is Gly (G), Asp (D), or Leu (L);
   Xaa⁵ is Ser (S), Asp (D), or Asn (N); and
   Xaa⁶ is Ser (S) or Tyr (Y);
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein:
CDR-H1 comprises the amino acid sequence of SEQ ID NO: 10, 1, or 9;
CDR-H2 comprises YIXaa³YDGXaa⁴Xaa⁵TXaa⁶YGDSVKG (SEQ ID NO: 36);
wherein
   Xaa³ is Asp (D) or Ser (S);
   Xaa⁴ is Gly (G), Asp (D), or Leu (L);
   Xaa⁵ is Ser (S), Asp (D), or Asn (N); and
   Xaa⁶ is Ser (S) or Tyr (Y);
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3;
wherein
   CDR-H1 comprises the amino acid sequence of SEQ ID NO: 10, 1, or 9;
   CDR-H2 comprises the amino acid sequence of SEQ ID NO: 2, 11, 12, or 13;
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises the amino acid sequence of SEQ ID NO: 2, 11, 12, or 13;
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises the amino acid sequence of SEQ ID NO: 2, 11, 12, or 13;
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises YIXaa³YDGXaa⁴Xaa⁵TXaa⁶YGDSVKG (SEQ ID NO: 36);
   wherein
      Xaa³ is Asp (D) or Ser (S);
      Xaa⁴ is Gly (G), Asp (D), or Leu (L);
      Xaa⁵ is Ser (S), Asp (D), or Asn (N); and
      Xaa⁶ is Ser (S) or Tyr (Y);
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3;
wherein
   CDR-H1 comprises the amino acid sequence of SEQ ID NO: 10, 1, or 9;
   CDR-H2 comprises YIXaa³YDGXaa⁴Xaa⁵TXaa⁶YGDSVKG (SEQ ID NO: 36);
   wherein
      Xaa³ is Asp (D) or Ser (S);
      Xaa⁴ is Gly (G), Asp (D), or Leu (L);
      Xaa⁵ is Ser (S), Asp (D), or Asn (N); and
      Xaa⁶ is Ser (S) or Tyr (Y);
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; in which CDR-H1 comprises, consists essentially of, or consists of the amino acid sequence of SEQ ID NO: 10, 1, or 9; CDR-H2 comprises, consists essentially of, or consists of the amino acid sequence of SEQ ID NO: 2, 11, 12, or 13; CDR-H3 comprises, consists essentially of, or consists of the amino acid sequence of SEQ ID NO: 16, 3, 14, or 15; CDR-L1 comprises, consists essentially of, or consists of the amino acid sequence of SEQ ID NO: 4; CDR-L2 comprises, consists essentially of, or consists of the amino acid sequence of SEQ ID NO: 5; and/or CDR-L3 comprises, consists essentially of, or consists of the amino acid sequence of SEQ ID NO: 6. In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some instances, the antigen-binding molecule comprises CDR-H1 comprising, consisting essentially of, or consisting of the amino acid sequence of SEQ ID NO: 1; CDR-H2 comprising, consisting essentially of, or consisting of the amino acid sequence of SEQ ID NO: 2; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 9; CDR-H2 comprising, consisting essentially of, or consisting of the amino acid sequence of SEQ ID NO: 11; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 9; CDR-H2 comprising, consisting essentially of, or consisting of the amino acid sequence of SEQ ID NO: 12; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 9; CDR-H2 comprising, consisting essentially of, or consisting of the amino acid sequence of SEQ ID NO: 12; CDR-H3 comprising, consisting essentially of, or consisting of the amino acid sequence of SEQ ID NO: 14; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 10; CDR-H2 comprising, consisting essentially of, or consisting of the amino acid sequence of SEQ ID NO: 13; CDR-H3 comprising, consisting essentially of, or consisting of the amino acid sequence of SEQ ID NO: 15; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 10; CDR-H2 comprising, consisting essentially of, or consisting of the amino acid sequence of SEQ ID NO: 2; CDR-H3 comprising, consisting essentially of, or consisting of the amino acid sequence of SEQ ID NO: 16; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein
CDR-H1 comprises GFTFSXaa¹⁰Xaa¹¹ (SEQ ID NO: 63);
wherein
   Xaa¹⁰ is Asn (N) or Asp (D); and
   Xaa¹¹ is Tyr (Y), Phe (F), or His (H);
CDR-H2 comprises Xaa¹²YDGXaa¹³Xaa¹⁴ (SEQ ID NO: 64);
wherein
   Xaa¹² is Asp (D) or Ser (S);
   Xaa¹³ is Gly (G), Asp (D), or Leu (L); and
   Xaa¹⁴ is Ser (S), Asp (D), or Asn (N);
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein
CDR-H1 comprises the amino acid sequence of SEQ ID NO: 56, 58, or 59;
CDR-H2 comprises Xaa¹²YDGXaa¹³Xaa¹⁴ (SEQ ID NO: 64);
wherein
   Xaa¹² is Asp (D) or Ser (S);
   Xaa¹³ is Gly (G), Asp (D), or Leu (L); and
   Xaa¹⁴ is Ser (S), Asp (D), or Asn (N);
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein
CDR-H1 comprises the amino acid sequence of SEQ ID NO: 56, 58, or 59;
CDR-H2 comprises the amino acid sequence of SEQ ID NO: 57, 60, 61, or 62;
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein
CDR-H1 comprises GFTFSXaa¹⁰Xaa¹¹ (SEQ ID NO: 63);
wherein
   Xaa¹⁰ is Asn (N) or Asp (D); and
   Xaa¹¹ is Tyr (Y), Phe (F), or His (H);
CDR-H2 comprises the amino acid sequence of SEQ ID NO: 57, 60, 61, or 62;
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein
CDR-H1 comprises GFTFSXaa¹⁰Xaa¹¹ (SEQ ID NO: 63);
wherein
   Xaa¹⁰ is Asn (N) or Asp (D); and
   Xaa¹¹ is Tyr (Y), Phe (F), or His (H);
CDR-H2 comprises the amino acid sequence of SEQ ID NO: 57, 60, 61, or 62;
CDR-H3 comprises the amino acid sequence of SEQ ID NO: 16, 3, 14, or 15;
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein
CDR-H1 comprises GFTFSXaa¹⁰Xaa¹¹ (SEQ ID NO: 63);
wherein
   Xaa¹⁰ is Asn (N) or Asp (D); and
   Xaa¹¹ is Tyr (Y), Phe (F), or His (H);
CDR-H2 comprises Xaa¹²YDGXaa¹³Xaa¹⁴ (SEQ ID NO: 64);
wherein
   Xaa¹² is Asp (D) or Ser (S);
   Xaa¹³ is Gly (G), Asp (D), or Leu (L); and
   Xaa¹⁴ is Ser (S), Asp (D), or Asn (N);
CDR-H3 comprises the amino acid sequence of SEQ ID NO: 16, 3, 14, or 15;
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises an anti-STEAP1 heavy chain variable region (VH) comprising CDR-H1, CDR-H2, and/or CDR-H3; and/or an anti-STEAP1 light chain variable region (VL) comprising CDR-L1, CDR-L2, and/or CDR-L3; wherein
CDR-H1 comprises the amino acid sequence of SEQ ID NO: 56, 58, or 59;
CDR-H2 comprises Xaa¹²YDGXaa¹³Xaa¹⁴ (SEQ ID NO: 64);
wherein
   Xaa¹² is Asp (D) or Ser (S);
   Xaa¹³ is Gly (G), Asp (D), or Leu (L); and
   Xaa¹⁴ is Ser (S), Asp (D), or Asn (N);
CDR-H3 comprises the amino acid sequence of SEQ ID NO: 16, 3, 14, or 15;
CDR-L1 comprises the amino acid sequence of SEQ ID NO: 4;
CDR-L2 comprises the amino acid sequence of SEQ ID NO: 5; and/or
CDR-L3 comprises the amino acid sequence of SEQ ID NO: 6.

In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some embodiments, the antigen-binding molecule that binds to STEAP1 comprises a heavy chain variable region (VH) comprising CDR-H1, CDR-H2, and/or CDR-H3; and/or an anti-STEAP1 light chain variable region (VL) comprising CDR-L1, CDR-L2, and/or CDR-L3; in which CDR-H1 comprises, consists essentially of, or consists of the amino acid sequence of SEQ ID NO: 56, 58, or 59; CDR-H2 comprises, consists essentially of, or consists of the amino acid sequence of SEQ ID NO: 57, 60, 61, or 62; CDR-H3 comprises, consists essentially of, or consists of the amino acid sequence of SEQ ID NO: 16, 3, 14, or 15; CDR-L1 comprises, consists essentially of, or consists of the amino acid sequence of SEQ ID NO: 4; CDR-L2 comprises, consists essentially of, or consists of the amino acid sequence of SEQ ID NO: 5; and/or CDR-L3 comprises, consists essentially of, or consists of the amino acid sequence of SEQ ID NO: 6. In some cases, the anti-STEAP1 antigen-binding molecule comprises at least two, at least three, at least four, at least five, or all six CDR-H1, CDR-H2, CDR-H3, CDR-L1, CDR-L2, and CDR-L3.

In some instances, the antigen-binding molecule comprises CDR-H1 comprising, consisting essentially of, or consisting of the amino acid sequence of SEQ ID NO: 56; CDR-H2 comprising, consisting essentially of, or consisting of the amino acid sequence of SEQ ID NO: 57; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 58; CDR-H2 comprising, consisting essentially of, or consisting of the amino acid sequence of SEQ ID NO: 60; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 58; CDR-H2 comprising, consisting essentially of, or consisting of the amino acid sequence of SEQ ID NO: 61; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 58; CDR-H2 comprising, consisting essentially of, or consisting of the amino acid sequence of SEQ ID NO: 61; CDR-H3 comprising, consisting essentially of, or consisting of the amino acid sequence of SEQ ID NO: 14; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 59; CDR-H2 comprising, consisting essentially of, or consisting of the amino acid sequence of SEQ ID NO: 62; CDR-H3 comprising, consisting essentially of, or consisting of the amino acid sequence of SEQ ID NO: 15; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the antigen-binding molecule comprises CDR-H1 comprising, consisting essentially of, or consisting of the amino acid sequence of SEQ ID NO: 59; CDR-H2 comprising, consisting essentially of, or consisting of the amino acid sequence of SEQ ID NO: 57; CDR-H3 comprising, consisting essentially of, or consisting of the amino acid sequence of SEQ ID NO: 16; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

The antigen-binding molecules of the present invention can be humanized antibodies. In some instances, an antigen-binding molecule of the present invention comprises a constant region derived from an IgG framework region. In some cases, the IgG framework region is an IgG1, IgG2, or IgG4 framework region. In some cases, the IgG framework region is an IgG1 framework region.

In some embodiments, the antigen-binding molecule comprises a heavy chain variable region comprising one or more (e.g., 1, 2, 3, or all 4) of a framework region (FR)-H1 sequence having at least 80%, 85%, 90%, 91%, 92%, 93%, 94%, 95%, 96%, 97%, 98%, 99%, or 100% sequence identity to the amino acid sequence of SEQ ID NO: 110; an FR-H2 having at least 80%, 85%, 90%, 91%, 92%, 93%, 94%, 95%, 96%, 97%, 98%, 99%, or 100% sequence identity to the amino acid sequence of SEQ ID NO: 118; an FR-H3 having at least 80%, 85%, 90%, 91%, 92%, 93%, 94%, 95%, 96%, 97%, 98%, 99%, or 100% sequence identity to the amino acid sequence of SEQ ID NO: 112; and/or an FR-H4 having at least 80%, 85%, 90%, 91%, 92%, 93%, 94%, 95%, 96%, 97%, 98%, 99%, or 100% sequence identity to the amino acid sequence of SEQ ID NO: 113. In some embodiments, the antigen-binding molecule comprises a heavy chain variable region comprising an FR-H2 having at least 80%, 85%, 90%, 91%, 92%, 93%, 94%, 95%, 96%, 97%, 98%, 99%, or 100% sequence identity to the amino acid sequence of SEQ ID NO: 118. In some embodiments, the antigen-binding molecule comprises a heavy chain variable region comprising an FR-H2 having the amino acid sequence of SEQ ID NO: 118. In other embodiments, the antigen-binding molecule comprises a heavy chain variable region comprising an FR-H2 having the amino acid sequence of SEQ ID NO: 120.

In some embodiments, the antigen-binding molecule comprises a heavy chain variable region comprising one or more (e.g., 1, 2, 3, or all 4) of an FR-H1 sequence having at least 80%, 85%, 90%, 91%, 92%, 93%, 94%, 95%, 96%, 97%, 98%, 99%, or 100% sequence identity to the amino acid sequence of SEQ ID NO: 110; an FR-H2 having at least 80%, 85%, 90%, 91%, 92%, 93%, 94%, 95%, 96%, 97%, 98%, 99%, or 100% sequence identity to the amino acid sequence of SEQ ID NO: 111; an FR-H3 having at least 80%, 85%, 90%, 91%, 92%, 93%, 94%, 95%, 96%, 97%, 98%, 99%, or 100% sequence identity to the amino acid sequence of SEQ ID NO: 112; and/or an FR-H4 having at least 80%, 85%, 90%, 91%, 92%, 93%, 94%, 95%, 96%, 97%, 98%, 99%, or 100% sequence identity to the amino acid sequence of SEQ ID NO: 113.

In some embodiments, the antigen-binding molecule comprises a light chain variable region comprising one or more (e.g., 1, 2, 3, or all 4) of an FR-L1 having at least 80%, 85%, 90%, 91%, 92%, 93%, 94%, 95%, 96%, 97%, 98%, 99%, or 100% sequence identity to the amino acid sequence of SEQ ID NO: 114; an FR-L2 having at least 80%, 85%, 90%, 91%, 92%, 93%, 94%, 95%, 96%, 97%, 98%, 99%, or 100% sequence identity to the amino acid sequence of SEQ ID NO: 119; an FR-L3 co having at least 80%, 85%, 90%, 91%, 92%, 93%, 94%, 95%, 96%, 97%, 98%, 99%, or 100% sequence identity to the amino acid sequence of SEQ ID NO: 116; and/or an FR-L4 having at least 80%, 85%, 90%, 91%, 92%, 93%, 94%, 95%, 96%, 97%, 98%, 99%, or 100% sequence identity to the amino acid sequence of SEQ ID NO: 117. In some embodiments, the antigen-binding molecule comprises a heavy chain variable region comprising an FR-L2 having at least 80%, 85%, 90%, 91%, 92%, 93%, 94%, 95%, 96%, 97%, 98%, 99%, or 100% sequence identity to the amino acid sequence of SEQ ID NO: 119. In some embodiments, the antigen-binding molecule comprises a heavy chain variable region comprising an FR-L2 having the amino acid sequence of SEQ ID NO: 119. In other embodiments, the antigen-binding molecule comprises a heavy chain variable region comprising an FR-L2 having the amino acid sequence of SEQ ID NO: 121.

In some embodiments, the antigen-binding molecule comprises a light chain variable region comprising one or more (e.g., 1, 2, 3, or all 4) of an FR-L1 having at least 80%, 85%, 90%, 91%, 92%, 93%, 94%, 95%, 96%, 97%, 98%, 99%, or 100% sequence identity to the amino acid sequence of SEQ ID NO: 114; an FR-L2 having at least 80%, 85%, 90%, 91%, 92%, 93%, 94%, 95%, 96%, 97%, 98%, 99%, or 100% sequence identity to the amino acid sequence of SEQ ID NO: 115; an FR-L3 co having at least 80%, 85%, 90%, 91%, 92%, 93%, 94%, 95%, 96%, 97%, 98%, 99%, or 100% sequence identity to the amino acid sequence of SEQ ID NO: 116; and/or an FR-L4 having at least 80%, 85%, 90%, 91%, 92%, 93%, 94%, 95%, 96%, 97%, 98%, 99%, or 100% sequence identity to the amino acid sequence of SEQ ID NO: 117.

For example, in some instances, the antigen-binding molecule comprises an FR-H1 comprising, consisting essentially of, or consisting of the amino acid sequence of SEQ ID NO: 110; an FR-H2 comprising, consisting essentially of, or consisting of the amino acid sequence of SEQ ID NO: 111; an FR-H3 comprising, consisting essentially of, or consisting of the amino acid sequence of SEQ ID NO: 112; an FR-H4 comprising, consisting essentially of, or consisting of the amino acid sequence of SEQ ID NO: 113; an FR-L1 comprising, consisting essentially of, or consisting of the amino acid sequence of SEQ ID NO: 114; an FR-L2 comprising, consisting essentially of, or consisting of the amino acid sequence of SEQ ID NO: 115; an FR-L3 comprising, consisting essentially of, or consisting of the amino acid sequence of SEQ ID NO: 116; and an FR-L4 comprising, consisting essentially of, or consisting of the amino acid sequence of SEQ ID NO: 117.

In some embodiments, the antigen-binding molecule comprises a heavy chain variable region comprising a VH sequence having at least 80%, 85%, 90%, 91%, 92%, 93%, 94%, 95%, 96%, 97%, 98%, 99%, or 100% sequence identity to SEQ ID NO: 7, 17-25, 30-34, 38, or 68. In some instances, the heavy chain variable region comprises a VH sequence having at least 90% sequence identity to SEQ ID NO: 7, 17-25, 30-34, 38, or 68. In some instances, the heavy chain variable region comprises a VH sequence having at least 95% sequence identity to SEQ ID NO: 7, 17-25, 30-34, 38, or 68. In some instances, the heavy chain variable region comprises a VH sequence having at least 98% sequence identity to SEQ ID NO: 7, 17-25, 30-34, 38, or 68. In some instances, the heavy chain variable region comprises a VH sequence having at least 99% sequence identity to SEQ ID NO: 7, 17-25, 30-34, 38, or 68. In some instances, the heavy chain variable region comprises a VH sequence having 100% sequence identity to SEQ ID NO: 7, 17-25, 30-34, 38, or 68. In certain aspects, a VH sequence having at least 90%, 91%, 92%, 93%, 94%, 95%, 96%, 97%, 98%, or 99% identity contains substitutions (e.g., conservative substitutions), insertions, or deletions relative to SEQ ID NO: 7, 17-25, 30-34, 38, or 68, but an anti-STEAP1 antibody comprising that sequence retains the ability to bind to STEAP1. In certain aspects, a total of 1 to 10 amino acids have been substituted, inserted and/or deleted in SEQ ID NO: 7, 17-25, 30-34, 38, or 68. In certain aspects, substitutions, insertions, or deletions occur in regions outside the CDRs (i.e., in the FRs).

In some embodiments, the antigen-binding molecule comprises a light chain variable region comprising a VL sequence having at least 80%, 85%, 90%, 91%, 92%, 93%, 94%, 95%, 96%, 97%, 98%, 99%, or 100% sequence identity to SEQ ID NO: 8, 26-29, 39, or 69. In some instances, the light chain variable region comprises a VL sequence having at least 90% sequence identity to SEQ ID NO: 8, 26-29, 39, or 69. In some instances, the light chain variable region comprises a VL sequence having at least 95% sequence identity to SEQ ID NO: 8, 26-29, 39, or 69. In some instances, the light chain variable region comprises a VL sequence having at least 98% sequence identity to SEQ ID NO: 8, 26-29, 39, or 69. In some instances, the light chain variable region comprises a VL sequence having at least 99% sequence identity to SEQ ID NO: 8, 26-29, 39, or 69. In some instances, the light chain variable region comprises a VL sequence having 100% sequence identity to SEQ ID NO: 8, 26-29, 39, or 69. In certain aspects, a VL sequence having at least 90%, 91%, 92%, 93%, 94%, 95%, 96%, 97%, 98%, or 99% identity contains substitutions (e.g., conservative substitutions), insertions, or deletions relative to SEQ ID NO: 8, 26-29, 39, or 69, but an anti-STEAP1 antibody comprising that sequence retains the ability to bind to STEAP1. In certain aspects, a total of 1 to 10 amino acids have been substituted, inserted and/or deleted in SEQ ID NO: 8, 26-29, 39, or 69. In certain aspects, the substitutions, insertions, or deletions occur in regions outside the CDRs (i.e., in the FRs).

In some embodiments, the antigen-binding molecule of any of the preceding embodiments features a VH and a VL region, in which the VH region comprises one, two, or all three of the following CDRs: (a) CDR-H1 comprising, consisting essentially of, or consisting of the amino acid sequence of SEQ ID NO: 9, (b) CDR-H2 comprising, consisting essentially of, or consisting of the amino acid sequence of SEQ ID NO: 11, or (c) CDR-H3 comprising, consisting essentially of, or consisting of the amino acid sequence of SEQ ID NO: 3. In some instances, the VH region comprises one, two, or all three of the following CDRs: (a) CDR-H1 comprising, consisting essentially of, or consisting of the amino acid sequence of SEQ ID NO: 58, (b) CDR-H2 comprising, consisting essentially of, or consisting of the amino acid sequence of SEQ ID NO: 60, or (c) CDR-H3 comprising, consisting essentially of, or consisting of the amino acid sequence of SEQ ID NO: 3. In some embodiments, the VH region comprises an amino acid sequence having at least 90% sequence identity to SEQ ID NO: 30 (e.g., at least 95% sequence identity to SEQ ID NO: 30, at least 96% sequence identity to SEQ ID NO: 30, at least 97% sequence identity to SEQ ID NO: 30, at least 98% sequence identity to SEQ ID NO: 30, at least 99% sequence identity to SEQ ID NO: 30, or 100% sequence identity to SEQ ID NO: 30). In some embodiments, the VL region comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27).

In some embodiments, the antigen-binding molecule of any of the preceding embodiments features a VH and a VL region, in which the VH region comprises one, two, or all three of the following CDRs: (a) CDR-H1 comprising, consisting essentially of, or consisting of the amino acid sequence of SEQ ID NO: 9, (b) CDR-H2 comprising, consisting essentially of, or consisting of the amino acid sequence of SEQ ID NO: 12, or (c) CDR-H3 comprising, consisting essentially of, or consisting of the amino acid sequence of SEQ ID NO: 3. In some instances, the VH region comprises one, two, or all three of the following CDRs: (a) CDR-H1 comprising, consisting essentially of, or consisting of the amino acid sequence of SEQ ID NO: 58, (b) CDR-H2 comprising, consisting essentially of, or consisting of the amino acid sequence of SEQ ID NO: 61, or (c) CDR-H3 comprising, consisting essentially of, or consisting of the amino acid sequence of SEQ ID NO: 3. In some embodiments, the VH region comprises an amino acid sequence having at least 90% sequence identity to SEQ ID NO: 31 (e.g., at least 95% sequence identity to SEQ ID NO: 31, at least 96% sequence identity to SEQ ID NO: 31, at least 97% sequence identity to SEQ ID NO: 31, at least 98% sequence identity to SEQ ID NO: 31, at least 99% sequence identity to SEQ ID NO: 31, or 100% sequence identity to SEQ ID NO: 31). In some embodiments, the VL region comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27).

In some embodiments, the antigen-binding molecule of any of the preceding embodiments features a VH and a VL region, in which the VH region comprises one, two, or all three of the following CDRs: (a) CDR-H1 comprising, consisting essentially of, or consisting of the amino acid sequence of SEQ ID NO: 9, (b) CDR-H2 comprising, consisting essentially of, or consisting of the amino acid sequence of SEQ ID NO: 12, or (c) CDR-H3 comprising, consisting essentially of, or consisting of the amino acid sequence of SEQ ID NO: 14. In some instances, the VH region comprises one, two, or all three of the following CDRs: (a) CDR-H1 comprising, consisting essentially of, or consisting of the amino acid sequence of SEQ ID NO: 58, (b) CDR-H2 comprising, consisting essentially of, or consisting of the amino acid sequence of SEQ ID NO: 61, or (c) CDR-H3 comprising, consisting essentially of, or consisting of the amino acid sequence of SEQ ID NO: 14. In some embodiments, the VH region comprises an amino acid sequence having at least 90% sequence identity to SEQ ID NO: 32 (e.g., at least 95% sequence identity to SEQ ID NO: 32, at least 96% sequence identity to SEQ ID NO: 32, at least 97% sequence identity to SEQ ID NO: 32, at least 98% sequence identity to SEQ ID NO: 32, at least 99% sequence identity to SEQ ID NO: 32, or 100% sequence identity to SEQ ID NO: 32). In some embodiments, the VL region comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27).

In some embodiments, the antigen-binding molecule of any of the preceding embodiments features a VH and a VL region, in which the VH region comprises one, two, or all three of the following CDRs: (a) CDR-H1 comprising, consisting essentially of, or consisting of the amino acid sequence of SEQ ID NO: 10, (b) CDR-H2 comprising, consisting essentially of, or consisting of the amino acid sequence of SEQ ID NO: 13, or (c) CDR-H3 comprising, consisting essentially of, or consisting of the amino acid sequence of SEQ ID NO: 15. In some instances, the VH region comprises one, two, or all three of the following CDRs: (a) CDR-H1 comprising, consisting essentially of, or consisting of the amino acid sequence of SEQ ID NO: 59, (b) CDR-H2 comprising, consisting essentially of, or consisting of the amino acid sequence of SEQ ID NO: 62, or (c) CDR-H3 comprising, consisting essentially of, or consisting of the amino acid sequence of SEQ ID NO: 15. In some embodiments, the VH region comprises an amino acid sequence having at least 90% sequence identity to SEQ ID NO: 33 (e.g., at least 95% sequence identity to SEQ ID NO: 33, at least 96% sequence identity to SEQ ID NO: 33, at least 97% sequence identity to SEQ ID NO: 33, at least 98% sequence identity to SEQ ID NO: 33, at least 99% sequence identity to SEQ ID NO: 33, or 100% sequence identity to SEQ ID NO: 33). In some embodiments, the VL region comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27).

In some embodiments, the antigen-binding molecule of any of the preceding embodiments features a VH and a VL region, in which the VH region comprises one, two, or all three of the following CDRs: (a) CDR-H1 comprising, consisting essentially of, or consisting of the amino acid sequence of SEQ ID NO: 10, (b) CDR-H2 comprising, consisting essentially of, or consisting of the amino acid sequence of SEQ ID NO: 2, or (c) CDR-H3 comprising, consisting essentially of, or consisting of the amino acid sequence of SEQ ID NO: 16. In some instances, the VH region comprises one, two, or all three of the following CDRs: (a) CDR-H1 comprising, consisting essentially of, or consisting of the amino acid sequence of SEQ ID NO: 59, (b) CDR-H2 comprising, consisting essentially of, or consisting of the amino acid sequence of SEQ ID NO: 57, or (c) CDR-H3 comprising, consisting essentially of, or consisting of the amino acid sequence of SEQ ID NO: 16. In some embodiments, the VH region comprises an amino acid sequence having at least 90% sequence identity to SEQ ID NO: 34 (e.g., at least 95% sequence identity to SEQ ID NO: 34, at least 96% sequence identity to SEQ ID NO: 34, at least 97% sequence identity to SEQ ID NO: 34, at least 98% sequence identity to SEQ ID NO: 34, at least 99% sequence identity to SEQ ID NO: 34, or 100% sequence identity to SEQ ID NO: 34). In some embodiments, the VL region comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27).

In some embodiments, the VH region comprises an amino acid sequence having at least 90% sequence identity to SEQ ID NO: 68 (e.g., at least 95% sequence identity to SEQ ID NO: 68, at least 96% sequence identity to SEQ ID NO: 68, at least 97% sequence identity to SEQ ID NO: 68, at least 98% sequence identity to SEQ ID NO: 68, at least 99% sequence identity to SEQ ID NO: 68, or 100% sequence identity to SEQ ID NO: 68). In some embodiments, the VL region comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region comprises an amino acid sequence having at least 90% sequence identity to SEQ ID NO: 69 (e.g., at least 95% sequence identity to SEQ ID NO: 69, at least 96% sequence identity to SEQ ID NO: 69, at least 97% sequence identity to SEQ ID NO: 69, at least 98% sequence identity to SEQ ID NO: 69, at least 99% sequence identity to SEQ ID NO: 69, or 100% sequence identity to SEQ ID NO: 69).

In some embodiments, the antigen-binding molecule of any of the preceding embodiments features a VH and a VL region, in which the VH region comprises one, two, or all three of the following CDRs: (a) CDR-H1 comprising, consisting essentially of, or consisting of the amino acid sequence of SEQ ID NO: 35, (b) CDR-H2 comprising, consisting essentially of, or consisting of the amino acid sequence of SEQ ID NO: 36, or (c) CDR-H3 comprising, consisting essentially of, or consisting of the amino acid sequence of SEQ ID NO: 37. In some instances, the VH region comprises one, two, or all three of the following CDRs: (a) CDR-H1 comprising, consisting essentially of, or consisting of the amino acid sequence of SEQ ID NO: 63, (b) CDR-H2 comprising, consisting essentially of, or consisting of the amino acid sequence of SEQ ID NO: 64, or (c) CDR-H3 comprising, consisting essentially of, or consisting of the amino acid sequence of SEQ ID NO: 37. In some embodiments, the VH region comprises an amino acid sequence having at least 90% sequence identity to SEQ ID NO: 38 (e.g., at least 95% sequence identity to SEQ ID NO: 38, at least 96% sequence identity to SEQ ID NO: 38, at least 97% sequence identity to SEQ ID NO: 38, at least 98% sequence identity to SEQ ID NO: 38, at least 99% sequence identity to SEQ ID NO: 38, or 100% sequence identity to SEQ ID NO: 38). In some embodiments, the VL region comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region comprises an amino acid sequence having at least 90% sequence identity to SEQ ID NO: 39 (e.g., at least 95% sequence identity to SEQ ID NO: 39, at least 96% sequence identity to SEQ ID NO: 39, at least 97% sequence identity to SEQ ID NO: 39, at least 98% sequence identity to SEQ ID NO: 39, at least 99% sequence identity to SEQ ID NO: 39, or 100% sequence identity to SEQ ID NO: 39).

In some embodiments, the antigen-binding molecule binds to an epitope on STEAP1 comprising one or more amino acid residues selected from Ser101, His102, Gln103, and Lys281 of STEAP1. In some embodiments, the epitope comprises amino acid residues Ser101 of ECL1 of STEAP1. In some embodiments, the epitope comprises amino acid residues His102 of ECL1 of STEAP1. In some embodiments, the epitope comprises amino acid residues Gln103 of ECL1 of STEAP1. In some embodiments, the epitope comprises amino acid residues Lys281 of ECL3 of STEAP1. In some embodiments, the epitope comprises Ser101, His102, Gln103 of ECL1 of STEAP1, and Lys281 of ECL3 of STEAP1. In some embodiments, the epitope further comprises one or more additional amino acid residues selected from Trp195, Gln198, Gln201, Gln202, Asn203 and Lys204 of ECL2 of STEAP1. In some embodiments, the epitope does not comprise amino acid residue Gln104 of STEAP1. In some embodiments, the epitope does not comprise amino acid residue Tyr107 of STEAP1. In some embodiments, the epitope does not comprise amino acid residue Asn194 of STEAP1. In some embodiments, the epitope does not comprise amino acid residue Glu205 of STEAP1. In some embodiments, the epitope does not comprise amino acid residues Ala207 of STEAP1.

In some embodiments, the antigen-binding molecule binds human STEAP1 with a K_{D} of about 100 nM or lower, as measured by Kinetic Exclusion Assay (KinExA®). In some embodiments, the antigen-binding molecule binds human STEAP1 with a K_{D} of between about 10 pM to about 100 nM. In some embodiments, the antigen-binding molecule binds human STEAP1 with a K_{D} of between about 100 pM to about 50 nM. In some embodiments, the antigen-binding molecule binds human STEAP1 with a K_{D} of between about 1 nM to about 30 nM. In some embodiments, the antigen-binding molecule binds cynomolgus STEAP1 with a K_{D} of about 100 nM or lower, as measured by Kinetic Exclusion Assay (KinExA®). In some embodiments, the antigen-binding molecule binds cynomolgus STEAP1 with a K_{D} of between about 10 pM to about 100 nM. In some embodiments, the antigen-binding molecule binds cynomolgus STEAP1 with a K_{D} of between about 100 pM to about 50 nM. In some embodiments, the antigen-binding molecule binds cynomolgus STEAP1 with a K_{D} of between about 1 nM to about 30 nM.

### Multispecific Antigen-Binding Molecules

In some embodiments, the invention provides isolated multispecific antigen-binding molecules that comprises a first antigen-binding domain that binds to STEAP1 as described above. In some instances, the multispecific antigen-binding molecule binds to STEAP1 monovalently. In some instances, the multispecific antigen-binding molecule further comprises a second binding domain that binds to a T cell receptor. In additional instances, the multispecific antigen-binding molecule comprises a third binding domain that binds to an additional antigen (e.g., a tumor-associated antigen).

In some embodiments, a multispecific antigen-binding molecule of the invention is a bispecific antigen-binding molecule (e.g., a bispecific antibody) comprising a first antigen-binding domain that binds to STEAP1 and a second antigen-binding domain that binds to a T cell receptor. Exemplary T cell receptors include cluster of differentiation 3 (CD3). In some instances, the second antigen-binding domain comprises an antibody or fragment thereof that binds to CD3. Exemplary antigen-binding domains that bind to CD3 include, but are not limited to 40G5c and 38E4v1.MD1. Additional antigen-binding domains that bind to CD3 (e.g., UCHT1.v9, 38E4v11, 30E4c, SP34v52, etc.) are described in U.S. Pat. No. 10,174,124, WO2015/095392, WO2016/205520, and International Patent Application No. PCT/US2020/064635, each of which is incorporated herein by reference in its entirety.

In some embodiments, the second antigen-binding domain binds to an epitope on CD3 comprising amino acid residue Glu6 of CD3. In some embodiments, the epitope further comprises one or more additional amino acid residues selected from Gln1, Asp2, and Met7 of CD3. In some embodiments, the epitope comprises amino acid residues Gln1, Asp2, and Glu6 of CD3. In some embodiments, the epitope comprises amino acid residues Gln1, Asp2, Glu6, and Met7 of CD3. In some embodiments, the epitope does not comprise amino acid residue Glu5 of CD3. In some embodiments, the epitope does not comprise amino acid residues Gly3 and Glu5 of CD3. In some embodiments, the epitope consists of amino acid residues Gln1, Asp2, Glu6, and Met7 of CD3. In some embodiments, the second antigen-binding domain is capable of binding to a human CD3 polypeptide or a cyno CD3 polypeptide. In some embodiments, the human CD3 polypeptide or the cyno CD3 polypeptide is a human CD3e polypeptide or a cyno CD3e polypeptide, respectively. In some embodiments, the human CD3 polypeptide or the cyno CD3 polypeptide is a human CD3y polypeptide or a cyno CD3y polypeptide, respectively. In some embodiments, the second antigen-binding domain binds the human CD3e polypeptide with a K_{D} of about 100 nM or lower. In some embodiments, the second antigen-binding domain binds the human CD3e polypeptide with a K_{D} of between about 10 pM to about 100 nM. In some embodiments, the second antigen-binding domain binds the human CD3e polypeptide with a K_{D} of between about 100 pM to about 50 nM. In some embodiments, the second antigen-binding domain binds the human CD3e polypeptide with a K_{D} of between about 1 nM to about 10 nM.

In some embodiments, the second antigen-binding domain comprises at least one, at least two, at least three, at least four, at least five, or all six CDR-H1, CDR-H2, CDR-H3, CDR-L1, CDR-L2, and CDR-L3 of 38E4v1.MD1. In some cases, the second antigen-binding domain comprises at least one, at least two, at least three, at least four, at least five, or all six CDRs, in which CDR-H1 comprises, consists essentially of, or consists of the amino acid sequence of SEQ ID NO: 40, CDR-H2 comprises, consists essentially of, or consists of the amino acid sequence of SEQ ID NO: 41, CDR-H3 comprises, consists essentially of, or consists of the amino acid sequence of SEQ ID NO: 42, CDR-L1 comprises, consists essentially of, or consists of the amino acid sequence of SEQ ID NO: 43, CDR-L2 comprises, consists essentially of, or consists of the amino acid sequence of SEQ ID NO: 44, or CDR-L3 comprises, consists essentially of, or consists of the amino acid sequence of SEQ ID NO: 45. In some cases, the second antigen-binding domain comprises a VH sequence comprising at least 80% (e.g., 90%, 91%, 92%, 93%, 94%, 95%, 96%, 97%, 98%, or 99%) sequence identity to SEQ ID NO: 46. In some cases, the second antigen-binding domain comprises a VL sequence comprising at least 80% (e.g., 90%, 91%, 92%, 93%, 94%, 95%, 96%, 97%, 98%, or 99%) sequence identity to SEQ ID NO: 47. In certain aspects, a VH or VL sequence having at least 90%, 91%, 92%, 93%, 94%, 95%, 96%, 97%, 98%, or 99% identity contains substitutions (e.g., conservative substitutions), insertions, or deletions relative to SEQ ID NO: 46 or 47, but a multispecific antibody comprising that sequence retains the ability to bind to CD3. In certain aspects, a total of 1 to 10 amino acids have been substituted, inserted and/or deleted in SEQ ID NO: 46 or 47. In certain aspects, the substitutions, insertions, or deletions occur in regions outside the CDRs (i.e., in the FRs).

In some embodiments, the second antigen-binding domain comprises at least one, at least two, at least three, at least four, at least five, or all six CDR-H1, CDR-H2, CDR-H3, CDR-L1, CDR-L2, and CDR-L3 of 40G5c. In some cases, the second antigen-binding domain comprises at least one, at least two, at least three, at least four, at least five, or all six CDRs, in which CDR-H1 comprises, consists essentially of, or consists of the amino acid sequence of SEQ ID NO: 48, CDR-H2 comprises, consists essentially of, or consists of the amino acid sequence of SEQ ID NO: 49, CDR-H3 comprises, consists essentially of, or consists of the amino acid sequence of SEQ ID NO: 50, CDR-L1 comprises, consists essentially of, or consists of the amino acid sequence of SEQ ID NO: 51, CDR-L2 comprises, consists essentially of, or consists of the amino acid sequence of SEQ ID NO: 52, or CDR-L3 comprises, consists essentially of, or consists of the amino acid sequence of SEQ ID NO: 53. In some cases, the second antigen-binding domain comprises a VH sequence comprising at least 80% (e.g., 90%, 91%, 92%, 93%, 94%, 95%, 96%, 97%, 98%, or 99%) sequence identity to SEQ ID NO: 54. In some cases, the second antigen-binding domain comprises a VL sequence comprising at least 80% (e.g., 90%, 91%, 92%, 93%, 94%, 95%, 96%, 97%, 98%, or 99%) sequence identity to SEQ ID NO: 55. In certain aspects, a VH or VL sequence having at least 90%, 91%, 92%, 93%, 94%, 95%, 96%, 97%, 98%, or 99% identity contains substitutions (e.g., conservative substitutions), insertions, or deletions relative to SEQ ID NO: 54 or 55, but a multispecific antibody comprising that sequence retains the ability to bind to CD3. In certain aspects, a total of 1 to 10 amino acids have been substituted, inserted and/or deleted in SEQ ID NO: 54 or 55. In certain aspects, the substitutions, insertions, or deletions occur in regions outside the CDRs (i.e., in the FRs).

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and/or CDR-H3 of a VH sequence selected from SEQ ID NO: 7, 17-25, 30-34, 38, and 68; and/or an anti-STEAP1 light chain variable region (VL) comprising CDR-L1, CDR-L2, and/or CDR-L3 of a VL sequence selected from SEQ ID NO: 8, 26-29, 39, and 69; and (B) a second antigen-binding domain that binds to a T cell receptor. In some instances, at least one, at least two, at least three, at least four, at least five, or all six CDRs are selected from the CDR(s) of the VH sequence selected from SEQ ID NO: 7, 17-25, 30-34, 38, and 68 and the VL sequence selected from SEQ ID NO: 8, 26-29, 39, and 69. In some instances, at least one, at least two, at least three, at least four, at least five, or all six CDRs are selected from the CDR(s) of 40G5c or 38E4v1.MD1. In some cases, the six CDRs are defined according to the Kabat numbering. In other cases, the six CDRs are defined according to the Chothia numbering. In additional cases, the six CDRs are defined according to the EU numbering.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises YIXaa³YDGXaa⁴Xaa⁵TXaa⁶YGDSVKG (SEQ ID NO: 36);
   wherein
      Xaa³ is Asp (D) or Ser (S);
      Xaa⁴ is Gly (G), Asp (D), or Leu (L);
      Xaa⁵ is Ser (S), Asp (D), or Asn (N); and
      Xaa⁶ is Ser (S) or Tyr (Y); and
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4v1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises the amino acid sequence of SEQ ID NO: 10, 1, or 9;
   CDR-H2 comprises YIXaa³YDGXaa⁴Xaa⁵TXaa⁶YGDSVKG (SEQ ID NO: 36);
   wherein
      Xaa³ is Asp (D) or Ser (S);
      Xaa⁴ is Gly (G), Asp (D), or Leu (L);
      Xaa⁵ is Ser (S), Asp (D), or Asn (N); and
      Xaa⁶ is Ser (S) or Tyr (Y);
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4v1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises the amino acid sequence of SEQ ID NO: 10, 1, or 9;
   CDR-H2 comprises the amino acid sequence of SEQ ID NO: 2, 11, 12, or 13;
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4v1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises the amino acid sequence of SEQ ID NO: 2, 11, 12, or 13;
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises the amino acid sequence of SEQ ID NO: 2, 11, 12, or 13;
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises YIXaa³YDGXaa⁴Xaa⁵TXaa⁶YGDSVKG (SEQ ID NO: 36);
   wherein
      Xaa³ is Asp (D) or Ser (S);
      Xaa⁴ is Gly (G), Asp (D), or Leu (L);
      Xaa⁵ is Ser (S), Asp (D), or Asn (N); and
      Xaa⁶ is Ser (S) or Tyr (Y);
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises the amino acid sequence of SEQ ID NO: 10, 1, or 9;
   CDR-H2 comprises YIXaa³YDGXaa⁴Xaa⁵TXaa⁶YGDSVKG (SEQ ID NO: 36);
   wherein
      Xaa³ is Asp (D) or Ser (S);
      Xaa⁴ is Gly (G), Asp (D), or Leu (L);
      Xaa⁵ is Ser (S), Asp (D), or Asn (N); and
      Xaa⁶ is Ser (S) or Tyr (Y);
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3; in which CDR-H1 comprises, consists essentially of, or consists of the amino acid sequence of SEQ ID NO: 10, 1, or 9; CDR-H2 comprises, consists essentially of, or consists of the amino acid sequence of SEQ ID NO: 2, 11, 12, or 13; CDR-H3 comprises, consists essentially of, or consists of the amino acid sequence of SEQ ID NO: 16, 3, 14, or 15; CDR-L1 comprises, consists essentially of, or consists of the amino acid sequence of SEQ ID NO: 4; CDR-L2 comprises, consists essentially of, or consists of the amino acid sequence of SEQ ID NO: 5; and/or CDR-L3 comprises, consists essentially of, or consists of the amino acid sequence of SEQ ID NO: 6; and (B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some instances, the first antigen-binding domain comprises CDR-H1 comprising, consisting essentially of, or consisting of the amino acid sequence of SEQ ID NO: 1; CDR-H2 comprising, consisting essentially of, or consisting of the amino acid sequence of SEQ ID NO: 2; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 9; CDR-H2 comprising, consisting essentially of, or consisting of the amino acid sequence of SEQ ID NO: 11; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 9; CDR-H2 comprising, consisting essentially of, or consisting of the amino acid sequence of SEQ ID NO: 12; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 9; CDR-H2 comprising, consisting essentially of, or consisting of the amino acid sequence of SEQ ID NO: 12; CDR-H3 comprising, consisting essentially of, or consisting of the amino acid sequence of SEQ ID NO: 14; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 10; CDR-H2 comprising, consisting essentially of, or consisting of the amino acid sequence of SEQ ID NO: 13; CDR-H3 comprising, consisting essentially of, or consisting of the amino acid sequence of SEQ ID NO: 15; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 10; CDR-H2 comprising, consisting essentially of, or consisting of the amino acid sequence of SEQ ID NO: 2; CDR-H3 comprising, consisting essentially of, or consisting of the amino acid sequence of SEQ ID NO: 16; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises GFTFSXaa¹⁰Xaa¹¹ (SEQ ID NO: 63);
   wherein
      Xaa¹⁰ is Asn (N) or Asp (D); and
      Xaa¹¹ is Tyr (Y), Phe (F), or His (H);
   CDR-H2 comprises Xaa¹²YDGXaa¹³Xaa¹⁴ (SEQ ID NO: 64);
   wherein
      Xaa¹² is Asp (D) or Ser (S);
      Xaa¹³ is Gly (G), Asp (D), or Leu (L); and
      Xaa¹⁴ is Ser (S), Asp (D), or Asn (N);
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises the amino acid sequence of SEQ ID NO: 56, 58, or 59;
   CDR-H2 comprises Xaa¹²YDGXaa¹³Xaa¹⁴ (SEQ ID NO: 64);
   wherein
      Xaa¹² is Asp (D) or Ser (S);
      Xaa¹³ is Gly (G), Asp (D), or Leu (L); and
      Xaa¹⁴ is Ser (S), Asp (D), or Asn (N);
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises the amino acid sequence of SEQ ID NO: 56, 58, or 59;
   CDR-H2 comprises the amino acid sequence of SEQ ID NO: 57, 60, 61, or 62;
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises GFTFSXaa¹⁰Xaa¹¹ (SEQ ID NO: 63);
   wherein
      Xaa¹⁰ is Asn (N) or Asp (D); and
      Xaa¹¹ is Tyr (Y), Phe (F), or His (H);
   CDR-H2 comprises the amino acid sequence of SEQ ID NO: 57, 60, 61, or 62;
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises GFTFSXaa¹⁰Xaa¹¹ (SEQ ID NO: 63);
   wherein
      Xaa¹⁰ is Asn (N) or Asp (D); and
      Xaa¹¹ is Tyr (Y), Phe (F), or His (H);
   CDR-H2 comprises the amino acid sequence of SEQ ID NO: 57, 60, 61, or 62;
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises GFTFSXaa¹⁰Xaa¹¹ (SEQ ID NO: 63);
   wherein
      Xaa¹⁰ is Asn (N) or Asp (D); and
      Xaa¹¹ is Tyr (Y), Phe (F), or His (H);
   CDR-H2 comprises Xaa¹²YDGXaa¹³Xaa¹⁴ (SEQ ID NO: 64);
   wherein
      Xaa¹² is Asp (D) or Ser (S);
      Xaa¹³ is Gly (G), Asp (D), or Leu (L); and
      Xaa¹⁴ is Ser (S), Asp (D), or Asn (N);
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises the amino acid sequence of SEQ ID NO: 56, 58, or 59;
   CDR-H2 comprises Xaa¹²YDGXaa¹³Xaa¹⁴ (SEQ ID NO: 64);
   wherein
      Xaa¹² is Asp (D) or Ser (S);
      Xaa¹³ is Gly (G), Asp (D), or Leu (L); and
      Xaa¹⁴ is Ser (S), Asp (D), or Asn (N);
   CDR-H3 comprises the amino acid sequence of SEQ ID NO: 16, 3, 14, or 15;
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some embodiments, the multispecific antigen-binding molecule comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3; in which CDR-H1 comprises, consists essentially of, or consists of the amino acid sequence of SEQ ID NO: 56, 58, or 59; CDR-H2 comprises, consists essentially of, or consists of the amino acid sequence of SEQ ID NO: 57, 60, 61, or 62; CDR-H3 comprises, consists essentially of, or consists of the amino acid sequence of SEQ ID NO: 16, 3, 14, or 15; CDR-L1 comprises, consists essentially of, or consists of the amino acid sequence of SEQ ID NO: 4; CDR-L2 comprises, consists essentially of, or consists of the amino acid sequence of SEQ ID NO: 5; and/or CDR-L3 comprises, consists essentially of, or consists of the amino acid sequence of SEQ ID NO: 6; and (B) a second antigen-binding domain that binds to a T cell receptor. In some cases, the first antigen-binding domain comprises at least two, at least three, at least four, at least five, or all six CDR-H1, CDR-H2, CDR-H3, CDR-L1, CDR-L2, and CDR-L3. In some cases, the second antigen-binding domains binds to CD3. In some cases, the second antigen-binding domain comprises at least one, at least two, at least three, at least four, at least five, or all six CDRs of 40G5c or 38E4V1.MD1.

In some instances, the first antigen-binding domain comprises CDR-H1 comprising, consisting essentially of, or consisting of the amino acid sequence of SEQ ID NO: 56; CDR-H2 comprising, consisting essentially of, or consisting of the amino acid sequence of SEQ ID NO: 57; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 58; CDR-H2 comprising, consisting essentially of, or consisting of the amino acid sequence of SEQ ID NO: 60; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 58; CDR-H2 comprising, consisting essentially of, or consisting of the amino acid sequence of SEQ ID NO: 61; CDR-H3 comprising, consisting essentially of, or consisting of the amino acid sequence of SEQ ID NO: 3; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 58; CDR-H2 comprising, consisting essentially of, or consisting of the amino acid sequence of SEQ ID NO: 61; CDR-H3 comprising, consisting essentially of, or consisting of the amino acid sequence of SEQ ID NO: 14; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 59; CDR-H2 comprising, consisting essentially of, or consisting of the amino acid sequence of SEQ ID NO: 62; CDR-H3 comprising, consisting essentially of, or consisting of the amino acid sequence of SEQ ID NO: 15; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

In some instances, the first antigen-binding domain comprises CDR-H1 comprising, consisting essentially of, or consisting of the amino acid sequence of SEQ ID NO: 59; CDR-H2 comprising, consisting essentially of, or consisting of the amino acid sequence of SEQ ID NO: 57; CDR-H3 comprising, consisting essentially of, or consisting of the amino acid sequence of SEQ ID NO: 16; CDR-L1 comprising, consisting essentially of, or consisting of the amino acid sequence of SEQ ID NO: 4; CDR-L2 comprising, consisting essentially of, or consisting of the amino acid sequence of SEQ ID NO: 5; and CDR-L3 comprising, consisting essentially of, or consisting of the amino acid sequence of SEQ ID NO: 6.

The multispecific antigen-binding molecules of the present invention can be humanized antibodies. In some instances, the first antigen-binding domain and/or the second antigen-binding domain each independently comprises a constant region derived from an IgG framework region. In some cases, the IgG framework region is an IgG1, IgG2, or IgG4 framework region. In some cases, the IgG framework region is an IgG1 framework region.

In some embodiments, the multispecific antigen-binding molecule comprises (A) a first antigen-binding domain comprising a heavy chain variable region comprising one or more (e.g., 1, 2, 3, or all 4) of an FR-H1 sequence having at least 80%, 85%, 90%, 91%, 92%, 93%, 94%, 95%, 96%, 97%, 98%, 99%, or 100% sequence identity to the amino acid sequence of SEQ ID NO: 110; an FR-H2 having at least 80%, 85%, 90%, 91%, 92%, 93%, 94%, 95%, 96%, 97%, 98%, 99%, or 100% sequence identity to the amino acid sequence of SEQ ID NO: 111; an FR-H3 having at least 80%, 85%, 90%, 91%, 92%, 93%, 94%, 95%, 96%, 97%, 98%, 99%, or 100% sequence identity to the amino acid sequence of SEQ ID NO: 112; and/or an FR-H4 having at least 80%, 85%, 90%, 91%, 92%, 93%, 94%, 95%, 96%, 97%, 98%, 99%, or 100% sequence identity to the amino acid sequence of SEQ ID NO: 113.

In some embodiments, the multispecific antigen-binding molecule comprises (A) a first antigen-binding domain comprising a light chain variable region comprising one or more (e.g., 1, 2, 3, or all 4) of an FR-L1 having at least 80%, 85%, 90%, 91%, 92%, 93%, 94%, 95%, 96%, 97%, 98%, 99%, or 100% sequence identity to the amino acid sequence of SEQ ID NO: 114; an FR-L2 having at least 80%, 85%, 90%, 91%, 92%, 93%, 94%, 95%, 96%, 97%, 98%, 99%, or 100% sequence identity to the amino acid sequence of SEQ ID NO: 115; an FR-L3 co having at least 80%, 85%, 90%, 91%, 92%, 93%, 94%, 95%, 96%, 97%, 98%, 99%, or 100% sequence identity to the amino acid sequence of SEQ ID NO: 116; and/or an FR-L4 having at least 80%, 85%, 90%, 91%, 92%, 93%, 94%, 95%, 96%, 97%, 98%, 99%, or 100% sequence identity to the amino acid sequence of SEQ ID NO: 117.

For example, in some embodiments, the multispecific antigen-binding molecule comprises (A) a first antigen-binding domain comprising an FR-H1 comprising, consisting essentially of, or consisting of the amino acid sequence of SEQ ID NO: 110; an FR-H2 comprising, consisting essentially of, or consisting of the amino acid sequence of SEQ ID NO: 111; an FR-H3 comprising, consisting essentially of, or consisting of the amino acid sequence of SEQ ID NO: 112; an FR-H4 comprising, consisting essentially of, or consisting of the amino acid sequence of SEQ ID NO: 113; an FR-L1 comprising, consisting essentially of, or consisting of the amino acid sequence of SEQ ID NO: 114; an FR-L2 comprising, consisting essentially of, or consisting of the amino acid sequence of SEQ ID NO: 115; an FR-L3 comprising, consisting essentially of, or consisting of the amino acid sequence of SEQ ID NO: 116; and an FR-L4 comprising, consisting essentially of, or consisting of the amino acid sequence of SEQ ID NO: 117.

In some embodiments, the multispecific antigen-binding molecule comprises (A) a first antigen-binding domain comprising a VH sequence having at least 80%, 85%, 90%, 91%, 92%, 93%, 94%, 95%, 96%, 97%, 98%, 99%, or 100% sequence identity to SEQ ID NO: 7, 17-25, 30-34, 38, or 68; and (B) a second antigen-binding domain that binds to a T cell receptor. In some instances, the VH sequence of the first antigen-binding domain comprises at least 90% sequence identity to SEQ ID NO: 7, 17-25, 30-34, 38, or 68. In some instances, the VH sequence of the first antigen-binding domain comprises at least 95% sequence identity to SEQ ID NO: 7, 17-25, 30-34, 38, or 68. In some instances, the VH sequence of the first antigen-binding domain comprises at least 98% sequence identity to SEQ ID NO: 7, 17-25, 30-34, 38, or 68. In some instances, the VH sequence of the first antigen-binding domain comprises at least 99% sequence identity to SEQ ID NO: 7, 17-25, 30-34, 38, or 68. In some instances, the VH sequence of the first antigen-binding domain comprises 100% sequence identity to SEQ ID NO: 7, 17-25, 30-34, 38, or 68. In some cases, the second antigen-binding domains binds to CD3. In some cases, the second antigen-binding domain comprises a VH sequence having at least 80%, 85%, 90%, 91%, 92%, 93%, 94%, 95%, 96%, 97%, 98%, 99%, or 100% sequence identity to SEQ ID NO: 46 or 54. In some cases, the VH sequence of the second antigen-binding domain comprises at least 95% sequence identity to SEQ ID NO: 46 or 54. In some cases, the VH sequence of the second antigen-binding domain comprises at least 99% sequence identity to SEQ ID NO: 46 or 54. In some cases, the VH sequence of the second antigen-binding domain comprises 100% sequence identity to SEQ ID NO: 46 or 54.

In some embodiments, the multispecific antigen-binding molecule comprises (A) a first antigen-binding domain comprising a VL sequence having at least 80%, 85%, 90%, 91%, 92%, 93%, 94%, 95%, 96%, 97%, 98%, 99%, or 100% sequence identity to SEQ ID NO: 8, 26-29, 39, or 69; and (B) a second antigen-binding domain that binds to a T cell receptor. In some instances, the VL sequence comprises at least 90% sequence identity to SEQ ID NO: 8, 26-29, 39, or 69. In some instances, the VL sequence comprises at least 95% sequence identity to SEQ ID NO: 8, 26-29, 39, or 69. In some instances, the VL sequence comprises at least 98% sequence identity to SEQ ID NO: 8, 26-29, 39, or 69. In some instances, the VL sequence comprises at least 99% sequence identity to SEQ ID NO: 8, 26-29, 39, or 69. In some instances, the VL sequence comprises 100% sequence identity to SEQ ID NO: 8, 26-29, 39, or 69. In some cases, the second antigen-binding domains binds to CD3. In some cases, the second antigen-binding domain comprises a VL sequence having at least 80%, 85%, 90%, 91%, 92%, 93%, 94%, 95%, 96%, 97%, 98%, 99%, or 100% sequence identity to SEQ ID NO: 47 or 55. In some cases, the VL sequence of the second antigen-binding domain comprises at least 95% sequence identity to SEQ ID NO: 47 or 55. In some cases, the VL sequence of the second antigen-binding domain comprises at least 99% sequence identity to SEQ ID NO: 47 or 55. In some cases, the VL sequence of the second antigen-binding domain comprises 100% sequence identity to SEQ ID NO: 47 or 55.

In some embodiments, a multispecific antigen-binding molecule of the present invention comprises a first antigen-binding domain that binds to STEAP1 and comprises at least one, at least two, at least three, at least four, at least five, or all six CDRs as illustrated in Tables 1 or 2. In some instances, the first antigen-binding domain comprises a VH and/or a VL as illustrated in Table 1. In some instances, the multispecific antigen-binding molecule comprises a second antigen-binding domain that binds to a T cell receptor (e.g., CD3). In some cases, the second antigen-binding domain that binds to CD3 comprises at least one, at least two, at least three, at least four, at least five, or all six CDRs as illustrated in Tables 1 or 2. In some instances, the second antigen-binding domain that binds to CD3 comprises a VH and/or a VL as illustrated in Table 1.

In some embodiments, the multispecific antigen-binding molecule of any of the preceding embodiments comprises a first antigen-binding domain that binds to STEAP1 and a second antigen-binding domain that binds to a T cell receptor (e.g., CD3). In some embodiments, the first antigen-binding domain features a VH and a VL region, in which the VH region comprises one, two, or all three of the following CDRs: (a) CDR-H1 comprising, consisting essentially of, or consisting of the amino acid sequence of SEQ ID NO: 9, (b) CDR-H2 comprising, consisting essentially of, or consisting of the amino acid sequence of SEQ ID NO: 11, or (c) CDR-H3 comprising, consisting essentially of, or consisting of the amino acid sequence of SEQ ID NO: 3. In some instances, the VH region of the first antigen-binding domain comprises one, two, or all three of the following CDRs: (a) CDR-H1 comprising, consisting essentially of, or consisting of the amino acid sequence of SEQ ID NO: 58, (b) CDR-H2 comprising, consisting essentially of, or consisting of the amino acid sequence of SEQ ID NO: 60, or (c) CDR-H3 comprising, consisting essentially of, or consisting of the amino acid sequence of SEQ ID NO: 3. In some embodiments, the VH region of the first antigen-binding domain comprises an amino acid sequence having at least 90% sequence identity to SEQ ID NO: 30 (e.g., at least 95% sequence identity to SEQ ID NO: 30, at least 96% sequence identity to SEQ ID NO: 30, at least 97% sequence identity to SEQ ID NO: 30, at least 98% sequence identity to SEQ ID NO: 30, at least 99% sequence identity to SEQ ID NO: 30, or 100% sequence identity to SEQ ID NO: 30). In some embodiments, the VL region of the first antigen-binding domain comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region of the first antigen-binding domain comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27). In some instances, the second antigen-binding domain features a VH and a VL region, in which the VH region comprises one, two, or all three of the following CDRs: (a) CDR-H1 comprising, consisting essentially of, or consisting of the amino acid sequence of SEQ ID NO: 40 or 48, (b) CDR-H2 comprising, consisting essentially of, or consisting of the amino acid sequence of SEQ ID NO: 41 or 49, or (c) CDR-H3 comprising, consisting essentially of, or consisting of the amino acid sequence of SEQ ID NO: 42 or 50. In some embodiments, the VH region of the second antigen-binding domain comprises an amino acid sequence having at least 90% sequence identity to SEQ ID NO: 46 or 54 (e.g., at least 95% sequence identity to SEQ ID NO: 46 or 54, at least 96% sequence identity to SEQ ID NO: 46 or 54, at least 97% sequence identity to SEQ ID NO: 46 or 54, at least 98% sequence identity to SEQ ID NO: 46 or 54, at least 99% sequence identity to SEQ ID NO: 46 or 54, or 100% sequence identity to SEQ ID NO: 46 or 54). In some embodiments, the VL region of the second antigen-binding domain comprises one, two, or all three of the following CDRs: (a) CDR-L1 comprising, consisting essentially of, or consisting of the amino acid sequence of SEQ ID NO: 43 or 51, (b) CDR-L2 comprising, consisting essentially of, or consisting of the amino acid sequence of SEQ ID NO:44 or 52, or (c) CDR-L3 comprising, consisting essentially of, or consisting of the amino acid sequence of SEQ ID NO: 45 or 53. In some embodiments, the VL region of the second antigen-binding domain comprises an amino acid sequence having at least 90% sequence identity to SEQ ID NO: 47 or 55 (e.g., at least 95% sequence identity to SEQ ID NO: 47 or 55, at least 96% sequence identity to SEQ ID NO: 47 or 55, at least 97% sequence identity to SEQ ID NO: 47 or 55, at least 98% sequence identity to SEQ ID NO: 47 or 55, at least 99% sequence identity to SEQ ID NO: 47 or 55, or 100% sequence identity to SEQ ID NO: 47 or 55).

In some embodiments, the multispecific antigen-binding molecule of any of the preceding embodiments comprises a first antigen-binding domain that binds to STEAP1 and a second antigen-binding domain that binds to a T cell receptor (e.g., CD3). In some embodiments, the first antigen-binding domain features a VH and a VL region, in which the VH region comprises one, two, or all three of the following CDRs: (a) CDR-H1 comprising, consisting essentially of, or consisting of the amino acid sequence of SEQ ID NO: 9, (b) CDR-H2 comprising, consisting essentially of, or consisting of the amino acid sequence of SEQ ID NO: 12, or (c) CDR-H3 comprising, consisting essentially of, or consisting of the amino acid sequence of SEQ ID NO: 3. In some instances, the VH region of the first antigen-binding domain comprises one, two, or all three of the following CDRs: (a) CDR-H1 comprising, consisting essentially of, or consisting of the amino acid sequence of SEQ ID NO: 58, (b) CDR-H2 comprising, consisting essentially of, or consisting of the amino acid sequence of SEQ ID NO: 61, or (c) CDR-H3 comprising, consisting essentially of, or consisting of the amino acid sequence of SEQ ID NO: 3. In some embodiments, the VH region of the first antigen-binding domain comprises an amino acid sequence having at least 90% sequence identity to SEQ ID NO: 31 (e.g., at least 95% sequence identity to SEQ ID NO: 31, at least 96% sequence identity to SEQ ID NO: 31, at least 97% sequence identity to SEQ ID NO: 31, at least 98% sequence identity to SEQ ID NO: 31, at least 99% sequence identity to SEQ ID NO: 31, or 100% sequence identity to SEQ ID NO: 31). In some embodiments, the VL region of the first antigen-binding domain comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region of the first antigen-binding domain comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27). In some instances, the second antigen-binding domain features a VH and a VL region, in which the VH region comprises one, two, or all three of the following CDRs: (a) CDR-H1 comprising, consisting essentially of, or consisting of the amino acid sequence of SEQ ID NO: 40 or 48, (b) CDR-H2 comprising, consisting essentially of, or consisting of the amino acid sequence of SEQ ID NO: 41 or 49, or (c) CDR-H3 comprising, consisting essentially of, or consisting of the amino acid sequence of SEQ ID NO: 42 or 50. In some embodiments, the VH region of the second antigen-binding domain comprises an amino acid sequence having at least 90% sequence identity to SEQ ID NO: 46 or 54 (e.g., at least 95% sequence identity to SEQ ID NO: 46 or 54, at least 96% sequence identity to SEQ ID NO: 46 or 54, at least 97% sequence identity to SEQ ID NO: 46 or 54, at least 98% sequence identity to SEQ ID NO: 46 or 54, at least 99% sequence identity to SEQ ID NO: 46 or 54, or 100% sequence identity to SEQ ID NO: 46 or 54). In some embodiments, the VL region of the second antigen-binding domain comprises one, two, or all three of the following CDRs: (a) CDR-L1 comprising, consisting essentially of, or consisting of the amino acid sequence of SEQ ID NO: 43 or 51, (b) CDR-L2 comprising, consisting essentially of, or consisting of the amino acid sequence of SEQ ID NO:44 or 52, or (c) CDR-L3 comprising, consisting essentially of, or consisting of the amino acid sequence of SEQ ID NO: 45 or 53. In some embodiments, the VL region of the second antigen-binding domain comprises an amino acid sequence having at least 90% sequence identity to SEQ ID NO: 47 or 55 (e.g., at least 95% sequence identity to SEQ ID NO: 47 or 55, at least 96% sequence identity to SEQ ID NO: 47 or 55, at least 97% sequence identity to SEQ ID NO: 47 or 55, at least 98% sequence identity to SEQ ID NO: 47 or 55, at least 99% sequence identity to SEQ ID NO: 47 or 55, or 100% sequence identity to SEQ ID NO: 47 or 55).

In some embodiments, the multispecific antigen-binding molecule of any of the preceding embodiments comprises a first antigen-binding domain that binds to STEAP1 and a second antigen-binding domain that binds to a T cell receptor (e.g., CD3). In some embodiments, the first antigen-binding domain features a VH and a VL region, in which the VH region comprises one, two, or all three of the following CDRs: (a) CDR-H1 comprising, consisting essentially of, or consisting of the amino acid sequence of SEQ ID NO: 9, (b) CDR-H2 comprising, consisting essentially of, or consisting of the amino acid sequence of SEQ ID NO: 12, or (c) CDR-H3 comprising, consisting essentially of, or consisting of the amino acid sequence of SEQ ID NO: 14. In some instances, the VH region of the first antigen-binding domain comprises one, two, or all three of the following CDRs: (a) CDR-H1 comprising, consisting essentially of, or consisting of the amino acid sequence of SEQ ID NO: 58, (b) CDR-H2 comprising, consisting essentially of, or consisting of the amino acid sequence of SEQ ID NO: 61, or (c) CDR-H3 comprising, consisting essentially of, or consisting of the amino acid sequence of SEQ ID NO: 14. In some embodiments, the VH region of the first antigen-binding domain comprises an amino acid sequence having at least 90% sequence identity to SEQ ID NO: 32 (e.g., at least 95% sequence identity to SEQ ID NO: 32, at least 96% sequence identity to SEQ ID NO: 32, at least 97% sequence identity to SEQ ID NO: 32, at least 98% sequence identity to SEQ ID NO: 32, at least 99% sequence identity to SEQ ID NO: 32, or 100% sequence identity to SEQ ID NO: 32). In some embodiments, the VL region of the first antigen-binding domain comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region of the first antigen-binding domain comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27). In some instances, the second antigen-binding domain features a VH and a VL region, in which the VH region comprises one, two, or all three of the following CDRs: (a) CDR-H1 comprising, consisting essentially of, or consisting of the amino acid sequence of SEQ ID NO: 40 or 48, (b) CDR-H2 comprising, consisting essentially of, or consisting of the amino acid sequence of SEQ ID NO: 41 or 49, or (c) CDR-H3 comprising, consisting essentially of, or consisting of the amino acid sequence of SEQ ID NO: 42 or 50. In some embodiments, the VH region of the second antigen-binding domain comprises an amino acid sequence having at least 90% sequence identity to SEQ ID NO: 46 or 54 (e.g., at least 95% sequence identity to SEQ ID NO: 46 or 54, at least 96% sequence identity to SEQ ID NO: 46 or 54, at least 97% sequence identity to SEQ ID NO: 46 or 54, at least 98% sequence identity to SEQ ID NO: 46 or 54, at least 99% sequence identity to SEQ ID NO: 46 or 54, or 100% sequence identity to SEQ ID NO: 46 or 54). In some embodiments, the VL region of the second antigen-binding domain comprises one, two, or all three of the following CDRs: (a) CDR-L1 comprising, consisting essentially of, or consisting of the amino acid sequence of SEQ ID NO: 43 or 51, (b) CDR-L2 comprising, consisting essentially of, or consisting of the amino acid sequence of SEQ ID NO:44 or 52, or (c) CDR-L3 comprising, consisting essentially of, or consisting of the amino acid sequence of SEQ ID NO: 45 or 53. In some embodiments, the VL region of the second antigen-binding domain comprises an amino acid sequence having at least 90% sequence identity to SEQ ID NO: 47 or 55 (e.g., at least 95% sequence identity to SEQ ID NO: 47 or 55, at least 96% sequence identity to SEQ ID NO: 47 or 55, at least 97% sequence identity to SEQ ID NO: 47 or 55, at least 98% sequence identity to SEQ ID NO: 47 or 55, at least 99% sequence identity to SEQ ID NO: 47 or 55, or 100% sequence identity to SEQ ID NO: 47 or 55).

In some embodiments, the multispecific antigen-binding molecule of any of the preceding embodiments comprises a first antigen-binding domain that binds to STEAP1 and a second antigen-binding domain that binds to a T cell receptor (e.g., CD3). In some embodiments, the first antigen-binding domain features a VH and a VL region, in which the VH region comprises one, two, or all three of the following CDRs: (a) CDR-H1 comprising, consisting essentially of, or consisting of the amino acid sequence of SEQ ID NO: 10, (b) CDR-H2 comprising, consisting essentially of, or consisting of the amino acid sequence of SEQ ID NO: 13, or (c) CDR-H3 comprising, consisting essentially of, or consisting of the amino acid sequence of SEQ ID NO: 15. In some instances, the VH region of the first antigen-binding domain comprises one, two, or all three of the following CDRs: (a) CDR-H1 comprising, consisting essentially of, or consisting of the amino acid sequence of SEQ ID NO: 59, (b) CDR-H2 comprising, consisting essentially of, or consisting of the amino acid sequence of SEQ ID NO: 62, or (c) CDR-H3 comprising, consisting essentially of, or consisting of the amino acid sequence of SEQ ID NO: 15. In some embodiments, the VH region of the first antigen-binding domain comprises an amino acid sequence having at least 90% sequence identity to SEQ ID NO: 33 (e.g., at least 95% sequence identity to SEQ ID NO: 33, at least 96% sequence identity to SEQ ID NO: 33, at least 97% sequence identity to SEQ ID NO: 33, at least 98% sequence identity to SEQ ID NO: 33, at least 99% sequence identity to SEQ ID NO: 33, or 100% sequence identity to SEQ ID NO: 33). In some embodiments, the VL region of the first antigen-binding domain comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region of the first antigen-binding domain comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27). In some instances, the second antigen-binding domain features a VH and a VL region, in which the VH region comprises one, two, or all three of the following CDRs: (a) CDR-H1 comprising, consisting essentially of, or consisting of the amino acid sequence of SEQ ID NO: 40 or 48, (b) CDR-H2 comprising, consisting essentially of, or consisting of the amino acid sequence of SEQ ID NO: 41 or 49, or (c) CDR-H3 comprising, consisting essentially of, or consisting of the amino acid sequence of SEQ ID NO: 42 or 50. In some embodiments, the VH region of the second antigen-binding domain comprises an amino acid sequence having at least 90% sequence identity to SEQ ID NO: 46 or 54 (e.g., at least 95% sequence identity to SEQ ID NO: 46 or 54, at least 96% sequence identity to SEQ ID NO: 46 or 54, at least 97% sequence identity to SEQ ID NO: 46 or 54, at least 98% sequence identity to SEQ ID NO: 46 or 54, at least 99% sequence identity to SEQ ID NO: 46 or 54, or 100% sequence identity to SEQ ID NO: 46 or 54). In some embodiments, the VL region of the second antigen-binding domain comprises one, two, or all three of the following CDRs: (a) CDR-L1 comprising, consisting essentially of, or consisting of the amino acid sequence of SEQ ID NO: 43 or 51, (b) CDR-L2 comprising, consisting essentially of, or consisting of the amino acid sequence of SEQ ID NO:44 or 52, or (c) CDR-L3 comprising, consisting essentially of, or consisting of the amino acid sequence of SEQ ID NO: 45 or 53. In some embodiments, the VL region of the second antigen-binding domain comprises an amino acid sequence having at least 90% sequence identity to SEQ ID NO: 47 or 55 (e.g., at least 95% sequence identity to SEQ ID NO: 47 or 55, at least 96% sequence identity to SEQ ID NO: 47 or 55, at least 97% sequence identity to SEQ ID NO: 47 or 55, at least 98% sequence identity to SEQ ID NO: 47 or 55, at least 99% sequence identity to SEQ ID NO: 47 or 55, or 100% sequence identity to SEQ ID NO: 47 or 55).

In some embodiments, the multispecific antigen-binding molecule of any of the preceding embodiments comprises a first antigen-binding domain that binds to STEAP1 and a second antigen-binding domain that binds to a T cell receptor (e.g., CD3). In some embodiments, the first antigen-binding domain features a VH and a VL region, in which the VH region comprises one, two, or all three of the following CDRs: (a) CDR-H1 comprising, consisting essentially of, or consisting of the amino acid sequence of SEQ ID NO: 10, (b) CDR-H2 comprising, consisting essentially of, or consisting of the amino acid sequence of SEQ ID NO: 2, or (c) CDR-H3 comprising, consisting essentially of, or consisting of the amino acid sequence of SEQ ID NO: 16. In some instances, the VH region of the first antigen-binding domain comprises one, two, or all three of the following CDRs: (a) CDR-H1 comprising, consisting essentially of, or consisting of the amino acid sequence of SEQ ID NO: 59, (b) CDR-H2 comprising, consisting essentially of, or consisting of the amino acid sequence of SEQ ID NO: 57, or (c) CDR-H3 comprising, consisting essentially of, or consisting of the amino acid sequence of SEQ ID NO: 16. In some embodiments, the VH region of the first antigen-binding domain comprises an amino acid sequence having at least 90% sequence identity to SEQ ID NO: 34 (e.g., at least 95% sequence identity to SEQ ID NO: 34, at least 96% sequence identity to SEQ ID NO: 34, at least 97% sequence identity to SEQ ID NO: 34, at least 98% sequence identity to SEQ ID NO: 34, at least 99% sequence identity to SEQ ID NO: 34, or 100% sequence identity to SEQ ID NO: 34). In some embodiments, the VL region of the first antigen-binding domain comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region of the first antigen-binding domain comprises an amino acid sequence having at least 90% sequence identity to SEQ ID NO: 27 (e.g., at least 95% sequence identity to SEQ ID NO: 27, at least 96% sequence identity to SEQ ID NO: 27, at least 97% sequence identity to SEQ ID NO: 27, at least 98% sequence identity to SEQ ID NO: 27, at least 99% sequence identity to SEQ ID NO: 27, or 100% sequence identity to SEQ ID NO: 27). In some instances, the second antigen-binding domain features a VH and a VL region, in which the VH region comprises one, two, or all three of the following CDRs: (a) CDR-H1 comprising, consisting essentially of, or consisting of the amino acid sequence of SEQ ID NO: 40 or 48, (b) CDR-H2 comprising, consisting essentially of, or consisting of the amino acid sequence of SEQ ID NO: 41 or 49, or (c) CDR-H3 comprising, consisting essentially of, or consisting of the amino acid sequence of SEQ ID NO: 42 or 50. In some embodiments, the VH region of the second antigen-binding domain comprises an amino acid sequence having at least 90% sequence identity to SEQ ID NO: 46 or 54 (e.g., at least 95% sequence identity to SEQ ID NO: 46 or 54, at least 96% sequence identity to SEQ ID NO: 46 or 54, at least 97% sequence identity to SEQ ID NO: 46 or 54, at least 98% sequence identity to SEQ ID NO: 46 or 54, at least 99% sequence identity to SEQ ID NO: 46 or 54, or 100% sequence identity to SEQ ID NO: 46 or 54). In some embodiments, the VL region of the second antigen-binding domain comprises one, two, or all three of the following CDRs: (a) CDR-L1 comprising, consisting essentially of, or consisting of the amino acid sequence of SEQ ID NO: 43 or 51, (b) CDR-L2 comprising, consisting essentially of, or consisting of the amino acid sequence of SEQ ID NO:44 or 52, or (c) CDR-L3 comprising, consisting essentially of, or consisting of the amino acid sequence of SEQ ID NO: 45 or 53. In some embodiments, the VL region of the second antigen-binding domain comprises an amino acid sequence having at least 90% sequence identity to SEQ ID NO: 47 or 55 (e.g., at least 95% sequence identity to SEQ ID NO: 47 or 55, at least 96% sequence identity to SEQ ID NO: 47 or 55, at least 97% sequence identity to SEQ ID NO: 47 or 55, at least 98% sequence identity to SEQ ID NO: 47 or 55, at least 99% sequence identity to SEQ ID NO: 47 or 55, or 100% sequence identity to SEQ ID NO: 47 or 55).

In some embodiments, the multispecific antigen-binding molecule of any of the preceding embodiments comprises a first antigen-binding domain that binds to STEAP1 and a second antigen-binding domain that binds to a T cell receptor (e.g., CD3). In some embodiments, the first antigen-binding domain features a VH and a VL region, in which the VH region comprises one, two, or all three of the following CDRs: (a) CDR-H1 comprising, consisting essentially of, or consisting of the amino acid sequence of SEQ ID NO: 35, (b) CDR-H2 comprising, consisting essentially of, or consisting of the amino acid sequence of SEQ ID NO: 36, or (c) CDR-H3 comprising, consisting essentially of, or consisting of the amino acid sequence of SEQ ID NO: 37. In some instances, the VH region of the first antigen-binding domain comprises one, two, or all three of the following CDRs: (a) CDR-H1 comprising, consisting essentially of, or consisting of the amino acid sequence of SEQ ID NO: 63, (b) CDR-H2 comprising, consisting essentially of, or consisting of the amino acid sequence of SEQ ID NO: 64, or (c) CDR-H3 comprising, consisting essentially of, or consisting of the amino acid sequence of SEQ ID NO: 37. In some embodiments, the VH region of the first antigen-binding domain comprises an amino acid sequence having at least 90% sequence identity to SEQ ID NO: 38 (e.g., at least 95% sequence identity to SEQ ID NO: 38, at least 96% sequence identity to SEQ ID NO: 38, at least 97% sequence identity to SEQ ID NO: 38, at least 98% sequence identity to SEQ ID NO: 38, at least 99% sequence identity to SEQ ID NO: 38, or 100% sequence identity to SEQ ID NO: 38). In some embodiments, the VL region of the first antigen-binding domain comprises one, two, or all three of the following CDRs: (a) CDR-L1 comprising, consisting essentially of, or consisting of the amino acid sequence of SEQ ID NO: 4, (b) CDR-L2 comprising, consisting essentially of, or consisting of the amino acid sequence of SEQ ID NO: 5, or (c) CDR-L3 comprising, consisting essentially of, or consisting of the amino acid sequence of SEQ ID NO: 6. In some embodiments, the VL region of the first antigen-binding domain comprises an amino acid sequence having at least 90% sequence identity to SEQ ID NO: 39 (e.g., at least 95% sequence identity to SEQ ID NO: 39, at least 96% sequence identity to SEQ ID NO: 39, at least 97% sequence identity to SEQ ID NO: 39, at least 98% sequence identity to SEQ ID NO: 39, at least 99% sequence identity to SEQ ID NO: 39, or 100% sequence identity to SEQ ID NO: 39). In some instances, the second antigen-binding domain features a VH and a VL region, in which the VH region comprises one, two, or all three of the following CDRs: (a) CDR-H1 comprising, consisting essentially of, or consisting of the amino acid sequence of SEQ ID NO: 40 or 48, (b) CDR-H2 comprising, consisting essentially of, or consisting of the amino acid sequence of SEQ ID NO: 41 or 49, or (c) CDR-H3 comprising, consisting essentially of, or consisting of the amino acid sequence of SEQ ID NO: 42 or 50. In some embodiments, the VH region of the second antigen-binding domain comprises an amino acid sequence having at least 90% sequence identity to SEQ ID NO: 46 or 54 (e.g., at least 95% sequence identity to SEQ ID NO: 46 or 54, at least 96% sequence identity to SEQ ID NO: 46 or 54, at least 97% sequence identity to SEQ ID NO: 46 or 54, at least 98% sequence identity to SEQ ID NO: 46 or 54, at least 99% sequence identity to SEQ ID NO: 46 or 54, or 100% sequence identity to SEQ ID NO: 46 or 54). In some embodiments, the VL region of the second antigen-binding domain comprises one, two, or all three of the following CDRs: (a) CDR-L1 comprising, consisting essentially of, or consisting of the amino acid sequence of SEQ ID NO: 43 or 51, (b) CDR-L2 comprising, consisting essentially of, or consisting of the amino acid sequence of SEQ ID NO:44 or 52, or (c) CDR-L3 comprising, consisting essentially of, or consisting of the amino acid sequence of SEQ ID NO: 45 or 53. In some embodiments, the VL region of the second antigen-binding domain comprises an amino acid sequence having at least 90% sequence identity to SEQ ID NO: 47 or 55 (e.g., at least 95% sequence identity to SEQ ID NO: 47 or 55, at least 96% sequence identity to SEQ ID NO: 47 or 55, at least 97% sequence identity to SEQ ID NO: 47 or 55, at least 98% sequence identity to SEQ ID NO: 47 or 55, at least 99% sequence identity to SEQ ID NO: 47 or 55, or 100% sequence identity to SEQ ID NO: 47 or 55).

In some embodiments, the multispecific antigen-binding molecule of any of the preceding embodiments comprises a first antigen-binding domain that binds to STEAP1 and a second antigen-binding domain that binds to a T cell receptor (e.g., CD3). In some embodiments, the VH region of the first antigen-binding domain comprises an amino acid sequence having at least 90% sequence identity to SEQ ID NO: 68 (e.g., at least 95% sequence identity to SEQ ID NO: 68, at least 96% sequence identity to SEQ ID NO: 68, at least 97% sequence identity to SEQ ID NO: 68, at least 98% sequence identity to SEQ ID NO: 68, at least 99% sequence identity to SEQ ID NO: 68, or 100% sequence identity to SEQ ID NO: 68). In some embodiments, the VL region of the first antigen-binding domain comprises an amino acid sequence having at least 90% sequence identity to SEQ ID NO: 69 (e.g., at least 95% sequence identity to SEQ ID NO: 69, at least 96% sequence identity to SEQ ID NO: 69, at least 97% sequence identity to SEQ ID NO: 69, at least 98% sequence identity to SEQ ID NO: 69, at least 99% sequence identity to SEQ ID NO: 69, or 100% sequence identity to SEQ ID NO: 69). In some embodiments, the VH region of the second antigen-binding domain comprises an amino acid sequence having at least 90% sequence identity to SEQ ID NO: 46 or 54 (e.g., at least 95% sequence identity to SEQ ID NO: 46 or 54, at least 96% sequence identity to SEQ ID NO: 46 or 54, at least 97% sequence identity to SEQ ID NO: 46 or 54, at least 98% sequence identity to SEQ ID NO: 46 or 54, at least 99% sequence identity to SEQ ID NO: 46 or 54, or 100% sequence identity to SEQ ID NO: 46 or 54). In some embodiments, the VL region of the second antigen-binding domain comprises an amino acid sequence having at least 90% sequence identity to SEQ ID NO: 47 or 55 (e.g., at least 95% sequence identity to SEQ ID NO: 47 or 55, at least 96% sequence identity to SEQ ID NO: 47 or 55, at least 97% sequence identity to SEQ ID NO: 47 or 55, at least 98% sequence identity to SEQ ID NO: 47 or 55, at least 99% sequence identity to SEQ ID NO: 47 or 55, or 100% sequence identity to SEQ ID NO: 47 or 55).

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 83, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 82.

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 85, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 84.

In some embodiments, the multispecific antigen-binding molecule of any of the preceding embodiments comprises an anti-CD3 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 83, and an anti-CD3 light chain constant region having at least 90%, at least 91%, at least 92%, at least 93%, at least 94%, at least 95%, at least 96%, at least 97%, at least 98%, at least 99%, or 100% sequence identity SEQ ID NO: 82.

In some embodiments, the multispecific antigen-binding molecule of any of the preceding embodiments comprises an anti-CD3 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 85, and an anti-CD3 light chain constant region having at least 90%, at least 91%, at least 92%, at least 93%, at least 94%, at least 95%, at least 96%, at least 97%, at least 98%, at least 99%, or 100% sequence identity SEQ ID NO: 84.

In some embodiments, the multispecific antigen-binding molecule of any of the preceding embodiments comprises an anti-STEAP1 heavy chain having at least 90%, at least 91%, at least 92%, at least 93%, at least 94%, at least 95%, at least 96%, at least 97%, at least 98%, at least 99%, or 100% sequence identity to SEQ ID NO: 71, and an anti-STEAP1 light chain having at least 90%, at least 91%, at least 92%, at least 93%, at least 94%, at least 95%, at least 96%, at least 97%, at least 98%, at least 99%, or 100% sequence identity SEQ ID NO: 70.

In some embodiments, the multispecific antigen-binding molecule of any of the preceding embodiments comprises an anti-STEAP1 heavy chain having at least 90%, at least 91%, at least 92%, at least 93%, at least 94%, at least 95%, at least 96%, at least 97%, at least 98%, at least 99%, or 100% sequence identity to SEQ ID NO: 73, and an anti-STEAP1 light chain having at least 90%, at least 91%, at least 92%, at least 93%, at least 94%, at least 95%, at least 96%, at least 97%, at least 98%, at least 99%, or 100% sequence identity SEQ ID NO: 72.

In some embodiments, the multispecific antigen-binding molecule of any of the preceding embodiments comprises an anti-STEAP1 heavy chain having at least 90%, at least 91%, at least 92%, at least 93%, at least 94%, at least 95%, at least 96%, at least 97%, at least 98%, at least 99%, or 100% sequence identity to SEQ ID NO: 75, and an anti-STEAP1 light chain having at least 90%, at least 91%, at least 92%, at least 93%, at least 94%, at least 95%, at least 96%, at least 97%, at least 98%, at least 99%, or 100% sequence identity SEQ ID NO: 74.

In some embodiments, the multispecific antigen-binding molecule of any of the preceding embodiments comprises an anti-STEAP1 heavy chain having at least 90%, at least 91%, at least 92%, at least 93%, at least 94%, at least 95%, at least 96%, at least 97%, at least 98%, at least 99%, or 100% sequence identity to SEQ ID NO: 77, and an anti-STEAP1 light chain having at least 90%, at least 91%, at least 92%, at least 93%, at least 94%, at least 95%, at least 96%, at least 97%, at least 98%, at least 99%, or 100% sequence identity SEQ ID NO: 76.

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 122, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 123.

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 126, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 127.

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 130, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 131.

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 134, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 135.

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 138, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 139.

In some embodiments, the multispecific antigen-binding molecule of any of the preceding embodiments comprises an anti-STEAP1 heavy chain constant region having at least 90%, at least 91%, at least 92%, at least 93%, at least 94%, at least 95%, at least 96%, at least 97%, at least 98%, at least 99%, or 100% sequence identity to SEQ ID NO: 142, and an anti-STEAP1 light chain constant region having at least 90%, at least 91%, at least 92%, at least 93%, at least 94%, at least 95%, at least 96%, at least 97%, at least 98%, at least 99%, or 100% sequence identity SEQ ID NO: 143.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 79, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 78.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 81, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 80.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 124, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 125.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 128, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 129.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 132, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 133.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 136, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 137.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 140, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 141.

In some embodiments, the multispecific antigen-binding molecule of any of the preceding embodiments comprises an anti-CD3 heavy chain having at least 90%, at least 91%, at least 92%, at least 93%, at least 94%, at least 95%, at least 96%, at least 97%, at least 98%, at least 99%, or 100% sequence identity to SEQ ID NO: 144, and an anti-CD3 light chain having at least 90%, at least 91%, at least 92%, at least 93%, at least 94%, at least 95%, at least 96%, at least 97%, at least 98%, at least 99%, or 100% sequence identity SEQ ID NO: 145.

In some embodiments, the multispecific antigen-binding molecule comprises an anti-STEAP1 heavy chain comprising SEQ ID NO: 122, an anti-STEAP1 light chain comprising SEQ ID NO: 123, an anti-CD3 heavy chain comprising SEQ ID NO: 124, and an anti-CD3 light chain comprising SEQ ID NO: 125.

In some embodiments, the multispecific antigen-binding molecule comprises an anti-STEAP1 heavy chain comprising SEQ ID NO: 126, an anti-STEAP1 light chain comprising SEQ ID NO: 127, an anti-CD3 heavy chain comprising SEQ ID NO: 128, and an anti-CD3 light chain comprising SEQ ID NO: 129.

In some embodiments, the multispecific antigen-binding molecule comprises an anti-STEAP1 heavy chain comprising SEQ ID NO: 130, an anti-STEAP1 light chain comprising SEQ ID NO: 131, an anti-CD3 heavy chain comprising SEQ ID NO: 132, and an anti-CD3 light chain comprising SEQ ID NO: 133.

In some embodiments, the multispecific antigen-binding molecule comprises an anti-STEAP1 heavy chain comprising SEQ ID NO: 134, an anti-STEAP1 light chain comprising SEQ ID NO: 135, an anti-CD3 heavy chain comprising SEQ ID NO: 136, and an anti-CD3 light chain comprising SEQ ID NO: 137.

In some embodiments, the multispecific antigen-binding molecule comprises an anti-STEAP1 heavy chain comprising SEQ ID NO: 138, an anti-STEAP1 light chain comprising SEQ ID NO: 139, an anti-CD3 heavy chain comprising SEQ ID NO: 140, and an anti-CD3 light chain comprising SEQ ID NO: 141.

In some embodiments, the multispecific antigen-binding molecule comprises an anti-STEAP1 heavy chain comprising SEQ ID NO: 142, an anti-STEAP1 light chain comprising SEQ ID NO: 143, an anti-CD3 heavy chain comprising SEQ ID NO: 144, and an anti-CD3 light chain comprising SEQ ID NO: 145.

In some embodiments, the multispecific antigen-binding molecule further comprises a third antigen-binding domain. The third antigen-binding domain can be an antigen expressed on the surface of a cell. The antigen can be expressed on the surface of a cell obtained from a solid tumor.

The antigen can be expressed on the surface of a cell of prostate cancer, Ewing sarcoma, lung cancer, colorectal cancer, breast cancer, bladder cancer, ovarian cancer, or cervical cancer.

In some embodiments, the antigen is a tumor-associated antigen (TAA). The TAA can be a receptor expressed on the surface of a cell of prostate cancer, Ewing sarcoma, lung cancer, colorectal cancer, breast cancer, bladder cancer, ovarian cancer, or cervical cancer. In some instances, the TAA is a receptor expressed on the surface of a prostate cancer cell. In some cases, the TAA is a receptor expressed on an Ewing sarcoma cell. Exemplary tumor-associated antigens include, but are not limited to, prostate-specific membrane antigen (PSMA), prostate stem cell antigen (PSCA), epithelial cell adhesion molecule (EpCAM), prostate-specific antigen (PSA), prostatic acid phosphatase (PAP), STEAP2, and HBA-71.

In some instances, the multispecific antigen-binding molecule further comprises a third antigen-binding domain that binds to a tumor-associated antigen. In some cases, the third antigen-binding domain binds to a TAA of prostate cancer, Ewing sarcoma, lung cancer, colorectal cancer, breast cancer, bladder cancer, ovarian cancer, or cervical cancer. In some cases, the third antigen-binding domain binds to prostate-specific membrane antigen (PSMA), prostate stem cell antigen (PSCA), epithelial cell adhesion molecule (EpCAM), prostate-specific antigen (PSA), prostatic acid phosphatase (PAP), STEAP2, or HBA-71. In some cases, the third antigen-binding domain binds to PSMA. In some cases, the third antigen-binding domain binds to PSCA. In some cases, the third antigen-binding domain binds to EpCAM. In some cases, the third antigen-binding domain binds to PSA. In some cases, the third antigen-binding domain binds to PAP. In some cases, the third antigen-binding domain binds to STEAP2. In some cases, the third antigen-binding domain binds to HBA-71.

In some embodiments, the multispecific antigen-binding molecule binds to human STEAP1 with a K_{D} of less than 100 nM, less than 75 nM, less than 50 nM, less than 40 nM, less than 30 nM, less than 20 nM, less than 10 nM, less than 1 nM, or lower.

In some embodiments, multispecific antigen-binding molecule binds to human STEAP1 with a K_{D} of about 1 nM to about 100 nM, about 1 nM to about 75 nM, about 1 nM to about 50 nM, about 1 nM to about 40 nM, about 1 nM to about 30 nM, about 1 nM to about 20 nM, about 1 nM to about 10 nM, about 1 nM to about 5 nM, about 5 nM to about 100 nM, about 5 nM to about 75 nM, about 5 nM to about 50 nM, about 5 nM to about 40 nM, about 5 nM to about 30 nM, about 5 nM to about 20 nM, about 5 nM to about 10 nM, about 10 nM to about 100 nM, about 10 nM to about 75 nM, about 10 nM to about 50 nM, about 10 nM to about 40 nM, about 10 nM to about 30 nM, about 10 nM to about 20 nM, about 15 nM to about 100 nM, about 15 nM to about 75 nM, about 15 nM to about 50 nM, about nM to about 40 nM, about 15 nM to about 30 nM, about 15 nM to about 20 nM, about 20 nM to about 100 nM, about 20 nM to about 75 nM, about 20 nM to about 50 nM, about 20 nM to about 40 nM, about 20 nM to about 30 nM, about 25 nM to about 100 nM, about 25 nM to about 75 nM, about nM to about 50 nM, about 25 nM to about 40 nM, about 25 nM to about 30 nM, about 30 nM to about 100 nM, about 30 nM to about 75 nM, about 30 nM to about 50 nM, about 30 nM to about 40 nM, about 35 nM to about 100 nM, about 35 nM to about 75 nM, about 35 nM to about 50 nM, about nM to about 40 nM, about 40 nM to about 100 nM, about 40 nM to about 75 nM, about 40 nM to about 50 nM, about 45 nM to about 100 nM, about 45 nM to about 75 nM, about 45 nM to about 50 nM, about 50 nM to about 100 nM, about 50 nM to about 75 nM, about 70 nM to about 100 nM, about 70 nM to about 75 nM, about 80 nM to about 100 nM, or about 90 nM to about 100 nM. In some embodiments, the multispecific antigen-binding molecule binds to human STEAP1 with a K_{D} of 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, about 20 nM, about 21 nM, about 22 nM, about 23 nM, about 24 nM, about 25 nM, about 26 nM, about 27 nM, about 28 nM, about 29 nM, about 30 nM, about 31 nM, about 32 nM, about 33 nM, about 34 nM, about 35 nM, about 36 nM, about 37 nM, about 38 nM, about 39 nM, about 40 nM, about 41 nM, about 42 nM, about 43 nM, about 44 nM, about 45 nM, about 46 nM, about 47 nM, about 48 nM, about 49 nM, or about 50 nM. In some embodiments, multispecific antigen-binding molecule binds to human STEAP1 with a K_{D} of about 29 nM. In some embodiments, the K_{D} is determined using a Kinetic Exclusion Assay (KinExA®), e.g., as described in Example 8.

In some embodiments, the multispecific antigen-binding molecule binds to cyno STEAP1 with a K_{D} of less than 100 nM, less than 75 nM, less than 50 nM, less than 40 nM, less than 30 nM, less than 20 nM, less than 10 nM, less than 1 nM, or lower.

In some embodiments, multispecific antigen-binding molecule binds to cyno STEAP1 with a K_{D} of about 1 nM to about 100 nM, about 1 nM to about 75 nM, about 1 nM to about 50 nM, about 1 nM to about 40 nM, about 1 nM to about 30 nM, about 1 nM to about 20 nM, about 1 nM to about 10 nM, about 1 nM to about 5 nM, about 5 nM to about 100 nM, about 5 nM to about 75 nM, about 5 nM to about 50 nM, about 5 nM to about 40 nM, about 5 nM to about 30 nM, about 5 nM to about 20 nM, about 5 nM to about 10 nM, about 10 nM to about 100 nM, about 10 nM to about 75 nM, about 10 nM to about 50 nM, about 10 nM to about 40 nM, about 10 nM to about 30 nM, about 10 nM to about 20 nM, about 15 nM to about 100 nM, about 15 nM to about 75 nM, about 15 nM to about 50 nM, about nM to about 40 nM, about 15 nM to about 30 nM, about 15 nM to about 20 nM, about 20 nM to about 100 nM, about 20 nM to about 75 nM, about 20 nM to about 50 nM, about 20 nM to about 40 nM, about 20 nM to about 30 nM, about 25 nM to about 100 nM, about 25 nM to about 75 nM, about nM to about 50 nM, about 25 nM to about 40 nM, about 25 nM to about 30 nM, about 30 nM to about 100 nM, about 30 nM to about 75 nM, about 30 nM to about 50 nM, about 30 nM to about 40 nM, about 35 nM to about 100 nM, about 35 nM to about 75 nM, about 35 nM to about 50 nM, about nM to about 40 nM, about 40 nM to about 100 nM, about 40 nM to about 75 nM, about 40 nM to about 50 nM, about 45 nM to about 100 nM, about 45 nM to about 75 nM, about 45 nM to about 50 nM, about 50 nM to about 100 nM, about 50 nM to about 75 nM, about 70 nM to about 100 nM, about 70 nM to about 75 nM, about 80 nM to about 100 nM, or about 90 nM to about 100 nM. In some embodiments, the multispecific antigen-binding molecule binds to cyno STEAP1 with a K_{D} of 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, about 20 nM, about 21 nM, about 22 nM, about 23 nM, about 24 nM, or about 25 nM. In some embodiments, multispecific antigen-binding molecule binds to cyno STEAP1 with a K_{D} of about 14 nM. In some embodiments, the K_{D} is determined using a Kinetic Exclusion Assay (KinExA®), e.g., as described in Example 8.

In some embodiments, the multispecific antigen-binding molecule has a Cₘₐₓ of up to or about 15, 20, 25, or 30 μg/mL. In some instances, the multispecific antigen-binding molecule has a Cₘₐₓ of about 11, 11.5, 12, 12.1, 12.6, 13, 13.2, 13.5, 15, 15.3, 18, 20, 23.4, 25, 27.9, 29.1, or 30 μg/mL.

In some embodiments, the multispecific antigen-binding molecule has a Cₘₐₓ of about 10 μg/mL to about 30 μg/mL (e.g., about 10 μg/mL to about 30 μg/mL, about 10 μg/mL to about 28 μg/mL, about 10 μg/mL to about 26 μg/mL, about 10 μg/mL to about 24 μg/mL, about 10 μg/mL to about 22 μg/mL, about 10 μg/mL to about 20 μg/mL, about 10 μg/mL to about 18 μg/mL, about 10 μg/mL to about 16 μg/mL, about 10 μg/mL to about 14 μg/mL, about 10 μg/mL to about 12 μg/mL, about 12 μg/mL to about 30 μg/mL, about 12 μg/mL to about 28 μg/mL, about 12 μg/mL to about 26 μg/mL, about 12 μg/mL to about 24 μg/mL, about 12 μg/mL to about 22 μg/mL, about 12 μg/mL to about 20 μg/mL, about 12 μg/mL to about 18 μg/mL, about 12 μg/mL to about 16 μg/mL, about 12 μg/mL to about 14 μg/mL, about 14 μg/mL to about 30 μg/mL, about 14 μg/mL to about 28 μg/mL, about 14 μg/mL to about 26 μg/mL, about 14 μg/mL to about 24 μg/mL, about 14 μg/mL to about 22 μg/mL, about 14 μg/mL to about 20 μg/mL, about 14 μg/mL to about 18 μg/mL, about 14 μg/mL to about 16 μg/mL, about 16 μg/mL to about 30 μg/mL, about 16 μg/mL to about 28 μg/mL, about 16 μg/mL to about 26 μg/mL, about 16 μg/mL to about 24 μg/mL, about 16 μg/mL to about 22 μg/mL, about 16 μg/mL to about 20 μg/mL, about 16 μg/mL to about 18 μg/mL, about 18 μg/mL to about 30 μg/mL, about 18 μg/mL to about 28 μg/mL, about 18 μg/mL to about 26 μg/mL, about 18 μg/mL to about 24 μg/mL, about 18 μg/mL to about 22 μg/mL, about 18 μg/mL to about 20 μg/mL, about 20 μg/mL to about 30 μg/mL, about 20 μg/mL to about 28 μg/mL, about 20 μg/mL to about 26 μg/mL, about 20 μg/mL to about 24 μg/mL, about 20 μg/mL to about 22 μg/mL, about 22 μg/mL to about 30 μg/mL, about 22 μg/mL to about 28 μg/mL, about 22 μg/mL to about 26 μg/mL, about 22 μg/mL to about 24 μg/mL, about 24 μg/mL to about 30 μg/mL, about 24 μg/mL to about 28 μg/mL, about 22 μg/mL to about 26 μg/mL, about 22 μg/mL to about 24 μg/mL, about 26 μg/mL to about 30 μg/mL, about 26 μg/mL to about 28 μg/mL or about 28 μg/mL to about 30 μg/mL). In some embodiments, the multispecific antigen-binding molecule has a Cₘₐₓ of about 20 μg/mL to about 28 μg/mL (e.g., about 20 μg/mL, about 21 μg/mL, about 22 μg/mL, about 23 μg/mL, about 24 μg/mL, about 25 μg/mL, about 26 μg/mL, about 27 μg/mL, or about 28 μg/mL). In some embodiments, the multispecific antigen-binding molecule has a Cₘₐₓ of about 24 μg/mL. In some embodiments, Cₘₐₓ may be determined as described in Example 9 (in female SCID mice). In some embodiments, Cₘₐₓ may be determined as described in Example 10 (in cynomolgus monkeys).

In some embodiments, the multispecific antigen-binding molecule has an EC₅₀ of about 0.6, 0.56, 0.5, 0.45, 0.4, 0.35, 0.3, 0.25, 0.2, 0.15, 0.1, 0.09, 0.05, or lower. In some cases, the multispecific antigen-binding molecule has an EC₅₀ of about 0.5 or lower. In some cases, the multispecific antigen-binding molecule has an EC₅₀ of about 0.4 or lower. In some cases, the multispecific antigen-binding molecule has an EC₅₀ of about 0.3 or lower. In some cases, the multispecific antigen-binding molecule has an EC₅₀ of about 0.2 or lower. In some cases, the multispecific antigen-binding molecule has an EC₅₀ of about 0.1 or lower.

In some embodiments, the multispecific antigen-binding molecule has an EC₅₀ of about 0.05 to about 0.8 (e.g., about 0.05 to about 0.8, about 0.05 to about 0.75, about 0.05 to about 0.70, about 0.05 to about 0.65, about 0.05 to about 0.6, about 0.05 to about 0.55, about 0.05 to about 0.5, about 0.05 to about 0.45, about 0.05 to about 0.4, about 0.05 to about 0.35, about 0.05 to about 0.3, about 0.05 to about 0.25, about 0.05 to about 0.2, about 0.05 to about 0.15, about 0.05 to about 0.1, about 0.05 to about 0.09, about 0.05 to about 0.08, about 0.05 to about 0.07, about 0.05 to about 0.06, about 0.06 to about 0.8, about 0.06 to about 0.75, about 0.06 to about 0.70, about 0.06 to about 0.65, about 0.06 to about 0.6, about 0.06 to about 0.55, about 0.06 to about 0.5, about 0.06 to about 0.45, about 0.06 to about 0.4, about 0.06 to about 0.35, about 0.06 to about 0.3, about 0.06 to about 0.25, about 0.06 to about 0.2, about 0.06 to about 0.15, about 0.06 to about 0.1, about 0.06 to about 0.09, about 0.06 to about 0.08, about 0.06 to about 0.07, about 0.07 to about 0.8, about 0.07 to about 0.75, about 0.07 to about 0.70, about 0.07 to about 0.65, about 0.07 to about 0.6, about 0.07 to about 0.55, about 0.07 to about 0.5, about 0.07 to about 0.45, about 0.07 to about 0.4, about 0.07 to about 0.35, about 0.07 to about 0.3, about 0.07 to about 0.25, about 0.07 to about 0.2, about 0.07 to about 0.15, about 0.07 to about 0.1, about 0.07 to about 0.09, about 0.07 to about 0.08, about 0.08 to about 0.8, about 0.08 to about 0.75, about 0.08 to about 0.70, about 0.08 to about 0.65, about 0.08 to about 0.6, about 0.08 to about 0.55, about 0.08 to about 0.5, about 0.08 to about 0.45, about 0.08 to about 0.4, about 0.08 to about 0.35, about 0.08 to about 0.3, about 0.08 to about 0.25, about 0.08 to about 0.2, about 0.08 to about 0.15, about 0.08 to about 0.1, about 0.08 to about 0.09, about 0.09 to about 0.8, about 0.09 to about 0.75, about 0.09 to about 0.70, about 0.09 to about 0.65, about 0.09 to about 0.6, about 0.09 to about 0.55, about 0.09 to about 0.5, about 0.09 to about 0.45, about 0.09 to about 0.4, about 0.09 to about 0.35, about 0.09 to about 0.3, about 0.09 to about 0.25, about 0.09 to about 0.2, about 0.09 to about 0.15, about 0.09 to about 0.1, about 0.1 to about 0.8, about 0.1 to about 0.75, about 0.1 to about 0.70, about 0.1 to about 0.65, about 0.1 to about 0.6, about 0.1 to about 0.55, about 0.1 to about 0.5, about 0.1 to about 0.45, about 0.1 to about 0.4, about 0.1 to about 0.35, about 0.1 to about 0.3, about 0.1 to about 0.25, about 0.1 to about 0.2, about 0.1 to about 0.15, about 0.15 to about 0.8, about 0.15 to about 0.75, about 0.15 to about 0.70, about 0.15 to about 0.65, about 0.15 to about 0.6, about 0.15 to about 0.55, about 0.15 to about 0.5, about 0.15 to about 0.45, about 0.15 to about 0.4, about 0.15 to about 0.35, about 0.15 to about 0.3, about 0.15 to about 0.25, about 0.15 to about 0.2, about 0.2 to about 0.8, about 0.2 to about 0.75, about 0.2 to about 0.70, about 0.2 to about 0.65, about 0.2 to about 0.6, about 0.2 to about 0.55, about 0.2 to about 0.5, about 0.2 to about 0.45, about 0.2 to about 0.4, about 0.2 to about 0.35, about 0.2 to about 0.3, about 0.2 to about 0.25, about 0.25 to about 0.8, about 0.25 to about 0.75, about 0.25 to about 0.70, about 0.25 to about 0.65, about 0.25 to about 0.6, about 0.25 to about 0.55, about 0.25 to about 0.5, about 0.25 to about 0.45, about 0.25 to about 0.4, about 0.25 to about 0.35, about 0.25 to about 0.3, about 0.3 to about 0.8, about 0.3 to about 0.75, about 0.3 to about 0.70, about 0.3 to about 0.65, about 0.3 to about 0.6, about 0.3 to about 0.55, about 0.3 to about 0.5, about 0.3 to about 0.45, about 0.3 to about 0.4, about 0.3 to about 0.35, about 0.35 to about 0.8, about 0.35 to about 0.75, about 0.35 to about 0.70, about 0.35 to about 0.65, about 0.35 to about 0.6, about 0.35 to about 0.55, about 0.35 to about 0.5, about 0.35 to about 0.45, about 0.35 to about 0.4, about 0.4 to about 0.8, about 0.4 to about 0.75, about 0.4 to about 0.70, about 0.4 to about 0.65, about 0.4 to about 0.6, about 0.4 to about 0.55, about 0.4 to about 0.5, about 0.4 to about 0.45, about 0.45 to about 0.8, about 0.45 to about 0.75, about 0.45 to about 0.70, about 0.45 to about 0.65, about 0.45 to about 0.6, about 0.45 to about 0.55, about 0.45 to about 0.5, about 0.5 to about 0.8, about 0.5 to about 0.75, about 0.5 to about 0.70, about 0.5 to about 0.65, about 0.5 to about 0.6, about 0.5 to about 0.55, about 0.55 to about 0.8, about 0.55 to about 0.75, about 0.55 to about 0.70, about 0.55 to about 0.65, about 0.55 to about 0.6, about 0.6 to about 0.8, about 0.6 to about 0.75, about 0.6 to about 0.70, about 0.6 to about 0.65, about 0.65 to about 0.8, about 0.65 to about 0.75, about 0.65 to about 0.70, about 0.7 to about 0.8, about 0.7 to about 0.75, or about 0.75 to about 0.8).

In some embodiments, the EC₅₀ is determined in a cell killing assay at 72 hours with human CD8+ T cells and STEAP1-expressing LNCaP-X1.2 cells. In some embodiments, the EC₅₀ is about 0.05 to about 0.4 (e.g., about 0.05, about 0.06, about 0.07, about 0.08, about 0.09, about 0.1, about 0.15, about 0.2, about 0.25, about 0.3, about 0.35, or about 0.4). In some embodiments, the EC₅₀ is about 0.08 or about 0.3. In some embodiments, the EC₅₀ is about 0.08. In some embodiments, the EC₅₀ is about 0.3.

In some embodiments, the EC₅₀ is determined in a cell killing assay at 72 hours with human CD8+ T cells and STEAP1-expressing LNCaPX1.2KO3-13 cells. In some embodiments, the EC₅₀ is about 0.1 to about 0.8 (e.g., about 0.1, about 0.15, about 0.2, about 0.25, about 0.3, about 0.35, about 0.4, about 0.45, about 0.5, about 0.55, about 0.6, about 0.65, about 0.7, about 0.75, or about 0.8). In some embodiments, the EC₅₀ is about 0.1 or about 0.7. In some embodiments, the EC₅₀ is about 0.1.

In some embodiments, the EC₅₀ is about 0.7.

In some embodiments, the multispecific antigen-binding molecule binds to STEAP1 monovalently. In other embodiments, the multispecific antigen-binding molecule binds to STEAP1 multivalently (e.g., bivalently).

Antibodies that Bind to a Specific STEAP1 Epitope

In certain embodiments, an antibody of the invention comprises a first antigen-binding domain that binds to a STEAP1 protein. In some instances, the first antigen-binding domain binds to a human STEAP1 protein. In other instances, the first antigen-binding domain binds to a primate STEAP1 protein, e.g., a STEAP1 protein from cynomolgus monkey or Pongo abelili.

In some embodiments, the antibody comprises a first antigen-binding domain that binds to human STEAP1 at one or more residues selected from Ser101, His102, Gln103, Trp195, Gln198, Gln202, and Lys281, wherein the residue positions 101, 102, 103, 195, 198, 202, and 281 correspond to positions 101, 102, 103, 195, 198, 202, and 281 set forth in SEQ ID NO: 65. In some instances, the first antigen-binding domain binds to at least one residue selected from Ser101, His102, Gln103, Trp195, Gln198, Gln202, and Lys281 of SEQ ID NO: 65.

In some instances, a residue from the first antigen-binding domain forms a hydrogen bond with at least one residue selected from Ser101, His102, Gln103, Trp195, Gln198, Gln202, and Lys281. In some cases, at least one residue selected from Leu56, Ser73, Asn74, Gly101, Tyr103, and Tyr107 of the VH of the first antigen-binding domain forms a hydrogen bond with at least one residue selected from Ser101, His102, Gln103, Trp195, Gln198, Gln202, and Lys281 of STEAP1, wherein the residue positions 56, 73, 74, 101, 103, and 107 correspond to positions 56, 73, 74, 101, 103, and 107 set forth in SEQ ID NO: 18. In some cases, at least one residue selected from Tyr35 or Tyr54 of the VL of the first antigen-binding domain forms a hydrogen bond with at least one residue selected from Gln202 or Gln201, wherein the residue positions 35 and 54 correspond to positions 35 and 54 set forth in SEQ ID NO: 8. In some cases, residue Asn203 and/or Lys204 of human STEAP1 form van der Waals interactions with one or more VL CDR residues. In some cases, the residues that form the hydrogen bonds are as illustrated in Table 3.

TABLE 3

| | First antigen-binding domain | Human STEAP1 |
|---|---|---|
| VH | Tyr103 | Trp195 |
| | Gly101 | Gln202 |
| | Tyr107 | Gln198 |
| | Leu56 | Lys281 |
| | Ser73 | His102 |
| | Asn74 | Ser101 |
| | Asn74 | Gln103 |
| VL | Tyr35 | Gln202 |
| | Tyr54 | Gln201 |

In some embodiments, the antibody of the invention is a monospecific antibody described supra under Section A “antigen-binding molecules that binds to STEAP1.” In some instances, the antibody comprises a VH region comprising CDR-H1, CDR-H2, and CDR-H3 of a VH sequence selected from SEQ ID NOs: 7, 17-25, 30-34, 38, and 68; and a VL region comprising CDR-L1, CDR-L2, and CDR-L3 of a VL sequence selected from SEQ ID NOs: 8, 26-29, 39, and 69. In some cases, the antibody comprises six CDRs in which:
CDR-H1 comprises Xaa¹Xaa²YMA (SEQ ID NO: 35);
wherein
   Xaa¹ is Asp (D) or Asn (N); and
   Xaa² is His (H), Tyr (Y), or Phe (F);
CDR-H2 comprises YIXaa³YDGXaa⁴Xaa⁵TXaa⁶YGDSVKG (SEQ ID NO: 36);
wherein
   Xaa³ is Asp (D) or Ser (S);
   Xaa⁴ is Gly (G), Asp (D), or Leu (L);
   Xaa⁵ is Ser (S), Asp (D), or Asn (N); and
   Xaa⁶ is Ser (S) or Tyr (Y);
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and
CDR-L3 comprises the amino acid sequence of SEQ ID NO: 6.

In some cases, the antibody comprises six CDRs, in which:
CDR-H1 comprises GFTFSXaa¹⁰Xaa¹¹ (SEQ ID NO: 63);
wherein
   Xaa¹⁰ is Asn (N) or Asp (D); and
   Xaa¹¹ is Tyr (Y), Phe (F), or His (H);
CDR-H2 comprises Xaa¹²YDGXaa¹³Xaa¹⁴ (SEQ ID NO: 64);
wherein
   Xaa¹² is Asp (D) or Ser (S);
   Xaa¹³ is Gly (G), Asp (D), or Leu (L); and
   Xaa¹⁴ is Ser (S), Asp (D), or Asn (N);
CDR-H3 comprises RSGXaa⁷YHVGYAMXaa⁸Xaa⁹ (SEQ ID NO: 37);
wherein
   Xaa⁷ is Phe (F) or Tyr (Y);
   Xaa⁸ is Asn (N) or Asp (D); and
   Xaa⁹ is Ala (A) or Gly (G);
CDR-L1 comprises the amino acid sequence of SEQ ID NO: 4;
CDR-L2 comprises the amino acid sequence of SEQ ID NO: 5; and
CDR-L3 comprises the amino acid sequence of SEQ ID NO: 6.

In some embodiments, the antibody of the invention is a multispecific antibody described supra under Section B “Multispecific antigen-binding molecules.” In some instances, the antibody comprises (A) a first antigen-binding domain that binds to STEAP1 and comprises an anti-STEAP1 heavy chain variable region (VH) comprising CDR-H1, CDR-H2, and/or CDR-H3 of a VH sequence selected from SEQ ID NO: 7, 17-25, 30-34, 38, and 68; and/or an anti-STEAP1 light chain variable region (VL) comprising CDR-L1, CDR-L2, and/or CDR-L3 of a VL sequence selected from SEQ ID NO: 8, 26-29, 39, and 69; and (B) a second antigen-binding domain that binds to a T cell receptor (e.g., CD3).

In some instances, the antibody comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises Xaa¹Xaa²YMA (SEQ ID NO: 35);
   wherein
      Xaa¹ is Asp (D) or Asn (N); and
      Xaa² is His (H), Tyr (Y), or Phe (F);
   CDR-H2 comprises YIXaa³YDGXaa⁴Xaa⁵TXaa⁶YGDSVKG (SEQ ID NO: 36);
   wherein
      Xaa³ is Asp (D) or Ser (S);
      Xaa⁴ is Gly (G), Asp (D), or Leu (L);
      Xaa⁵ is Ser (S), Asp (D), or Asn (N); and
      Xaa⁶ is Ser (S) or Tyr (Y); and
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor (e.g., CD3).

In some embodiments, the antibody comprises (A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
   CDR-H1 comprises GFTFSXaa¹⁰Xaa¹¹ (SEQ ID NO: 63);
   wherein
      Xaa¹⁰ is Asn (N) or Asp (D); and
      Xaa¹¹ is Tyr (Y), Phe (F), or His (H);
   CDR-H2 comprises Xaa¹²YDGXaa¹³Xaa¹⁴ (SEQ ID NO: 64);
   wherein
      Xaa¹² is Asp (D) or Ser (S);
      Xaa¹³ is Gly (G), Asp (D), or Leu (L); and
      Xaa¹⁴ is Ser (S), Asp (D), or Asn (N);
   CDR-H3 comprises RSGXaa⁷YHVGYAMXaa⁸Xaa⁹ (SEQ ID NO: 37);
   wherein
      Xaa⁷ is Phe (F) or Tyr (Y);
      Xaa⁸ is Asn (N) or Asp (D); and
      Xaa⁹ is Ala (A) or Gly (G);
   CDR-L1 comprises the amino acid sequence of SEQ ID NO: 4;
   CDR-L2 comprises the amino acid sequence of SEQ ID NO: 5; and/or
   CDR-L3 comprises the amino acid sequence of SEQ ID NO: 6; and
(B) a second antigen-binding domain that binds to a T cell receptor (e.g., CD3).

### Peptide Linkers Fusing the First and Second Antigen-Binding Domains

In some embodiments, a multispecific antigen-binding molecule (e.g., a bispecific or trispecific antigen-binding molecule) of the invention features a structure wherein the C-terminus of the first antigen-binding domain is fused to the N-terminus of the second antigen-binding domain via a peptide linker. The peptide linker can be 5-20 amino acids in length (e.g., 5-10, 10-15, or 15-20, e.g., 5, 6, 7, 8, 9, 10, 11, 12, 13, 14, 15, 16, 17, 18, 19, or 20 amino acids in length). In some embodiments, the peptide linker comprises the natural amino acid sequence of the variable heavy chain hinge region (e.g., DKTHT). In some embodiments, the peptide linker comprises a (Gly₄Ser)ₙ linker (or (G₄S)ₙ linker), in which n is from 1 to 10, 2 to 10, 3 to 10, 4 to 10, 5 to 10, 6 to 10, 2 to 6, 2 to 6, 3 to 6, or 4 to 6. In some instances, n is 1, 2, 3, 4, 5, 6, 7, 8, 9, or 10. In some embodiments, the peptide linker comprises a G₄SG₂ linker. In some embodiments, the peptide linker comprises the G₄SG₂ linker and the hinge region (e.g., DKTHT). In some embodiments, the peptide linker comprises a plurality of glycines, alanines, or a combination thereof.

### Fc Domains

Antigen-binding molecules (e.g., monospecific and/or multispecific antigen-binding molecules such as bispecific or trispecific antigen-binding molecules) of the invention may feature an Fc domain. The Fc domain may be an IgG Fc domain (e.g., an IgG₁, IgG₂, or IgG₄ Fc domain). For example, the Fc domain can be a human Fc domain. In some embodiments, the Fc domain comprises one or more amino acid substitution(s) that reduces binding to an Fc receptor and/or effector function. For example, in some embodiments, the one or more amino acid substitutions that reduces binding to an Fc receptor and/or effector function is at one or more position selected from the group of L234, L235, and P329 (e.g., wherein the first Fc subunit and the second Fc subunit each comprises the amino acid substitutions of L234A, L235A and P329G). The Fc receptor may be, for example, an Fcγ receptor. Thus, the antigen-binding molecules (e.g., monospecific and/or multispecific antigen-binding molecules such as bispecific or trispecific antigen-binding molecules) of the invention can be configured to reduce antibody-dependent cell-mediated cytotoxicity (ADCC).

In some instances, the Fc domain comprises a modification configured to promote the association of the first Fc subunit with the second Fc subunit. “Knob-in-hole” engineering of bispecific antibodies may be utilized to generate a first arm containing a knob and a second arm containing the hole into which the knob of the first arm may bind. The knob of the multispecific antigen-binding molecules of the invention may be an anti-STEAP1 arm in one embodiment. Alternatively, the knob of the multispecific antigen-binding molecules of the invention may be an anti-CD3 arm. The hole of the multispecific antigen-binding molecules of the invention may be an anti-STEAP1 arm in one embodiment. Alternatively, the hole of the multispecific antigen-binding molecules of the invention may be an anti-CD3 arm. Multispecific antigen-binding molecules such as bispecific antibodies may also be engineered using immunoglobulin crossover (also known as Fab domain exchange or CrossMab format) technology (see e.g., WO2009/080253; Schaefer et al., Proc. Natl. Acad. Sci. USA, 108:11187-11192 (2011)). Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); or by using leucine zippers to produce bi-specific antibodies (see, e.g., Kostelny et al., J. Immunol., 148(5):1547-1553 (1992)).

An amino acid residue in the CH3 domain of the second Fc subunit may be replaced with an amino acid residue having a larger side chain volume, thereby generating a protuberance (e.g., a knob) within the CH3 domain of the second Fc subunit which is positionable in a cavity (e.g., a hole) within the CH3 domain of the first Fc subunit, and an amino acid residue in the CH3 domain of the first Fc subunit may be replaced with an amino acid residue having a smaller side chain volume, thereby generating a cavity (e.g., a hole) within the CH3 domain of the first Fc subunit within which the protuberance (e.g., a knob) within the CH3 domain of the second Fc subunit may be positionable. In some embodiments, the CH3 domain of the second Fc subunit comprises the amino acid substitution of T366, and the CH3 domain of the first Fc subunit comprises amino acid substitutions at one, two, or all three of T366, L368, and/or Y407 (all EU numbering). In some embodiments, the CH3 domain of the second Fc subunit comprises the amino acid substitution of T366W, and the CH3 domain of the first Fc subunit comprises one, two, or all three amino acid substitutions of T366S, L368A, and/or Y407V (all EU numbering).

In some embodiments, the multispecific antigen-binding molecule described herein comprises an anti-STEAP1 arm comprising N297G (EU numbering) and T366W (EU numbering) substitutions in the heavy chain, and an anti-CD3 arm comprising N297G (EU numbering), T366S (EU numbering), L368A (EU numbering), and Y407V (EU numbering) substitutions in the heavy chain. In some embodiments, the multispecific antigen-binding molecule described herein comprises an anti-STEAP1 arm comprising N297G (EU numbering) and T366W (EU numbering) substitutions in the heavy chain, and an anti-CD3 arm comprising N297G (EU numbering), T366S (EU numbering), L368A (EU numbering), and Y407V (EU numbering) substitutions in the heavy chain.

In some embodiments, the multispecific antigen-binding molecule described herein comprises a) a first heavy chain/light chain pair binding to a first antigen which comprises a first heavy chain polypeptide (H1) and a first light chain polypeptide (L1), and b) a second heavy chain/light chain pair binding to a second antigen which comprises a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a positively charged residue, an amino acid at Q39 (Kabat numbering) in the VH domain of H1 is replaced with a negatively charged residue, an amino acid at V133 (EU numbering) in the CL domain of L1 is replaced with a negatively charged residue, and an amino acid at Q38 (Kabat numbering) in the VL domain of L1 is replaced with a positively charged residue; and an amino acid at Q39 (Kabat numbering) in the VH domain of H2 is replaced with a positively charged residue and an amino acid at Q38 (Kabat numbering) in the VL domain of L2 is replaced with a negatively charged residue; or (ii) an amino acid at S183 (EU numbering) in the CH1 domain of H1 is replaced with a negatively charged residue, an amino acid at Q39 (Kabat numbering) in the VH domain of H1 is replaced with a positively charged residue, an amino acid at V133 (EU numbering) in the CL domain of L1 is replaced with a positively charged residue, and an amino acid at Q38 (Kabat numbering) in the VL domain of L1 is replaced with a negatively charged residue; and an amino acid at Q39 (Kabat numbering) in the VH domain of H2 is replaced with a negatively charged residue and an amino acid at Q38 (Kabat numbering) in the VL domain of L2 is replaced with a positively charged residue. In some embodiments, the positively charged residue is selected from R and K and the negatively charged residue is selected from D and E. In some embodiments, the positively charged residue is R. In other embodiments, the positively charged residue is K. In some embodiments, the negatively charged residue is D. In other embodiments, the negatively charged residue is E. In some embodiments, the first antigen is STEAP1 and the second antigen is CD3. In other embodiments, the first antigen is CD3 and the second antigen is STEAP1.

For example, in some embodiments, the multispecific antigen-binding molecule described herein comprises an anti-STEAP1 arm comprising a first heavy chain polypeptide (H1) and a first light chain polypeptide (L1), and an anti-CD3 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with an R or K residue, an amino acid at Q39 (Kabat numbering) in the VH domain of H1 is replaced with a D or E residue, an amino acid at V133 (EU numbering) in the CL domain of L1 is replaced with a D or E residue, and an amino acid at Q38 (Kabat numbering) in the VL domain of L1 is replaced with an R or K residue; and an amino acid at Q39 (Kabat numbering) in the VH domain of H2 is replaced with an R or K residue and an amino acid at Q38 (Kabat numbering) in the VL domain of L2 is replaced with a D or E residue; or (ii) an amino acid at S183 (EU numbering) in the CH1 domain of H1 is replaced with a D or E residue, an amino acid at Q39 (Kabat numbering) in the VH domain of H1 is replaced with an R or K residue, an amino acid at V133 (EU numbering) in the CL domain of L1 is replaced with an R or K residue, and an amino acid at Q38 (Kabat numbering) in the VL domain of L1 is replaced with a D or E residue; and an amino acid at Q39 (Kabat numbering) in the VH domain of H2 is replaced with a D or E residue and an amino acid at Q38 (Kabat numbering) in the VL domain of L2 is replaced with an R or K residue.

In some embodiments, the multispecific antigen-binding molecule described herein comprises an anti-STEAP1 arm comprising a first heavy chain polypeptide (H1) and a first light chain polypeptide (L1), and an anti-CD3 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a K residue, an amino acid at Q39 (Kabat numbering) in the VH domain of H1 is replaced with an E residue, an amino acid at V133 (EU numbering) in the CL domain of L1 is replaced with an E residue, and an amino acid at Q38 (Kabat numbering) in the VL domain of L1 is replaced with a K residue; and an amino acid at Q39 (Kabat numbering) in the VH domain of H2 is replaced with a K residue and an amino acid at Q38 (Kabat numbering) in the VL domain of L2 is replaced with an E residue; or (ii) an amino acid at S183 (EU numbering) in the CH1 domain of H1 is replaced with an E residue, an amino acid at Q39 (Kabat numbering) in the VH domain of H1 is replaced with a K residue, an amino acid at V133 (EU numbering) in the CL domain of L1 is replaced with a K residue, and an amino acid at Q38 (Kabat numbering) in the VL domain of L1 is replaced with an E residue; and an amino acid at Q39 (Kabat numbering) in the VH domain of H2 is replaced with an E residue and an amino acid at Q38 (Kabat numbering) in the VL domain of L2 is replaced with a K residue.

For example, in other embodiments, the multispecific antigen-binding molecule described herein comprises an anti-CD3 arm comprising a first heavy chain polypeptide (H1) and a first light chain polypeptide (L1), and an anti-STEAP1 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with an R or K residue, an amino acid at Q39 (Kabat numbering) in the VH domain of H1 is replaced with a D or E residue, an amino acid at V133 (EU numbering) in the CL domain of L1 is replaced with a D or E residue, and an amino acid at Q38 (Kabat numbering) in the VL domain of L1 is replaced with an R or K residue; and an amino acid at Q39 (Kabat numbering) in the VH domain of H2 is replaced with an R or K residue and an amino acid at Q38 (Kabat numbering) in the VL domain of L2 is replaced with a D or E residue; or (ii) an amino acid at S183 (EU numbering) in the CH1 domain of H1 is replaced with a D or E residue, an amino acid at Q39 (Kabat numbering) in the VH domain of H1 is replaced with an R or K residue, an amino acid at V133 (EU numbering) in the CL domain of L1 is replaced with an R or K residue, and an amino acid at Q38 (Kabat numbering) in the VL domain of L1 is replaced with a D or E residue; and an amino acid at Q39 (Kabat numbering) in the VH domain of H2 is replaced with a D or E residue and an amino acid at Q38 (Kabat numbering) in the VL domain of L2 is replaced with an R or K residue.

In some embodiments, the multispecific antigen-binding molecule described herein comprises an anti-CD3 arm comprising a first heavy chain polypeptide (H1) and a first light chain polypeptide (L1), and an anti-STEAP1 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a K residue, an amino acid at Q39 (Kabat numbering) in the VH domain of H1 is replaced with an E residue, an amino acid at V133 (EU numbering) in the CL domain of L1 is replaced with an E residue, and an amino acid at Q38 (Kabat numbering) in the VL domain of L1 is replaced with a K residue; and an amino acid at Q39 (Kabat numbering) in the VH domain of H2 is replaced with a K residue and an amino acid at Q38 (Kabat numbering) in the VL domain of L2 is replaced with an E residue; or (ii) an amino acid at S183 (EU numbering) in the CH1 domain of H1 is replaced with an E residue, an amino acid at Q39 (Kabat numbering) in the VH domain of H1 is replaced with a K residue, an amino acid at V133 (EU numbering) in the CL domain of L1 is replaced with a K residue, and an amino acid at Q38 (Kabat numbering) in the VL domain of L1 is replaced with an E residue; and an amino acid at Q39 (Kabat numbering) in the VH domain of H2 is replaced with an E residue and an amino acid at Q38 (Kabat numbering) in the VL domain of L2 is replaced with a K residue.

In some embodiments, the multispecific antigen-binding molecule described herein comprises an anti-STEAP1 arm comprising Q39K (Kabat numbering) and S183E (EU numbering) substitutions in the heavy chain and Q38E (Kabat numbering) and V133K (EU numbering) substitutions in the light chain; and an anti-CD3 arm comprising a Q39E (Kabat numbering) substitution in the heavy chain and a Q38K (Kabat numbering) substitution in the light chain.

In some embodiments, the multispecific antigen-binding molecule described herein comprises an anti-CD3 arm comprising Q39K (Kabat numbering) and S183E (EU numbering) substitutions in the heavy chain and Q38E (Kabat numbering) and V133K (EU numbering) substitutions in the light chain; and an anti-STEAP1 arm comprising a Q39E (Kabat numbering) substitution in the heavy chain and a Q38K (Kabat numbering) substitution in the light chain.

In some embodiments, the multispecific antigen-binding molecule described herein comprises an anti-STEAP1 arm comprising Q39E (Kabat numbering) and S183K (EU numbering) substitutions in the heavy chain and Q38K (Kabat numbering) and V133E (EU numbering) substitutions in the light chain; and an anti-CD3 arm comprising a Q39K (Kabat numbering) substitution in the heavy chain and a Q38E (Kabat numbering) substitution in the light chain.

In some embodiments, the multispecific antigen-binding molecule described herein comprises an anti-CD3 arm comprising Q39E (Kabat numbering) and S183K (EU numbering) substitutions in the heavy chain and Q38K (Kabat numbering) and V133E (EU numbering) substitutions in the light chain; and an anti-STEAP1 arm comprising a Q39K (Kabat numbering) substitution in the heavy chain and a Q38E (Kabat numbering) substitution in the light chain.

In some embodiments, the multispecific antigen-binding molecule described herein comprises a) a first heavy chain/light chain pair binding to a first antigen which comprises a first heavy chain polypeptide (H1) and a first light chain polypeptide (L1), and b) a second heavy chain/light chain pair binding to a second antigen which comprises a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a positively charged residue, an amino acid at Q39 (Kabat numbering) in the VH domain of H1 is replaced with a negatively charged residue, an amino acid at V133 (EU numbering) in the CL domain of L1 is replaced with a negatively charged residue, an amino acid at Q38 (Kabat numbering) in the VL domain of L1 is replaced with a positively charged residue, an amino acid at S183 (EU numbering) in the CH1 domain of H2 is replaced with a negatively charged residue, an amino acid at Q39 (Kabat numbering) in the VH domain of H2 is replaced with a positively charged residue, an amino acid at Q38 (Kabat numbering) in the VL domain of L2 is replaced with a negatively charged residue, and an amino acid at V133 (EU numbering) in the CL domain of L2 is replaced with a positively charged residue; or (ii) an amino acid at S183 (EU numbering) in the CH1 domain of H1 is replaced with a negatively charged residue, an amino acid at Q39 (Kabat numbering) in the VH domain of H1 is replaced with a positively charged residue, an amino acid at V133 (EU numbering) in the CL domain of L1 is replaced with a positively charged residue, an amino acid at Q38 (Kabat numbering) in the VL domain of L1 is replaced with a negatively charged residue, an amino acid at S183 (EU numbering) in the CH1 domain of H2 is replaced with a positively charged residue, an amino acid at Q39 (Kabat numbering) in the VH domain of H2 is replaced with a negatively charged residue, an amino acid at Q38 (Kabat numbering) in the VL domain of L2 is replaced with a positively charged residue, and an amino acid at V133 (EU numbering) in the CL domain of L2 is replaced with a negatively charged residue. In some embodiments, the positively charged residue is selected from R and K and the negatively charged residue is selected from D and E. In some embodiments, the positively charged residue is R. In other embodiments, the positively charged residue is K. In some embodiments, the negatively charged residue is D. In other embodiments, the negatively charged residue is E. In some embodiments, the first antigen is STEAP1 and the second antigen is CD3. In other embodiments, the first antigen is CD3 and the second antigen is STEAP1.

For example, in some embodiments, the multispecific antigen-binding molecule described herein comprises an anti-STEAP1 arm comprising a first heavy chain polypeptide (H1) and a first light chain polypeptide (L1), and an anti-CD3 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with an R or K residue, an amino acid at Q39 (Kabat numbering) in the VH domain of H1 is replaced with a D or E residue, an amino acid at V133 (EU numbering) in the CL domain of L1 is replaced with a D or E residue, an amino acid at Q38 (Kabat numbering) in the VL domain of L1 is replaced with an R or K residue, an amino acid at S183 (EU numbering) in the CH1 domain of H2 is replaced with a D or E residue, an amino acid at Q39 (Kabat numbering) in the VH domain of H2 is replaced with an R or K residue, an amino acid at Q38 (Kabat numbering) in the VL domain of L2 is replaced with a D or E residue, and an amino acid at V133 (EU numbering) in the CL domain of L2 is replaced with an R or K residue; or (ii) an amino acid at S183 (EU numbering) in the CH1 domain of H1 is replaced with a D or E residue, an amino acid at Q39 (Kabat numbering) in the VH domain of H1 is replaced with an R or K residue, an amino acid at V133 (EU numbering) in the CL domain of L1 is replaced with an R or K residue, an amino acid at Q38 (Kabat numbering) in the VL domain of L1 is replaced with a D or E residue, an amino acid at S183 (EU numbering) in the CH1 domain of H2 is replaced with an R or K residue, an amino acid at Q39 (Kabat numbering) in the VH domain of H2 is replaced with a D or E residue, an amino acid at Q38 (Kabat numbering) in the VL domain of L2 is replaced with an R or K residue, and an amino acid at V133 (EU numbering) in the CL domain of L2 is replaced with a D or E residue.

For example, in some embodiments, the multispecific antigen-binding molecule described herein comprises an anti-STEAP1 arm comprising a first heavy chain polypeptide (H1) and a first light chain polypeptide (L1), and an anti-CD3 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a K residue, an amino acid at Q39 (Kabat numbering) in the VH domain of H1 is replaced with an E residue, an amino acid at V133 (EU numbering) in the CL domain of L1 is replaced with an E residue, an amino acid at Q38 (Kabat numbering) in the VL domain of L1 is replaced with a K residue, an amino acid at S183 (EU numbering) in the CH1 domain of H2 is replaced with an E residue, an amino acid at Q39 (Kabat numbering) in the VH domain of H2 is replaced with a K residue, an amino acid at Q38 (Kabat numbering) in the VL domain of L2 is replaced with an E residue, and an amino acid at V133 (EU numbering) in the CL domain of L2 is replaced with a K residue; or (ii) an amino acid at S183 (EU numbering) in the CH1 domain of H1 is replaced with an E residue, an amino acid at Q39 (Kabat numbering) in the VH domain of H1 is replaced with a K residue, an amino acid at V133 (EU numbering) in the CL domain of L1 is replaced with a K residue, an amino acid at Q38 (Kabat numbering) in the VL domain of L1 is replaced with an E residue, an amino acid at S183 (EU numbering) in the CH1 domain of H2 is replaced with a K residue, an amino acid at Q39 (Kabat numbering) in the VH domain of H2 is replaced with an E residue, an amino acid at Q38 (Kabat numbering) in the VL domain of L2 is replaced with a K residue, and an amino acid at V133 (EU numbering) in the CL domain of L2 is replaced with an E residue.

In other examples, in some embodiments, the multispecific antigen-binding molecule described herein comprises an anti-CD3 arm comprising a first heavy chain polypeptide (H1) and a first light chain polypeptide (L1), and an anti-STEAP1 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with an R or K residue, an amino acid at Q39 (Kabat numbering) in the VH domain of H1 is replaced with a D or E residue, an amino acid at V133 (EU numbering) in the CL domain of L1 is replaced with a D or E residue, an amino acid at Q38 (Kabat numbering) in the VL domain of L1 is replaced with an R or K residue, an amino acid at S183 (EU numbering) in the CH1 domain of H2 is replaced with a D or E residue, an amino acid at Q39 (Kabat numbering) in the VH domain of H2 is replaced with an R or K residue, an amino acid at Q38 (Kabat numbering) in the VL domain of L2 is replaced with a D or E residue, and an amino acid at V133 (EU numbering) in the CL domain of L2 is replaced with an R or K residue; or (ii) an amino acid at S183 (EU numbering) in the CH1 domain of H1 is replaced with a D or E residue, an amino acid at Q39 (Kabat numbering) in the VH domain of H1 is replaced with an R or K residue, an amino acid at V133 (EU numbering) in the CL domain of L1 is replaced with an R or K residue, an amino acid at Q38 (Kabat numbering) in the VL domain of L1 is replaced with a D or E residue, an amino acid at S183 (EU numbering) in the CH1 domain of H2 is replaced with an R or K residue, an amino acid at Q39 (Kabat numbering) in the VH domain of H2 is replaced with a D or E residue, an amino acid at Q38 (Kabat numbering) in the VL domain of L2 is replaced with an R or K residue, and an amino acid at V133 (EU numbering) in the CL domain of L2 is replaced with a D or E residue.

For example, in some embodiments, the multispecific antigen-binding molecule described herein comprises an anti-CD3 arm comprising a first heavy chain polypeptide (H1) and a first light chain polypeptide (L1), and an anti-STEAP1 arm comprising a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a K residue, an amino acid at Q39 (Kabat numbering) in the VH domain of H1 is replaced with an E residue, an amino acid at V133 (EU numbering) in the CL domain of L1 is replaced with an E residue, an amino acid at Q38 (Kabat numbering) in the VL domain of L1 is replaced with a K residue, an amino acid at S183 (EU numbering) in the CH1 domain of H2 is replaced with an E residue, an amino acid at Q39 (Kabat numbering) in the VH domain of H2 is replaced with a K residue, an amino acid at Q38 (Kabat numbering) in the VL domain of L2 is replaced with an E residue, and an amino acid at V133 (EU numbering) in the CL domain of L2 is replaced with a K residue; or (ii) an amino acid at S183 (EU numbering) in the CH1 domain of H1 is replaced with an E residue, an amino acid at Q39 (Kabat numbering) in the VH domain of H1 is replaced with a K residue, an amino acid at V133 (EU numbering) in the CL domain of L1 is replaced with a K residue, an amino acid at Q38 (Kabat numbering) in the VL domain of L1 is replaced with an E residue, an amino acid at S183 (EU numbering) in the CH1 domain of H2 is replaced with a K residue, an amino acid at Q39 (Kabat numbering) in the VH domain of H2 is replaced with an E residue, an amino acid at Q38 (Kabat numbering) in the VL domain of L2 is replaced with a K residue, and an amino acid at V133 (EU numbering) in the CL domain of L2 is replaced with an E residue.

In some embodiments, the multispecific antigen-binding molecule described herein comprises an anti-STEAP1 arm comprising Q39K (Kabat numbering) and S183E (EU numbering) substitutions in the heavy chain and Q38E (Kabat numbering) and V133K (EU numbering) substitutions in the light chain; and an anti-CD3 arm comprising a Q39E (Kabat numbering) substitution and an S183K (EU numbering) substitution in the heavy chain and a Q38K (Kabat numbering) substitution and a V133E (EU numbering) substitution in the light chain.

In some embodiments, the multispecific antigen-binding molecule described herein comprises an anti-CD3 arm comprising Q39K (Kabat numbering) and S183E (EU numbering) substitutions in the heavy chain and Q38E (Kabat numbering) and V133K (EU numbering) substitutions in the light chain; and an anti-STEAP1 arm comprising a Q39E (Kabat numbering) substitution and an S183K (EU numbering) substitution in the heavy chain and a Q38K (Kabat numbering) substitution and a V133E (EU numbering) substitution in the light chain.

In some embodiments, the multispecific antigen-binding molecule described herein comprises an anti-STEAP1 arm comprising Q39E (Kabat numbering) and S183K (EU numbering) substitutions in the heavy chain and Q38K (Kabat numbering) and V133E (EU numbering) substitutions in the light chain; and an anti-CD3 arm comprising a Q39K (Kabat numbering) substitution and an S183E (EU numbering) substitution in the heavy chain and a Q38E (Kabat numbering) substitution and a V133K (EU numbering) substitution in the light chain.

In some embodiments, the multispecific antigen-binding molecule described herein comprises an anti-CD3 arm comprising Q39E (Kabat numbering) and S183K (EU numbering) substitutions in the heavy chain and Q38K (Kabat numbering) and V133E (EU numbering) substitutions in the light chain; and an anti-STEAP1 arm comprising a Q39K (Kabat numbering) substitution and an S183E (EU numbering) substitution in the heavy chain and a Q38E (Kabat numbering) substitution and a V133K (EU numbering) substitution in the light chain.

In some embodiments, the multispecific antigen-binding molecule described herein comprises an anti-STEAP1 arm comprising S183K (EU numbering), N297G (EU numbering) and T366W (EU numbering) substitutions in the heavy chain constant region and V133E (EU numbering) substitution in the light chain constant region, and an anti-CD3 arm comprising S183E (EU numbering), N297G (EU numbering), T366S (EU numbering), L368A (EU numbering), and Y407V (EU numbering) substitutions in the heavy chain constant region and V133K (EU numbering) substitution in the light chain constant region. In some embodiments, the multispecific antigen-binding molecule described herein comprises an anti-STEAP1 arm comprising S183E (EU numbering), N297G (EU numbering), T366S (EU numbering), L368A (EU numbering), and Y407V (EU numbering) substitutions in the heavy chain constant region and V133K (EU numbering) substitution in the light chain constant region, and an anti-CD3 arm comprising S183K (EU numbering), N297G (EU numbering) and T366W (EU numbering) substitutions in the heavy chain constant region and V133E (EU numbering) substitution in the light chain constant region.

In some embodiments, the multispecific antigen-binding molecule described herein comprises an anti-STEAP1 arm comprising Q39E (Kabat numbering), S183K (EU numbering), N297G (EU numbering) and T366W (EU numbering) substitutions in the heavy chain and Q38K (Kabat numbering) and V133E (EU numbering) substitutions in the light chain, and an anti-CD3 arm comprising Q39K (Kabat numbering), S183E (EU numbering), N297G (EU numbering), T366S (EU numbering), L368A (EU numbering), and Y407V (EU numbering) substitutions in the heavy chain and Q38E (Kabat numbering) and V133K (EU numbering) substitutions in the light chain. In some embodiments, the multispecific antigen-binding molecule described herein comprises an anti-STEAP1 arm comprising Q39K (Kabat numbering), S183E (EU numbering), N297G (EU numbering), T366S (EU numbering), L368A (EU numbering), and Y407V (EU numbering) substitutions in the heavy chain and Q38E (Kabat numbering) and V133K (EU numbering) substitutions in the light chain, and an anti-CD3 arm comprising Q39E (Kabat numbering), S183K (EU numbering), N297G (EU numbering) and T366W (EU numbering) substitutions in the heavy chain and Q38K (Kabat numbering) and V133E (EU numbering) substitutions in the light chain.

It is expressly contemplated that antigen-binding molecules described herein for use in any of the instances enumerated herein may have any of the features, singly or in combination, described in Sections 1-6 below.

### 1. Antibody Affinity

In certain instances, an antigen-binding molecule (e.g., monospecific and/or multispecific antigen-binding molecules such as bispecific or trispecific antigen-binding molecules) of the invention has an equilibrium dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one instance, K_{D} is measured by a radiolabeled antigen binding assay (RIA). In one instance, an RIA is performed with the Fab version of an antigen-binding molecule of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (about 23 °C). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™. Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, K_{D} is measured using a BIACORE® surface plasmon resonance assay. For example, an assay using a BIACORE®-2000 or a BIACORE®-3000 (BIACORE®, Inc., Piscataway, NJ) is performed at 25 °C with immobilized antigen CM5 chips at ~10 response units (RU). In one instance, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (~0.2 μM) before injection at a flow rate of 5 μl/minute to achieve about 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 °C at a flow rate of about 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio kon/kon. See, for example, Chen et al., J. Mol Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 °C of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

According to a further instance, K_{D} is measured by Kinetic Exclusion Assay (KinExA®), which can be performed by Sapidyne Instruments. The Kinetic Exclusion Assay (KinExA®) measures the equilibrium binding affinity and kinetics in solution. The equilibrium dissociation constant, K_{D}, and association, kₐ, are experimentally determined, while the rate of dissociation, k_{d}, is calculated based on the equation kd=K_{D}×kₐ. In one embodiment, the K_{D} is measured with human STEAP1. In another embodiment, the K_{D} is measured with cynomolgus STEAP1. In one embodiment, an anti-STEAP1/CD3 TDB described herein has a K_{D} for human STEAP1 of 0.01-1 nM, or 1-10 nM, or 10-100 nM, or 100-1,000 nM. In another embodiment, an anti-STEAP1/CD3 TDB described herein has a K_{D} for cynomolgus STEAP1 of 0.01-1 nM, or 1-10 nM, or 10-100 nM, or 100-1,000 nM. In one embodiment, an anti-STEAP1/CD3 TDB described herein has a ka for human STEAP1 of 100-1,000 M⁻¹S⁻¹, or 1,000-10,000 M⁻¹S⁻¹, or 10,000-100,000 M⁻¹S⁻¹, or 100,000-1,000,000 M⁻¹S⁻¹. In another embodiment, an anti-STEAP1/CD3 TDB described herein has a kₐ for cynomolgus STEAP1 of 100-1,000 M⁻¹S⁻¹, or 1,000-10,000 M⁻¹S⁻¹, or 10,000-100,000 M⁻¹S⁻¹, or 100,000-1,000,000 M⁻¹S⁻¹. In one embodiment, an anti-STEAP1/CD3 TDB described herein has a k_{d} for human STEAP1 of 0.0001-0.001 S⁻¹, 0.001-0.01 S⁻¹, or 0.01-0.1 S⁻¹, or 0.1-1 S⁻¹. In another embodiment, an anti-STEAP1/CD3 TDB described herein has a k_{d} for cynomolgus STEAP1 of 0.0001-0.001 S⁻¹, 0.001-0.01 S⁻¹, or 0.01-0.1 S⁻¹, or 0.1-1 S⁻¹.

### 2. Antibody Fragments and Multispecific Antibodies

In certain instances, an antigen-binding molecule provided herein includes one or more antibody fragments. Antibody fragments include, but are not limited to, Fab, Fab′, Fab′-SH, F(ab′)₂, Fv, Fd, single-chain Fv (scFv), single-chain Fab fragment (scFab), trispecific (Fab₃), bispecific (Fab₂), diabody ((V_{L}-V_{H})₂ or (V_{H}-V_{L})₂), triabody (trivalent), tetrabody (tetravalent), minibody ((scFv-C_{H})₂), bispecific single-chain Fv (Bis-scFv), IgGdeltaCH2, scFv-Fc, or (scFv)₂-Fc, and other fragments described below. For a review of certain antibody fragments, see Hudson et al. Nat. Med. 9:129-134 (2003). For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

In some instances, the antibody fragment is a single-chain variable fragment (scFv).

In some instances, the antibody fragment is a single chain Fab fragment.

In certain instances, an antigen-binding molecule provided herein includes a multispecific antigen-binding molecule. In certain aspects, a multispecific antigen-binding molecule provided herein is a multispecific antibody, e.g., a bispecific antibody or a trispecific antibody. “In certain aspects, the multispecific antibody is a bispecific antibody. In certain aspects, the multispecific antibody has three or more binding specificities. In certain aspects, one of the binding specificities is for STEAP1 and the other specificity is for any other antigen (e.g., CD3). Multispecific (e.g., bispecific or trispecific) antibodies may also be used to localize cytotoxic agents or cells to cells which express STEAP1. Multispecific antibodies may be prepared as full length antibodies or antibody fragments.

Various molecular formats for multispecific antibodies are known in the art and are included herein (see e.g., Spiess et al., Mol. Immunol. 67 (2015) 95-106; and Brinkmann et al., MAbs 9 (2017) 182-212).

In some embodiments, a multispecific antibody described herein is a bispecific antibody, designed to simultaneously bind to a surface antigen on a target cell, e.g., a tumor cell, and to an activating, invariant component of the T cell receptor (TCR) complex, such as CD3, for retargeting of T cells to kill target cells. In certain aspects, an antibody provided herein is a multispecific antibody, particularly a bispecific antibody, wherein one of the binding specificities is for STEAP1 and the other is for CD3.

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp. Cell Res. 317, 1255-1260 (2011)); diabodies (Holliger et al., Prot. Eng. 9, 299-305 (1996)) and derivatives thereof, such as tandem diabodies (“TandAb”; Kipriyanov et al., J. Mol. Biol. 293, 41-56 (1999)); “DART” (dual affinity retargeting) molecules which are based on the diabody format but feature a C-terminal disulfide bridge for additional stabilization (Johnson et al., J. Mol. Biol. 399, 436-449 (2010)), and so-called triomabs, which are whole hybrid mouse/rat IgG molecules (reviewed in Seimetz et al., Cancer Treat. Rev. 36, 458-467 (2010)). Particular T cell bispecific antibody formats included herein are described in WO 2013/026833, WO 2013/026839, WO 2016/020309; and Bacac et al., Oncoimmunology 5(8):e1203498 (2016).

Additional multispecific antibody formats include diabodies, antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Multispecific antibodies may also be provided in an asymmetric form with a domain crossover in one or more binding arms of the same antigen specificity, i.e., by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al., Proc. Natl. Acad. Sci. USA 108: 1187-1191 (2011), and Klein et al., MAbs 8:1010-20 (2016)). In one aspect, the multispecific antibody comprises a cross-Fab fragment.

Engineered antibodies with three or more antigen binding sites, including for example, “Octopus antibodies,” or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to STEAP1 as well as another different antigen, e.g., to CD3 (see, e.g., US 2008/0069820 and WO 2015/095539).

Techniques for making multispecific antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al., J. Immunol. 147:60 (1991).

### 3. Chimeric and Humanized Antibodies

In certain instances, an antigen-binding molecule provided herein (e.g., monospecific and/or multispecific antigen-binding molecules such as bispecific or trispecific antigen-binding molecules) is a chimeric antibody. Certain chimeric antibodies are described, e.g., in U.S. Pat. No. 4,816,567; and Morrison et al. Proc. Nat. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the CDR/HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat. Acad. Sci. USA 86:10029-10033 (1989); U.S. Pat. No. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Nat. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain instances, an antigen-binding molecule provided herein (e.g., monospecific and/or multispecific antigen-binding molecules such as bispecific or trispecific antigen-binding molecules) is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

In certain embodiments, an antigen-binding molecule provided herein e.g., monospecific and/or multispecific antigen-binding molecules such as bispecific or trispecific antigen-binding molecules) may include antibodies derived from a library. Antibodies of the invention may be isolated by screening combinatorial libraries for antibodies with the desired activity or activities. Methods for screening combinatorial libraries are reviewed, e.g., in Lerner et al., Nature Reviews 16:498-508 (2016). For example, a variety of methods are known in the art for generating phage display libraries and screening such libraries for antibodies possessing the desired binding characteristics. Such methods are reviewed, e.g., in Frenzel et al., mAbs 8:1177-1194 (2016); Bazan et al., Human Vaccines and Immunotherapeutics 8:1817-1828 (2012) and Zhao et al., Critical Reviews in Biotechnology 36:276-289 (2016) as well as in Hoogenboom et al., Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol. 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO Journal 12: 725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol. 227: 381-388 (1992). Patent publications describing human antibody phage libraries include, for example: U.S. Pat. Nos. 5,750,373; 7,985,840; 7,785,903 and 8,679,490 as well as U.S. Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells, or yeast cells. Methods for yeast surface display are reviewed, e.g., in Scholler et al., Methods Mol. Biol. 503:135-56 (2012) and in Cherf et al. in Methods Mol. Biol. 1319:155-175 (2015) as well as in Zhao et al., Methods Mol. Biol. 889:73-84 (2012). Methods for ribosome display are described, e.g., in He et al., Nucleic Acids Research 25:5132-5134 (1997) and in Hanes et al., Proc. Natl. Acad. Sci. USA 94:4937-4942 (1997).

Antibodies or antibody fragments isolated from human antibody libraries are considered human antibodies or human antibody fragments herein.

### 6. Antigen-Binding Molecule Variants

In certain instances, amino acid sequence variants of the antigen-binding molecules (e.g., monospecific and/or multispecific antigen-binding molecules such as bispecific or trispecific antigen-binding molecules) of the invention are contemplated. For example, it may be desirable to improve the binding affinity and/or other biological properties of the antigen-binding molecule. Amino acid sequence variants of an antigen-binding molecule may be prepared by introducing appropriate modifications into the nucleotide sequence encoding the antigen-binding molecule, or by peptide synthesis. Such modifications include, for example, deletions from, and/or insertions into and/or substitutions of residues within the amino acid sequences of the antigen-binding molecule. Any combination of deletion, insertion, and substitution can be made to arrive at the final construct, provided that the final construct possesses the desired characteristics, for example, antigen-binding.

In certain embodiments, an antigen-binding molecule (e.g., monospecific and/or multispecific antigen-binding molecules such as bispecific or trispecific antigen-binding molecules) of the invention comprises one or more modifications in the VH/VL region and/or CH1/CL region to facilitate correct heavy/light chain pairing. In some embodiments, an antigen-binding molecule (e.g., monospecific and/or multispecific antigen-binding molecules such as bispecific or trispecific antigen-binding molecules) of the invention comprises one or more modifications in the Fc region to facilitate heterodimerization of the two arms of the antigen-binding molecule. Such modifications in the VH/VL region, CH1/CL region, and/or FC region are described in International Patent Publication No. WO 2016/172485, which is herein incorporated by reference in its entirety.

### a) Substitution, Insertion, and Deletion Variants

In certain instances, antigen-binding domain variants having one or more amino acid substitutions are provided. Sites of interest for substitutional mutagenesis include the CDRs and FRs. Conservative substitutions are shown in Table 4 under the heading of “preferred substitutions.” More substantial changes are provided in Table 4 under the heading of “exemplary substitutions,” and as further described below in reference to amino acid side chain classes. Amino acid substitutions may be introduced into an antigen-binding molecule of interest and the products screened for a desired activity, for example, retained/improved antigen binding, decreased immunogenicity, or improved Antibody-Dependent Cell-Mediated Cytotoxicity (ADCC) or Complement Dependent Cytotoxicity (CDC).

TABLE 4
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro; or
(6) aromatic: Trp, Tyr, Phe.

Non-conservative substitutions will entail exchanging a member of one of these classes for another class.

One type of substitutional variant involves substituting one or more hypervariable region residues of a parent antigen-binding molecule (e.g., a humanized or human antibody). Generally, the resulting variant(s) selected for further study will have modifications (e.g., improvements) in certain biological properties (e.g., increased affinity and/or reduced immunogenicity) relative to the parent antigen-binding molecule and/or will have substantially retained certain biological properties of the parent antigen-binding molecule. An exemplary substitutional variant is an affinity matured antibody, which may be conveniently generated, for example, using phage display-based affinity maturation techniques such as those described herein. Briefly, one or more CDR/HVR residues are mutated and the variant antibodies displayed on phage and screened for a particular biological activity (e.g., binding affinity).

Alterations (e.g., substitutions) may be made in CDRs/HVRs, e.g., to improve antibody affinity. Such alterations may be made in CDR/HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al., Methods Mol. Biol. 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR/HVR-directed approaches, in which several CDR/HVR residues (e.g., 4-6 residues at a time) are randomized. CDR/HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more CDRs/HVRs so long as such alterations do not substantially reduce the ability of the antigen-binding molecule to bind antigen. For example, conservative alterations (e.g., conservative substitutions as provided herein) that do not substantially reduce binding affinity may be made in CDRs/HVRs. Such alterations may, for example, be outside of antigen-contacting residues in the CDRs/HVRs. In certain instances of the variant VH and VL sequences provided above, each CDRs/HVR either is unaltered, or contains no more than one, two or three amino acid substitutions.

A useful method for identification of residues or regions of an antigen-binding molecule that may be targeted for mutagenesis is called “alanine scanning mutagenesis” as described by Cunningham and Wells Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antigen-binding molecule with antigen is affected. Further substitutions may be introduced at the amino acid locations demonstrating functional sensitivity to the initial substitutions. Alternatively, or additionally, a crystal structure of an antigen-antigen-binding molecule complex to identify contact points between the antigen-binding molecule and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antigen-binding molecule with an N-terminal methionyl residue. Other insertional variants of the antigen-binding molecule include the fusion to the N- or C-terminus of the antigen-binding molecule to an enzyme (e.g., for ADEPT) or a polypeptide which increases the serum half-life of the antigen-binding molecule.

### b) Glycosylation Variants

In certain instances, antibodies of the invention can be altered to increase or decrease the extent to which the antibody is glycosylated. Addition or deletion of glycosylation sites to an antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention may be made in order to create variants with certain improved properties.

In one instance, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, for example, U.S. Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Appl. No. US 2003/0157108 A1; and WO 2004/056312 A1, Adams et al., especially at Example 11), and knockout cell lines, such as alpha-1,6-fucosyltransferase gene, FUTB, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107). In some examples, any antigen-binding molecule (e.g., monospecific and/or multispecific antigen-binding molecules such as bispecific or trispecific antigen-binding molecules (e.g., antibodies)) described herein may be afucosylated.

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain instances, one or more amino acid modifications may be introduced into the Fc region of an antibody of the invention, thereby generating an Fc region variant. The Fc region variant may comprise a human Fc region sequence (e.g., a human IgG1, IgG2, IgG3, or IgG4 Fc region) comprising an amino acid modification (e.g., a substitution) at one or more amino acid positions.

In certain instances, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g., Hellstrom et al., Proc. Natl. Acad. Sci. USA 83:7059-7063 (1986)) and Hellstrom et al., Proc. Nat. Acad. Sci. USA 82:1499-1502 (1985); U.S. Pat. No. 5,821,337 (see Bruggemann et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI))). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Natl. Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg et al., Blood. 101:1045-1052 (2003); and Cragg et al., Blood. 103:2738-2743 (2004)). FcRn binding and in vivo clearance/half-life determinations can also be performed using methods known in the art (see, e.g., Petkova et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

Certain antibody variants with improved or diminished binding to FcRs are described. See, e.g., U.S. Pat. No. 6,737,056; WO 2004/056312, and Shields et al., J. Biol. Chem. 9(2): 6591-6604 (2001).

In certain instances, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. See, e.g., WO 2012/130831. In another aspect, the substitutions are L234A, L235A and D265A (LALA-DA) in an Fc region derived from a human IgG₁ Fc region.

In some instances, alterations are made in the Fc region that result in altered (i.e., either improved or diminished) C1q binding and/or CDC, e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua et al., J. Biol. Chem. 281:23514-23524(2006)).

Fc region residues critical to the mouse Fc-mouse FcRn interaction have been identified by site-directed mutagenesis (see e.g., Dall'Acqua et al., J. Immunol. 169:5171-5180 (2002)). Residues I253, H310, H433, N434, and H435 (EU numbering of residues) are involved in the interaction (Medesan et al., Eur. J. Immunol. 26:2533 (1996); Firan et al., Int. Immunol. 13:993 (2001); Kim et al., Eur. J. Immunol. 24:542 (1994)). Residues I253, H310, and H435 were found to be critical for the interaction of human Fc with murine FcRn (Kim et al., Eur. J. Immunol. 29:2819 (1999)). Studies of the human Fc-human FcRn complex have shown that residues 1253, S254, H435, and Y436 are crucial for the interaction (Firan et al., Int. Immunol. 13:993 (2001); Shields et al., J. Biol. Chem. 276:6591-6604 (2001)). In Yeung et al., J. Immunol. 182:7667-7671 (2009)), various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are 1253A, H310A and H435A in an Fc region derived from a human IgG1 Fc-region. See, e.g., Grevys et al., J. Immunol. 194: 5497-5508 (2015).

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG₁ Fc-region. (See, e.g., WO 2014/177460).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan and Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain instances, it may be desirable to create cysteine engineered antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541, which is incorporated herein by reference in its entirety.

### e) Antibody Derivatives

In certain instances, an antigen-binding molecule provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, and the like.

In another instance, conjugates of an antigen-binding molecule and nonproteinaceous moiety that may be selectively heated by exposure to radiation are provided. In one instance, the nonproteinaceous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the nonproteinaceous moiety to a temperature at which cells proximal to the antigen-binding molecule-nonproteinaceous moiety are killed.

### f) Immunoconjugates

The invention also provides immunoconjugates comprising antigen-binding molecule conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one instance, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bloorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an antigen-binding molecule as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and tricothecenes.

In another instance, an immunoconjugate comprises an antigen-binding molecule as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I¹²³, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antigen-binding molecule and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate) which are commercially available (e.g., from Pierce Biotechnology, Inc., Rockford, IL., U.S.A).

### III. Recombinant Methods and Compositions

Antigen-binding molecules of the invention may be produced using recombinant methods and compositions, for example, as described in U.S. Pat. No. 4,816,567 and in U.S. Patent Application Publication No. 2013/0078249, which are incorporated herein by reference in their entireties. In one embodiment, an isolated nucleic acid (e.g., a polynucleotide) encoding an antigen-binding molecule described herein is provided. Such a nucleic acid may encode an amino acid sequence comprising the VL and/or an amino acid sequence comprising a VH of a bispecific antigen-binding molecule (e.g., the light and/or heavy chains of either arm of the bispecific antigen-binding molecule). In a further embodiment, one or more vectors (e.g., expression vectors) comprising such a nucleic acid are provided.

Polynucleotides encoding antigen-binding molecules of the invention may be expressed as a single polynucleotide molecule or as multiple (e.g., two or more) polynucleotides that are co-expressed. Polypeptides encoded by polynucleotides that are co-expressed may associate through, e.g., disulfide bonds or other means to form a functional bispecific antigen-binding molecule. For example, a light chain portion of an antigen-binding molecule may be encoded by a separate polynucleotide from the portion of the antigen-binding molecule comprising the heavy chain portion of the antigen binding moiety, an Fc domain subunit and optionally another antigen-binding domain. When co-expressed, the heavy chain polypeptides will associate with the light chain polypeptides to form the antigen-binding molecule. In another example, the portion of the antigen-binding molecule comprising one of the two Fc domain subunits and optionally one or more antigen-binding domains may be encoded by a separate polynucleotide from the portion of the T cell activating antigen-binding molecule comprising the other of the two Fc domain subunits and optionally an antigen binding domain. When co-expressed, the Fc domain subunits will associate to form the Fc domain.

In case of a native antibody or native antibody fragment, two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains, four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e., one vector can comprise more than one of these nucleic acids. Examples of these bispecific antibodies are CrossMabs (see, e.g., Schaefer et al., Proc. Natl. Acad. Sci. USA 108:11187-11191 (2011)). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter et al., Immunotechnol. 2:73, 1996) according to EU index numbering.

In a further embodiment, a host cell comprising such a nucleic acid is provided. In one such embodiment, a host cell comprises (e.g., has been transformed with): (1) a vector comprising a nucleic acid that encodes an amino acid sequence comprising at least one VL of the antigen-binding molecule and an amino acid sequence comprising at least one VH of the antigen-binding molecule, or (2) a first vector comprising a nucleic acid that encodes an amino acid sequence comprising a VL of the antigen-binding molecule and a second vector comprising a nucleic acid that encodes an amino acid sequence comprising a VH of the antigen-binding molecule. In one embodiment, the host cell is eukaryotic, e.g., a Chinese Hamster Ovary (CHO) cell or lymphoid cell.

In one embodiment, a method of making an anti-STEAP1 antigen-binding molecule is provided, wherein the method comprises culturing a host cell comprising a nucleic acid encoding the anti-STEAP1 antigen-binding molecule, as provided above, under conditions suitable for expression of the anti-STEAP1 antigen-binding molecule, and optionally recovering the anti-STEAP1 antigen-binding molecule from the host cell (or host cell culture medium). In another embodiment, the method further comprises culturing a second host cell comprising a second nucleic acid encoding an anti-CD3 antigen-binding molecule that comprises a binding domain that binds CD3. In some embodiments, the host cells are co-cultured. A further embodiment comprises recovering the bispecific antibody from the host cell or the culture medium. In some embodiments, the anti-STEAP1 and anti-CD3 antigen-binding molecules are produced in the same host cell (e.g., a one-cell approach). In some embodiments, the anti-STEAP1 and anti-CD3 antigen-binding molecules are produced in separate host cells (e.g., a two-cell approach).

In the one-cell and two-cell approaches, one or more plasmids encoding the anti-STEAP1/CD3 TDB (e.g., an anti-STEAP1 half-antibody and an anti-CD3 half-antibody) are introduced into one or more host cells for culture and expression of the TDB. In one instance, a single plasmid may encode both the anti-STEAP1 half-antibody and the anti-CD3 half-antibody. Alternatively, the half-antibodies can be encoded by separate plasmids. In another instance, the heavy chain of each half-antibody is encoded on a first plasmid, while the light chain of each half-antibody is encoded on a second plasmid. In the one-cell approach, the anti-STEAP1/CD3 TDB is produced in a single host. In the two-cell approach, the anti-STEAP1/CD3 TDB is produced by expressing the half-antibodies in separate hosts (e.g., separate cultures of the same host cells, or separate cultures of different host cells). In the two-cell approach, the two hosts can be cultured in the same vessel or in different vessels. The two host cultures can be combined prior to lysis and purification of the anti-STEAP1/CD3 TDB or the two half-antibodies can be purified separately.

In some embodiments, an anti-STEAP1/CD3 TDB of the invention that has been modified to include asymmetrical modifications (e.g., mutations of the VH, VL, CH1, CL and/or Fc domains described above) is produced using a one-cell approach, which results in improved correct heavy chain/light chain pairing and/or improved yield of the anti-STEAP1/CD3 TDB as compared to an anti-STEAP1/CD3 TDB that has not been modified to include the asymmetrical modifications.

For recombinant production of an antigen-binding molecule, a polynucleotide encoding an antigen-binding molecule, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antigen-binding molecule).

Suitable host cells for cloning or expression of antigen-binding molecule-encoding vectors include prokaryotic or eukaryotic cells described herein. For example, antigen-binding molecules may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.) After expression, the antigen-binding molecule may be isolated from the bacterial cell paste in a soluble fraction and can be further purified.

In addition to prokaryotes, eukaryotic microbes such as filamentous fungi or yeast are suitable cloning or expression hosts for antigen-binding molecule-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004); and Li et al., Nat. Biotech. 24:210-215 (2004).

Suitable host cells for the expression of glycosylated antigen-binding molecule are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J. Gen Virol. 36:59-74 (1977)); baby hamster kidney cells (BHK); mouse sertoli cells (TM4 cells as described, e.g., in Mather, Biol. Reprod. 23:243-252 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather et al., Annals N.Y. Acad. Sci. 383:44-68 (1982)); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216-4220 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248, Lo, B.K.C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### One-Cell Approach for Anti-STEAP1/CD3 TDB Production

In one approach, the anti-STEAP1/CD3 TDBs can be produced by culturing host cells that have been co-transfected with two plasmids, each encoding one of the two arms of the anti-STEAP1/CD3 TDB (e.g., a first plasmid encoding an anti-STEAP1 half-antibody and a second plasmid encoding an anti-CD3 half-antibody). Transfection of host cells (e.g., bacterial, mammalian, or insect cells) may be performed in a 96-well plate format. To screen for STEAP1 TDB production, approximately 2,000 to 3,000 clones may be picked and assessed by ELISA and intact IgG homogeneous time resolved fluorescence (HTRF) for their ability to bind the target antigen, STEAP1. Clones producing anti-STEAP1/CD3 TDBs capable of binding to STEAP1, or a fragment thereof, may be selected for expansion and further screening (e.g., for binding to CD3). Top clones can then be selected for further analysis based on percent bispecific antibodies (bsAbs) produced, titer, and performance qualification (PQ).

An exemplary one-cell approach that can be used to produce anti-STEAP1/CD3 TBDs of the invention is described in International Patent Application No. PCT/US16/28850 or U.S. Patent Application Publication No. 2018/0177873, which are incorporated herein by reference in its entirety.

For example, provided herein is a method for producing a multispecific antigen-binding molecule disclosed herein, comprising (a) introducing one or more polynucleotide molecules encoding a multispecific antigen-binding molecule into a host cell, wherein the multispecific antigen-binding molecule comprises a) a first heavy chain/light chain pair binding to a first antigen which comprises a first heavy chain polypeptide (H1) and a first light chain polypeptide (L1), and b) a second heavy chain/light chain pair binding to a second antigen which comprises a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a positively charged residue, an amino acid at Q39 (Kabat numbering) in the VH domain of H1 is replaced with a negatively charged residue, an amino acid at V133 (EU numbering) in the CL domain of L1 is replaced with a negatively charged residue, and an amino acid at Q38 (Kabat numbering) in the VL domain of L1 is replaced with a positively charged residue; and an amino acid at Q39 (Kabat numbering) in the VH domain of H2 is replaced with a positively charged residue and an amino acid at Q38 (Kabat numbering) in the VL domain of L2 is replaced with a negatively charged residue; or (ii) an amino acid at S183 (EU numbering) in the CH1 domain of H1 is replaced with a negatively charged residue, an amino acid at Q39 (Kabat numbering) in the VH domain of H1 is replaced with a positively charged residue, an amino acid at V133 (EU numbering) in the CL domain of L1 is replaced with a positively charged residue, and an amino acid at Q38 (Kabat numbering) in the VL domain of L1 is replaced with a negatively charged residue; and an amino acid at Q39 (Kabat numbering) in the VH domain of H2 is replaced with a negatively charged residue and an amino acid at Q38 (Kabat numbering) in the VL domain of L2 is replaced with a positively charged residue; and (b) culturing the host cell under conditions and for a time sufficient to produce the antigen-binding protein. In some embodiments, the positively charged residue is selected from R and K and the negatively charged residue is selected from D and E. In some embodiments, the positively charged residue is R. In other embodiments, the positively charged residue is K. In some embodiments, the negatively charged residue is D. In other embodiments, the negatively charged residue is E. In some embodiments, the first antigen is STEAP1 and the second antigen is CD3. In other embodiments, the first antigen is CD3 and the second antigen is STEAP1.

For example, provided herein is a method for producing a multispecific antigen-binding molecule disclosed herein, comprising (a) providing a host cell comprising one or more polynucleotide molecules encoding a multispecific antigen-binding molecule, wherein the multispecific antigen-binding molecule comprises a) a first heavy chain/light chain pair binding to a first antigen which comprises a first heavy chain polypeptide (H1) and a first light chain polypeptide (L1), and b) a second heavy chain/light chain pair binding to a second antigen which comprises a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a positively charged residue, an amino acid at Q39 (Kabat numbering) in the VH domain of H1 is replaced with a negatively charged residue, an amino acid at V133 (EU numbering) in the CL domain of L1 is replaced with a negatively charged residue, and an amino acid at Q38 (Kabat numbering) in the VL domain of L1 is replaced with a positively charged residue; and an amino acid at Q39 (Kabat numbering) in the VH domain of H2 is replaced with a positively charged residue and an amino acid at Q38 (Kabat numbering) in the VL domain of L2 is replaced with a negatively charged residue; or (ii) an amino acid at S183 (EU numbering) in the CH1 domain of H1 is replaced with a negatively charged residue, an amino acid at Q39 (Kabat numbering) in the VH domain of H1 is replaced with a positively charged residue, an amino acid at V133 (EU numbering) in the CL domain of L1 is replaced with a positively charged residue, and an amino acid at Q38 (Kabat numbering) in the VL domain of L1 is replaced with a negatively charged residue; and an amino acid at Q39 (Kabat numbering) in the VH domain of H2 is replaced with a negatively charged residue and an amino acid at Q38 (Kabat numbering) in the VL domain of L2 is replaced with a positively charged residue; and (b) culturing the host cell under conditions and for a time sufficient to produce the antigen-binding protein. In some embodiments, the positively charged residue is selected from R and K and the negatively charged residue is selected from D and E. In some embodiments, the positively charged residue is R. In other embodiments, the positively charged residue is K. In some embodiments, the negatively charged residue is D. In other embodiments, the negatively charged residue is E. In some embodiments, the first antigen is STEAP1 and the second antigen is CD3. In other embodiments, the first antigen is CD3 and the second antigen is STEAP1.

In some embodiments, the multispecific antigen-binding molecule described herein comprises an anti-STEAP1 arm comprising Q39K (Kabat numbering) and S183E (EU numbering) substitutions in the heavy chain and Q38E (Kabat numbering) and V133K (EU numbering) substitutions in the light chain; and an anti-CD3 arm comprising a Q39E (Kabat numbering) substitution in the heavy chain and a Q38K (Kabat numbering) substitution in the light chain.

In some embodiments, the multispecific antigen-binding molecule described herein comprises an anti-CD3 arm comprising Q39K (Kabat numbering) and S183E (EU numbering) substitutions in the heavy chain and Q38E (Kabat numbering) and V133K (EU numbering) substitutions in the light chain; and an anti-STEAP1 arm comprising a Q39E (Kabat numbering) substitution in the heavy chain and a Q38K (Kabat numbering) substitution in the light chain.

In some embodiments, the multispecific antigen-binding molecule described herein comprises an anti-STEAP1 arm comprising Q39E (Kabat numbering) and S183K (EU numbering) substitutions in the heavy chain and Q38K (Kabat numbering) and V133E (EU numbering) substitutions in the light chain; and an anti-CD3 arm comprising a Q39K (Kabat numbering) substitution in the heavy chain and a Q38E (Kabat numbering) substitution in the light chain.

In some embodiments, the multispecific antigen-binding molecule described herein comprises an anti-CD3 arm comprising Q39E (Kabat numbering) and S183K (EU numbering) substitutions in the heavy chain and Q38K (Kabat numbering) and V133E (EU numbering) substitutions in the light chain; and an anti-STEAP1 arm comprising a Q39K (Kabat numbering) substitution in the heavy chain and a Q38E (Kabat numbering) substitution in the light chain.

In another example, provided herein is a method for producing a multispecific antigen-binding molecule disclosed herein, comprising (a) introducing one or more polynucleotide molecules encoding a multispecific antigen-binding molecule into a host cell, wherein the multispecific antigen-binding molecule comprises a) a first heavy chain/light chain pair binding to a first antigen which comprises a first heavy chain polypeptide (H1) and a first light chain polypeptide (L1), and b) a second heavy chain/light chain pair binding to a second antigen which comprises a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a positively charged residue, an amino acid at Q39 (Kabat numbering) in the VH domain of H1 is replaced with a negatively charged residue, an amino acid at V133 (EU numbering) in the CL domain of L1 is replaced with a negatively charged residue, an amino acid at Q38 (Kabat numbering) in the VL domain of L1 is replaced with a positively charged residue, an amino acid at S183 (EU numbering) in the CH1 domain of H2 is replaced with a negatively charged residue, an amino acid at Q39 (Kabat numbering) in the VH domain of H2 is replaced with a positively charged residue, an amino acid at Q38 (Kabat numbering) in the VL domain of L2 is replaced with a negatively charged residue, and an amino acid at V133 (EU numbering) in the CL domain of L2 is replaced with a positively charged residue; or (ii) an amino acid at S183 (EU numbering) in the CH1 domain of H1 is replaced with a negatively charged residue, an amino acid at Q39 (Kabat numbering) in the VH domain of H1 is replaced with a positively charged residue, an amino acid at V133 (EU numbering) in the CL domain of L1 is replaced with a positively charged residue, an amino acid at Q38 (Kabat numbering) in the VL domain of L1 is replaced with a negatively charged residue, an amino acid at S183 (EU numbering) in the CH1 domain of H2 is replaced with a positively charged residue, an amino acid at Q39 (Kabat numbering) in the VH domain of H2 is replaced with a negatively charged residue, an amino acid at Q38 (Kabat numbering) in the VL domain of L2 is replaced with a positively charged residue, and an amino acid at V133 (EU numbering) in the CL domain of L2 is replaced with a negatively charged residue; and (b) culturing the host cell under conditions and for a time sufficient to produce the antigen-binding protein. In some embodiments, the positively charged residue is selected from R and K and the negatively charged residue is selected from D and E. In some embodiments, the positively charged residue is R. In other embodiments, the positively charged residue is K. In some embodiments, the negatively charged residue is D. In other embodiments, the negatively charged residue is E. In some embodiments, the first antigen is STEAP1 and the second antigen is CD3. In other embodiments, the first antigen is CD3 and the second antigen is STEAP1.

For example, provided herein is a method for producing a multispecific antigen-binding molecule disclosed herein, comprising (a) providing a host cell comprising one or more polynucleotide molecules encoding a multispecific antigen-binding molecule, wherein the multispecific antigen-binding molecule comprises a) a first heavy chain/light chain pair binding to a first antigen which comprises a first heavy chain polypeptide (H1) and a first light chain polypeptide (L1), and b) a second heavy chain/light chain pair binding to a second antigen which comprises a second heavy chain polypeptide (H2) and a second light chain polypeptide (L2), wherein each H1 and H2 comprises a heavy chain variable domain (VH) and a heavy chain constant domain (CH1), and each L1 and L2 comprises a light chain variable domain (VL) and a light chain constant domain (VL), wherein: (i) an amino acid at S183 (EU numbering) in the CH1 domain of H1 is replaced with a positively charged residue, an amino acid at Q39 (Kabat numbering) in the VH domain of H1 is replaced with a negatively charged residue, an amino acid at V133 (EU numbering) in the CL domain of L1 is replaced with a negatively charged residue, an amino acid at Q38 (Kabat numbering) in the VL domain of L1 is replaced with a positively charged residue, an amino acid at S183 (EU numbering) in the CH1 domain of H2 is replaced with a negatively charged residue, an amino acid at Q39 (Kabat numbering) in the VH domain of H2 is replaced with a positively charged residue, an amino acid at Q38 (Kabat numbering) in the VL domain of L2 is replaced with a negatively charged residue, and an amino acid at V133 (EU numbering) in the CL domain of L2 is replaced with a positively charged residue; or (ii) an amino acid at S183 (EU numbering) in the CH1 domain of H1 is replaced with a negatively charged residue, an amino acid at Q39 (Kabat numbering) in the VH domain of H1 is replaced with a positively charged residue, an amino acid at V133 (EU numbering) in the CL domain of L1 is replaced with a positively charged residue, an amino acid at Q38 (Kabat numbering) in the VL domain of L1 is replaced with a negatively charged residue, an amino acid at S183 (EU numbering) in the CH1 domain of H2 is replaced with a positively charged residue, an amino acid at Q39 (Kabat numbering) in the VH domain of H2 is replaced with a negatively charged residue, an amino acid at Q38 (Kabat numbering) in the VL domain of L2 is replaced with a positively charged residue, and an amino acid at V133 (EU numbering) in the CL domain of L2 is replaced with a negatively charged residue; and (b) culturing the host cell under conditions and for a time sufficient to produce the antigen-binding protein. In some embodiments, the positively charged residue is selected from R and K and the negatively charged residue is selected from D and E. In some embodiments, the positively charged residue is R. In other embodiments, the positively charged residue is K. In some embodiments, the negatively charged residue is D. In other embodiments, the negatively charged residue is E. In some embodiments, the first antigen is STEAP1 and the second antigen is CD3. In other embodiments, the first antigen is CD3 and the second antigen is STEAP1.

In some embodiments, the multispecific antigen-binding molecule described herein comprises an anti-STEAP1 arm comprising Q39K (Kabat numbering) and S183E (EU numbering) substitutions in the heavy chain and Q38E (Kabat numbering) and V133K (EU numbering) substitutions in the light chain; and an anti-CD3 arm comprising a Q39E (Kabat numbering) substitution and an S183K (EU numbering) substitution in the heavy chain and a Q38K (Kabat numbering) substitution and a V133E (EU numbering) substitution in the light chain.

In some embodiments, the multispecific antigen-binding molecule described herein comprises an anti-CD3 arm comprising Q39K (Kabat numbering) and S183E (EU numbering) substitutions in the heavy chain and Q38E (Kabat numbering) and V133K (EU numbering) substitutions in the light chain; and an anti-STEAP1 arm comprising a Q39E (Kabat numbering) substitution and an S183K (EU numbering) substitution in the heavy chain and a Q38K (Kabat numbering) substitution and a V133E (EU numbering) substitution in the light chain.

In some embodiments, the multispecific antigen-binding molecule described herein comprises an anti-STEAP1 arm comprising Q39E (Kabat numbering) and S183K (EU numbering) substitutions in the heavy chain and Q38K (Kabat numbering) and V133E (EU numbering) substitutions in the light chain; and an anti-CD3 arm comprising a Q39K (Kabat numbering) substitution and an S183E (EU numbering) substitution in the heavy chain and a Q38E (Kabat numbering) substitution and a V133K (EU numbering) substitution in the light chain.

In some embodiments, the multispecific antigen-binding molecule described herein comprises an anti-CD3 arm comprising Q39E (Kabat numbering) and S183K (EU numbering) substitutions in the heavy chain and Q38K (Kabat numbering) and V133E (EU numbering) substitutions in the light chain; and an anti-STEAP1 arm comprising a Q39K (Kabat numbering) substitution and an S183E (EU numbering) substitution in the heavy chain and a Q38E (Kabat numbering) substitution and a V133K (EU numbering) substitution in the light chain. In one example, anti-STEAP1/CD3 TDBs of the invention may be produced as described in Dillon et al. MAbs 9:213-230, 2017.

### Two-Cell Approach for Anti-STEAP1/CD3 TDB Production

Alternatively, anti-STEAP1/CD3 TDBs can be produced by culturing the antibody hemimers (e.g., half-antibodies) separately (i.e., in two different cell lines) using high-cell density fermentation and then isolating each half-antibody independently by Protein A chromatography. The purified half-antibodies can then be combined, for example, at a 1:1 molar ratio and incubated in 50 mM Tris, pH 8.5 in the presence of 2 mM DTT for 4 hours to allow annealing and the reduction of disulfides in the hinge region. Dialysis against the same buffer without DTT for 24-48 hours resulted in the formation of the inter-chain disulfide bonds.

TDBs may be alternatively produced by transfection of two plasmids, each encoding the distinct arms of the TDB, into separate host cells. The host cells may be co-cultured or cultured separately. Transfection of host cells may be performed in a 96-well plate format. To screen for TDB production, 2,000 to 3,000 clones may be picked and assessed by ELISA and intact IgG homogeneous time resolved fluorescence (HTRF) for their ability to bind a selected antigen (e.g., STEAP1). Clones producing TDBs capable of binding to STEAP1, or a fragment thereof, may be selected for expansion and further screening. Top clones are selected for further analysis based on percent bispecific antibodies (bsAbs) produced, titer, and PQ.

### Exemplary Production Methods

In one example, TDBs were produced by a co-culture strategy using E. coli cells expressing one half-antibody (hole) and E. coli cells expressing the second half-antibody (knob) were grown together in shaker flasks at a predetermined ratio such that it produced similar amounts of each half-antibody (see, Spiess et al., Nat. Biotechnol. 31(8):753-8 (2013); PCT Pub. No. WO 2011/069104, which is incorporated herein by reference in its entirety). The co-cultured bacterial broth was then harvested, the cells disrupted in a microfluidizer and the antibodies purified by Protein A affinity. It has been observed that during microfluidizing and protein A capture the two arms annealed and formed the hinge inter-chain disulfide bridges.

In another example, full-length bispecific antibodies were produced as previously described (Junttila et al. Cancer Res. 74:5561-5571, 2014; Sun et al. Science Trans. Med. 7:287ra270, 2015). Briefly, the two half-antibodies (e.g., anti-STEAP1 such as huAb44.v6.05 and anti-CD3 such as 40G5c or 38E4v1.MD1) containing “knob” or “hole” mutations in their CH3 domains were expressed by transient transfection of CHO cells and then affinity purified with Protein A. Equal amounts of the two half-antibodies were incubated with a 200 molar excess of reduced glutathione at pH 8.5 overnight at 32 °C to drive the formation of the knob-hole disulfide bonds. The assembled bispecific antibody (e.g., anti-STEAP1/CD3 TDB) was purified from contaminants through hydrophobic interaction chromatography. The purified anti-STEAP1/CD3 TDBs were characterized for purity by mass spectrometry, size exclusion chromatography (SEC), and gel electrophoresis.

### IV. Assays

Antigen-binding molecules provided herein may be identified, screened for, or characterized for their physical/chemical properties and/or biological activities by various assays known in the art.

In one aspect, an antigen-binding molecule of the invention is tested for its antigen binding activity, e.g., by known methods such as ELISA, Western blot, and the like.

In another aspect, competition assays may be used to identify an antigen-binding molecule that competes with a reference antibody for binding to STEAP1. In certain aspects, such a competing antigen-binding molecule binds to the same epitope (e.g., a linear or a conformational epitope) that is bound by the reference antibody. Detailed exemplary methods for mapping an epitope to which an antibody binds are provided in Morris “Epitope Mapping Protocols”, in Methods in Molecular Biology vol. 66 (Humana Press, Totowa, NJ) (1996).

In an exemplary competition assay, immobilized STEAP1 is incubated in a solution comprising a first labeled antibody that binds to STEAP1 and a second unlabeled antibody that is being tested for its ability to compete with the first antibody for binding to STEAP1. The second antibody may be present in a hybridoma supernatant. As a control, immobilized STEAP1 is incubated in a solution comprising the first labeled antibody but not the second unlabeled antibody. After incubation under conditions permissive for binding of the first antibody to STEAP1, excess unbound antibody is removed, and the amount of label associated with immobilized STEAP1 is measured. If the amount of label associated with immobilized STEAP1 is substantially reduced in the test sample relative to the control sample, then that indicates that the second antibody is competing with the first antibody for binding to STEAP1. See Harlow and Lane (1988) Antibodies: A Laboratory Manual ch. 14 (Cold Spring Harbor Laboratory, Cold Spring Harbor, NY).

### V. Pharmaceutical Formulations

Pharmaceutical formulations of the antigen-binding molecules (e.g., monospecific and/or multispecific antigen-binding molecules such as bispecific or trispecific antigen-binding molecules) of the present invention are prepared for storage by mixing the antigen-binding molecule having the desired degree of purity with optional pharmaceutically acceptable carriers, excipients, or stabilizers in the form of lyophilized formulations or aqueous solutions. For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 119, Marcel Dekker, 2002.

Pharmaceutical compositions of an antigen-binding molecule (e.g., monospecific and/or multispecific antigen-binding molecule such as a bispecific or trispecific antigen-binding molecule) as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The formulation herein may also contain more than one active compound, preferably those with complementary activities that do not adversely affect each other. The type and effective amounts of such medicaments depend, for example, on the amount and type of antagonist present in the formulation, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### VI. Therapeutic Methods

Any of the antigen-binding molecules (e.g., monospecific and/or multispecific antigen-binding molecules such as bispecific or trispecific antigen-binding molecules) of the invention or compositions thereof may be used in any of the therapeutic methods described herein. In some embodiments, the antigen-binding molecules (e.g., monospecific and/or multispecific antigen-binding molecules such as bispecific or trispecific antigen-binding molecules) are useful for treating or delaying the progression of a cancer, e.g., a STEAP1-expressing cancer. In additional embodiments, the antigen-binding molecules (e.g., monospecific and/or multispecific antigen-binding molecules such as bispecific or trispecific antigen-binding molecules) are useful for inhibiting or reducing the proliferation of a cancer cell, e.g., a STEAP1-expressing cell.

In some embodiments, the antigen-binding molecules (e.g., monospecific and/or multispecific antigen-binding molecules such as bispecific or trispecific antigen-binding molecules) are useful for treating or delaying progression of a STEAP1-expressing cancer in a subject in need thereof. In some instances, the STEAP1-expressing cancer is a solid tumor. In some instances, the STEAP1-expressing cancer is a prostate cancer, Ewing sarcoma, lung cancer, colorectal cancer, breast cancer, bladder cancer, ovarian cancer, or cervical cancer. In some instances, the STEAP1-expressing cancer is a prostate cancer. In some instances, the STEAP1-expressing cancer is Ewing sarcoma.

In some embodiments, the antigen-binding molecules (e.g., monospecific and/or multispecific antigen-binding molecules such as bispecific or trispecific antigen-binding molecules) are administered to the subject in combination with an additional therapeutic agent or an additional therapeutic regimen. In some cases, the additional therapeutic agent comprises a chemotherapeutic agent, an immunotherapeutic agent, a targeted therapy, hormone therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the additional therapeutic agent comprises a first-line therapy. In some cases, the additional therapeutic agent comprises a second-line therapy, a third-line therapy, a fourth-line therapy, or a fifth-line therapy.

In some cases, the additional therapeutic agent comprises a salvage therapy.

In some cases, the additional therapeutic agent comprises a palliative therapy.

In some cases, the additional therapeutic agent comprises a hormone therapy. Exemplary hormone therapy include, but are not limited to, leuprolide, enzalutamide, apalutamide, and abiraterone.

In some cases, the additional therapeutic agent comprises an immune checkpoint inhibitor, e.g., an inhibitor of PD-L1, PD-L2, PD-1, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. In some cases, the immune checkpoint inhibitor is pembrolizumab, nivolumab, tremelimumab, or ipilimumab.

In some cases, the additional therapeutic agent comprises an antibody such as alemtuzumab, trastuzumab, ibritumomab tiuxetan, brentuximab vedotin, ado-trastuzumab emtansine, or blinatumomab.

In some cases, the additional therapeutic agent comprises an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprises a cytokine. Exemplary cytokines include, but are not limited to, IL-1β, IL-6, IL-7, IL-10, IL-12, IL-15, IL-21, or TNFα.

In some embodiments, the additional therapeutic agent comprises a receptor agonist. In some instances, the receptor agonist comprises a Toll-like receptor (TLR) ligand. In some cases, the TLR ligand comprises TLR1, TLR2, TLR3, TLR4, TLR5, TLR6, TLR7, TLR8, or TLR9. In some cases, the TLR ligand comprises a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some instances, the additional therapeutic regimen is a radiation therapy.

In some instances, the additional therapeutic regimen is surgery.

Such combination therapies noted above encompass combined administration (where two or more therapeutic agents are included in the same or separate formulations), and separate administration, in which case, administration of the antigen-binding molecule of the invention can occur prior to, simultaneously, and/or following, administration of the additional therapeutic agent or agents. In one embodiment, administration of the antigen-binding molecule and administration of an additional therapeutic agent occur within about one month, or within about one, two or three weeks, or within about one, two, three, four, five, or six days, of each other.

An antigen-binding molecule of the invention (and/or any additional therapeutic agent) can be administered by any suitable means, including subcutaneously, intravenously, intramuscularly, topically, orally, transdermally, intraperitoneally, intraorbitally, by implantation, by inhalation, intrathecally, intraventricularly, or intranasally. For example, in some embodiments, the antigen-binding molecule is administered subcutaneously. In other embodiments, the antigen-binding molecule is administered intravenously. In some embodiments, an antigen-binding molecule administered by subcutaneous injection exhibits a less toxic response in a patient than the same antigen-binding molecule administered by intravenous injection. Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antigen-binding molecules of the invention can be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners.

The antigen-binding molecule need not be, but is optionally formulated with one or more agents currently used to prevent or treat the cancer in question. The effective amount of such other agents depends on the amount of antigen-binding molecule present in the formulation, the type of cancer or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of cancer, the appropriate dosage of an antigen-binding molecule of the invention (when used alone or in combination with one or more other additional therapeutic agents) will depend on the type of cancer to be treated, the severity and course of the cancer, whether the antigen-binding molecule is administered for preventive or therapeutic purposes, previous therapy, the patient's clinical history and response to the antigen-binding molecule, and the discretion of the attending physician. The antigen-binding molecule is suitably administered to the patient at one time or over a series of treatments.

As a general proposition, the therapeutically effective amount of the antigen-binding molecule administered to a human will be in the range of about 0.01 to about 100 mg/kg of patient body weight whether by one or more administrations. In some embodiments, the antigen-binding molecule used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, an antigen-binding molecule described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. For repeated administrations over several days or longer, depending on the condition, the treatment would generally be sustained until a desired suppression of disease symptoms occurs.

One exemplary dosage of the antigen-binding molecule would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the subject. Such doses may be administered intermittently, for example, every week or every three weeks (e.g., such that the patient receives from about two to about twenty, or, for example, about six doses of the anti-CD3 antibody). An initial higher loading dose, followed by one or more lower doses may be administered. The progress of this therapy is easily monitored by conventional techniques and assays. In some embodiments of any of the preceding aspects of the invention, the subject, patient, or individual is a human.

In some embodiments, the antigen-binding molecules (e.g., monospecific and/or multispecific antigen-binding molecules such as bispecific or trispecific antigen-binding molecules) are useful for inhibiting or reducing the proliferation of a STEAP1 expressing cell. In some instances, the cell is a cell of prostate cancer, Ewing sarcoma, lung cancer, colorectal cancer, breast cancer, bladder cancer, ovarian cancer, or cervical cancer. In some cases, the cell is a prostate cancer cell or an Ewing sarcoma cell. In some cases, the antigen-binding molecules (e.g., monospecific and/or multispecific antigen-binding molecules such as bispecific or trispecific antigen-binding molecules) are used in an in vivo method. In additional cases, the antigen-binding molecules (e.g., monospecific and/or multispecific antigen-binding molecules such as bispecific or trispecific antigen-binding molecules) are used in an in vitro or ex vivo method.

### VII. Kits/Articles of Manufacture

Provided herein is a kit or an article of manufacture including one or more compositions described herein (e.g., a composition including any one or more of the antigen-binding molecules described herein and a pharmaceutically acceptable carrier) and a package insert for administering the composition to a subject to treat or delay progression of a cancer (e.g., prostate cancer or Ewing sarcoma).

The kit or article of manufacture may comprise a container and a label or package insert on or associated with the container. Suitable containers include, for example, bottles, vials, syringes, IV solution bags, etc. The containers may be formed from a variety of materials such as glass or plastic. The container may hold a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antigen-binding molecule of the invention. The label or package insert may indicate that the composition is used for treating any disorder disclosed herein (e.g., a cancer (e.g., prostate cancer or Ewing sarcoma)).

Moreover, the kit or article of manufacture may comprise (a) a first container with a composition contained therein, wherein the composition comprises an antigen-binding molecule of the invention; and (b) a second container with a composition contained therein, wherein the composition comprises a further cytotoxic or otherwise therapeutic agent. The kit or article of manufacture in this aspect of the invention may further comprise a package insert indicating that the compositions can be used to treat a particular condition. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### VIII. Tocilizumab for Treatment of Cytokine Release Syndrome (CRS)

### Background

Bispecific antibody therapeutics involving T-cell activation have been associated with cytokine release syndrome (CRS). CRS is a potentially life-threatening symptom complex caused by the excessive release of cytokines by immune effector or target cells during an exaggerated and sustained immune response. CRS can be triggered by a variety of factors, including infection with virulent pathogens, or by medications that activate or enhance the immune response, resulting in a pronounced and sustained immune response.

Regardless of the inciting agent, severe or life-threatening CRS is a medical emergency. If unsuccessfully managed, it can result in significant disability or fatal outcome. Current clinical management focuses on treating the individual signs and symptoms, providing supportive care, and attempting to dampen down the inflammatory response using high-dose corticosteroids. However, this approach is not always successful, especially in the case of late intervention. Moreover, steroids may negatively impact T-cell function, which may diminish the clinical benefit of immune modulating therapies in the treatment of cancer.

CRS is associated with high IL-6 levels (Panelli et al., J Transl. Med., 2:17, 2004; Lee et al., Blood, 124:188-195, 2014; Doessegger and Banholzer, Clin. Transl. Immunology, 4:e39, 2015), and IL-6 correlates with the severity of CRS, with patients who experience severe or life-threatening CRS (NCI CTCAE Grades 4 or 5) having much higher IL-6 levels compared with their counterparts who do not experience CRS or experience milder CRS reactions (NCI CTCAE Grades 0-3) (Chen et al., J. Immunol. Methods, 434:1-8, 2016).

Tocilizumab (ACTEMRA®/ROACTEMRA®) is a recombinant, humanized, anti-human monoclonal antibody directed against soluble and membrane-bound IL-6R, which inhibits IL-6 mediated signaling (see, e.g., WO 1992/019579, which is incorporated herein by reference in its entirety). Tocilizumab has been approved by the U.S. Food and Drug Administration for the treatment of severe or life-threatening CAR-T cell-induced CRS in adults and in pediatric patients 2 years of age and older. Initial clinical data (Locke et al., Blood, 130:1547, 2017) suggests that tocilizumab prophylaxis may reduce the severity of CAR-T cell-induced CRS by blocking IL-6 receptors from signaling prior to cytokine release. Consequently, tocilizumab premedication may also reduce the frequency or lower the severity of CRS associated with bispecific antibody therapy. Other anti-IL-6R antibodies that could be used in combination with tocilizumab include sarilumab, vobarilizumab (ALX-0061), SA-237, and variants thereof.

### CRS Symptoms and Grading

CRS may be graded according to the Modified Cytokine Release Syndrome Grading System established by Lee et al., Blood 124:188-195, 2014 or Lee et al., Biol Blood Marrow Transplant 25(4):625-638, 2019, as described in Table A. In addition to diagnostic criteria, recommendations on management of CRS based on its severity, including early intervention with corticosteroids and/or anti-cytokine therapy, are provided and referenced in Tables A and B.

TABLE A
Cytokine release syndrome grading systems

| | Modified Cytokine Release | ASTCT Consensus |
|---|---|---|
| Grade | Syndrome Grading System | Grading System |
| Grade 1 | Symptoms are not life threatening | Temperature ≥ 38°C |
| | and require symptomatic | No hypotension |
| | treatment only (e.g., fever, | No hypoxia |
| | nausea, fatigue, headache, | |
| | myalgia, malaise) | |
| Grade 2 | Symptoms require and respond to | Temperature ≥ 38 °C* with |
| | moderate intervention | hypotension not requiring |
| | Oxygen requirement < 40%; or | vasopressors and/or^{†} |
| | Hypotension responsive to fluids | hypoxia requiring low-flow |
| | or low dose ^{a} of one vasopressor; | nasal cannula^{‡} or blow-by |
| | or | |
| | Grade 2 organ toxicity | |
| Grade 3 | Symptoms require and respond to | Temperature ≥ 38 °C* with |
| | aggressive intervention | hypotension requiring a |
| | Oxygen requirement ≥ 40%; or | vasopressor with or without |
| | Hypotension requiring high dose ^{b} | vasopressin and/or^{†} hypoxia |
| | or multiple vasopressors; or | requiring high-flow nasal |
| | Grade 3 organ toxicity or Grade 4 | cannula^{‡}, facemask, |
| | transaminitis | nonrebreather mask, or |
| | | Venturi mask |
| Grade 4 | Life-threatening symptoms | Temperature ≥ 3 8 °C* with |
| | Requirement for ventilation | hypotension requiring |
| | support or | multiple vasopressors |
| | Grade 4 organ toxicity (excluding | (excluding vasopressin) |
| | transaminitis) | and/or^{†} hypoxia |
| | | requiring positive pressure |
| | | (e.g., CPAP, BiPAP, |
| | | intubation and mechanical |
| | | ventilation) |
| Grade 5 | Death | Death |

Lee 2014 criteria: Lee et al., Blood, 124: 188-195, 2014.
ASTCT consensus grading: Lee et al., Biol Blood Marrow Transplant, 25(4): 625-638, 2019.
^{a} Low-dose vasopressor: single vasopressor at doses below that shown in Table B.
^{b} High-dose vasopressor: as defined in Table B.
*Fever is defined as temperature ≥ 38 °C not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5 °C, hypotension requiring 1 vasopressor, and hypoxia requiring low-flow nasal cannula is classified as grade 3 CRS.
^{‡}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE B
High-dose vasopressors
High-Dose Vasopressors (duration ≥ 3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |
| If on vasopressin | Vasopressin + norepinephrine | |
| | equivalent of ≥10 μg/min ^{a} | |
| If on combination or vasopressors | Norepinephrine equivalent of ≥20 μg/min ^{a} | |
| (not vasopressin) | | |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life threatening CRS resulting from immune-based therapy has not been established; case reports and recommendations using anti-cytokine therapy such as tocilizumab have been published (Teachey et al., Blood 121:5154-5157 (2013); Lee et al., Blood, 124:188-195 (2014); Maude et al., New Engl. J. Med., 371:1507-1517 (2014)).

As noted in Table A, even moderate presentations of CRS in subjects with extensive comorbidities should be monitored closely, with consideration given to intensive care unit admission and tocilizumab administration.

### Tocilizumab as a Premedication

In some aspects, an effective amount of tocilizumab is administered as a premedication, e.g., is administered to the subject prior to the administration of a multispecific antigen-binding molecule described herein (e.g., a bispecific or trispecific antigen-binding molecule, e.g., a bispecific antibody). Administration of tocilizumab as a premedication may reduce the frequency or severity of CRS. In some aspects, tocilizumab is administered as a premedication in Cycle 1, e.g., is administered prior to a first dose (Cl D1), a second dose (Cl D2), and/or a third dose (Cl D3) of the multispecific antigen-binding molecule (e.g., the bispecific antibody). In some aspects, the tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

For example, in one aspect, the multispecific antigen-binding molecule (e.g., the bispecific antibody) is co-administered with tocilizumab (ACTEMRA®/ROACTEMRA®), wherein the subject is first administered with tocilizumab (ACTEMRA®/ROACTEMRA®) and then separately administered with the bispecific antibody (e.g., the subject is pre-treated with tocilizumab (ACTEMRA®/ROACTEMRA®)).

In some aspects, the disclosure features the use of a multispecific antigen-binding molecule (e.g., a bispecific antibody) of the invention in the manufacture of a medicament for the treatment of a subject having prostate cancer or Ewing sarcoma in combination with one or more additional therapeutic agents (e.g., tocilizumab).

In some aspects, the multispecific antigen-binding molecule (e.g., the bispecific antibody) and the one or more additional therapeutic agents are formulated separately. In some aspects, the multispecific antigen-binding molecule (e.g., the bispecific antibody) is to be administered to the subject prior to the one or more additional therapeutic agents. In other aspects, the multispecific antigen-binding molecule (e.g., the bispecific antibody) is to be administered to the subject subsequent to the one or more additional therapeutic agents, e.g., administered to the subject subsequent to administration of an effective amount of tocilizumab.

In some aspects, the multispecific antigen-binding molecule (e.g., the bispecific antibody) and the one or more additional therapeutic agents are formulated together.

In some aspects, the disclosure features a multispecific antigen-binding molecule (e.g., a bispecific antibody) of the invention for use in treating a subject having prostate cancer or Ewing sarcoma in combination with one or more additional therapeutic agents.

In some aspects, the multispecific antigen-binding molecule (e.g., the bispecific antibody) and the one or more additional therapeutic agents are formulated separately. In some aspects, the multispecific antigen-binding molecule (e.g., the bispecific antibody) is to be administered to the subject prior to the one or more additional therapeutic agents. In other aspects, the multispecific antigen-binding molecule (e.g., the bispecific antibody) is to be administered to the subject subsequent to the one or more additional therapeutic agents, e.g., administered to the subject subsequent to administration of an effective amount of tocilizumab.

In some aspects, the multispecific antigen-binding molecule (e.g., the bispecific antibody) and the one or more additional therapeutic agents are formulated together.

### Tocilizumab to Treat CRS

In some aspects, the subject experiences a CRS event during treatment with the therapeutic multispecific antigen-binding molecule (e.g., the bispecific antibody) and an effective amount of tocilizumab is administered to manage the CRS event.

In some aspects, the subject has a CRS event (e.g., has a CRS event following treatment with the multispecific antigen-binding molecule (e.g., the bispecific antibody), e.g., has a CRS event following a first dose or a subsequent dose of the multispecific antigen-binding molecule, and the method further includes treating the symptoms of the CRS event while suspending treatment with the multispecific antigen-binding molecule.

In some aspects, the subject experiences a CRS event, and the method further includes administering to the subject an effective amount of an interleukin-6 receptor (IL-6R) antagonist (e.g., an anti-IL-6R antibody, e.g., tocilizumab (ACTEMRA®/ROACTEMRA®)) to manage the CRS event while suspending treatment with the multispecific antigen-binding molecule (e.g., the bispecific antibody). In some aspects, the IL-6R antagonist (e.g., tocilizumab) is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

In some aspects, the CRS event does not resolve or worsens within 24 hours of treating the symptoms of the CRS event, and the method further includes administering to the subject one or more additional doses of the IL-6R antagonist (e.g., an anti-IL-6R antibody, e.g., tocilizumab) to manage the CRS event, e.g., administering one or more additional doses of tocilizumab intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the one or more additional doses of tocilizumab are administered intravenously to the subject as a single dose of about 8 mg/kg.

In some aspects, the method further includes administering to the subject an effective amount of a corticosteroid. The corticosteroid may be administered intravenously to the subject. In some aspects, the corticosteroid is methylprednisone (methylprednisolone). In some instances, the methylprednisone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

The subject may be administered a corticosteroid, such as methylprednisolone or dexamethasone, if the CRS event is not managed with administration of the IL-6R antagonist (e.g., tocilizumab) alone. In some aspects, treating the symptoms of the CRS event further includes treatment with a high-dose vasopressor (e.g., norepinephrine, dopamine, phenylephrine, epinephrine, or vasopressin and norepinephrine), e.g., as described in Tables A, B, and C. Tables C and D provide details about tocilizumab treatment of severe or life-threatening CRS.

In some aspects, the disclosure features the use of tocilizumab in the manufacture of a medicament for the treatment of a subject having a CRS event, wherein the CRS event arises during treatment of the subject with the bispecific antibody of the invention.

In some aspects, the medicament is to be administered to the subject while treatment with the multispecific antigen-binding molecule (e.g., the bispecific antibody) of the invention is suspended.

In some aspects, the medicament is formulated for intravenous administration of tocilizumab as a single dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, the CRS event does not resolve or worsens within 24 hours of treating the symptoms of the CRS event and one or more additional doses of tocilizumab are to be administered to the subject. The one or more additional doses of tocilizumab may be to be administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, the medicament is for use in combination with an effective amount of a corticosteroid to treat the CRS event. Tocilizumab and the corticosteroid may be formulated separately.

In some aspects, the corticosteroid is to be administered intravenously to the subject. In some aspects, the corticosteroid is methylprednisolone, e.g., methylprednisolone is to be administered to the subject at a dose of about 1 mg/kg per day to about 5 mg/kg per day, e.g., about 2 mg/kg per day. In some aspects, the corticosteroid is dexamethasone, e.g., dexamethasone is to be administered to the subject at a dose of about 10 mg or at a dose of about 0.5 mg/kg per day.

In some aspects, the disclosure features tocilizumab for use in treating a subject having a CRS event, wherein the CRS event arises during treatment of the subject with a multispecific antigen-binding molecule (e.g., a bispecific antibody) of the invention.

In some aspects, tocilizumab is to be administered to the subject while treatment with the bispecific antibody of the invention is suspended.

In some aspects, tocilizumab is formulated for intravenous administration as a single dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, the CRS event does not resolve or worsens within 24 hours of treating the symptoms of the CRS event and one or more additional doses of tocilizumab are to be administered to the subject. The one or more additional doses of tocilizumab may be to be administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, tocilizumab is for use in combination with an effective amount of a corticosteroid to treat the CRS event. Tocilizumab and the corticosteroid may be formulated separately. In some aspects, the corticosteroid is to be administered intravenously to the subject. In some aspects, the corticosteroid is methylprednisolone, e.g., methylprednisolone is to be administered to the subject at a dose of about 1 mg/kg per day to about 5 mg/kg per day, e.g., about 2 mg/kg per day. In some aspects, the corticosteroid is dexamethasone, e.g., dexamethasone is to be administered to the subject at a dose of about 10 mg or at a dose of about 0.5 mg/kg per day.

TABLE C
Tocilizumab treatment of severe or lift-threatening cytokine release syndrome (CRS)

| | Pre-TCZ | | | | | | |
|---|---|---|---|---|---|---|---|
| | Treatment | | | | | | |
| | (within 24 | TCZ | Post-TCZ Treatment ª | | | | |
| Assessment/Procedure | hours) | Administration | 6 hours | 1 day | 2 days | 3 days | 8 days |
| TCZ Administration (8 mg/kg) | | x | | | | | |

| | | |
|---|---|---|
| Vital signs ^{b} | x ^{c} | Measure at least every 6 hours until resolution |
| | | to baseline, then every 12 hours through Day 8 ^{c} |
| | | or until discharge from ICU |
| Pressor documentation ^{d} | x ^{c} | Record at least every 6 hours until pressors are |
| | | discontinued ^{c} |
| FiO2 | x ^{c} | Record at least every 6 hours until patient on |
| | | room air ^{c} |
| Pulse oximetry, resting | x ^{c} | Measure at least every 6 hours until resolution |
| | | to baseline, then every 12 hours through Day 8 ^{c} |
| | | or until discharge from ICU |

Local Laboratory Assessments

| | | | | | | |
|---|---|---|---|---|---|---|
| Hematology | x | x | x | x | x | x |
| Liver function tests (AST, ALT, | x | x | x | x | x | x |
| total bilirubin) | | | | | | |
| Serum chemistry and creatine ^{e} | x | x | x | x | x | x |
| CRP, LDH, and serum ferritin | x | x | x | x | x | x |
| Coagulation (aPTT, PT/INR, | x | x | x | x | x | x |
| fibrinogen) | | | | | | |
| Infection workup ^{f} | x | | | | | |

Central Laboratory Assessments

| | | | | | | | |
|---|---|---|---|---|---|---|---|
| Plasma cytokines | x | | x | x | x | x | x |
| Plasma IL-6 PD markers ^{g} | x | x ^{h} | x | x | x | x | x |
| Serum TCZ pharmacokinetics | x | x ^{h} | x | x | x | x | x |

CRP: C-reactive protein; eCRF: electronic Case Report Form; IL: interleukin; PD: pharmacodynamic; PK: pharmacokinetic; TCZ: tocilizumab.
Note:
Record abnormalities or worsened clinically significant abnormalities on the Adverse Event eCRF.
^{a} If TCZ dose is repeated, follow schedule of activities following the second TCZ dose.
^{b} Includes respiratory rate, heart rate, and systolic and diastolic blood pressure while the patient is in a seated or supine position, and temperature.
^{c} The maximum and minimum values for any 24-hour period should be recorded in the clinical database.
^{d} Document vasopressor type and dose in the concomitant medication eCRF.
^{e} Includes sodium, potassium, chloride, bicarbonate, glucose and blood urea nitrogen
^{f} Includes assessment for bacterial, fungal, and viral infections.
^{g} Includes IL-6, soluble IL-6R, and sgp130.
^{h} Blood draws for serum TCZ PK and plasma IL-6 PD markers will be performed at the end of TCZ infusion, and will be drawn from the arm which was not used to administer TCZ.

### Management of CR8 Events by Grade

Management of the CRS events may be tailored based on the grade of the CRS (Tables A and D) and the presence of comorbidities. Table D provides recommendations for the management of CRS syndromes by grade.

TABLE D
Recommendations for management of cytokine release syndrome

| Event ^{a, b} | Action to be taken ^{b} |
|---|---|
| Grade 1 | Immediate actions: |
| Fever, constitutional symptoms | If infusion is still ongoing, slow the infusion rate up to 50% or |
| | interrupt infusion. |
| | Treat symptomatically as indicated, including antihistamines, |
| | antipyretics, and/or analgesics as needed. |
| | Treat fever and neutropenia if present. |
| | Monitor fluid balance; administer IV fluids as clinically indicated. |
| | Restarting infusion: |
| | If therapeutic bispecific antibody infusion was interrupted, wait |
| | until 30 minutes after the event has resolved before restarting |
| | the infusion at 50% of the original infusion rate. |
| Grade 2 | Immediate actions: |
| Hypotension: responds to fluids | Follow all Grade 1 recommendations. |
| or a single low-dose pressor ^{c} | Hold further bispecific antibody treatment until symptoms |
| Hypoxia: requires <40% FiO2 | completely resolved. |
| to maintain adequate | Consider treatment with IV corticosteroids (such as |
| hemoglobin oxygen saturation | methylprednisolone 2 mg/kg/day or, if neurologic symptoms are |
| Organ toxicity: Grade 2 | present, dexamethasone 0.5 mg/kg/day). ^{b} |
| | Consider administering tocilizumab 8 mg/kg IV as a single |
| | dose. |
| | Monitor cardiac and other organ function closely. |
| | Provide hemodynamic support as indicated. |
| | Provide oxygen for hypoxia. |
| | Admit to ICU as appropriate. |
| | If no improvement within 24 hours, manage as a Grade 3 |
| | event: |
| | Initiate workup and assess for signs and symptoms of |
| | MAS/HLH. |
| | May receive the next dose of bispecific antibody if symptoms |
| | resolve to Grade ≤ 1 for 3 consecutive days. |
| | Restarting infusion: |
| | Wait until 30 minutes after the event has resolved before |
| | restarting the infusion at up to 25% of the original infusion rate. |
| | If hypotension or hypoxia recurs, stop infusion immediately. |
| | Bispecific antibody should not be re-administered (restarted) |
| | again during this cycle. |
| | If hypotension or hypoxia recurs, manage as a Grade 3 event. |
| | Next cycle: |
| | May receive the next dose of bispecific antibody if symptoms |
| | resolve to Grade ≤ 1 for 3 consecutive days, as follows: |
| | Administer bispecific antibody at 50% of the initial infusion rate of |
| | the previous cycle if the event occurred during or within 24 hours |
| | of the infusion. ^{d} |
| | Subsequent cycles: |
| | If there is an occurrence of IRR or CRS Grade ≥ 3 in any of the |
| | subsequent cycles, permanently discontinue bispecific antibody |
| | regardless of recovery (see Grade 3 management guidelines). |
| | If there is an occurrence of a Grade ≤ 2 CRS in subsequent |
| | cycles, manage as indicated by severity (see Grade 1 or 2 |
| | management guidelines). |
| Grade 3 | Immediate actions: |
| Hypotension: requires multiple | Stop further infusion of bispecific antibody. |
| pressor or high-dose pressor ^{c} | Treat symptomatically as indicated, including antihistamines, |
| Hypoxia: requires ≥40% FiO2 | antipyretics, and/or analgesics as needed. |
| to maintain adequate | Provide other supportive care as clinically indicated (e.g., fever |
| hemoglobin oxygen saturation | and neutropenia, infection). |
| Organ toxicity: Grade 3 (e.g., | Monitor fluid balance; administer IV fluids as clinically indicated. |
| Grade 4 transaminitis) | Hospitalize patient for at least 24 hours. |
| | Treat with IV corticosteroids (such as methylprednisolone 2 |
| | mg/kg/day or, if neurologic symptoms are present, |
| | dexamethasone 0.5 mg/kg/day). |
| | Administer tocilizumab 8 mg/kg IV. |
| | If there is no improvement after 24 hours, repeat tocilizumab |
| | administration. |
| | Initiate work up and assess for signs and symptoms of |
| | MAS/HLH. |
| | Monitor cardiopulmonary and organ function in ICU. |
| | Provide oxygen for hypoxia. |
| | Admission to ICU is recommended. |
| | Restarting infusion: |
| | Bispecific antibody should not be administered again during |
| | this cycle. |
| | Next cycle: |
| | If the patient had a Grade ≥ 2 IRR or CRS in any previous |
| | cycle, permanently discontinue bispecific antibody. |
| | If patient does not recover (is febrile or still on vasopressors) |
| | within 8 hours after corticosteroid and tocilizumab treatment, |
| | permanently discontinue bispecific antibody. |
| | If patient recovers (is afebrile and off vasopressors) within 8 |
| | hours following corticosteroid and tocilizumab treatment, |
| | bispecific antibody can be administered in next cycle, as follows: |
| | Hospitalize patient for at least 24 hours. |
| | Administer bispecific antibody at 50% of the initial infusion rate |
| | of the previous cycle if the event occurred during or within 24 |
| | hours of the infusion. ^{d} |
| | Subsequent cycles: |
| | If a Grade ≥ 3 CRS recurs, permanently discontinue bispecific |
| | antibody. |
| | If there is an occurrence of a Grade ≤ 2 CRS in subsequent |
| | cycles, manage as indicated by severity (i.e., Grade 1 or 2 |
| | management guidelines). |
| Grade 4 | Follow all Grade 3 management guidelines. |
| Mechanical ventilation | Permanently discontinue bispecific antibody treatment. |
| required; | |
| Organ toxicity: Grade 4 | |
| (excluding transaminitis) | |

CRS = cytokine release syndrome; HLH = hemophagocytic lymphohistiocytosis; ICU = intensive care unit; IV = intravenous; MAS = macrophage activation syndrome.
Note:
CRS is a disorder characterized by nausea, headache, tachycardia, hypotension, rash, shortness of breath, and renal, coagulation, hepatic and neurologic disorders; it is caused by the release of cytokines from cells (Lee et al., Blood, 124: 188-195, 2014).
^{a} Refer to Table A for description of grading of symptoms.
^{b} Guidance for CRS management based on Lee et al., Blood, 124: 188-195, 2014.
^{c} Refer to Table B for a description and calculation of high-dose vasopressors.
^{d} If the patient does not experience CRS during the next infusion at the 50% reduced rate, the infusion rate can be increased to the initial rate in subsequent cycles. However, if this patient experiences another CRS event, the infusion rate should be reduced by 25%-50% depending on the severity of the event.

### Management of Grade 2 CRS Events

If the subject has a grade 2 CRS event (e.g., a grade 2 CRS event in the absence of comorbidities or in the presence of minimal comorbidities) following administration of the therapeutic multispecific antigen-binding molecule (e.g., the bispecific antibody), the method may further include treating the symptoms of the grade 2 CRS event while suspending treatment with the multispecific antigen-binding molecule (e.g., the bispecific antibody). If the grade 2 CRS event then resolves to a grade ≤1 CRS event for at least three consecutive days, the method may further include resuming treatment with the multispecific antigen-binding molecule (e.g., the bispecific antibody) without altering the dose. On the other hand, if the grade 2 CRS event does not resolve or worsens to a grade ≥3 CRS event within 24 hours of treating the symptoms of the grade 2 CRS event, the method may further involve administering to the subject an effective amount of an interleukin-6 receptor (IL-6R) antagonist (e.g., an anti-IL-6R antibody, e.g., tocilizumab (ACTEMRA®/ROACTEMRA®)) to manage the grade 2 or grade 3 CRS event. In some instances, tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

If the subject has a grade 2 CRS event in the presence of extensive comorbidities following administration of the therapeutic multispecific antigen-binding molecule (e.g., the bispecific antibody), the method may further include administering to the subject a first dose of an IL-6R antagonist (e.g., an anti-IL-6R antibody, e.g., tocilizumab (ACTEMRA®/ROACTEMRA®)) to manage the grade 2 CRS event while suspending treatment with the multispecific antigen-binding molecule. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, if the grade 2 CRS event resolves to a grade ≤1 CRS event within two weeks, the method further includes resuming treatment with the multispecific antigen-binding molecule (e.g., the bispecific antibody) at a reduced dose. In some instances, the reduced dose is 50% of the initial infusion rate of the previous cycle if the event occurred during or within 24 hours of the infusion. If, on the other hand, the grade 2 CRS event does not resolve or worsens to a grade 3 CRS event within 24 hours of treating the symptoms of the grade 2 CRS event, the method may further include administering to the subject one or more (e.g., one, two, three, four, or five or more) additional doses of an IL-6R antagonist (e.g., an anti-IL-6R antibody, e.g., tocilizumab) to manage the grade 2 or grade 3 CRS event. In some particular instances, the grade 2 CRS event does not resolve or worsens to a grade 3 CRS event within 24 hours of treating the symptoms of the grade 2 CRS event, and the method may further include administering to the subject one or more additional doses of tocilizumab to manage the grade 2 or grade 3 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

### Management of Grade 3 CRS Events

If the subject has a grade 3 CRS event following administration of the therapeutic multispecific antigen-binding molecule (e.g., the bispecific antibody), the method may further include administering to the subject a first dose of an IL-6R antagonist (e.g., an anti-IL-6R antibody, e.g., tocilizumab (ACTEMRA®/ROACTEMRA®)) to manage the grade 3 CRS event while suspending treatment with the multispecific antigen-binding molecule. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, the subject recovers (e.g., is afebrile and off vasopressors) within 8 hours following treatment with the multispecific antigen-binding molecule (e.g., the bispecific antibody), and the method further includes resuming treatment with the multispecific antigen-binding molecule (e.g., the bispecific antibody) at a reduced dose. In some instances, the reduced dose is 50% of the initial infusion rate of the previous cycle if the event occurred during or within 24 hours of the infusion. In other instances, if the grade 3 CRS event does not resolve or worsens to a grade 4 CRS event within 24 hours of treating the symptoms of the grade 3 CRS event, the method may further include administering to the subject one or more (e.g., one, two, three, four, or five or more) additional doses of an IL-6R antagonist (e.g., an anti-IL-6R antibody, e.g., tocilizumab) to manage the grade 3 or grade 4 CRS event. In some particular instances, the grade 3 CRS event does not resolve or worsens to a grade 4 CRS event within 24 hours of treating the symptoms of the grade 3 CRS event, and the method further includes administering to the subject one or more additional doses of tocilizumab to manage the grade 3 or grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

### Management of Grade 4 CRS Events

If the subject has a grade 4 CRS event following administration of the therapeutic multispecific antigen-binding molecule (e.g., the bispecific antibody), the method may further include administering to the subject a first dose of an IL-6R antagonist (e.g., an anti-IL-6R antibody, e.g., tocilizumab (ACTEMRA®/ROACTEMRA®)) to manage the grade 4 CRS event and permanently discontinuing treatment with the multispecific antigen-binding molecule. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. The grade 4 CRS event may, in some instances, resolve within 24 of treating the symptoms of the grade 4 CRS event. If the grade 4 CRS event does not resolve within 24 hours of treating the symptoms of the grade 4 CRS event, the method may further include administering to the subject one or more additional doses of an IL-6R antagonist (e.g., an anti-IL-6R antibody, e.g., tocilizumab (ACTEMRA®/ROACTEMRA®)) to manage the grade 4 CRS event. In some particular instances, the grade 4 CRS event does not resolve within 24 hours of treating the symptoms of the grade 4 CRS event, and the method further includes administering to the subject one or more (e.g., one, two, three, four, or five or more) additional doses of tocilizumab to manage the grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

### EXAMPLES

The following are examples of methods and compositions of the invention. It is understood that various other embodiments may be practiced, given the general description provided above.

### Example 1. Recombinant STEAP1 Expression and Purification

### Recombinant STEAP1 Expression

Full length human STEAP1 DNA sequence (M1-L339) was synthesized (Genescript) and cloned into a modified pAcGP67A vector downstream of the polyhedron promoter. Recombinant baculovirus was generated using the BACULOGOLD™ system (BD Biosciences) following standard protocols. Recombinant STEAP1 was expressed with a C-terminal FLAG affinity tag followed by an Avi-tag; the sequence of the construct is shown below (the underlined sequence is the FLAG affinity tag while the Avi-tag is in bold):

MGSMESRKDITNQEELWKMKPRRNLEEDDYLHKDTGETSMLKRPV
LLHLHQTAHADEFDCPSELQHTQELFPQWHLPIKIAAIIASLTFL
YTLLREVIHPLATSHQQYFYKIPILVINKVLPMVSITLLALVYLP
GVIAAIVQLHNGTKYKKFPHWLDKWMLTRKQFGLLSFFFAVLHAI
YSLSYPMRRSYRYKLLNWAYQQVQQNKEDAWIEHDVWRMEIYVSL
GIVGLAILALLAVTSIPSVSDSLTWREFHYIQSKLGIVSLLLGTI
HALIFAWNKWIDIKQFVWYTPPTFMIAVFLPIVVLIFKSILFLPC
LRKKILKIRHGWEDVTKINKTEICSQLGNSGLNDIFEAQKIEWHE
GENLYFQSDYKDDDDK

2 L of Sf9 cells were split to 2×10⁶/ml and added to 5 L Thompson Optimum Growth flasks. Flasks were infected with 2.5 mL/L P3 virus at an approximate multiplicity of infection (MOI) of 0.5. After 6 hours, the following additives were added to the cultures (note: additives were prepared fresh on day of use): 1 mM 5-aminolevulinic acid (Sigma Chemicals; for 1 M stock, resuspend 167.5 gm/L in H₂O and filter sterilize) and 5 μM ferric chloride (Sigma Chemicals; for 1 mM stock, resuspend 162 mg/L in H₂O and filter sterilize). Cultures were grown at 37 °C under 120 rpm agitation and harvested by centrifugation after 72 hours.

Table 5 shows primers used to amplify IGHV5 sequences from immunized rat.

TABLE 5
RT step primer mix:
G1R.1
TCCAGGGGCCAGTGGATAGAC
G2R.1
CACACMGGGGCCAGTGGATAGAC
G3R.1
GCAGCCAGGGACCAAGGGATAGAC
IgAR1
GGAAGTAATCRTGAATCAGGCAGCC
PCR primer mix:
G1R.2
AGATGGGGGTGTCGTTTTGGC
G2R.2
CGATGGGGSTGTTGTTTTGGC
G3R.2
GACAGATGGGGCTGTTGTTGTAGC
IgAR2
TGTCAGTGGGTAGATGGTGGG
VH5-For
TGTCCTTTTCATAAAAGGTGTCCAGTGT

### Purification of Recombinant Soluble STEAP1

Pelleted cells were resuspended in 25 mM Tris pH 7.5, 150 mM NaCl, 5 mM MgCl₂ (Buffer A) supplemented with COMPLETE™ EDTA-free protease-inhibitor cocktail tablets (Roche) and benzonase (prepared in-house). Cell suspension was passed through a microfluidizer once at a pressure setting of 15,000 psi. Following cell lysis, suspension was spun down at 40,000 rpm at 4 °C for 1 hour. Isolated membranes were resuspended into Buffer A supplemented with 1% (w/v) LMNG (lauryl maltose neopentyl glycol) and 0.1% (w/v) cholesterol hemisuccinate (CHS) from Anatrace (#NG310-CH210) and solubilization was carried out with gentle agitation for 2 hours at 4 °C. After ultracentrifugation at 40,000 rpm at 4 °C for 30 min, clarified supernatant was mixed with anti-M2 FLAG resin (Sigma) pre-equilibrated with Buffer B (25 mM Tris pH 8, 150 mM NaCl, 0.02% LMNG, 0.002% CHS) for 1 h at 4 °C. Anti-FLAG resin was collected by gravity flow and washed with 5 column volumes of Buffer B. FLAG-tagged STEAP1 was eluted with Buffer C (25 mM Tris pH 8, 150 mM NaCl, 0.02% LMNG, 0.002% CHS supplemented with 150 μg/mL 1× FLAG peptide). Concentrated eluate was passed over a SUPERDEX® S200 16/60 GL column (GE Healthcare) equilibrated in Buffer B. Peak fractions were collected and concentrated using a AMICON® Ultra-15 Centrifugal Filter Units (100K MWCO, Millipore Sigma) down to 1 mg/mL. 800 μL of the solution was mixed with 100 μL of BiomixA and 100 μL BiomixB from BirA500 kit (Avidity). 15 μg of BirA biotin-protein ligase was added and mixture was incubated at 4 °C for 24h. Mixture was finally polished over a SUPERDEX® S200 16/60 GL column (GE Healthcare) equilibrated in Buffer B. Protein purity was evaluated by SDS-PAGE.

### Example 2. Development and Characterization of Rat Anti-Human STEAP1 Antibodies

Sprague Dawley rats (Charles River, Hollister, CA) were immunized with 100 μg of plasmid DNA encoding human STEAP1 along with plasmid DNA encoding rat Flt3L and rat GM-CSF (Genentech, South San Francisco, CA) divided among sites: i.p., s.c. at base of tail, s.c. at nape of neck, and s.c. in both hocks. Following four doses of DNA, the rats received two doses of 100 μg of a plurality of extracellular vesicles expressing human STEAP1 on the surface, divided among multiple sites. Some rats were instead immunized with a priming dose of 30 μg human STEAP1 protein solubilized in detergent mixed with MPL+TDM adjuvant (Sigma-Aldrich, St. Louis, MO) or mixed with a combination of TLR agonists: 50 μg MPL (Sigma-Aldrich), 20 μg R848 (Invivogen, San Diego, CA), 10 μg PolyI:C (Invivogen), and 10 μg CpG (Invivogen) divided among multiple sites. For additional protein boosts, the rats received 15 μg protein diluted in PBS. Rats were dosed every two weeks. Polyclonal antisera from these rats were purified and tested by FACS for binding to human STEAP1 on the surface of 293 or RBA cells. Multiple lymph nodes were harvested three days after the last immunization from the rats that showed detectable FACS reactivity against human STEAP1. Protein-immunized rats showed a weaker FACS response than that observed with the DNA/EV-immunized rats, thus the DNA/EV-immunized rats were selected for harvest. IgM-negative B-cells from these rats were purified from whole lymphocytes using magnetic separation (Miltenyi Biotec, San Diego, CA) and were fused with Sp2ab mouse myeloma cells (Abeome, Athens, GA) via electrofusion (Harvard Apparatus, Holliston, MA). Fused cells were incubated at 37 °C, 7% CO₂, overnight in CLONACELL™-HY Medium C (StemCell Technologies, Vancouver, BC, Canada), before centrifugation and being resuspended in CLONACELL™-HY Medium E (StemCell Technologies) supplemented with hypoxanthine, aminopterin, and thymidine (HAT) (Sigma-Aldrich), dispensed into 12-well plates and allowed to grow at 37 °C, 7% CO₂. Four days after plating, rat hybridoma cells were stained with anti-rat IgM antibody (Jackson ImmunoResearch, West Grove, PA) and extracellular vesicles expressing fluorescent-labeled viral proteins, with or without expression of human STEAP1.

Alternatively, IgM-negative B-cells from these rats were purified from whole lymphocytes using magnetic separation (Miltenyi Biotec, San Diego, CA) and stained with anti-rat IgM antibody (Jackson ImmunoResearch, West Grove, PA) and fluorescent-labeled STEAP1 protein incorporated into nanodiscs (Genentech). Cells showing minimal rat IgM expression while binding to the STEAP1-expressing extracellular vesicles were sorted and deposited into 96-well plates containing Medium E (StemCell Technologies) using a FACSAria™ III sorter (BD, Franklin Lakes, NJ). Rat B-cells showing minimal rat IgM expression while binding to the STEAP1 protein were sorted and deposited into 96-well plates containing a culture medium containing feeder cells and supplemented with cytokines using a FACSAria™ III sorter (BD, Franklin Lakes, NJ). Supernatants were screened by ELISA against anti-rat IgG seven days after sorting. Cell lines demonstrating rat IgG expression were scaled-up and supernatants were harvested and purified by protein G (GAMMABIND™ Plus, GE Healthcare, Pittsburgh, PA). The purified rat IgG samples were tested by flow cytometry (FACS) for binding to human STEAP1 expressed on the surface of 293 cells, as well as multiple cell lines endogenously expressing human STEAP1. B cell clone supernatants were screened by ELISA against anti-rat IgG seven days after sorting. Supernatants demonstrating rat IgG binding were tested by FACS for binding to human STEAP1 expressed on the surface of 293 cells. RNA was extracted from B-cells that showed STEAP1 FACS binding for molecular cloning and recombinant expression. Recombinant antibodies were tested by FACS for binding to human STEAP1 expressed on the surface of 293 cells, as well as multiple cell lines endogenously expressing human STEAP1.

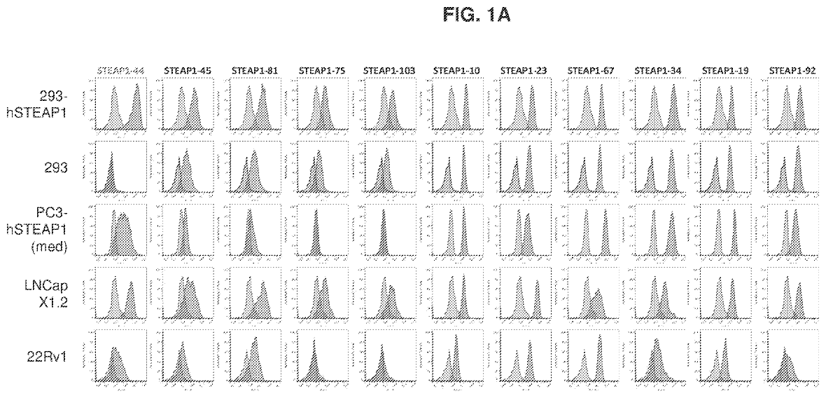

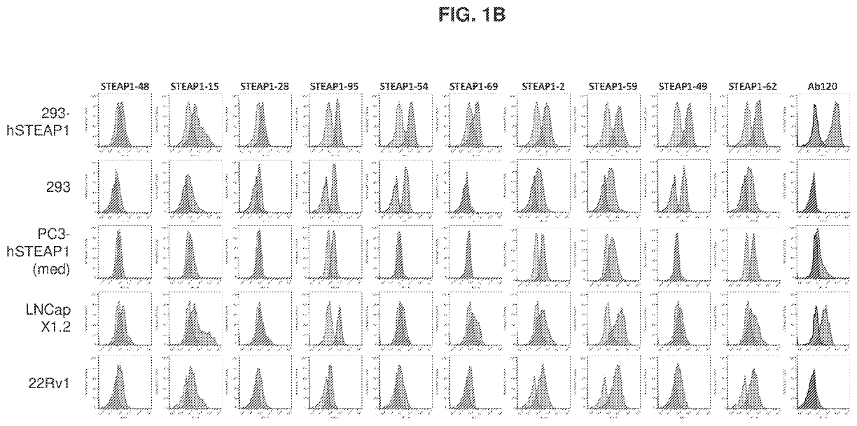

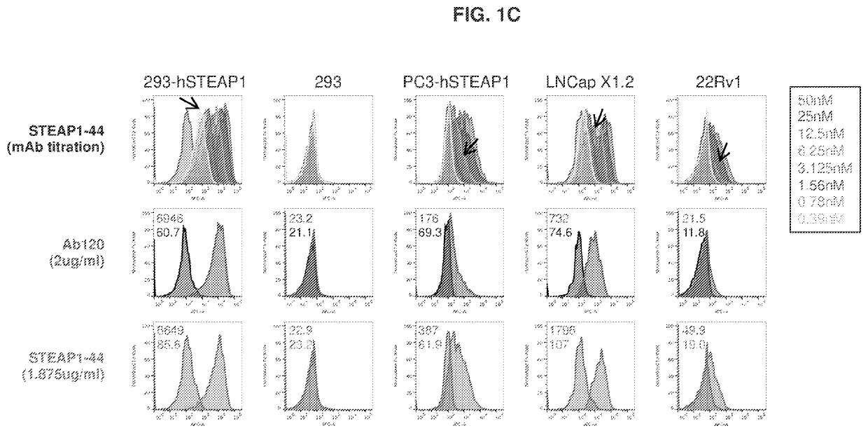

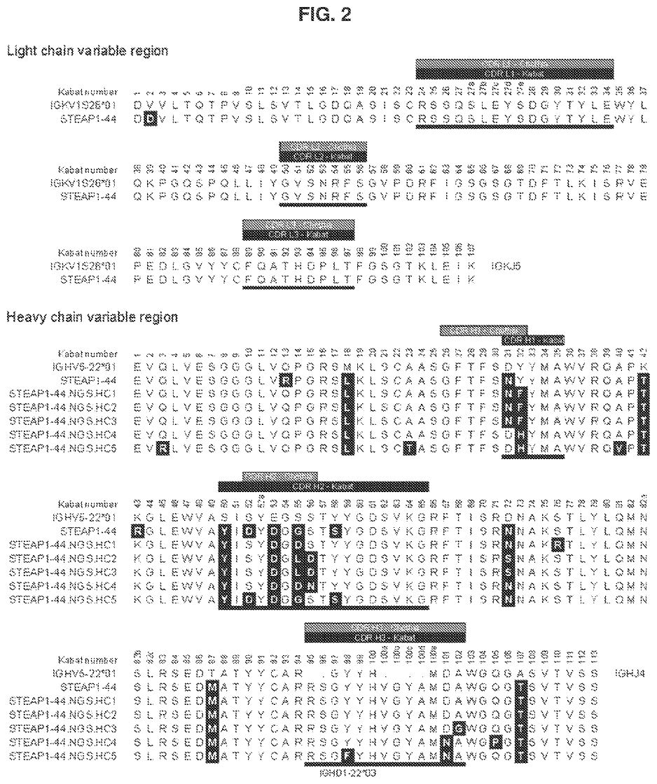

Of the 21 rat antibodies with binding to 293 cells expressing human STEAP1, 2 clones show specific binding without any reactivity against parental 293 cells, clones STEAP1-44 and STEAP1-69 (FIG. 1A and FIG. 1B). Of these two antibodies, antibody STEAP1-44 (also referred to herein as Ab44) showed strong binding to STEAP-1-expressing cells at a concentration of 7.5 μg/ml, comparable or better than the Ab120 control at 2 μg/ml. Antibody STEAP1-44 was tested in titrations on cells expressing STEAP1 at different levels (293-hSTEAP1>LNCaP X1.2>PC3-hSTEAP1 >22Rv1) and in control 293 cells, alongside control Ab120 tested at 2 μg/ml. The minimal concentration in which binding of STEAP1-44 to 293-hSTEAP1, LNCaP X1.2, PC3-hSTEAP1 and 22Rv1 cells was observed was 0.39 nM, 3.125 nM, 6.25 nM and 12.5 nM, respectively (FIG. 1C). Binding of STEAP1-44 to the different cell lines at 1.875 μg/ml was similar to binding of control Ab120 to the same cells at 2 μg/ml. The variable regions of the antibody STEAP1-44 were sequenced (FIG. 2).

### Example 3. Development and Characterization of Rabbit Anti-Human STEAP1 Antibodies

New Zealand White rabbits (Charles River, Hollister, CA) were immunized with 600 μg of plasmid DNA encoding human STEAP1 along with plasmid DNA encoding rabbit Flt3L and rabbit GM-CSF (Genentech, South San Francisco, CA) divided among multiple sites along the back. Following four doses of DNA, the rabbits received three doses of 250 μg of a plurality of extracellular vesicles expressing human STEAP1 on the surface, divided among multiple sites. Following three doses of vesicles, the rabbits received three doses of 50 μg of detergent-solubilized STEAP1 protein, divided among multiple sites. Rabbits were dosed every two weeks. Polyclonal antisera from these rabbits were purified and tested by FACS for binding to human STEAP1 on the surface of 293 or RK13 cells. Twenty mL of blood was drawn three days after the last immunization from the rabbits that showed detectable FACS reactivity against human STEAP1. IgM-negative B-cells from these rabbits were purified from PBMCs using magnetic separation (Miltenyi Biotec, San Diego, CA). Rabbit cells were stained with anti-rabbit IgG antibody (Jackson ImmunoResearch, West Grove, PA) and fluorescent-labeled STEAP1 protein incorporated into nanodiscs (Genentech). B-cells showing rabbit IgG expression while binding to STEAP1 protein were sorted and deposited into 96-well plates with a culture medium containing feeder cells and supplemented with cytokines using a FACSAria™ I∥ sorter (BD, Franklin Lakes, NJ). Supernatants were screened by ELISA against anti-rabbit IgG seven days after sorting. Supernatants demonstrating rabbit IgG binding were tested by FACS for binding to human STEAP1 expressed on the surface of 293 cells. RNA was extracted from B-cells that showed STEAP1 FACS binding for molecular cloning and recombinant expression. Recombinant antibodies were tested by FACS for binding to human STEAP1 expressed on the surface of 293 cells, as well as multiple cell lines endogenously expressing human STEAP1.

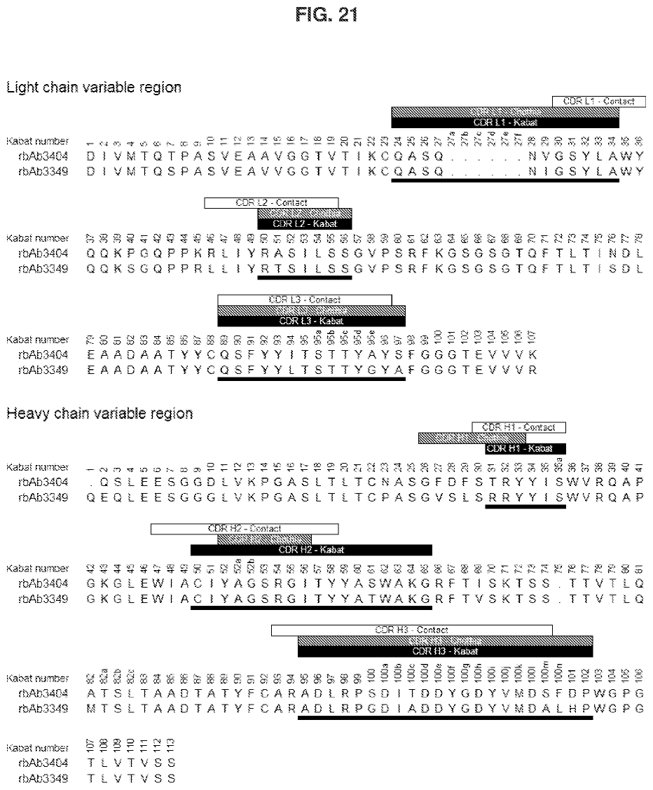

FIG. 21 shows variable region sequences of rbAb3404 and rbAb3349.

### Example 4. Affinity Maturation of STEAP1-44 by Repertoire Next-Generation Sequencing (NGS)

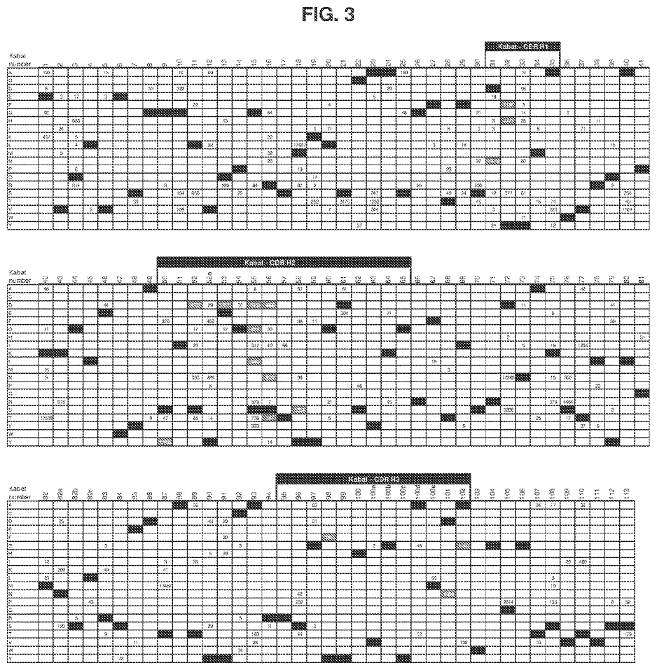

Antibody STEAP1-44 was affinity matured by rat NGS repertoire data mining (Hsiao et al., 2019). Briefly, bone marrow and spleen tissues from the same rat from which STEAP1-44 was derived by hybridoma using lymph node tissues were used to extract total RNA and used in an RT-PCR step to amplify VH segments with the rat IGHV5-22 germline segment, the same germline segment used by STEAP1-44. Amplicons were submitted to paired-end sequencing in an ILLUMINA® MiSeq® instrument. Sequence reads were assembled into full-length VH sequences and parsed for germline segment and CDR boundaries using IgBlast (Goldstein et al., 2019). A total of 17,902 sequence reads with the IGHV5-22 and IGHJ4 germline segment sequences and a Kabat CDR H3 length of 13 amino acid residues belonging to the same clonotype as STEAP1-44 antibody were selected from the dataset. These were aligned to the IGHV5-22 germline segment sequence and mutations were scored (FIG. 3). A total of 5 variants including the most prevalent somatic mutations in CDR regions in the dataset (FIGS. 2 and 3) were selected for expression of full-length IgG by combining with the STEAP1-44 light chain. IgGs were expressed by transient transfection of Expi293 cells and purified by protein A chromatography (Bos et al., 2015; Luan et al., 2018).

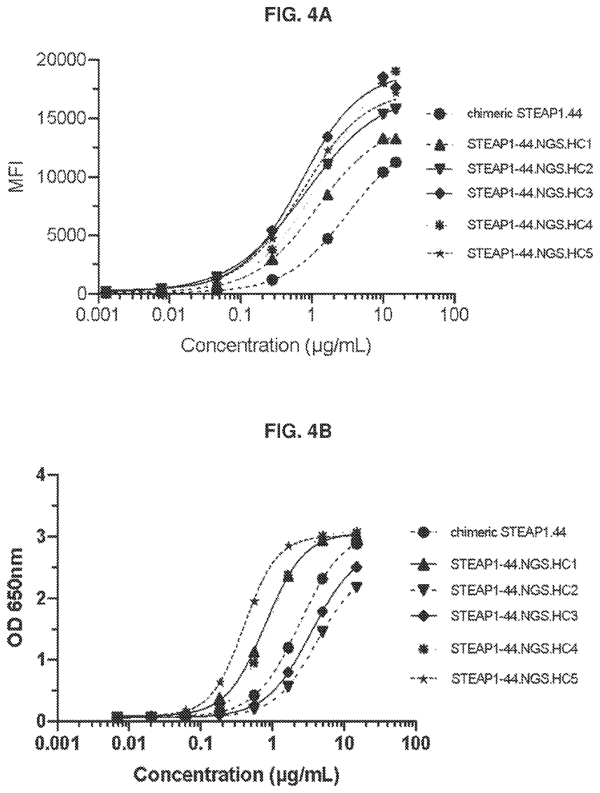

Clonal variants were tested by FACS on STEAP1-expressing cells and by ELISA with soluble biotinylated recombinant STEAP1. Serial dilutions show all variants binding more strongly than STEAP1-44 (FIGS. 4A and 4B). However, the ranking of the variants varies between assays, with STEAP1-44.NGS.HC3 showing the strongest binding by FACS, STEAP1-44.NGS.HC5 showing the strongest binding by ELISA to recombinant STEAP1 and STEAP1-44.NGS.HC4 showing strong binding in both assays. Therefore, these 3 variants were further examined in more detail upon humanization.

### Example 5. Humanization of STEAP1-44

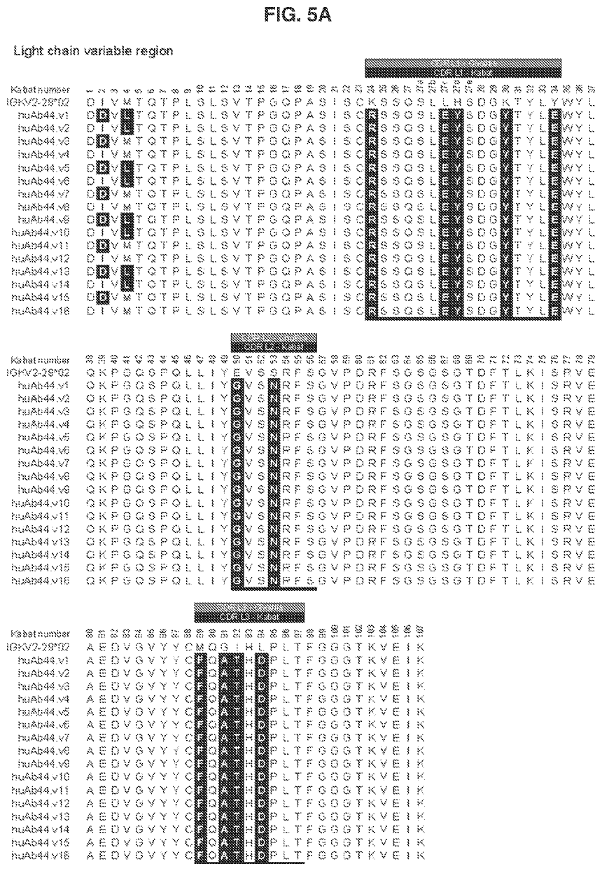

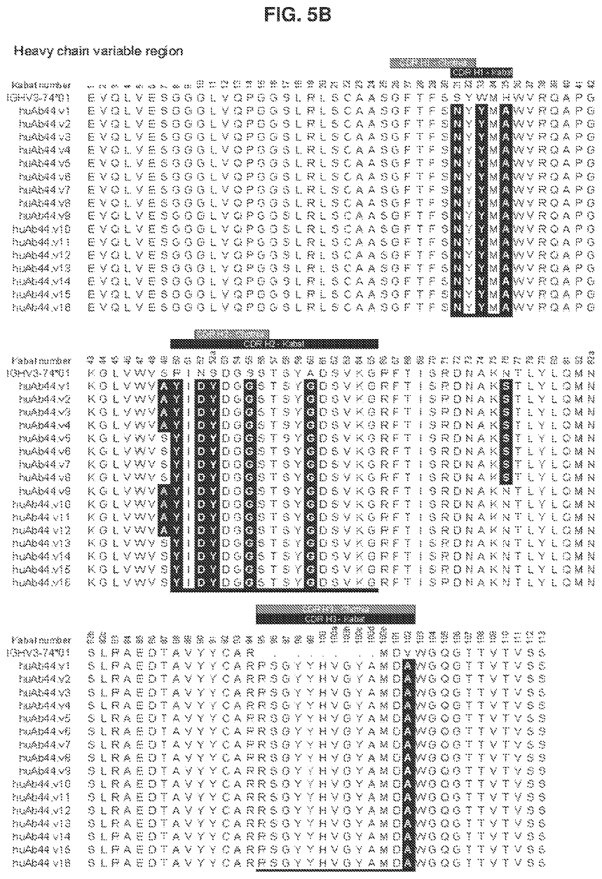

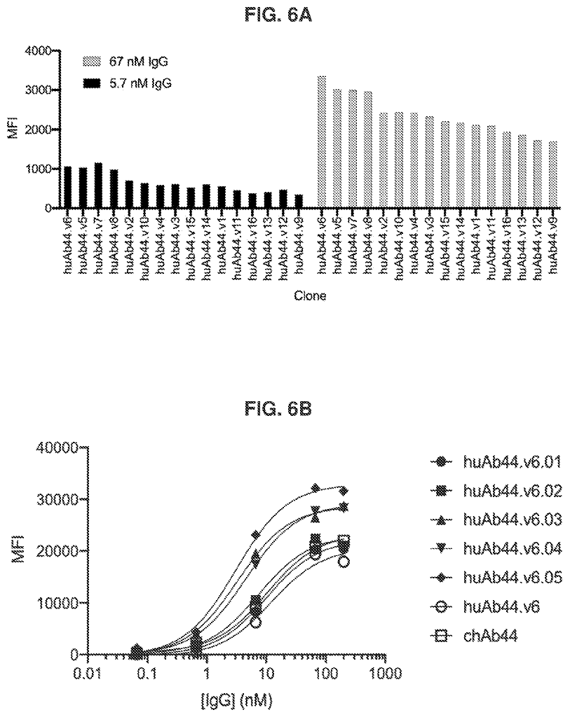

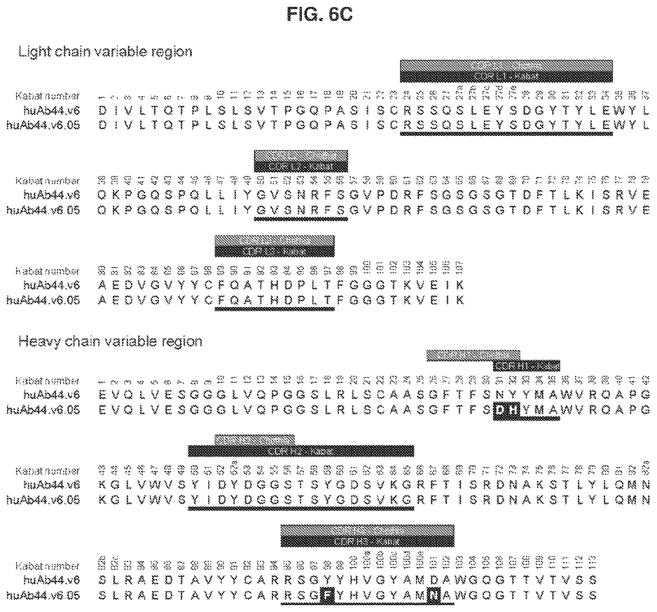

Antibody STEAP1-44 was humanized in VK2 and VH3 frameworks by CDR grafting. Briefly, CDR regions of the rat STEAP1-44 antibody were grafted into light chain IGKV2-29 and heavy chain IGKV3-74 frameworks, the closest human germline segments of STEAP1-44. CDR regions included, for the light chain, Kabat positions 24-34 (CDR L1), 50-56 (CDR L2) and 89-97 (CDR L3), and, for the heavy chain, Kabat positions 26-35 (CDR H1), 50-65 (CDR H2) and 93-102 (CDR H3). Rat framework residues known as “Vernier” positions and in the domain interface that differed between the rat STEAP1-44 and human germlines were added to the CDR graft to rescue binding to antigen, as it is normally done in CDR graft humanizations (see, e.g., U.S. Pat. No. 8,426,147). These included in this case light chain residues 2 and 4 and heavy chain residues 49 and 76. The final CDR graft including framework changes to rat residues was named huAb44.v1 (FIGS. 5A and 5B). Variants with different combinations of the Vernier positions, a total of 16 variants, were expressed as human IgG1, purified by protein A and size exclusion chromatography and tested in FACS for binding to cells expressing STEAP1. Of all the variants, huAb44.v6 gave the highest binding to LNCaP-X1.2 cells and was selected as the final humanized variant (FIG. 6A).

Mutations from the NGS repertoire deep sequencing were incorporated into huAb44.v6. Somatic mutations from STEAP1.NGS.HC1 to 5 were added to huAb44.v6 to create variants huAb44.v6.01 to 05, respectively. These variants were tested for binding by FACS on STEAP1-expressing cells. (FIG. 6B). All NGS mutants incorporated into huAb44.v6 showed improved binding to LNCaP-X1.2 cells compared to huAb44.v6.

FIG. 6C shows alignment of humanized antibody variant huAb44.v6.05 against humanized antibody huAb44.v6. Differences in huAb44.v6.05 relative huAb44.v6 are shown in black background. CDR boundaries according to the Kabat and Chothia systems shown above the alignments, with Kabat CDRs boundaries underlined. Residue numbering is according to the Kabat system.

### Example 6. T Cell Mediated Cell Killing by STEAP1-TDB Molecules

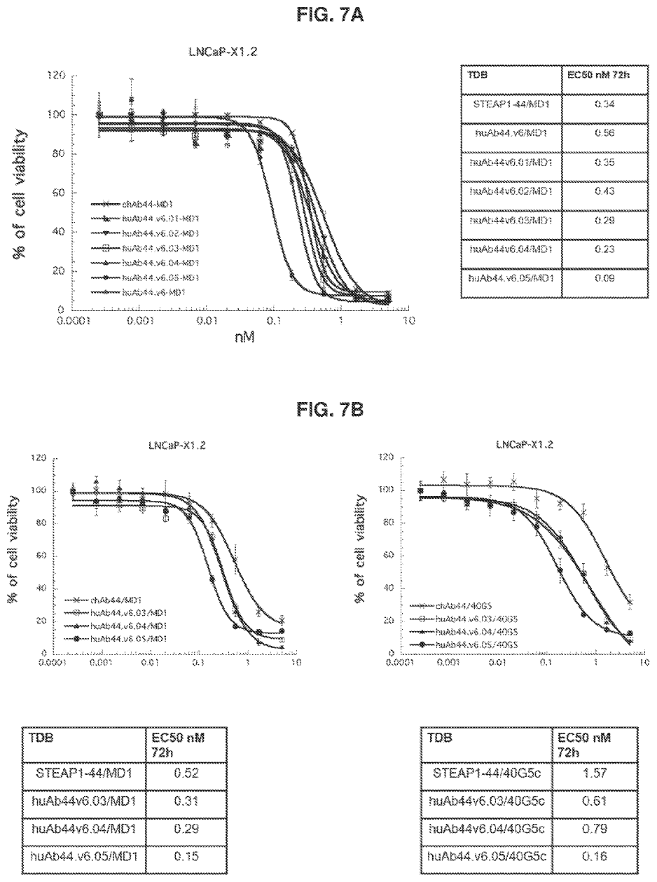

Variants of the humanized antibody huAb44.v6 were assembled as bispecific molecules with anti-CD3 antibodies MD1 (higher affinity for CD3) and 40G5c (lower affinity for CD3) and evaluated in T-cell mediated target cell killing assays. T cell mediated cell killing was assessed on the high STEAP1 expressing LNCaP-X1.2 cell line. Briefly, human blood samples were obtained from healthy volunteers through the Genentech employee donation program. PBMCs were separated from the blood of healthy volunteers using FICOLL®-Paque and a LEUCOSEP™ tube. CD8+ T cells were isolated from PBMCs using a Miltenyi CD8+ T cell isolation kit (cat #130-096-495) and Miltenyi XS column (cat #130-041-202). LNCaP-X1.2 cells (4000 cells/well) were plated in clear-bottomed 96-well plates (Corning, cat #3904). The next day target cells were co-cultured with freshly isolated CD8+ cells at a 1:4 ratio. The STEAP1 TDBs assembled with the anti-CD3 antibody MD1 were added at the indicated concentrations (range of 0-5 nM) in triplicates and cell killing was assessed after 72 hour incubation at 37 °C. Cell viability was measured using CELLTITER-GLO® luminescent cell viability reagent (Promega, cat #G7570). The EC₅₀ values for STEAP1-TDB mediated cell killing was in the picomolar range for all variants. Antibody huAb44.v6.05 demonstrated the highest potency with an EC₅₀ of 90 pM an apparent improvement of about 6-fold relative to parental huAb44.v6 (FIG. 7A).

The three most potent antibodies (huAb44v6.03, huAb44v6.04, huAb44v6.05) were also combined with the anti-CD3 antibody 40G5c and cell killing of all anti-STEAP1 TDBs was measured in LNCaP-X1.2 cells using a different PBMC donor. The experimental set up was identical as described above. Again, the same ranking was observed when testing the huAb44.v6.03, huAb44.v6.04 and huAb44.v6.05 antibodies with either anti-CD3 arm. The most potent cell killing was observed with huAb44.v6.05-MD1 and huAb44.v6.05-40G5c with EC₅₀ values of 150 pM and 160 pM, respectively (FIG. 7B).

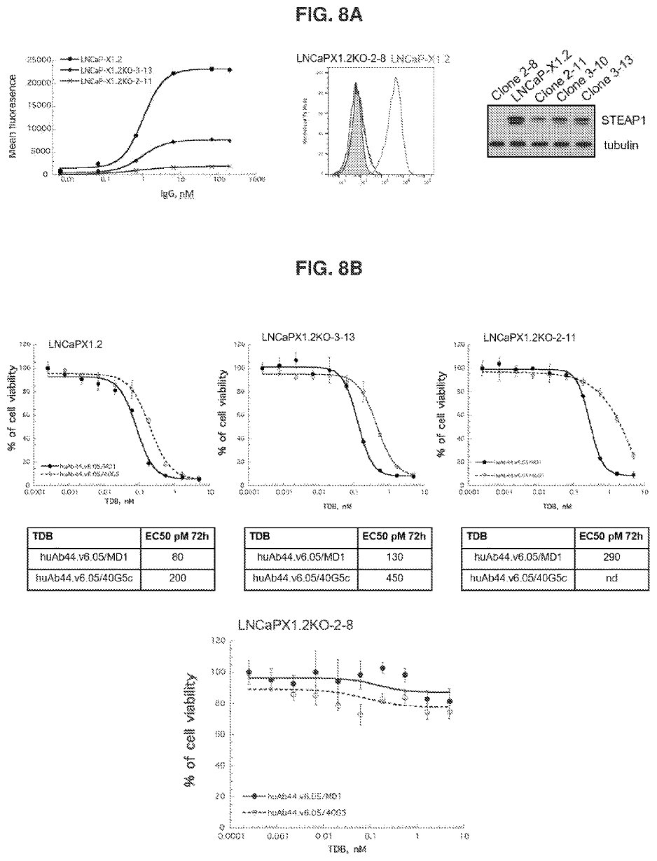

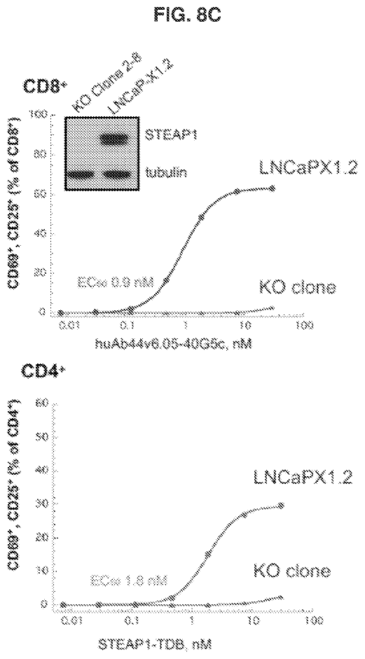

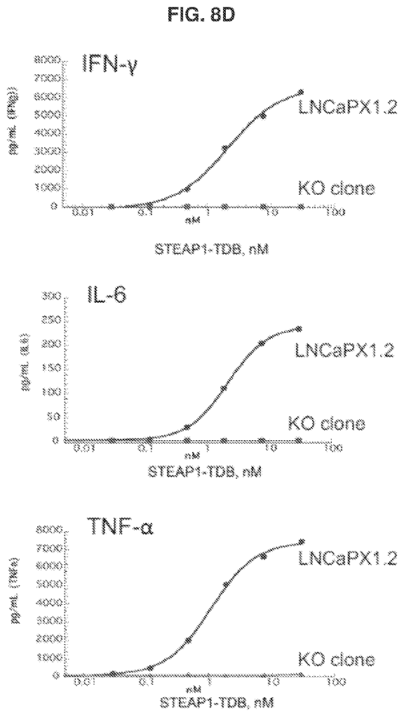

To determine cell killing potency in cells with lower STEAP1 expression, LNCaP-X1.2 derivatives (LNCaP-X1.2 KO-3-13 and LNCaP-X1.2 KO-2-11) were generated using gene editing technology. These lines represent various STEAP1 levels observed in prostate cancer. Additionally, a cell line devoid of STEAP1 expression (LNCaP-X1.2KO-2-8) was created as a control line (FIG. 8A). HuAb44.v6.05-MD1 and huAb44.v6.05-40G5c were tested in these cell lines. Potent cell killing was observed in all STEAP1 expressing cell lines but not in the STEAP1 negative cell line LNCaP-X1.2KO-2-8. The potency of huAb44.v6.05-MD1 decreased slightly in the lower STEAP1 expressing cell lines (LNCaP-X1.2, LNCaP-X1.2 KO-3-13, LNCaP-X1.2 KO-2-11) with EC₅₀ values ranging from 80 pM to 200 pM and 450 pM respectively. In general, huAb44.v6.05-40G5c showed reduced efficacy compared to huAb44.v6.05-MD1, however, 100% cell killing was still observed in LNCaP-X1.2 and LNCaP-X1.2-KO-3-13 under these conditions (FIG. 8B). Additional experiments confirming that STEAP1-TDB activity is target-dependent are shown in FIG. 8C (target-dependent T cell activation at 24 h) and FIG. 8D (cytokine secretion at 24 h).

### Example 7. In Vivo Activity of STEAP1-TDB

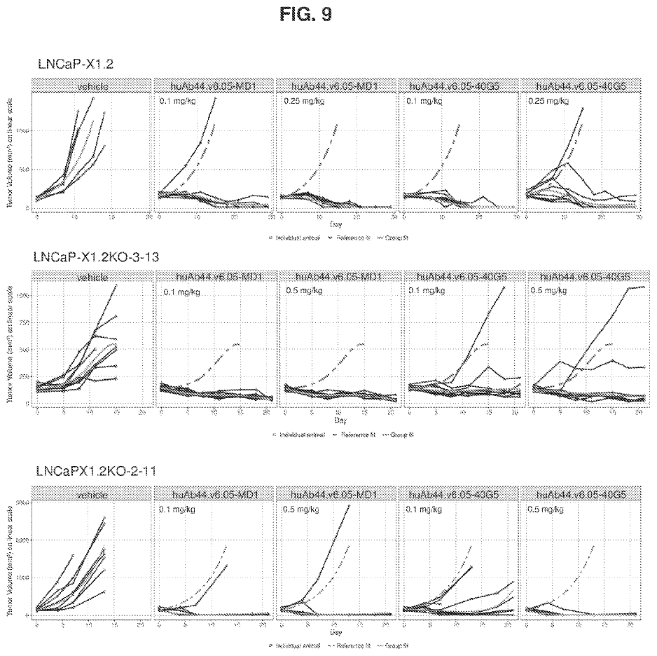

The antitumor activity of STEAP1-TDBs was assessed using NSG mice inoculated with LNCaP-X1.2, LNCaP-X1.2KO-3-13 or LNCaP-X1.2KO-2-11 human prostate cancer cells with or without PBMCs. Briefly, female NOD SCID gamma (NSG) mice, 8-9 weeks of age, were obtained from the Jackson Laboratory (Sacramento, CA). These cells exhibit a range of STEAP1 expression that is representative of prostate cancer patients: low STEAP1 (LNCaPX1.2KO-2-11), medium STEAP1 (LNCaPX1.2 KO-3-13), and high STEAP1 (LNCaPX1.2). Mice were housed at Genentech, Inc., and all experimental procedures conformed to the guiding principles of the American Physiology Society and were approved by Genentech's Institutional Animal Care and Use Committee. Mice were each inoculated subcutaneously in the right flank with 10 million LNCAP-X1.2 cells or subclones suspended in 0.1 mL Hank's Balanced Salt Solution (HBSS). One day after cell inoculation, the mice were given an intraperitoneal injection of 10 million human PBMCs cultured overnight in non-activating conditions. Seven to 10 days post cell inoculation, the tumor bearing mice were randomized into control and treatment groups based on similar sized tumors (average volume of 200 mm³) and dosing initiated at day 0. All treatments were given a single intravenous (IV) injection of either vehicle or STEAP1-TDB, at a concentration range of 0.1 mg/kg to 0.5 mg/kg, in a volume of 100 μL per mouse. Tumor volumes and body weights were measured two times per week over the course of the study. Mice were euthanized when tumor volume exceeded ~2000 mm³ or if the tumor became ulcerated. Individual tumor volume responses to STEAP1-TDBs are shown in FIG. 9. Complete tumor regression was seen with either huAb44.v6.05-MD1 or huAb44.v6.05-40G5c at all concentration tested in LNCaP-X1.2, LNCaPX1.2-KO3-13 and LNCaP-X1.2-2-11 xenograft models. None of tumors in the vehicle control groups showed signs of regression demonstrating that efficacy was dependent on the presence of STEAP1-TDB.

### Example 8. Affinity of huAb44.v6.05

The monovalent affinity of antibody huAb44.v6.05-40G5c for STEAP1 was determined in a label-free system in solution with live 293 cells stably expressing either human or cynomolgus STEAP1. The Kinetic Exclusion Assay (KinExA®), performed by Sapidyne Instruments (Boise, ID), measures the equilibrium binding affinity and kinetics in solution. The equilibrium dissociation constant, K_{D}, and association, kₐ, are experimentally determined, while the rate of dissociation, k_{d}, is calculated based on the equation k_{d}=K_{D}×kₐ. The monovalent binding kinetics of huAb44.v6.05-40G5c for human and cynomolgus STEAP1 on cells determined by the KinExA® assay are:

| Species | K_{D} (nM) | kₐ (M⁻¹s⁻¹) | k^{d} (s⁻¹) |
|---|---|---|---|
| Human STEAP1 | 29 | 3.7 × 10⁵ | 1.0 × 10⁻² |
| Cynomolgus STEAP1 | 14 | 7.4 × 10⁵ | 1.1 × 10⁻² |

### Example 9. Pharmacokinetics of Anti-STEAP1/CD3 T-Cell Dependent Bispecific Antibodies Following Intravenous Dosing in Female SCID Mice

In a first study, pharmacokinetics (PK) of anti-STEAP1 antibodies following administration of a single intravenous (IV) bolus dose of 1 mg/kg was assessed in severe combined immunodeficiency (SCID) mice. This study included chimeric Ab44 (chAb44) as well as the humanized version of Ab44 (huAb44.v6). The in-life phase of the study was conducted at Charles River Laboratories (CRL) South San Francisco, CA in accordance with local Institutional Animal Care and Use Committee guidelines. Naïve female SCID mice weighing 17 to 22 g and approximately 7-9 weeks old at the time of dosing were assigned to dose groups (n=4 animals per group). The test articles were administered as single IV bolus doses via tail vein (dose volume set at 5 mL/kg). Animal restrainers were used for dosing. Serial blood samples (30-40 μL) were collected in serum separator tubes at 0.167, 1, 6, 24, 72, 168, 240, 336 and 504 h post dose via tail nicks. Blood samples were stored at room temperature for 30-60 min post collection and processed by centrifugation to decant serum into 2D barcoded tubes. All samples were stored at −80 °C until analysis for drug concentrations using GRIP (generic) ELISA assay (Yang 2008). Limit of quantification were set at 0.0156 μg/mL for the test articles.

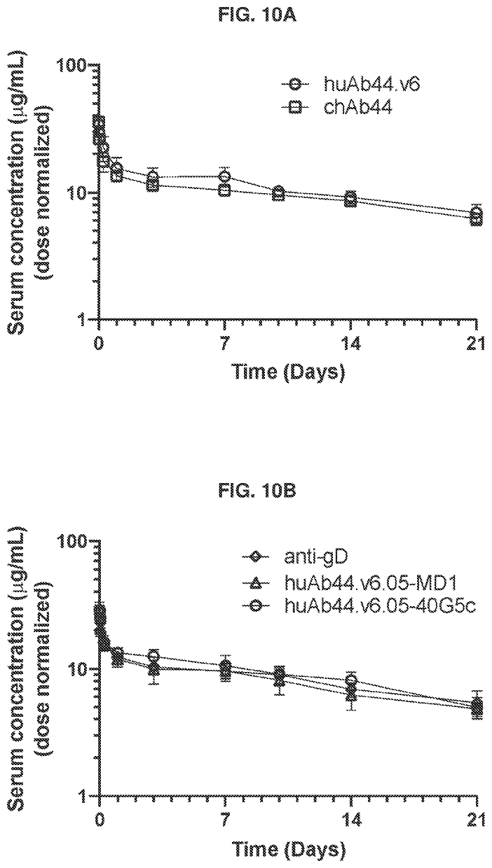

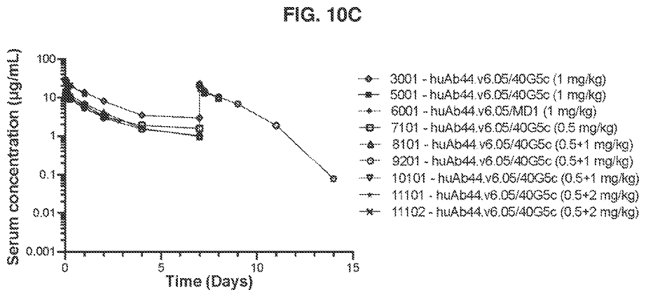

The PK profiles of chAb44 and huAb44.v6 are shown in FIG. 10A and the PK parameters are listed in Table 6. In general, both antibodies showed a bi-exponential PK behavior with slow systemic clearance (CL) ranging from about 2.6-2.7 mL/day/kg and relatively long terminal elimination half-life (t_{1/2}) in the range of 16-19 days. Maximum concentration in serum (Cₘₐₓ) was comparable for the two antibodies (approximately 24 μg/mL). The PK characteristics of chAb44 and huAb44.v6 were considered suitable for further development.

In a second study, PK of anti-STEAP1/CD3 T-cell Dependent Bispecific (TDB) antibodies following administration of a single intravenous (IV) bolus dose of 1 mg/kg was assessed in severe combined immunodeficiency (SCID) mice. The study included huAb44.v6.05-MD1 and huAb44.v6.05-40G5c TDBs, and anti-gD IgG as a control (dosed at 1 mg/kg). The in-life phase of the study was conducted at Charles River Laboratories (CRL) South San Francisco, CA in accordance with local Institutional Animal Care and Use Committee guidelines. Naïve female SCID mice weighing 17 to 22 g and approximately 7-9 weeks old at the time of dosing were assigned to treatment arms (n=4 animals per group). The test and control articles were administered as single IV bolus doses via tail vein (dose volume set at 5 mL/kg). Animal restrainers were used for IV dosing. Serial blood samples (30-40 μL) were collected in serum separator tubes at 0.167, 1, 6, 24, 72, 168, 240, 336 and 504 h post dose via tail nicks. Blood samples were kept at room temperature for 30-60 min post collection and processed by centrifugation to decant serum into 2D barcoded tubes. All samples were stored at −80 °C until analysis for drug concentrations using GRIP ELISA assay (Yang 2008). Limit of quantification of both the test articles and control were set at 0.0156 μg/mL.

The PK profiles of anti-gD, huAb44.v6.05-MD1 TDB and huAb44.v6.05-40G5c TDB are shown in FIG. 10B and the PK parameters are listed in Table 7. The PK characteristics of huAb44.v6.05-MD1 and huAb44.v6.05-40G5c TDBs were comparable to that of anti-gD control. All the 3 test articles exhibited a bi-exponential PK behavior with slow CL ranging from approximately 3.30-3.74 mL/day/kg and t_{1/2} in the range of 13.5-17.1 days. For the test articles, maximum concentration in serum (Cₘₐₓ) were in the range of 20.3-26.2 μg/mL, and dose normalized Cₘₐₓ (Cₘₐₓ/actual dose) were comparable (28.6-29.2 μg/mL). In summary, the PK characteristics of huAb44.v6.05-MD1 TDB and huAb44.v6.05-40G5c TDB were as expected for a typical IgG antibody (Ovacik et al., MAbs 11(2):422-433 (2019)) and were further evaluated in a cynomolgus monkey study.

The PK analysis of serum concentration-time data was performed using a non-compartmental analysis (NCA) approach with Phoenix™ WinNonlin® version 6.4 software (Pharsight Corp, Mountain View, CA). For both the studies, iv bolus input and actual doses were used for analysis.

TABLE 6
Pharmacokinetic parameters of anti-STEAP1 antibodies following a single intravenous dose
of 1 mg/kg in female SCID mouse

| | Cₘₐₓ | AUC_{INF} | CL | V_{SS} | t_{1/2} |
|---|---|---|---|---|---|
| Group | (μg/mL) | (day*μg/mL) | (mL/day/kg) | (mL/kg) | (days) |
| huAb44.v6 | 23.7 ± 2.09 | 273 ± 48.9 | 2.59 ± 0.51 | 59.2 ± 8.49 | 16.0 ± 2.07 |
| chAb44 | 23.9 ± 0.25 | 250 ± 29.3 | 2.71 ± 0.34 | 70.7 ± 2.56 | 18.6 ± 2.58 |

Cₘₐₓ = maximum observed concentration;
AUC_{INF}: area under the curve from time zero to infinity;
CL = clearance;
V_{SS} = volume of distribution at steady state;
t_{1/2} = elimination half life

TABLE 7
Pharmacokinetic parameters of anti-STEAP1/CD3 TDBs and anti-gD following a single intravenous
dose of 1 mg/kg in female SCID mouse

| | Cₘₐₓ | AUC_{INF} | CL | Vss | t_{1/2} |
|---|---|---|---|---|---|
| Group | (μg/mL) | (day*ug/mL) | (mL/day/kg) | (mL/kg) | (days) |
| anti-gD | 26.2 ± 2.54 | 293 ± 89.9 | 3.30 ± 0.84 | 76.2 ± 9.03 | 17.1 ± 6.23 |
| huAb44.v6.05-MD1 | 23.1 ± 3.15 | 214 ± 29.5 | 3.74 ± 0.51 | 78.1 ± 14.5 | 14.4 ± 2.84 |
| huAb44.v6.05-40G5c | 20.3 ± 1.72 | 211 ± 40.2 | 3.47 ± 0.71 | 65.2 ± 11.6 | 13.5 ± 3.29 |

Cₘₐₓ = maximum observed concentration;
AUC_{INF}: area under the curve from time zero to infinity;
CL = clearance;
V_{SS} = volume of distribution at steady state;
t_{1/2} = elimination half life

### Example 10. Intravenous Pharmacokinetics of Anti-STEAP1 T-Cell Dependent Bispecific (TDB) Antibodies in Male Cynomolgus Monkey

Pharmacokinetics of huAb44.v6.05/40G5c and huAb44.v6.05/MD1, anti-STEAP1 T-cell dependent bispecific (TDB) antibodies, was assessed in male cynomolgus monkey following a single IV infusion (1 h duration) of 1 mg/kg or 0.5 mg/kg single dose, as well as repeat dose schedule of 0.5 mg/kg (on day 1) followed by 1 or 2 mg/kg after 7 days. The study was conducted at Charles River Laboratories (CRL) Reno, NV and animal care was in accordance with the Animal Welfare Act, the Guide for the Care and Use of Laboratory Animals, and the Office of Laboratory Animal Welfare of CRL. Naïve male cynomolgus monkeys weighing 2.3 to 3 kg and approximately 2.5 to 6 years old at the time of dosing were assigned to treatment groups. The animals were observed for up to 7 days in single dose groups and 14 days in repeat dose groups. Blood samples (maximum of 1 mL whole blood) were collected in serum separator tubes via venipuncture at 0.25 h, 2 h, 6 h, 24 h, 2d, 4 d, and 7 d post dose for PK estimation. In the repeat dose groups, additional blood samples were collected at 0.25 h, 2 h, 6 h, 24 h, 2 d, 4 d, and 7 d post second dose for PK estimation. The collected blood samples were allowed to sit for 60 min post collection at room temperature and processed by centrifugation to decant serum into 2D barcoded tubes. All serum samples were stored at −70 °C until analysis for antibody concentrations using GRIP ELISA assay (Yang 2008). Limit of quantification of the test articles was set at 0.0156 μg/mL. Serum samples for anti-drug antibody (ADA) assessment were also collected at −14 d, predose and 7 d from single dose groups, and at predose and 15 d from repeat dose groups.

PK analysis of concentration-time data was performed using non-compartmental analysis (NCA) (Phoenix™ WinNonlin® version 6.4, Pharsight Corp, Mountain View, CA) with IV infusion input. The concentration-time profiles are shown in FIG. 10C and the corresponding PK parameter estimates are tabulated in Table 8. HuAb44.v6.05-40G5c TDB demonstrated expected bi-exponential disposition (Ferl et al., 2018, Sun et al., 2015, Staflin et al., 2020) with systemic clearance (CL) in the range of 14-19 mL/day/kg across the 0.5 (animal 3001) and 1 mg/kg (animal 7101, 8101, 9201) dose groups. The Cₘₐₓ values were consistent with expectations at both 0.5 and 1 mg/kg doses, and were comparable across the 0.5 mg/kg groups. The dose normalized exposures (AUC₀₋₇/Dose) for huAb44.v6.05-40G5c were comparable for 0.5 and 1 mg/kg dose groups. The serum samples were analyzed for anti-drug antibodies (ADA). ADAs were observed in two animals (7101, 9201), and the occurrence of ADAs correlated with reduced systemic exposure in one animal (9201).

In summary, the PK characteristics of huAb44.v6.05-MD1 TDB and huAb44.v6.05-40G5c TDB were as expected for a typical IgG antibody. It is expected that administration of huAb44.v6.05 TDBs will be safe and tolerable for treatment of STEAP1-expressing cancers, including but not limited to prostate cancer or Ewing sarcoma.

TABLE 8
Pharmacokinetic parameters of huAb44.v6.05/40G5c and huAb44.v6.05/MD1 TDBs
following intravenous administration in cynomolgus monkeys

| | | | | Post First-Dose | | | | Post Second- | |
|---|---|---|---|---|---|---|---|---|---|
| | | | | | | | CL | Dose | |
| | | Dose(s) | | Cₘₐₓ | AUC₀₋₇ | AUC₀₋₇/Dose | (mL/ | Cmax | AUC₇₋₁₄ |
| | | (mg/kg) | | (μg/ | (day*μg/ | (day*μg*kg/ | day/ | (μg/ | (day*μg/ |
| Animal | Treatment | Day 1 | Day 8 | mL) | mL) | mL/mg) | kg) | mL) | mL) |
| 3001 | huAb44.v | 1 | NA | 27.9 | 49.5 | 49.5 | 15.3 | NA | NA |
| | 6.05- | | | | | | | | |
| | 40G5c | | | | | | | | |
| 5001 | huAb44.v | 1 | NA | 29.1 | ND | ND | ND | NA | NA |
| | 6.05- | | | | | | | | |
| | 40G5c | | | | | | | | |
| 6001 | huAb44.v | 1 | NA | 23.4 | ND | ND | ND | NA | NA |
| | 6.05-MD1 | | | | | | | | |
| 7101\hat{ } | huAb44.v | 0.5 | NA | 11.5 | 23.3 | 46.6 | 14.0 | NA | NA |
| | 6.05- | | | | | | | | |
| | 40G5c | | | | | | | | |
| 8101 | huAb44.v | 0.5 | 1 | 15.3 | 25.0 | 50.0 | 17.5 | 22.4 | ND |
| | 6.05- | | | | | | | | |
| | 40G5c | | | | | | | | |
| 9201\hat{ } | huAb44.v | 0.5 | 1 | 12.6 | 21.2 | 42.4 | 19.1 | 21.9 | 33.5 |
| | 6.05- | | | | | | | | |
| | 40G5c | | | | | | | | |
| 10101 | huAb44.v | 0.5 | 1 | 13.5 | ND | ND | ND | ND | ND |
| | 6.05- | | | | | | | | |
| | 40G5c | | | | | | | | |
| 11101 | huAb44.v | 0.5 | 2 | 12.1 | ND | ND | ND | ND | ND |
| | 6.05- | | | | | | | | |
| | 40G5c | | | | | | | | |
| 11102 | huAb44.v | 0.5 | 2 | 13.2 | ND | ND | ND | ND | ND |
| | 6.05- | | | | | | | | |
| | 40G5c | | | | | | | | |

Cₘₐₓ = maximum observed concentration;
CL = clearance;
AUC₀₋₇/Dose: Area under the serum concentration-time curve from time zero to 7 days post-dose normalized by dose;
AUC₇₋₁₄/Dose: Area under the serum concentration-time curve from time 7 to 14 days post-dose normalized by second dose;
NA = not applicable;
ND = not determined due to insufficient data;
\hat{ }ADA positive

### Example 11. Structure of Ab-44 Bound to STEAP1

Recombinant STEAP1 was incubated with a Fab fragment of antibody STEAP1-44.NGS.HC2 with the STEAP1-44 light chain at 1:1.2 molar ratio and incubated on ice for 30 min. Mixture was injected over a SUPEROSE®6 Increase 3.2/300 column (GE Healthcare) equilibrated in 25 mM Tris pH 8, 150 mM NaCl, 0.02% LMNG, 0.002% CHS. 3.5 μL of the peak fraction of complex at a concentration of 1 mg/mL was applied to a glow-discharged holey carbon grid (25 nm ultrafoil grids 2/2 holes, 200 mesh from Quantifoil) coated with a thin layer of gold. Grids were blotted in VITROBOT™ Mark IV (Thermo Fisher) using 5 s blotting time with 100% humidity, and then plunge-frozen in liquid ethane cooled by liquid nitrogen.

A total of 6225 movie stacks were collected with SerialEM (Mastronarde, 2005) on a Titan KRIOS™ electron microscope (Thermo Fisher) operated at 300 kV and equipped with a BioQuantum energy filter operated with a 20 eV energy slit with a K2 Summit direct electron detector camera (Gatan). Images were recorded at a nominal magnification of 165,000×, corresponding to a pixel size of 0.824 Å per pixel. Each image stack contains 50 frames recorded every 0.2 s giving an accumulated dose of 46 e−/A² and a total exposure time of 10 s. Images were recorded with a set defocus range of 0.5 to 1.5 μm. In order to overcome preferential orientation of the complex and reduce directional resolution anisotropy, 1288 movies out of the full dataset were collected with a tilted angle of 40 degree (Tan, 2017).

Image processing from stacks of frames to refined 3D volumetric map was performed with cisTEM (Grant, 2018). For the 3D structure of STEAP1 bound by Fab44, 163759 particles were used; C3 symmetry was applied during reconstruction and led to the generation of a 3 Å model. For atomic model building, models for both STEAP1 and Fab44 were built using Swiss Model (swissmodel.expasy.org/) and the complete model was then rebuilt using interactive molecular dynamics (Croll, 2018) and refined in real space (Afonine, 2018).

In the cryo-EM structure of Ab44 in complex with STEAP1, the human STEAP1 protein subunit is found to form six transmembrane helices, as predicted based on hydrophobicity analysis. Based on the cryo-EM structure and electrostatic surface analysis, transmembrane helix 1 (TM1) of STEAP1 spans from Trp71-Ile95, TM2 of STEAP1 spans from Ile109-Tyr130, TM3 of STEAP1 spans from Lys162-Met184, TM4 of STEAP1 spans from Trp214-Trp236, TM5 of STEAP1 spans from Trp247-Phe272, and TM6 of STEAP1 spans from Phe291-Phe309. Accordingly, three distinct extracellular loops (ECLs) are therefore also present within each STEAP1 protein subunit, and can be defined as extracellular loop 1 (ECL1) of STEAP1 spans from His96-Lys108, ECL2 of STEAP1 spans from Arg185-Val213, ECL3 of STEAP1 spans from Ala273-Thr290. Overall, STEAP1 forms a six-transmembrane helix bundle between which a solvent accessible cleft is formed and exposed to the extracellular side. A heme co-factor is found bound deep within this cleft, contacted and coordinated primarily by TM2, TM3, TM4 and TM5, with important contributions from TM6. ECL2 extends over the extracellular cleft with the bound heme in a near V-like conformation, where the apex of ECL2 is ~25 Å above the most proximal group of the heme ligand.

In the cryo-EM structure, the human STEAP1 protein subunit forms homotrimeric assembly with two equivalent STEAP1 protein subunits. An approximate three-fold symmetry axis runs down the STEAP1 trimer perpendicular to the plan of the membrane bilayer. An extensive and composite trimeric interface is formed between the STEAP1 subunits which is mediated primarily by the ECL2 on the extracellular side. ECL1 and TM2 make smaller but important contributions to the overall trimeric subunit interface of STEAP1, whereas ECL3 and TM5 and TM6 are most peripheral from the interface. When viewed from the extracellular side of the membrane, the ECL2s from each of the three subunits form a central cap-like structure at the center of the trimer, where subunits are arranged in a counterclockwise orientation when considering N-terminus to C-terminus of each protein chain. One consequence of the trimeric STEAP1 assembly and the arcing V-like conformation of each ECL2 is that there is an interdigitation of one subunit into each neighboring subunit. Overall, in effect, the extracellular cleft in any one protomer is formed in a composite way with important contributions from both neighboring subunits. Specifically, a region of TM4 from subunit B inserts itself (and a few side-chains) in-between TM2 and TM3 of subunit A, whereas TM4 from subunit C inserts itself (and a few side-chains) in-between TM4 and TM5 of subunit A.

### Overall Structure Description of the Ab44-STEAP1 Complex

In the cryo-EM structure, the Fab of Ab44 is found to dock onto the extracellular surface of the STEAP1 trimer and binds mainly although not exclusively to one STEAP1 promoter. From the perspective of binding a single STEAP1 promoter, the heavy chain (HC) of Ab44 makes contacts with ECL1 (most extensively), ECL2, and ECL3. From the perspective of binding a single STEAP1 promoter, the light chain (LC) of Ab44 makes contact only with ECL2. In addition to the expected Ab44-STEAP1 subunit interactions, each Ab44 also makes specific albeit minor contacts through its heavy chain with the neighboring subunit of the STEAP1 trimer.

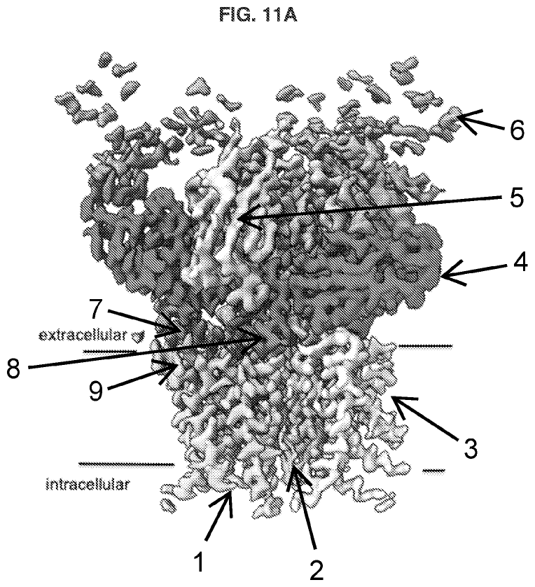

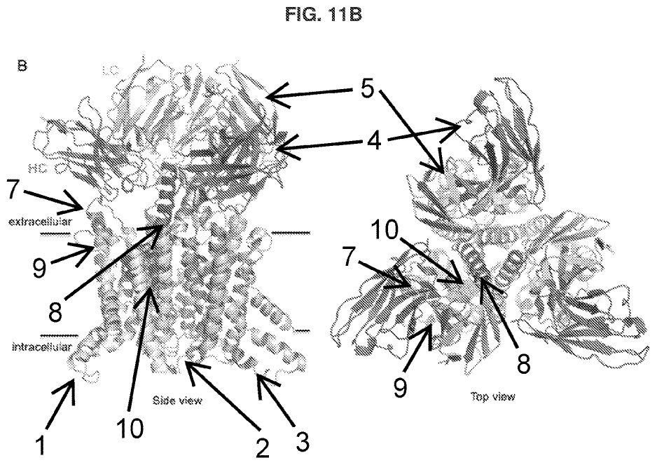

Beyond the Ab44-STEAP1 interactions, the Fab of Ab44 also forms an extensive homotypic Ab44-Ab44 interface. Multiple polar homotypic Fab interactions are made between LC-LC-LC pairings along the three-fold axis of the structure. In addition to these LC-LC homotypic Ab44-Ab44 interactions, HC-LC homotypic interactions are also observed in the cryo-EM structure. See FIGS. 11A and 11B.

### Residue and Surface Area Contact Between Ab44 and STEAP1:

A summary of interactions mediated by the Ab44 HC CDRs is illustrated as follows: hydrogen bond between the Tyr103-HC hydroxyl group and NH group from Trp195 side chain (ECL2); hydrogen bond between the Gly101-HC backbone carbonyl and amide side chain of Gln202 (ECL2); hydrogen bond between the Tyr107-HC hydroxyl group and amide side chain of Gln198 (ECL2); hydrogen bond between the Leu56-HC backbone carbonyl and amine side chain of Lys281 (ECL3); hydrogen bond between the Ser73-HC hydroxyl group and imidazole side chain of His102 (ECL1); hydrogen bond between the Asn74-HC backbone amide and backbone carbonyl of Ser101 (ECL1); hydrogen bond between Asn74-HC and Gln103 side chains (ECL1). Also see Table 3. Overall, the HC:STEAP1 interaction buries 680 Å² of solvent exposed area.

A summary of interactions mediated by the Ab44 LC CDRs is illustrated as follows: hydrogen bond between the Tyr35-LC hydroxyl group and backbone carbonyl from Gln202 (ECL2); hydrogen bond between the Tyr54-LC hydroxyl group and backbone carbonyl from Gln201 (ECL2); residues Asn203 and Lys204 are involved in van der Waals interactions with LC CDR residues. Overall, the LC:STEAP1 interaction buries a modest 170 Λ² of solvent exposed area.

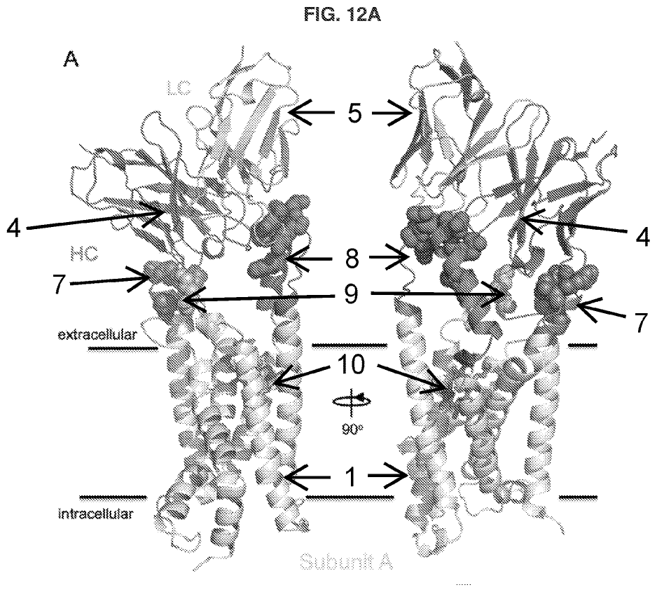

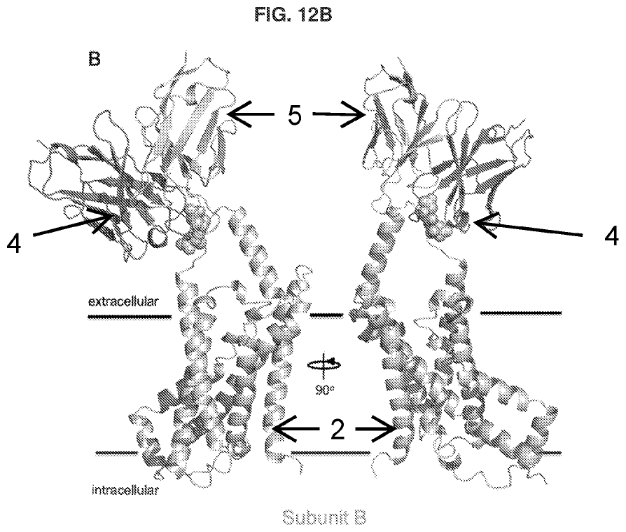

Each Ab44 also makes contacts with the neighboring subunit of the STEAP1 trimer. Specifically, Thr28-HC of Ab44 (bound mainly to subunit A) makes a direct hydrogen bond interaction with the side-chain of Asp206 from subunit B while Arg98-HC appears to engage in an electrostatic interaction with the carboxylic group of Asp205. This interaction contributes to an additional 88 Å² of solvent exposed area. Also see FIGS. 12A and 12B.

### Residue and Surface Area Contact Between Homotypic Ab44 Interface

Arg82-LC from Ab44 Fab A reaches across to form salt bridge interactions with Glu84-LC and Glu86-LC from a neighboring Ab44 (Fab C), and this tripartite ionic interaction is seen across all LC promoters in the cryo-EM structure. Asp65-LC from Ab44 Fab A also forms a hydrogen-bonding network directly on the three-fold symmetry access with the Asp65-LC from the Fab bound over subunit B and subunit C of STEAP, respectively. The interaction between LC from Ab44 Fab A and Ab44 Fab C buries ~180 Å² of solvent exposed area (HC from Ab44 Fab A doesn't interact with Ab44 Fab C).

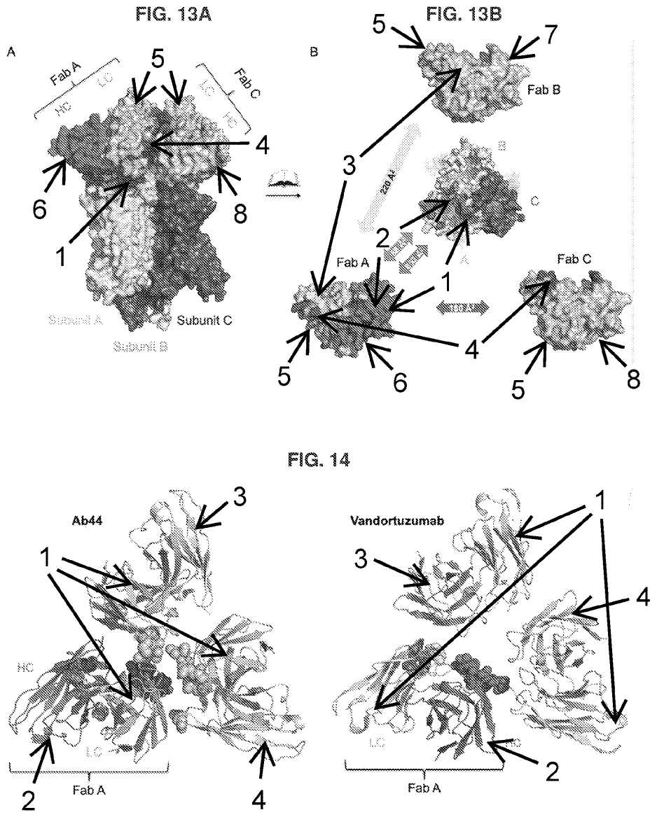

Glu1-HC forms van der Waals interactions and surface complementarity with Ser72-LC from neighboring Ab44 Fab B, and there may also be a water-mediated hydrogen bond between these two side-chains. The interaction between Fab A and LC-Fab B buries a total of ~220 Å² of solvent exposed area. See FIGS. 13A and 13B.

It is expected that other Ab44-derived variants, including huAb44.v6.05, bind to STEAP1 in a similar manner as the antibody STEAP1-44.NGS.HC2 with the STEAP1-44 light chain as described in the present Example. As is discussed in Hsiao et al. supra, upon antigen binding, somatic mutation and clonal expansion lead to lineages of clonally related but distinct B cells expressing antibodies that bind antigen with affinities that may differ among clones. Such somatic mutations (e.g., as evaluated in Example 4) would not be expected to alter an antibody's overall binding mode, but instead affect details in specific residue interactions, for instance, by adding a new beneficial interaction or removing an unfavorable one, or by reducing entropic penalties in binding (Schmidt et al. Proc. Nat. Acad. Sci. USA 110:264-269, 2013; Vajda et al., Curr. Opin. Struct. Biol. 67; 226-231, 2021).

A comparison between Ab44 and mAb120.545 (also known as Vandortuzumab) binding to STEAP1 (Oosterheert et al., J. Biol. Chem. 295:9502-9512 (2020)) reveals different binding modes. Vandortuzumab's main epitope is centered around ECL2 where both heavy and light chains contribute almost equally to binding (410 Å² and 340 Å² respectively). Vandortuzumab CDR H3 dives into a pocket formed by basic residues located just above the heme; this basic ring is expected to coordinate the chelated iron Fe³⁺ substrate based on structural data obtained with STEAP4 (Oosterheert, 2018). While Vandortuzumab is expected to compete for substrate binding, Ab44 doesn't seem to share this feature. See FIG. 14.

A summary of the key interactions mediated by mAb120.545 HC CDRs, as reported by Oosterheert et al., J. Biol. Chem. 295:9502-9512 (2020), is illustrated as follows: hydrogen bond between the backbone carbonyl of Tyr104-HC and the NH from the indole side chain of Trp195 (ECL2); hydrogen bond between the Tyr108-HC hydroxyl group side chain and Gln202 side chain (ECL2); hydrogen bond between the backbone carbonyl of Thr58-HC and the side chain of Asn203 (ECL2); hydrogen bond between the Tyr104-HC hydroxyl group side chain and the NH from the side chain of Lys204; hydrogen bond between the Tyr108-HC hydroxyl group side chain and the backbone carbonyl of Gln198 (ECL2); hydrogen bond between the Tyr102-HC hydroxyl group side chain and the Gln198 side chain (ECL2); hydrogen bond between the hydroxyl group side chain of Ser59-HC and the backbone carbonyl of Gln201 (ECL2); hydrogen bond between the Tyr51-HC hydroxyl group side chain and the backbone carbonyl of Gln202 (ECL2); hydrogen bond between the Ser57-HC hydroxyl group side chain and the carboxylic side chain of Glu205 (ECL2); hydrogen bond between the Tyr104-HC hydroxyl group side chain and the backbone carbonyl of Ala207. Overall, the HC:STEAP1 interaction buries 444 Å² of solvent exposed area.

A summary of interactions mediated by the mAb120.545 LC CDRs, as reported by Oosterheert et al., J. Biol. Chem. 295:9502-9512 (2020), is illustrated as follows: hydrogen bond between the backbone carbonyl group of Ser33-LC and the hydroxyl group of Tyr107 side chain (ECL1); hydrogen bond between the Ser33-LC hydroxyl group side chain and the NH group from Asn194 side chain (ECL2); hydrogen bond between the Gln35-LC side chain and the backbone amide of Gln104 (ECL2); hydrogen bond between the backbone carbonyl group of Tyr100-LC and the side chain of Gln201 (ECL2); hydrogen bond between the Tyr100-LC hydroxyl group side chain and the side chain of Gln202 (ECL2); hydrogen bond between the side chains of Gln35-LC and Gln104 (ECL2). Overall, the LC:STEAP1 interaction buries 367 Å² of solvent exposed area.

The mAb120.545 light chain from one of the symmetry related Fab′ contributes an additional interaction of 270 Å² of solvent exposed area through the following interactions: salt bridge between Arg32-LC′ side chain and the side chain of Asp206 (ECL2); hydrogen bond between the Tyr98-LC′ hydroxyl group side chain and the carboxyl chain of Glu205 (ECL2); hydrogen bond between the side chain of Gln27-LC′ and the carboxyl chain of Glu205 (ECL2); hydrogen bond between the side chain of Asn99-LC′ and the carboxyl chain of Glu205 (ECL2); hydrogen bond between the guanidium side chain of Arg32-LC′ and the backbone carbonyl of Asp206 (ECL2); hydrogen bond between the backbone amide of Ser33-LC and the carboxyl chain of Glu205 (ECL2); hydrogen bond between the Ser33-LC hydroxyl group side chain and the carboxyl chain of Glu205 (ECL2); hydrogen bond between the backbone amide of Arg32-LC and the carboxyl chain of Glu205 (ECL2).

In summary, the data described in this Example demonstrate that Ab44 and other anti-STEAP1 antibodies disclosed herein bind to a unique epitope of STEAP1 that is different from existing anti-STEAP1 antibodies described in the literature.

### Example 12. Expected Binding of Ab44 to Cyno STEAP1

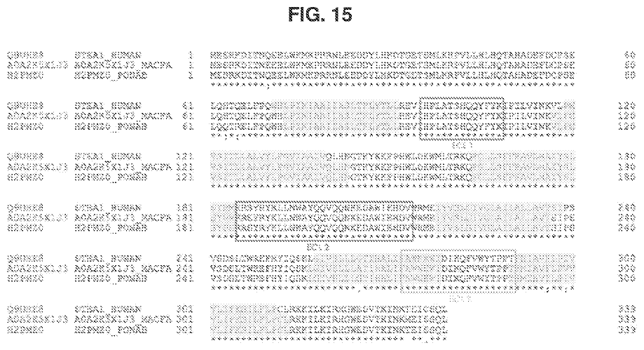

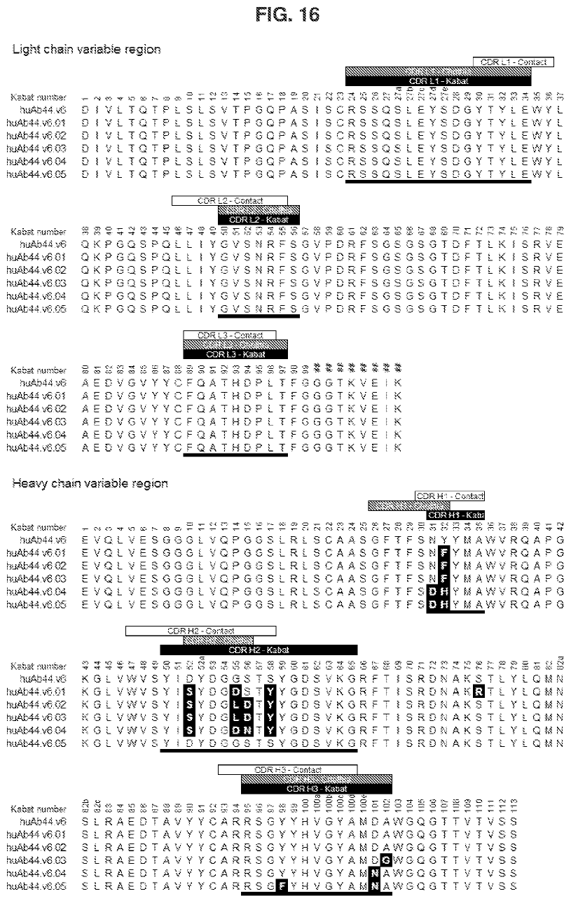

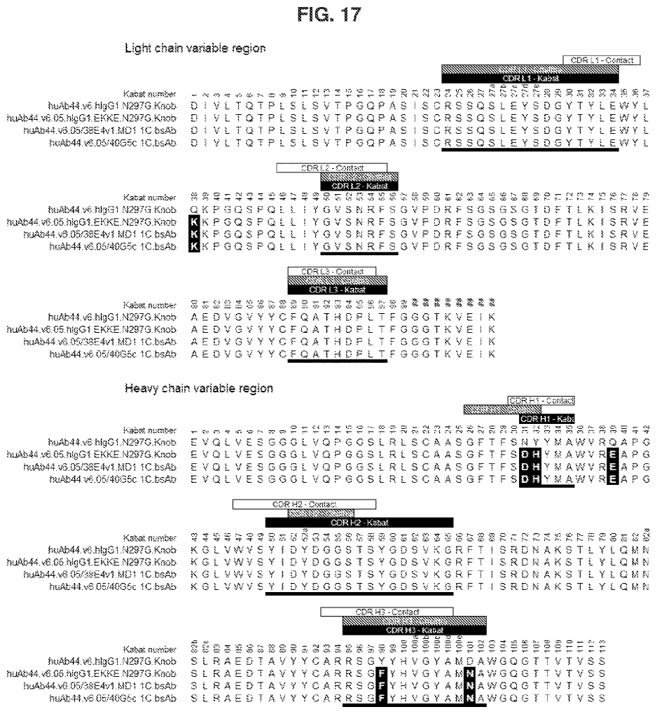

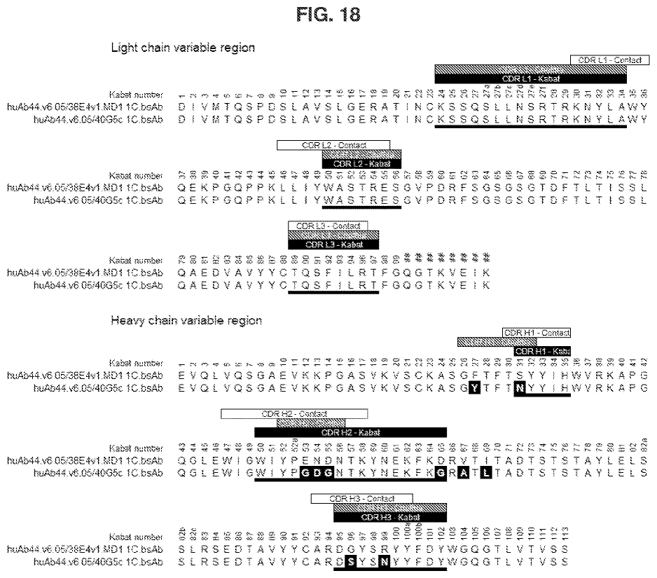

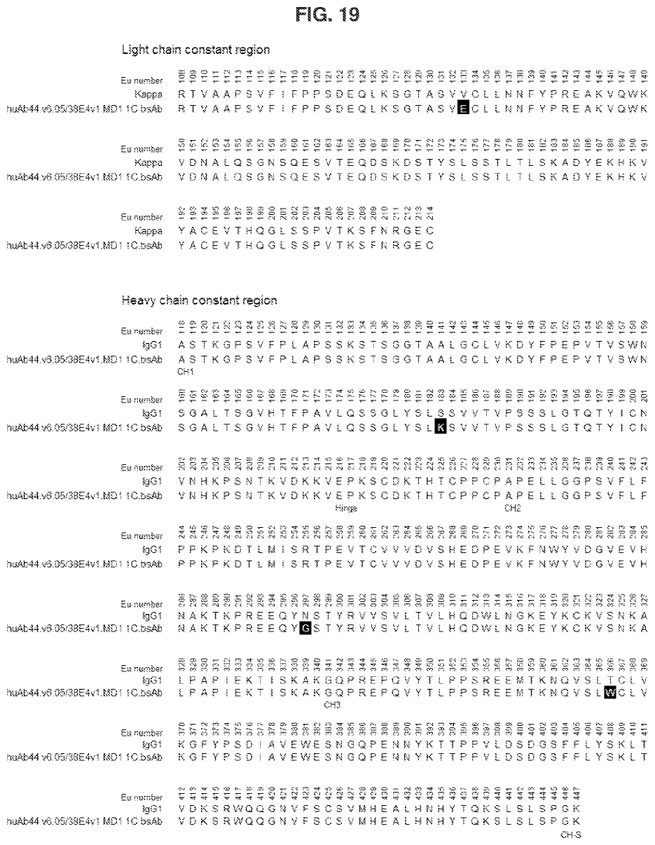

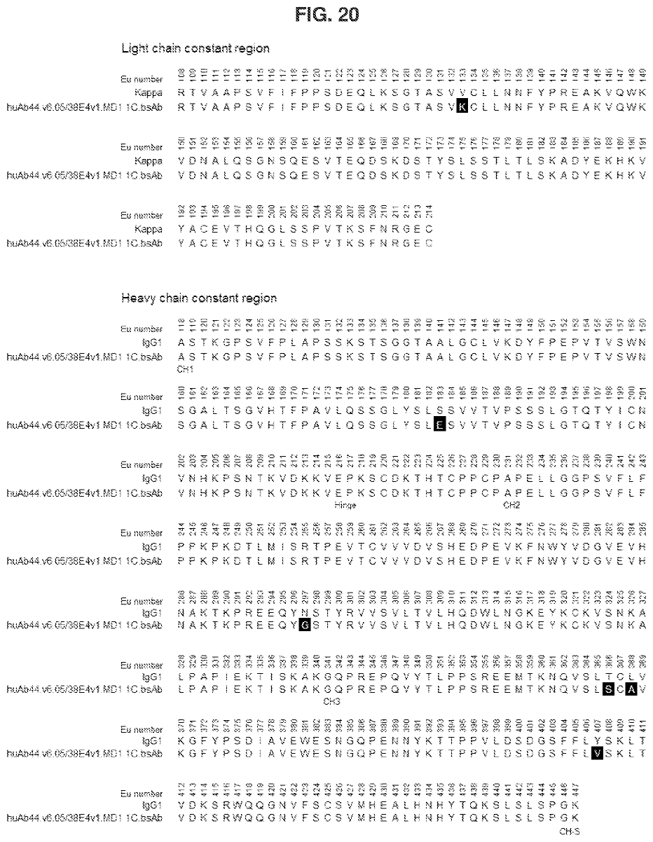

Degree of sequence homology between human and primate STEAP1 is high. All three extracellular loops are strictly conserved suggesting cross reactivity of Ab44 towards these proteins. FIG. 15 shows the sequence alignment of human, Macaca fascicularis and Pongo abelli. Additionally, STEAP1 was found to exhibit comparable expression patterns in cyno and human normal tissues.

### Example 13. huAb44.v6.05 Exhibits Unexpectedly Superior Properties Compared to Existing Anti-STEAP1 Antibodies

The huAb44.v6.05 anti-STEAP1 antibody described herein was compared with the existing anti-STEAP1 antibody vandortuzumab in terms of binding affinity and cell killing potency in the TDB context.

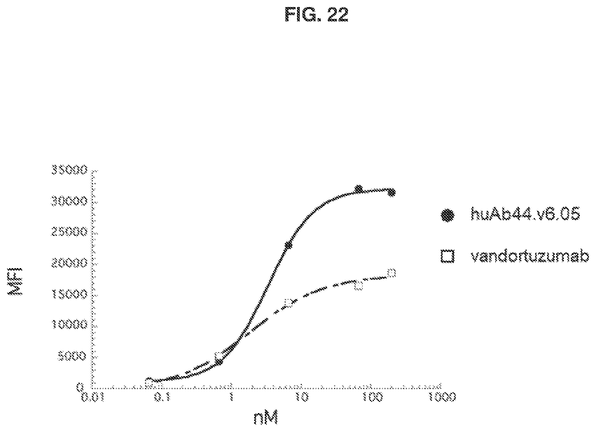

First, the binding affinity of huAb44.v6.05 was compared with vandortuzumab in a direct head-to-head comparison. FIG. 22 shows a comparison of binding of huAb44.v6.05 and vandortuzumab IgG for binding to STEAP1-expressing LNCaP-X1.2 cells as assessed by FACS. huAb44.v6.05 exhibited stronger binding compared to vandortuzumab. The EC50 for huAb44.v6.05 was 1.9 nM, which was approximately 1.8-fold improved compared to the EC50 of 3.5 nM for vandortuzumab. More strikingly, huAb44.v6.05 showed a higher maximal binding to cells compared to vandortuzumab, which is also a function of affinity. These data demonstrate that huAb44.v6.05 binds to STEAP1-expressing cells with higher binding affinity compared to existing anti-STEAP1 antibodies such as vandortuzumab.

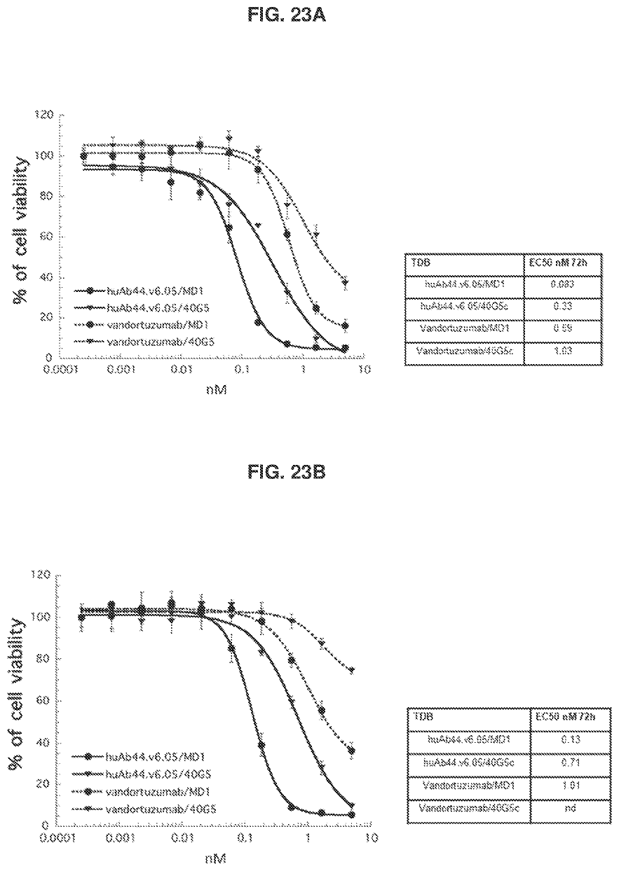

Next, the cell killing potency of huAb44.v6.05 TDBs was compared with vandortuzumab TDBs. FIGS. 23A and 23B show a direct head-to-head comparison of cell killing potency of bispecific anti-STEAP1/anti-CD3 antibodies tested in cell killing assays with human CD8+ T cells and STEAP1-expressing LNCaP-X1.2 cells and LNCaPX1.2KO3-13 cells, respectively. The LNCaP-X1.2 cell line is a subline from publicly available LNCaP clone FGC cells. The LNCaPX1.2 cell line was established from a xenograft tumor piece of LNCaP clone FGC cells grown as a xenograft in immune compromised mice. LNCaPX1.2KO3-13 is a cell line derived from LNCaP-X1.2 cells by gene editing technology to reduce STEAP1 expression. In these experiments, both the MD1 and 40G5c anti-CD3 arms were tested.

FIGS. 23A and 23B show that the cell killing efficacy of vandortuzumab-TDBs as shown in EC50 values was substantially lower compared to huAb44.v6.05-TDBs. The improvement of huAb44.v6.05-TDBs was observed with both the MD1 and 40G5c anti-CD3 arms. Moreover, vandortuzumab-TDBs did not reach 100% cell killing potency compared to huAb44.v6.05-TDBs. Thus, these results demonstrate that huAb44.v6.05 TDBs possess strikingly improved cell killing potency compared to TDBs utilizing existing anti-STEAP1 antibodies such as vandortuzumab.

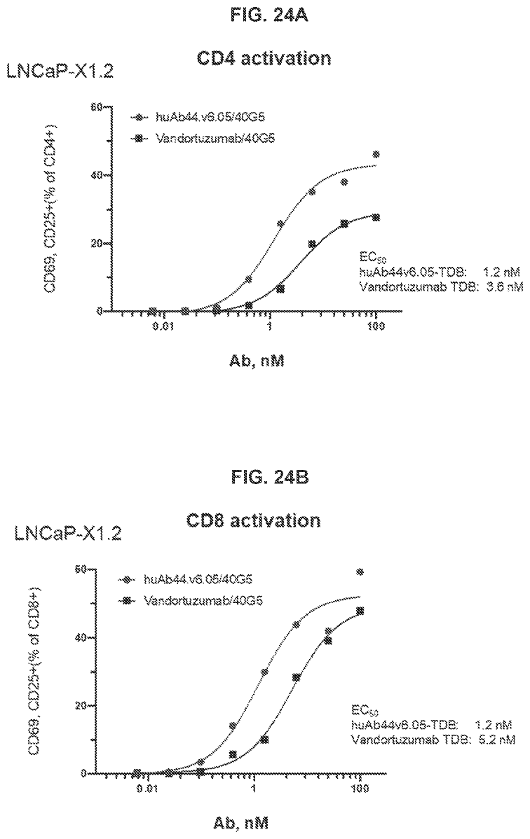

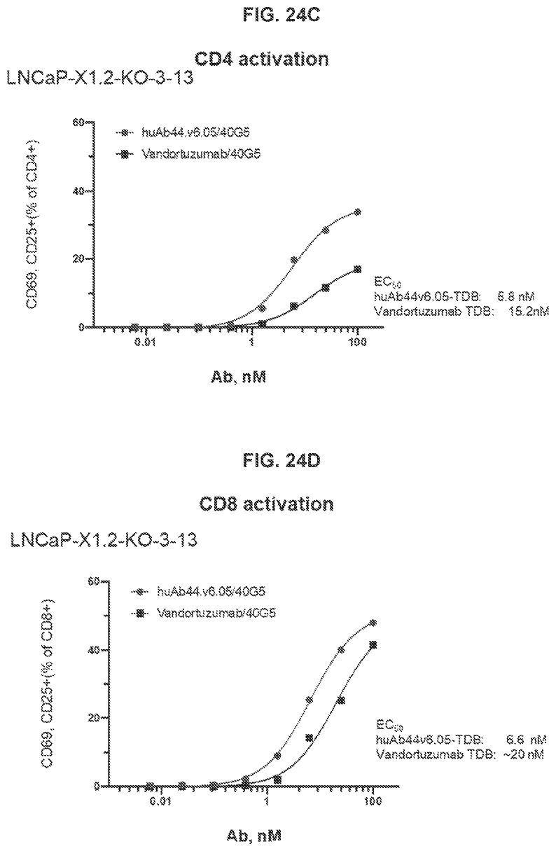

Additionally, the activity of huAb44.v6.05-TDBs was compared to vandortuzumab TDBs in a T-cell activation assay in a direct head-to-head comparison. As is shown in FIGS. 24A-24D, huAb44.v6.05-TDBs resulted in higher activation of both CD4 and CD8 T-cells compared to vandortuzumab-TDBs.

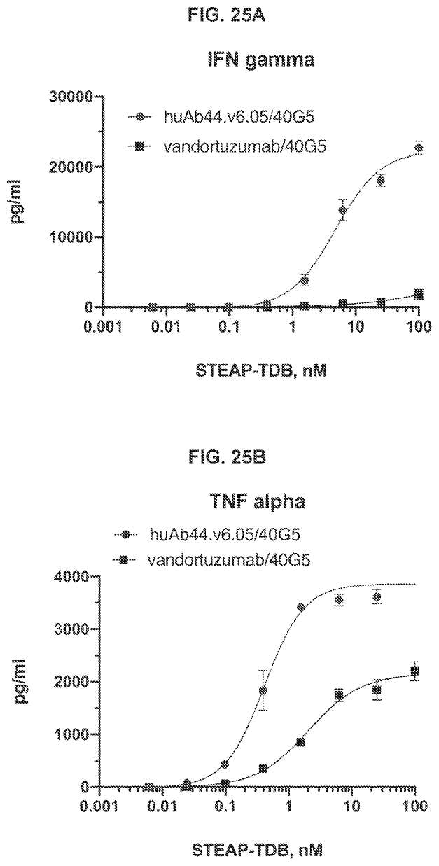

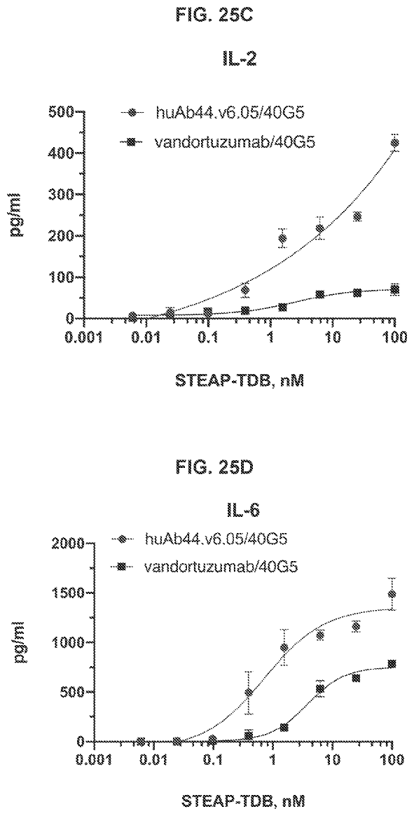

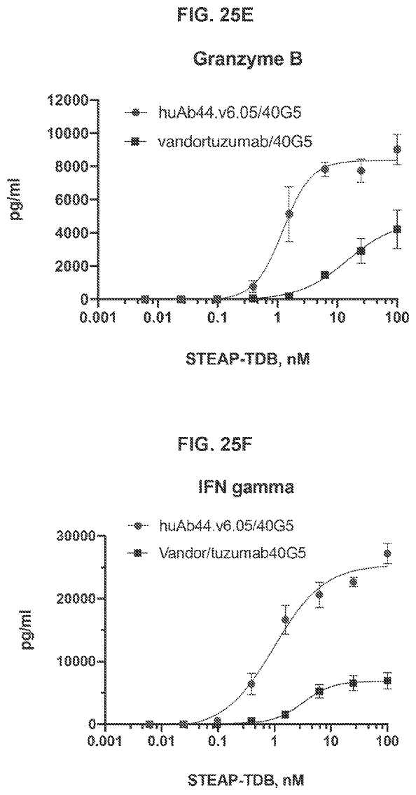

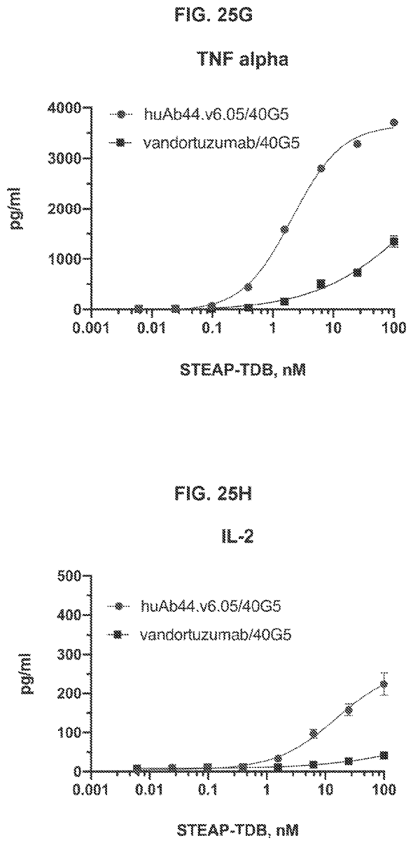

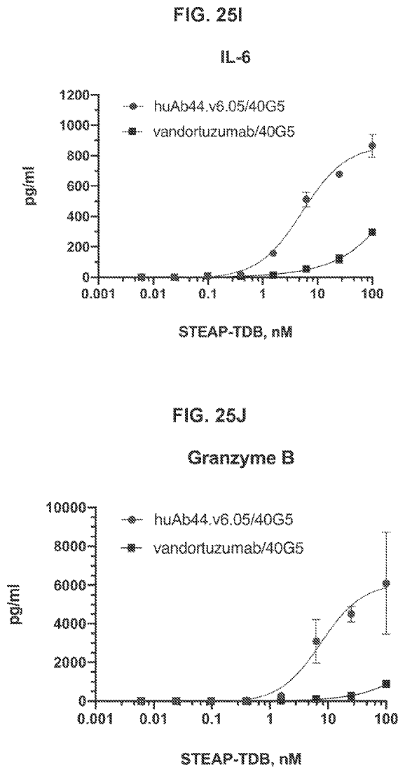

Moreover, the activity of huAb44.v6.05-TDBs was compared to vandortuzumab TDBs in terms of target-dependent cytokine secretion, including secretion of IFN gamma, TNF alpha, IL-2, IL-6, and granzyme B. As is shown in FIGS. 25A-25J, huAb44.v. 05-TDBs resulted in significantly higher levels of cytokine secretion from T-cells incubated for 24 h with LNCaP-X1.2 (FIGS. 25A-25E) or LNCaP-X1.2-KO-3-13 cells (FIGS. 25F-25J) compared to vandortuzumab-TDBs.

These unexpectedly superior properties of huAb44.v6.05 are expected to result in advantages in terms of clinical efficacy of huAb44.v6.05 TDBs for treatment of STEAP1-expressing cancers, including but not limited to prostate cancer or Ewing sarcoma. Moreover, unlike other anti-STEAP1 bispecific antibody constructs described in the literature, which bind to STEAP1 bivalently, the huAb44.v6.05 TDBs utilized in this Example bind to STEAP1 in a monovalent manner. Therefore, the anti-STEAP1 TDBs disclosed herein are able to achieve unexpectedly favorable binding affinity and potent cell killing of STEAP1-expressing cells (including tumor cells) even in constructs that bind to STEAP1 in a monovalent manner.

### REFERENCES

1. Bos A B, Luan P, Duque J N, Reilly D, Harms P D, Wong A W. Optimization and automation of an end-to-end high throughput microscale transient protein production process. Biotechnol Bioeng. 2015; 112:1832-42. doi:10.1002/bit.25601.
2. Luan P, Lee S, Arena T A, Paluch M, Kansopon J, Viajar S, Begum Z, Chiang N, Nakamura G, Hass P E, et al. Automated high throughput microscale antibody purification workflows for accelerating antibody discovery. MAbs. 2018; 10:624-35. doi:10.1080/19420862.2018.1445450.
3. Hsiao Y C, Shang Y, DiCara D., Yee A, Lai J, Kim S H, Ellerman D, Corpuz R, Chen Y, Rajan S, Cai H, Wu Y, Seshasayee D, Hötzel I. Immune repertoire mining for rapid affinity optimization of mouse monoclonal antibodies. MAbs. 2019; 11:735-746. doi: 10.1080/19420862.2019.1584517.
4. Goldstein L D, Chen Y J J, Wu J, Chaudhuri S, Hsiao Y C, Schneider K, Hoi K H, Lin Z, Guerrero S, Jaiswal B S, Stinson J, Antony A, Pahuja K B, Seshasayee D, Modrusan Z, Hötzel I, Seshagiri S. Massively parallel single-cell B-cell receptor sequencing enables rapid discovery of diverse antigen-reactive antibodies. Commun. Biol. 2019; 2:304. doi: 10.1038/s42003-019-0551-y.
5. Yang J et al. Quantitative determination of humanized monoclonal antibody rhuMAb2H7 in cynomolgus monkey serum using a Generic Immunoglobulin Pharmacokinetic (GRIP) assay. J of Immunology Methods. 335, 8-20 (2008).
6. Ovacik A M et al. Single cell-produced and in vitro-assembled anti-FcRH5/CD3 T-cell dependent bispecific antibodies have similar in vitro and in vivo properties. MAbs. 11(2), 422-433 (2019).
7. Ferl G et al. A preclinical population pharmacokinetic model for anti-CD20/CD3 T-cell-dependent bispecific antibodies. Clin Transl Sci. 11, 296-304 (2018).
8. Sun L. L et al. Anti-CD20/CD3 T cell-dependent bispecific antibody for the treatment of B cell malignancies. Sci. Transl. Med. 7, 287-270 (2015).
9. Staflin K et al. Target arm affinities determine preclinical efficacy and safety of anti-HER2/CD3 bispecific antibody. JCI Insight. 5(7):e133757 (2020).
10. Oosterheert W, Gros P. Cryo-electron microscopy structure and potential enzymatic function of human six-transmembrane epithelial antigen of the prostate 1 (STEAP1). J Biol Chem. 2020 Jul. 10; 295(28):9502-9512. doi: 10.1074/jbc.RA120.013690.
11. Oosterheert, W., van Bezouwen, L. S., Rodenburg, R. N. P. et al. Cryo-E M structures of human STEAP4 reveal mechanism of iron(III) reduction. Nat Commun 9, 4337 (2018). doi: 10.1038/s41467-018-06817-7.
12. D. N. Mastronarde, Automated electron microscope tomography using robust prediction of specimen movements. J. Struct. Biol. 152, 36-51 (2005). doi:10.1016/j.jsb.2005.07.007.
13. Tan Y Z, Baldwin P R, Davis J H, Williamson J R, Potter C S, Carragher B, Lyumkis D. Addressing preferred specimen orientation in single-particle cryo-EM through tilting. Nat Methods. 2017 August; 14(8):793-796. doi: 10.1038/nmeth.4347.
14. T. Grant, A. Rohou, N. Grigorieff, cisTEM, user-friendly software for single-particle image processing. eLife 7, e35383 (2018). doi:10.7554/eLife.35383.
15. T. I. Croll, ISOLDE: A physically realistic environment for model building into low-resolution electron-density maps. Acta Crystallogr. Sect. D Struct. Biol. 74, 519-530 (2018). doi:10.1107/S2059798318002425.
16. P. V. Afonine, B. K. Poon, R. J. Read, O. V. Sobolev, T. C. Terwilliger, A. Urzhumtsev, P. D. Adams, Real-space refinement in PHENIX for cryo-E M and crystallography. Acta Crystallogr. Sect. D Struct. Biol. 74, 531-544 (2018). doi:10.1107/S2059798318006551.

What is claimed is:

1. A multispecific antigen-binding molecule comprising: (A) a first antigen-binding domain that binds to STEAP1 and comprises an anti-STEAP1 heavy chain variable region (VH) comprising CDR-H1, CDR-H2, and CDR-H3 of a VH sequence selected from SEQ ID NOs: 7, 17-25, 30-34, 38 and 68; and an anti-STEAP1 light chain variable region (VL) comprising CDR-L1, CDR-L2, and CDR-L3 of a VL sequence selected from SEQ ID NOs: 8, 26-29, 39 and 69; and (B) a second antigen-binding domain that binds to a T cell receptor.

2. A multispecific antigen-binding molecule comprising:
(A) a first antigen-binding domain that binds to STEAP1 and comprises an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
CDR-H1 comprises Xaa¹Xaa²YMA (SEQ ID NO: 35);
wherein
Xaa¹ is Asp (D) or Asn (N); and
Xaa² is His (H), Tyr (Y), or Phe (F);
CDR-H2 comprises YIXaa³YDGXaa⁴Xaa⁵TXaa⁶YGDSVKG (SEQ ID NO: 36);
wherein
Xaa³ is Asp (D) or Ser (S);
Xaa⁴ is Gly (G), Asp (D), or Leu (L);
Xaa⁵ is Ser (S), Asp (D), or Asn (N); and
Xaa⁶ is Ser (S) or Tyr (Y); and
CDR-H3 comprises RSGXaa⁷YHVGYAMXaa⁸Xaa⁹ (SEQ ID NO: 37);
wherein
Xaa⁷ is Phe (F) or Tyr (Y);
Xaa⁸ is Asn (N) or Asp (D); and
Xaa⁹ is Ala (A) or Gly (G);
CDR-L1 comprises the amino acid sequence of SEQ ID NO: 4;
CDR-L2 comprises the amino acid sequence of SEQ ID NO: 5; and
CDR-L3 comprises the amino acid sequence of SEQ ID NO: 6; and
(B) a second antigen-binding domain that binds to a T cell receptor.

3. The multispecific antigen-binding molecule of claim 1 or 2, wherein the CDR-H1 comprises the amino acid sequence of SEQ ID NO: 10, 1, or 9.

4. The multispecific antigen-binding molecule of any one of claims 1-3, wherein the CDR-H2 comprises the amino acid sequence of SEQ ID NO: 2, 11, 12, or 13.

5. The multispecific antigen-binding molecule of any one of claims 1-4, wherein the CDR-H3 comprises the amino acid sequence of SEQ ID NO: 16, 3, 14, or 15.

6. The multispecific antigen-binding molecule of any one of claims 1-5, wherein the first antigen-binding domain comprises:
CDR-H1 comprising the amino acid sequence of SEQ ID NO: 10;
CDR-H2 comprising the amino acid sequence of SEQ ID NO: 2;
CDR-H3 comprising the amino acid sequence of SEQ ID NO: 16;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

7. The multispecific antigen-binding molecule of any one of claims 1-5, wherein the first antigen-binding domain comprises:
CDR-H1 comprising the amino acid sequence of SEQ ID NO: 1;
CDR-H2 comprising the amino acid sequence of SEQ ID NO: 2;
CDR-H3 comprising the amino acid sequence of SEQ ID NO: 3;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

8. The multispecific antigen-binding molecule of any one of claims 1-5, wherein the first antigen-binding domain comprises:
CDR-H1 comprising the amino acid sequence of SEQ ID NO: 9;
CDR-H2 comprising the amino acid sequence of SEQ ID NO: 11;
CDR-H3 comprising the amino acid sequence of SEQ ID NO: 3;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

9. The multispecific antigen-binding molecule of any one of claims 1-5, wherein the first antigen-binding domain comprises:
CDR-H1 comprising the amino acid sequence of SEQ ID NO: 9;
CDR-H2 comprising the amino acid sequence of SEQ ID NO: 12;
CDR-H3 comprising the amino acid sequence of SEQ ID NO: 3;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

10. The multispecific antigen-binding molecule of any one of claims 1-5, wherein the first antigen-binding domain comprises:
CDR-H1 comprising the amino acid sequence of SEQ ID NO: 9;
CDR-H2 comprising the amino acid sequence of SEQ ID NO: 12;
CDR-H3 comprising the amino acid sequence of SEQ ID NO: 14;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

11. The multispecific antigen-binding molecule of any one of claims 1-5, wherein the first antigen-binding domain comprises:
CDR-H1 comprising the amino acid sequence of SEQ ID NO: 10;
CDR-H2 comprising the amino acid sequence of SEQ ID NO: 13;
CDR-H3 comprising the amino acid sequence of SEQ ID NO: 15;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

12. The multispecific antigen-binding molecule of any one of claims 1-11, wherein the multispecific antigen-binding molecule is a humanized antibody.

13. The multispecific antigen-binding molecule of any one of claims 1-12, wherein the first antigen-binding domain and the second antigen-binding domain each independently comprises an IgG framework region, optionally an IgG₁ or IgG₄ framework region.

14. The multispecific antigen-binding molecule of any one of claims 1-13, wherein the first antigen-binding domain comprises an anti-STEAP1 heavy chain variable region comprising a VH sequence having at least 80%, 85%, 90%, 95%, or 100% sequence identity to SEQ ID NO: 7, 17-25, 30-34, 38, or 68.

15. The multispecific antigen-binding molecule of any one of claims 1-14, wherein the first antigen-binding domain comprises an anti-STEAP1 light chain variable region comprising a VL sequence having at least 80%, 85%, 90%, 95%, or 100% sequence identity to SEQ ID NO: 8, 26-29, 39, or 69.

16. The multispecific antigen-binding molecule of any one of claims 1-15, wherein the first antigen-binding domain comprises an anti-STEAP1 heavy chain variable region comprising a VH sequence having at least 80%, 85%, 90%, 95%, or 100% sequence identity to SEQ ID NO: 68, and an anti-STEAP1 light chain variable region comprising a VL sequence having at least 80%, 85%, 90%, 95%, or 100% sequence identity to SEQ ID NO: 69.

17. The multispecific antigen-binding molecule of any one of claims 1-15, wherein the first antigen-binding domain comprises an anti-STEAP1 heavy chain variable region comprising a VH sequence having at least 80%, 85%, 90%, 95%, or 100% sequence identity to SEQ ID NO: 34, and an anti-STEAP1 light chain variable region comprising a VL sequence having at least 80%, 85%, 90%, 95%, or 100% sequence identity to SEQ ID NO: 27.

18. The multispecific antigen-binding molecule of any one of claims 1-15, wherein the first antigen-binding domain comprises an anti-STEAP1 heavy chain variable region comprising a VH sequence having at least 80%, 85%, 90%, 95%, or 100% sequence identity to SEQ ID NO: 30, and an anti-STEAP1 light chain variable region comprising a VL sequence having at least 80%, 85%, 90%, 95%, or 100% sequence identity to SEQ ID NO: 27.

19. The multispecific antigen-binding molecule of any one of claims 1-15, wherein the first antigen-binding domain comprises an anti-STEAP1 heavy chain variable region comprising a VH sequence having at least 80%, 85%, 90%, 95%, or 100% sequence identity to SEQ ID NO: 31, and an anti-STEAP1 light chain variable region comprising a VL sequence having at least 80%, 85%, 90%, 95%, or 100% sequence identity to SEQ ID NO: 27.

20. The multispecific antigen-binding molecule of any one of claims 1-15, wherein the first antigen-binding domain comprises an anti-STEAP1 heavy chain variable region comprising a VH sequence having at least 80%, 85%, 90%, 95%, or 100% sequence identity to SEQ ID NO: 32, and an anti-STEAP1 light chain variable region comprising a VL sequence having at least 80%, 85%, 90%, 95%, or 100% sequence identity to SEQ ID NO: 27.

21. The multispecific antigen-binding molecule of any one of claims 1-15, wherein the first antigen-binding domain comprises an anti-STEAP1 heavy chain variable region comprising a VH sequence having at least 80%, 85%, 90%, 95%, or 100% sequence identity to SEQ ID NO: 33, and an anti-STEAP1 light chain variable region comprising a VL sequence having at least 80%, 85%, 90%, 95%, or 100% sequence identity to SEQ ID NO: 27.

22. The multispecific antigen-binding molecule of any one of claims 1-21, wherein the T cell receptor is cluster of differentiation 3 (CD3).

23. The multispecific antigen-binding molecule of claim 22, wherein the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the sequences of SEQ ID NOs: 48-53, respectively.

24. The multispecific antigen-binding molecule of claim 23, wherein the second antigen-binding domain that binds to CD3 comprises an anti-CD3 heavy chain variable region comprising a VH sequence having at least 80%, 85%, 90%, 95%, or 100% sequence identity to SEQ ID NO: 54, and a VL sequence having at least 80%, 85%, 90%, 95%, or 100% sequence identity to SEQ ID NO: 55.

25. The multispecific antigen-binding molecule of claim 22, wherein the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the sequences of SEQ ID NOs: 40-45, respectively.

26. The multispecific antigen-binding molecule of claim 25, wherein the second antigen-binding domain that binds to CD3 comprises an anti-CD3 heavy chain variable region comprising a VH sequence having at least 80%, 85%, 90%, 95%, or 100% sequence identity to SEQ ID NO: 46, and an anti-CD3 light chain variable region comprising a VL sequence having at least 80%, 85%, 90%, 95%, or 100% sequence identity to SEQ ID NO: 47.

27. The multispecific antigen-binding molecule of any one of claims 1-26, wherein the multispecific antigen binding molecule comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, a second CH2 (CH2₂) domain, and a second CH3 (CH3₂) domain.

28. The multispecific antigen-binding molecule of claim 27, wherein at least one of the one or more heavy chain constant domains is paired with another heavy chain constant domain.

29. The multispecific antigen-binding molecule of claim 28, wherein the CH3₁ and CH3₂ domains each comprise a protuberance or cavity, and wherein the protuberance or cavity in the CH3₁ domain is positionable in the cavity or protuberance, respectively, in the CH3₂ domain.

30. The multispecific antigen-binding molecule of claim 29, wherein the CH3₁ and CH3₂ domains meet at an interface between said protuberance and cavity.

31. The multispecific antigen-binding molecule of any one of claims 27-30, wherein the CH2₁ and CH2₂ domains each comprise a protuberance or cavity, and wherein the protuberance or cavity in the CH2₁ domain is positionable in the cavity or protuberance, respectively, in the CH2₂ domain.

32. The multispecific antigen-binding molecule of claim 31, wherein the CH2₁ and CH2₂ domains meet at an interface between said protuberance and cavity.

33. The multispecific antigen-binding molecule of any one of claims 22 and 27-32, wherein the first antigen-binding domain comprises an anti-STEAP1 heavy chain constant region sequence having at least 80%, 85%, 90%, 95%, or 100% sequence identity to SEQ ID NO: 83 and an anti-STEAP1 light chain constant region sequence having at least 80%, 85%, 90%, 95%, or 100% sequence identity to SEQ ID NO: 82; and wherein the second antigen-binding domain comprises an anti-CD3 heavy chain constant region sequence having at least 80%, 85%, 90%, 95%, or 100% sequence identity to SEQ ID NO: 85 and an anti-CD3 light chain constant region sequence having at least 80%, 85%, 90%, 95%, or 100% sequence identity to SEQ ID NO: 84.

34. The multispecific antigen-binding molecule of any one of claims 22 and 27-32, wherein the first antigen-binding domain comprises an anti-STEAP1 heavy chain constant region sequence having at least 80%, 85%, 90%, 95%, or 100% sequence identity to SEQ ID NO: 85 and an anti-STEAP1 light chain constant region sequence having at least 80%, 85%, 90%, 95%, or 100% sequence identity to SEQ ID NO: 84; and wherein the second antigen-binding domain comprises an anti-CD3 heavy chain constant region sequence having at least 80%, 85%, 90%, 95%, or 100% sequence identity to SEQ ID NO: 83 and an anti-CD3 light chain constant region sequence having at least 80%, 85%, 90%, 95%, or 100% sequence identity to SEQ ID NO: 82.

35. The multispecific antigen-binding molecule of any one of claims 22 and 27-32, wherein the multispecific antigen-binding molecule comprises an anti-STEAP1 heavy chain comprising SEQ ID NO: 73, an anti-STEAP1 light chain comprising SEQ ID NO: 72, an anti-CD3 heavy chain comprising SEQ ID NO: 81, and an anti-CD3 light chain comprising SEQ ID NO: 80.

36. The multispecific antigen-binding molecule of any one of claims 22 and 27-32, wherein the multispecific antigen-binding molecule comprises an anti-STEAP1 heavy chain comprising SEQ ID NO: 73, an anti-STEAP1 light chain comprising SEQ ID NO: 72, an anti-CD3 heavy chain comprising SEQ ID NO: 79, and an anti-CD3 light chain comprising SEQ ID NO: 78.

37. The multispecific antigen-binding molecule of any one of claims 1-36, wherein the antigen-binding molecule is a single-chain Fv (scFv), trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H})₂), bispecific single-chain Fv (Bis-scFv), IgGdeltaCH2, scFv-Fc, or (scFv)₂-Fc.

38. The multispecific antigen-binding molecule of any one of claims 1-37, wherein the multispecific antigen-binding molecule is a multispecific antibody, preferably a bispecific or trispecific antibody.

39. The multispecific antigen-binding molecule of any one of claims 1-38, wherein the multispecific antigen-binding molecule further comprises a third antigen-binding domain that binds to a tumor-associated antigen.

40. The multispecific antigen-binding molecule of claim 39, wherein the tumor-associated antigen is a receptor expressed on a prostate cancer cell or Ewing sarcoma.

41. The multispecific antigen-binding molecule of claim 39 or 40, wherein the tumor-associated antigen is prostate-specific membrane antigen (PSMA), STEAP2, prostate stem cell antigen (PSCA), epithelial cell adhesion molecule (EpCAM), prostate-specific antigen (PSA), prostatic acid phosphatase (PAP), or HBA-71.

42. The multispecific antigen-binding molecule of any one of claims 1-41, wherein the multispecific antigen-binding molecule binds to human STEAP1, cynomolgus monkey STEAP1, or a combination thereof, preferably wherein the multispecific antigen-binding molecule binds to the human STEAP1 of SEQ ID NO: 65.

43. The multispecific antigen-binding molecule of any one of claims 1-42, wherein the first antigen-binding domain binds to at least one residue selected from Ser101, His102, Gln103, and Lys281 of STEAP1, wherein the residue positions 101, 102, 103, and 281 correspond to positions 101, 102, 103, and 281 set forth in SEQ ID NO: 65.

44. The multispecific antigen-binding molecule of any one of claims 1-43, wherein the first antigen-binding domain binds to Ser101, His102, Gln103, and Lys281 of STEAP1.

45. The multispecific antigen-binding molecule of any one of claims 1-44, wherein the multispecific antigen-binding molecule has a Cₘₐₓ of about 11, 11.5, 12, 12.6, 13, 13.5, 15, 18, 20, 23.4, 25, 27.9, 29.1, or 30 μg/mL.

46. The multispecific antigen-binding molecule of any one of claims 1-44, wherein the multispecific antigen-binding molecule has a Cₘₐₓ of about 11 μg/mL to about 30 μg/mL.

47. The multispecific antigen-binding molecule of claim 46, wherein the multispecific antigen-binding molecule has a Cₘₐₓ of about 20 μg/mL to about 28 μg/mL.

48. The multispecific antigen-binding molecule of claim 47, wherein the multispecific antigen-binding molecule has a Cₘₐₓ of about 24 μg/mL.

49. The multispecific antigen-binding molecule of any one of claims 1-48, wherein the multispecific antigen-binding molecule has an EC₅₀ of about 0.6, 0.56, 0.5, 0.45, 0.4, 0.35, 0.3, 0.25, 0.2, 0.15, 0.1, 0.09, 0.05, or lower.

50. The multispecific antigen-binding molecule of any one of claims 1-48, wherein the multispecific antigen-binding molecule has an EC₅₀ of about 0.05 to about 0.8.

51. The multispecific antigen-binding molecule of claim 50, wherein the EC₅₀ is determined in a cell killing assay at 72 hours with human CD8+ T cells and STEAP1-expressing LNCaP-X1.2 cells, and the EC₅₀ is about 0.05 to about 0.4.

52. The multispecific antigen-binding molecule of claim 51, wherein the EC₅₀ is about 0.08 or about 0.3.

53. The multispecific antigen-binding molecule of claim 50, wherein the EC₅₀ is determined in a cell killing assay at 72 hours with human CD8+ T cells and STEAP1-expressing LNCaPX1.2KO3-13 cells, and the EC₅₀ is about 0.1 to about 0.8.

54. The multispecific antigen-binding molecule of claim 53, wherein the EC₅₀ is about 0.1 or about 0.7.

55. An antigen-binding molecule that binds to STEAP1, comprising an anti-STEAP1 heavy chain variable region (VH) comprising CDR-H1, CDR-H2, and CDR-H3 of a VH sequence selected from SEQ ID NO: 7, 17-25, 30-34, 38, and 68; and an anti-STEAP1 light chain variable region (VL) comprising CDR-L1, CDR-L2, and CDR-L3 of a VL sequence selected from SEQ ID NOs: 8, 26-29, 39, and 69.

56. An antigen-binding molecule that binds to STEAP1, comprising: an anti-STEAP1 heavy chain variable region (VH) comprising CDR-H1, CDR-H2, and CDR-H3; and an anti-STEAP1 light chain variable region (VL) comprising CDR-L1, CDR-L2, and CDR-L3;
wherein
CDR-H1 comprises Xaa¹Xaa²YMA (SEQ ID NO: 35);
wherein
Xaa¹ is Asp (D) or Asn (N); and
Xaa² is His (H), Tyr (Y), or Phe (F);
CDR-H2 comprises YIXaa³YDGXaa⁴Xaa⁵TXaa⁶YGDSVKG (SEQ ID NO: 36);
wherein
Xaa³ is Asp (D) or Ser (S);
Xaa⁴ is Gly (G), Asp (D), or Leu (L);
Xaa⁵ is Ser (S), Asp (D), or Asn (N); and
Xaa⁶ is Ser (S) or Tyr (Y);
CDR-H3 comprises RSGXaa⁷YHVGYAMXaa⁸Xaa⁹ (SEQ ID NO: 37);
wherein
Xaa⁷ is Phe (F) or Tyr (Y);
Xaa⁸ is Asn (N) or Asp (D); and
Xaa⁹ is Ala (A) or Gly (G);
CDR-L1 comprises the amino acid sequence of SEQ ID NO: 4;
CDR-L2 comprises the amino acid sequence of SEQ ID NO: 5; and
CDR-L3 comprises the amino acid sequence of SEQ ID NO: 6.

57. The antigen-binding molecule of claim 55 or 56, wherein the CDR-H1 comprises the amino acid sequence of SEQ ID NO: 10, 1, or 9.

58. The antigen-binding molecule of any one of claims 55-57, wherein the CDR-H2 comprises the amino acid sequence of SEQ ID NO: 2, 11, 12, or 13.

59. The antigen-binding molecule of any one of claims 55-58, wherein the CDR-H3 comprises the amino acid sequence of SEQ ID NO: 16, 3, 14, or 15.

60. The antigen-binding molecule of any one of claims 55-59, wherein the antigen-binding molecule comprises:
CDR-H1 comprising the amino acid sequence of SEQ ID NO: 10;
CDR-H2 comprising the amino acid sequence of SEQ ID NO: 2;
CDR-H3 comprising the amino acid sequence of SEQ ID NO: 16;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

61. The antigen-binding molecule of any one of claims 55-59, wherein the antigen-binding molecule comprises:
CDR-H1 comprising the amino acid sequence of SEQ ID NO: 1;
CDR-H2 comprising the amino acid sequence of SEQ ID NO: 2;
CDR-H3 comprising the amino acid sequence of SEQ ID NO: 3;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

62. The antigen-binding molecule of any one of claims 55-59, wherein the antigen-binding molecule comprises:
CDR-H1 comprising the amino acid sequence of SEQ ID NO: 9;
CDR-H2 comprising the amino acid sequence of SEQ ID NO: 11;
CDR-H3 comprising the amino acid sequence of SEQ ID NO: 3;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

63. The antigen-binding molecule of any one of claims 55-59, wherein the antigen-binding molecule comprises:
CDR-H1 comprising the amino acid sequence of SEQ ID NO: 9;
CDR-H2 comprising the amino acid sequence of SEQ ID NO: 12;
CDR-H3 comprising the amino acid sequence of SEQ ID NO: 3;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

64. The antigen-binding molecule of any one of claims 55-59, wherein the antigen-binding molecule comprises:
CDR-H1 comprising the amino acid sequence of SEQ ID NO: 9;
CDR-H2 comprising the amino acid sequence of SEQ ID NO: 12;
CDR-H3 comprising the amino acid sequence of SEQ ID NO: 14;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

65. The antigen-binding molecule of any one of claims 55-59, wherein the antigen-binding molecule comprises:
CDR-H1 comprising the amino acid sequence of SEQ ID NO: 10;
CDR-H2 comprising the amino acid sequence of SEQ ID NO: 13;
CDR-H3 comprising the amino acid sequence of SEQ ID NO: 15;
CDR-L1 comprising the amino acid sequence of SEQ ID NO: 4;
CDR-L2 comprising the amino acid sequence of SEQ ID NO: 5; and
CDR-L3 comprising the amino acid sequence of SEQ ID NO: 6.

66. The antigen-binding molecule of any one of claims 55-65, wherein the antigen-binding molecule is a humanized antibody.

67. The antigen-binding molecule of any one of claims 55-66, wherein the antigen-binding molecule comprises an IgG framework region, optionally an IgG₁ framework region.

68. The antigen-binding molecule of any one of claims 55-67, comprising an anti-STEAP1 heavy chain variable region comprising a VH sequence having at least 80%, 85%, 90%, 95%, or 100% sequence identity to SEQ ID NO: 7, 17-25, 30-34, 38, or 68.

69. The antigen-binding molecule of any one of claims 55-68, comprising an anti-STEAP1 light chain variable region comprising a VL sequence having at least 80%, 85%, 90%, 95%, or 100% sequence identity to SEQ ID NO: 8, 26-29, 39, or 69.

70. The antigen-binding molecule of any one of claims 55-69, comprising an anti-STEAP1 heavy chain variable region comprising a VH sequence having at least 80%, 85%, 90%, 95%, or 100% sequence identity to SEQ ID NO: 68, and an anti-STEAP1 light chain variable region comprising a VL sequence having at least 80%, 85%, 90%, 95%, or 100% sequence identity to SEQ ID NO: 69.

71. The antigen-binding molecule of any one of claims 55-69, comprising an anti-STEAP1 heavy chain variable region comprising a VH sequence having at least 80%, 85%, 90%, 95%, or 100% sequence identity to SEQ ID NO: 34, and an anti-STEAP1 light chain variable region comprising a VL sequence having at least 80%, 85%, 90%, 95%, or 100% sequence identity to SEQ ID NO: 27.

72. The antigen-binding molecule of any one of claims 55-69, comprising an anti-STEAP1 heavy chain variable region comprising a VH sequence having at least 80%, 85%, 90%, 95%, or 100% sequence identity to SEQ ID NO: 30, and an anti-STEAP1 light chain variable region comprising a VL sequence having at least 80%, 85%, 90%, 95%, or 100% sequence identity to SEQ ID NO: 27.

73. The antigen-binding molecule of any one of claims 55-69, comprising an anti-STEAP1 heavy chain variable region comprising a VH sequence having at least 80%, 85%, 90%, 95%, or 100% sequence identity to SEQ ID NO: 31, and an anti-STEAP1 light chain variable region comprising a VL sequence having at least 80%, 85%, 90%, 95%, or 100% sequence identity to SEQ ID NO: 27.

74. The antigen-binding molecule of any one of claims 55-69, comprising an anti-STEAP1 heavy chain variable region comprising a VH sequence having at least 80%, 85%, 90%, 95%, or 100% sequence identity to SEQ ID NO: 32, and an anti-STEAP1 light chain variable region comprising a VL sequence having at least 80%, 85%, 90%, 95%, or 100% sequence identity to SEQ ID NO: 27.

75. The antigen-binding molecule of any one of claims 55-69, comprising an anti-STEAP1 heavy chain variable region comprising a VH sequence having at least 80%, 85%, 90%, 95%, or 100% sequence identity to SEQ ID NO: 33, and an anti-STEAP1 light chain variable region comprising a VL sequence having at least 80%, 85%, 90%, 95%, or 100% sequence identity to SEQ ID NO: 27.

76. The antigen-binding molecule of any one of claims 55-75, wherein the antigen-binding molecule comprises an anti-STEAP1 heavy chain constant region sequence having at least 80%, 85%, 90%, 95%, or 100% sequence identity to SEQ ID NO: 83 and an anti-STEAP1 light chain constant region sequence having at least 80%, 85%, 90%, 95%, or 100% sequence identity to SEQ ID NO: 82; or wherein the antigen-binding molecule comprises an anti-STEAP1 heavy chain constant region sequence having at least 80%, 85%, 90%, 95%, or 100% sequence identity to SEQ ID NO: 85 and an anti-STEAP1 light chain constant region sequence having at least 80%, 85%, 90%, 95%, or 100% sequence identity to SEQ ID NO: 84.

77. The antigen-binding molecule of claim 76, wherein the antigen-binding molecule comprises an anti-STEAP1 heavy chain comprising SEQ ID NO: 73 and an anti-STEAP1 light chain comprising SEQ ID NO: 72.

78. The antigen-binding molecule of any one of claims 55-77, wherein the antigen-binding molecule is a full-length antibody or a fragment thereof.

79. The antigen-binding molecule of any one of claims 55-78, wherein the antigen-binding molecule is a Fab, Fab′, F(ab′)₂, Fv, Fd, single-chain Fv (scFv), trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H})₂), bispecific single-chain Fv (Bis-scFv), IgGdeltaCH2, scFv-Fc, or (scFv)₂-Fc.

80. The antigen-binding molecule of any one of claims 55-79, wherein the antigen-binding molecule further comprises an antigen-binding domain that binds to a T cell receptor.

81. The antigen-binding molecule of any one of claims 55-80, wherein the antigen-binding molecule is a multispecific antibody, preferably wherein the antigen-binding molecule is a bispecific antibody or a trispecific antibody.

82. The antigen-binding molecule of claim 80 or 81, wherein the T cell receptor is cluster of differentiation 3 (CD3).

83. The antigen-binding molecule of claim 82, wherein the antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein CDR-H1, CDR-H2, CDR-H3, CDR-L1, CDR-L2, and CDR-L3 comprise the amino acid sequences of SEQ ID NO: 48-53, respectively.

84. The antigen-binding molecule of claim 83, wherein the antigen-binding domain that binds to CD3 comprises an anti-CD3 heavy chain variable region comprising a VH sequence having at least 80%, 85%, 90%, 95%, or 100% sequence identity to SEQ ID NO: 54, and an anti-CD3 light chain variable region comprising a VL sequence having at least 80%, 85%, 90%, 95%, or 100% sequence identity to SEQ ID NO: 55.

85. The antigen-binding molecule of claim 82, wherein the antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein CDR-H1, CDR-H2, CDR-H3, CDR-L1, CDR-L2, and CDR-L3 comprise the amino acid sequences of SEQ ID NO: 40-45, respectively.

86. The antigen-binding molecule of claim 83, wherein the antigen-binding domain that binds to CD3 comprises an anti-CD3 heavy chain variable region comprising a VH sequence having at least 80%, 85%, 90%, 95%, or 100% sequence identity to SEQ ID NO: 46, and an anti-CD3 light chain variable region comprising a VL sequence having at least 80%, 85%, 90%, 95%, or 100% sequence identity to SEQ ID NO: 47.

87. The antigen-binding molecule of claim 82, wherein the antigen-binding molecule comprises an anti-CD3 heavy chain comprising SEQ ID NO: 79 and an anti-CD3 light chain comprising SEQ ID NO: 78.

88. The antigen-binding molecule of claim 82, wherein the antigen-binding molecule comprises an anti-CD3 heavy chain comprising SEQ ID NO: 81 and an anti-CD3 light chain comprising SEQ ID NO: 80.

89. The antigen-binding molecule of any one of claims 55-88, wherein the antigen-binding molecule further comprises an additional antigen-binding domain that binds to a tumor-associated antigen.

90. The antigen-binding molecule of claim 89, wherein the tumor-associated antigen is a receptor expressed on a prostate cancer cell or Ewing sarcoma.

91. The antigen-binding molecule of claim 89 or 90, wherein the tumor-associated antigen is prostate-specific membrane antigen (PSMA), STEAP2, prostate stem cell antigen (PSCA), epithelial cell adhesion molecule (EpCAM), prostate-specific antigen (PSA), prostatic acid phosphatase (PAP), or HBA-71.

92. The antigen-binding molecule of any one of claims 55-91, wherein the antigen-binding molecule binds to human STEAP1, cynomolgus monkey STEAP1, or a combination thereof, preferably wherein the antigen-binding molecule binds to the human STEAP1 of SEQ ID NO: 65.

93. The antigen-binding molecule of any one of claims 55-92, wherein the antigen-binding molecule binds to at least one residue selected from Ser101, His102, Gln103, and Lys281 of STEAP1, wherein the residue positions 101, 102, 103, and 281 correspond to positions 101, 102, 103, and 281 set forth in SEQ ID NO: 65.

94. The antigen-binding molecule of any one of claims 55-93, wherein the antigen-binding molecule binds to Ser101, His102, Gln103, and Lys281 of SEQ ID NO: 65.

95. An antibody comprising a first antigen-binding domain that binds to human STEAP1 at one or more residues selected from Ser101, His102, Gln103, and Lys281, wherein the residue positions 101, 102, 103, and 281 correspond to positions 101, 102, 103, and 281 set forth in SEQ ID NO: 65.

96. The antibody of claim 95, wherein the first antigen-binding domain binds to Ser101, His102, Gln103, and Lys281 of SEQ ID NO: 65.

97. The antibody of claim 95 or 96, wherein the heavy chain variable region of the first antigen-binding domain forms a hydrogen bond with at least one residue selected from Ser101, His102, Gln103, and Lys281.

98. The antibody of any one of claims 95-97, wherein Leu56, Ser73, and Asn74 of the heavy chain variable region of the first antigen-binding domain forms a hydrogen bond with Ser101, His102, Gln103, and Lys281 of STEAP1, wherein the residue positions of the heavy chain variable region correspond to positions 56, 73, and 74 set forth in SEQ ID NO: 18.

99. The antibody of any one of claims 95-98, wherein the first antigen-binding domain comprises an anti-STEAP1 heavy chain variable region (VH) comprising CDR-H1, CDR-H2, and CDR-H3 of a VH sequence selected from SEQ ID NO: 7, 17-25, 30-34, 38, and 68; and three CDRs of an anti-STEAP1 light chain variable region (VL) comprising CDR-L1, CDR-L2, and CDR-L3 of a VL sequence selected from SEQ ID NO: 8, 26-29, 39, and 69.

100. The antibody of any one of claims 95-99, wherein the first antigen-binding domain comprises an anti-STEAP1 heavy chain variable region comprising a VH sequence comprising at least 80%, 85%, 90%, 95%, or 100% sequence identity to SEQ ID NO: 7, 17-25, 30-34, 38, or 68.

101. The antibody of any one of claims 95-100, wherein the first antigen-binding domain comprises an anti-STEAP1 light chain variable region comprising a VL sequence comprising at least 80%, 85%, 90%, 95%, or 100% sequence identity to SEQ ID NO: 8, 26-29, 39, or 69.

102. The antibody of any one of claims 95-101, wherein the antibody further comprises a second antigen-binding domain that binds to a T cell receptor.

103. The antibody of claim 102, wherein the T cell receptor is cluster of differentiation 3 (CD3).

104. The antibody of claim 103, wherein the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the amino acid sequences of SEQ ID NOs: 48-53, respectively.

105. The antibody of claim 104, wherein the second antigen-binding domain that binds to CD3 comprises an anti-CD3 heavy chain variable region comprising a VH sequence having at least 80%, 85%, 90%, 95%, or 100% sequence identity to SEQ ID NO: 54 and an anti-CD3 light chain variable region comprising a VL sequence having at least 80%, 85%, 90%, 95%, or 100% sequence identity to SEQ ID NO: 55.

106. The antibody of claim 103, wherein the second antigen-binding domain that binds to CD3 comprises an anti-CD3 heavy chain variable region (VH) comprising CDR-H1, CDR-H2, and CDR-H3, and an anti-CD3 light chain variable region (VL) comprising CDR-L1, CDR-L2, and CDR-L3, wherein the CDR-H1, CDR-H2, CDR-H3, CDR-L1, CDR-L2, and CDR-L3 comprise the amino acid sequences of SEQ ID NOs: 40-45, respectively.

107. The antibody of claim 106, wherein the second antigen-binding domain that binds to CD3 comprises an anti-CD3 heavy chain variable region comprising a VH sequence having at least 80%, 85%, 90%, 95%, or 100% sequence identity to SEQ ID NO: 46 and an anti-CD3 light chain variable region comprising a VL sequence having at least 80%, 85%, 90%, 95%, or 100% sequence identity to SEQ ID NO: 47.

108. The antibody of any one of claims 95-107, wherein the multispecific antigen-binding molecule further comprises a third antigen-binding domain that binds to a tumor-associated antigen.

109. The antibody of claim 108, wherein the tumor-associated antigen is a receptor expressed on a prostate cancer cell.

110. The antibody of claim 108 or 109, wherein the tumor-associated antigen is prostate-specific membrane antigen (PSMA), STEAP2, prostate stem cell antigen (PSCA), epithelial cell adhesion molecule (EpCAM), prostate-specific antigen (PSA), or prostatic acid phosphatase (PAP).

111. One or more isolated nucleic acids individually or together encoding the multispecific antigen-binding molecule of any one of claims 1-54, the antigen-binding molecule of any one of claims 55-94, or the antibody of any one of claims 95-110.

112. One or more vectors individually or together comprising the isolated nucleic acid(s) of claim 111.

113. One or more host cells individually or together comprising the isolated nucleic acid(s) of claim 111, or the vector(s) of claim 112.

114. The host cell(s) of claim 113, wherein the host cell is a mammalian cell.

115. The host cell(s) of claim 114, wherein the mammalian cell is a Chinese hamster ovary (CHO) cell.

116. The host cell(s) of claim 113, wherein the host cell is an insect cell.

117. The host cell(s) of claim 113, wherein the host cell is a prokaryotic cell.

118. A method of producing the multispecific antigen-binding molecule of any one of claims 1-54, the antigen-binding molecule of any one of claims 55-94, or the antibody of any one of claims 95-110 comprising culturing the host cell(s) of any one of claims 113-117 in a culture medium.

119. The method of claim 118, further comprising harvesting the multispecific antigen-binding molecule of any one of claims 1-54, the antigen-binding molecule of any one of claims 55-94, or the antibody of any one of claims 95-110 from the host cell(s) or the culture medium.

120. A pharmaceutical composition comprising the multispecific antigen-binding molecule of any one of claims 1-54, the antigen-binding molecule of any one of claims 55-94, or the antibody of any one of claims 95-110.

121. The pharmaceutical composition of claim 120, further comprising a pharmaceutically acceptable carrier, excipient, or diluent.

122. The pharmaceutical composition of claim 120 or 121 for use as a medicament.

123. The pharmaceutical composition of claim 120 or 121 for use in treating or delaying progression of a STEAP1-expressing cancer.

124. The pharmaceutical composition of claim 120 or 121 for use in treating or delaying progression of prostate cancer or Ewing sarcoma.

125. The multispecific antigen-binding molecule of any one of claims 1-54, the antigen-binding molecule of any one of claims 55-94, or the antibody of any one of claims 95-110 for use in treating or delaying progression of a STEAP1-expressing cancer in a subject in need thereof.

126. The multispecific antigen-binding molecule of any one of claims 1-54, the antigen-binding molecule of any one of claims 55-94, or the antibody of any one of claims 95-110 for use in inhibiting or reducing the proliferation of a STEAP1-expressing cancer cell.

127. The multispecific antigen-binding molecule for use, the antigen-binding molecule for use, or the antibody for use of claim 125 or 126, wherein the STEAP1-expressing cancer is a solid tumor.

128. The multispecific antigen-binding molecule for use, the antigen-binding molecule for use, or the antibody for use of any one of claims 125-127, wherein the STEAP1-expressing cancer is prostate cancer or Ewing sarcoma.

129. The multispecific antigen-binding molecule for use, the antigen-binding molecule for use, or the antibody for use of any one of claims 125-128, which is to be used in combination with an additional therapeutic agent or an additional therapeutic regimen.

130. The multispecific antigen-binding molecule for use, the antigen-binding molecule for use, or the antibody for use of claim 129, wherein the additional therapeutic agent comprises a chemotherapeutic agent, an immunotherapeutic agent, a targeted therapy, radiation therapy, or a combination thereof.

131. The multispecific antigen-binding molecule for use, the antigen-binding molecule for use, or the antibody for use of claim 129 or 130, wherein the additional therapeutic agent comprises a first-line, second-line, or third-line therapy.

132. The multispecific antigen-binding molecule for use, the antigen-binding molecule for use, or the antibody for use of claim 129, wherein the additional therapeutic regimen comprises surgery.

133. The multispecific antigen-binding molecule for use, the antigen-binding molecule for use, or the antibody for use of any one of claims 129-131, wherein the multispecific antigen binding molecule, the antigen binding molecule, or the antibody and the additional therapeutic agent are to be administered simultaneously.

134. The multispecific antigen-binding molecule for use, the antigen-binding molecule for use, or the antibody for use of any one of claims 129-131, wherein the multispecific antigen binding molecule, the antigen binding molecule, or the antibody and the additional therapeutic agent are to be administered sequentially.

135. The multispecific antigen-binding molecule for use, the antigen-binding molecule for use, or the antibody for use of any one of claims 129-131, wherein the multispecific antigen binding molecule, the antigen binding molecule, or the antibody is to be administered first prior to administering the additional therapeutic agent.

136. The multispecific antigen-binding molecule for use, the antigen-binding molecule for use, or the antibody for use of any one of claims 129-131, wherein the multispecific antigen binding molecule, the antigen binding molecule, or the antibody is to be administered after administering the additional therapeutic agent.

137. The multispecific antigen-binding molecule for use, the antigen-binding molecule for use, or the antibody for use of any one of claims 125-136, wherein the multispecific antigen binding molecule, the antigen binding molecule, or the antibody is to be administered systemically.

138. The multispecific antigen-binding molecule for use, the antigen-binding molecule for use, or the antibody for use of any one of claims 125-136, wherein the multispecific antigen binding molecule, the antigen binding molecule, or the antibody is to be administered locally.

139. The multispecific antigen-binding molecule for use, the antigen-binding molecule for use, or the antibody for use of any one of claims 125-136, wherein the multispecific antigen binding molecule, the antigen binding molecule, or the antibody is to be administered by parenteral administration.

140. The multispecific antigen-binding molecule for use, the antigen-binding molecule for use, or the antibody for use of any one of claims 125-136, wherein the multispecific antigen binding molecule, the antigen binding molecule, or the antibody is to be administered intravenously or subcutaneously.

141. The multispecific antigen-binding molecule for use, the antigen-binding molecule for use, or the antibody for use of any one of claims 125-136, wherein the subject is a human.

142. Use of the multispecific antigen-binding molecule of any one of claims 1-54, the antigen-binding molecule of any one of claims 55-94, or the antibody of any one of claims 95-110 in the manufacture of a medicament for treating or delaying progression of a STEAP1-expressing cancer in a subject in need thereof, optionally for treating or delaying progression of prostate cancer or Ewing sarcoma.

143. Use of the multispecific antigen-binding molecule of any one of claims 1-54, the antigen-binding molecule of any one of claims 55-94, or the antibody of any one of claims 95-110 in the manufacture of a medicament 16, optionally a prostate cancer cell or Ewing sarcoma cell.

144. A method for treating or delaying progression of a STEAP1-expressing cancer in a subject in need thereof, comprising administering to the subject an effective amount of the multispecific antigen-binding molecule of any one of claims 1-54, the antigen-binding molecule of any one of claims 55-94, or the antibody of any one of claims 95-110.

145. The method of claim 144, wherein the STEAP1-expressing cancer is a solid tumor.

146. The method of claim 144 or 145, wherein the STEAP1-expressing cancer is prostate cancer or Ewing sarcoma.

147. The method of any one of claims 144-146, further comprising administering to the subject an additional therapeutic agent or an additional therapeutic regimen.

148. The method of claim 147, wherein the additional therapeutic agent comprises a chemotherapeutic agent, an immunotherapeutic agent, a targeted therapy, radiation therapy, or a combination thereof.

149. The method of claim 148, wherein the additional therapeutic agent comprises a first-line, second-line, or third-line therapy.

150. The method of claim 148, wherein the additional therapeutic regimen comprises surgery.

151. The method of any one of claims 144-150, wherein the multispecific antigen-binding molecule, the antigen-binding molecule, or the antibody and the additional therapeutic agent are administered simultaneously.

152. The method of any one of claims 144-150, wherein the multispecific antigen-binding molecule, the antigen-binding molecule, or the antibody and the additional therapeutic agent are administered sequentially.

153. The method of claim 152, wherein the multispecific antigen-binding molecule, the antigen-binding molecule, or the antibody is administered first prior to administering the additional therapeutic agent.

154. The method of claim 152, wherein the multispecific antigen-binding molecule, the antigen-binding molecule, or the antibody is administered after administering the additional therapeutic agent.

155. The method of any one of claims 144-154, wherein the multispecific antigen-binding molecule, the antigen-binding molecule, or the antibody is administered systemically.

156. The method of any one of claims 144-154, wherein the multispecific antigen-binding molecule, the antigen-binding molecule, or the antibody is administered locally.

157. The method of any one of claims 144-156, wherein the multispecific antigen-binding molecule, the antigen-binding molecule, or the antibody is administered by parenteral administration.

158. The method of any one of claims 144-157, wherein the multispecific antigen-binding molecule, the antigen-binding molecule, or the antibody is administered intravenously or subcutaneously.

159. The method of any one of claims 144-158, wherein the subject is a human.

160. A method of inhibiting or reducing the proliferation of a STEAP1-expressing cell, comprising contacting the cell with the multispecific antigen-binding molecule of any one of claims 1-54, the antigen-binding molecule of any one of claims 55-94, or the antibody of any one of claims 95-110 for a time sufficient to inhibit proliferation of the cell.

161. The method of claim 160, wherein the cell is a prostate cancer cell or Ewing sarcoma cell.

162. The method of claim 160 or 161, wherein the method is an in vivo method.

163. The method of claim 160 or 161, wherein the method is an in vitro or an ex vivo method.

164. A kit comprising the multispecific antigen-binding molecule of any one of claims 1-54, the antigen-binding molecule of any one of claims 55-94, or the antibody of any one of claims 95-110, the isolated nucleic acid(s) of claim 111, the vector(s) of claim 112, the host cell(s) of any one of claims 113-117, or the pharmaceutical composition of any one of claims 120-124, optionally comprising a set of instructions.


SEQUENCE LISTING (164 sequences)

SEQ ID NO: 1 - protein, 5 aa, synthetic construct
NYYMA

SEQ ID NO: 2 - protein, 17 aa, synthetic construct
YIDYDGGSTSYGDSVKG

SEQ ID NO: 3 - protein, 13 aa, synthetic construct
RSGYYHVGYAMDA

SEQ ID NO: 4 - protein, 16 aa, synthetic construct
RSSQSLEYSDGYTYLE

SEQ ID NO: 5 - protein, 7 aa, synthetic construct
GVSNRFS

SEQ ID NO: 6 - protein, 9 aa, synthetic construct
FQATHDPLT

SEQ ID NO: 7 - protein, 122 aa, synthetic construct
EVQLVESGGGLVRPGRSLKLSCAASGFTFSNYYMAWVRQAPTRGLEWVAYIDYDGGSTSYGDSVKGRFTISRNNAKSTLYLQMNSLRSEDMATYYCARRSGYYHVGYAMDAWGQGTSVTVSS

SEQ ID NO: 8 - protein, 112 aa, synthetic construct
DDVLTQTPVSLSVTLGDQASISCRSSQSLEYSDGYTYLEWYLQKPGQSPQLLIYGVSNRFSGVPDRFIGSGSGTDFTLKISRVEPEDLGVYYCFQATHDPLTFGSGTKLEIK

SEQ ID NO: 9 - protein, 5 aa, synthetic construct
NFYMA

SEQ ID NO: 10 - protein, 5 aa, synthetic construct
DHYMA

SEQ ID NO: 11 - protein, 17 aa, synthetic construct
YISYDGDSTYYGDSVKG

SEQ ID NO: 12 - protein, 17 aa, synthetic construct
YISYDGLDTYYGDSVKG

SEQ ID NO: 13 - protein, 17 aa, synthetic construct
YISYDGDNTYYGDSVKG

SEQ ID NO: 14 - protein, 13 aa, synthetic construct
RSGYYHVGYAMDG

SEQ ID NO: 15 - protein, 13 aa, synthetic construct
RSGYYHVGYAMNA

SEQ ID NO: 16 - protein, 13 aa, synthetic construct
RSGFYHVGYAMNA

SEQ ID NO: 17 - protein, 122 aa, synthetic construct
EVQLVESGGGLVQPGRSLKLSCAASGFTFSNFYMAWVRQAPTKGLEWVAYISYDGDSTYYGDSVKGRFTISRNNAKRTLYLQMNSLRSEDMATYYCARRSGYYHVGYAMDAWGQGTSVTVSS

SEQ ID NO: 18 - protein, 122 aa, synthetic construct
EVQLVESGGGLVQPGRSLKLSCAASGFTFSNFYMAWVRQAPTKGLEWVAYISYDGLDTYYGDSVKGRFTISRSNAKSTLYLQMNSLRSEDMATYYCARRSGYYHVGYAMDAWGQGTSVTVSS

SEQ ID NO: 19 - protein, 122 aa, synthetic construct
EVQLVESGGGLVQPGRSLKLSCAASGFTFSNFYMAWVRQAPTKGLEWVAYISYDGLDTYYGDSVKGRFTISRSNAKSTLYLQMNSLRSEDMATYYCARRSGYYHVGYAMDGWGQGTSVTVSS

SEQ ID NO: 20 - protein, 122 aa, synthetic construct
EVQLVESGGGLVQPGRSLKLSCAASGFTFSDHYMAWVRQAPTKGLEWVAYISYDGDNTYYGDSVKGRFTISRNNAKSTLYLQMNSLRSEDMATYYCARRSGYYHVGYAMNAWGPGTSVTVSS

SEQ ID NO: 21 - protein, 122 aa, synthetic construct
EVRLVESGGGLVQPGRSLKLSCTASGFTFSDHYMAWVRQVPTKGLEWVAYIDYDGGSTSYGDSVKGRFTISRNNAKSTLYLQMNSLRSEDMATYYCARRSGFYHVGYAMNAWGQGTSVTVSS

SEQ ID NO: 22 - protein, 122 aa, synthetic construct
EVQLVESGGGLVQPGGSLRLSCAASGFTFSNYYMAWVRQAPGKGLVWVAYIDYDGGSTSYGDSVKGRFTISRDNAKSTLYLQMNSLRAEDTAVYYCARRSGYYHVGYAMDAWGQGTTVTVSS

SEQ ID NO: 23 - protein, 122 aa, synthetic construct
EVQLVESGGGLVQPGGSLRLSCAASGFTFSNYYMAWVRQAPGKGLVWVSYIDYDGGSTSYGDSVKGRFTISRDNAKSTLYLQMNSLRAEDTAVYYCARRSGYYHVGYAMDAWGQGTTVTVSS

SEQ ID NO: 24 - protein, 122 aa, synthetic construct
EVQLVESGGGLVQPGGSLRLSCAASGFTFSNYYMAWVRQAPGKGLVWVAYIDYDGGSTSYGDSVKGRFTISRDNAKNTLYLQMNSLRAEDTAVYYCARRSGYYHVGYAMDAWGQGTTVTVSS

SEQ ID NO: 25 - protein, 122 aa, synthetic construct
EVQLVESGGGLVQPGGSLRLSCAASGFTFSNYYMAWVRQAPGKGLVWVSYIDYDGGSTSYGDSVKGRFTISRDNAKNTLYLQMNSLRAEDTAVYYCARRSGYYHVGYAMDAWGQGTTVTVSS

SEQ ID NO: 26 - protein, 112 aa, synthetic construct
DDVLTQTPLSLSVTPGQPASISCRSSQSLEYSDGYTYLEWYLQKPGQSPQLLIYGVSNRFSGVPDRFSGSGSGTDFTLKISRVEAEDVGVYYCFQATHDPLTFGGGTKVEIK

SEQ ID NO: 27 - protein, 112 aa, synthetic construct
DIVLTQTPLSLSVTPGQPASISCRSSQSLEYSDGYTYLEWYLQKPGQSPQLLIYGVSNRFSGVPDRFSGSGSGTDFTLKISRVEAEDVGVYYCFQATHDPLTFGGGTKVEIK

SEQ ID NO: 28 - protein, 112 aa, synthetic construct
DDVMTQTPLSLSVTPGQPASISCRSSQSLEYSDGYTYLEWYLQKPGQSPQLLIYGVSNRFSGVPDRFSGSGSGTDFTLKISRVEAEDVGVYYCFQATHDPLTFGGGTKVEIK

SEQ ID NO: 29 - protein, 112 aa, synthetic construct
DIVMTQTPLSLSVTPGQPASISCRSSQSLEYSDGYTYLEWYLQKPGQSPQLLIYGVSNRFSGVPDRFSGSGSGTDFTLKISRVEAEDVGVYYCFQATHDPLTFGGGTKVEIK

SEQ ID NO: 30 - protein, 122 aa, synthetic construct
EVQLVESGGGLVQPGGSLRLSCAASGFTFSNFYMAWVRQAPGKGLVWVSYISYDGDSTYYGDSVKGRFTISRDNAKRTLYLQMNSLRAEDTAVYYCARRSGYYHVGYAMDAWGQGTTVTVSS

SEQ ID NO: 31 - protein, 122 aa, synthetic construct
EVQLVESGGGLVQPGGSLRLSCAASGFTFSNFYMAWVRQAPGKGLVWVSYISYDGLDTYYGDSVKGRFTISRDNAKSTLYLQMNSLRAEDTAVYYCARRSGYYHVGYAMDAWGQGTTVTVSS

SEQ ID NO: 32 - protein, 122 aa, synthetic construct
EVQLVESGGGLVQPGGSLRLSCAASGFTFSNFYMAWVRQAPGKGLVWVSYISYDGLDTYYGDSVKGRFTISRDNAKSTLYLQMNSLRAEDTAVYYCARRSGYYHVGYAMDGWGQGTTVTVSS

SEQ ID NO: 33 - protein, 122 aa, synthetic construct
EVQLVESGGGLVQPGGSLRLSCAASGFTFSDHYMAWVRQAPGKGLVWVSYISYDGDNTYYGDSVKGRFTISRDNAKSTLYLQMNSLRAEDTAVYYCARRSGYYHVGYAMNAWGQGTTVTVSS

SEQ ID NO: 34 - protein, 122 aa, synthetic construct
EVQLVESGGGLVQPGGSLRLSCAASGFTFSDHYMAWVRQAPGKGLVWVSYIDYDGGSTSYGDSVKGRFTISRDNAKSTLYLQMNSLRAEDTAVYYCARRSGFYHVGYAMNAWGQGTTVTVSS

SEQ ID NO: 35 - skipped

SEQ ID NO: 36 - protein, 17 aa, synthetic construct
  VARIANT 3: note = aspartic acid or serine
  VARIANT 7: note = glycine, aspartic acid, or leucine
  VARIANT 8: note = serine, aspartic acid, or asparagine
  VARIANT 10: note = serine or tyrosine
YIXYDGXXTXYGDSVKG

SEQ ID NO: 37 - protein, 13 aa, synthetic construct
  VARIANT 4: note = phenylalanine or tyrosine
  VARIANT 12: note = asparagine or aspartic acid
  VARIANT 13: note = alanine or glycine
RSGXYHVGYAMXX

SEQ ID NO: 38 - protein, 122 aa, synthetic construct
  VARIANT 31: note = asparagine or aspartic acid
  VARIANT 32: note = tyrosine, phenylalanine, or histidine
  VARIANT 39: note = aspartic acid or serine
  VARIANT 49: note = glycine, aspartic acid, or leucine
  VARIANT 52: note = serine, aspartic acid, or asparagine
  VARIANT 56: note = serine or tyrosine
  VARIANT 57: note = tyrosine or phenylalanine
  VARIANT 59: note = aspartic acid or asparagine
  VARIANT 77: note = alanine or glycine
  VARIANT 102: note = glutamine, glutamic acid, or lysine
  VARIANT 110: note = alanine or serine
  VARIANT 111: note = serine, arginine, or asparagine
EVQLVESGGGLVQPGGSLRLSCAASGFTFSXXYMAWVRXAPGKGLVWVXYIXYDGXXTXYGDSVKGRFTISRDNAKXTLYLQMNSLRAEDTAVYYCARRSGXYHVGYAMXXWGQGTTVTVSS

SEQ ID NO: 39 - protein, 112 aa, synthetic construct
  VARIANT 2: note = isoleucine or aspartic acid
  VARIANT 4: note = leucine or methionine
  VARIANT 43: note = glutamine, lysine, or glutamic acid
DXVXTQTPLSLSVTPGQPASISCRSSQSLEYSDGYTYLEWYLXKPGQSPQLLIYGVSNRFSGVPDRFSGSGSGTDFTLKISRVEAEDVGVYYCFQATHDPLTFGGGTKVEIK

SEQ ID NO: 40 - protein, 5 aa, synthetic construct
SYYIH

SEQ ID NO: 41 - protein, 17 aa, synthetic construct
WIYPENDNTKYNEKFKD

SEQ ID NO: 42 - protein, 10 aa, synthetic construct
DGYSRYYFDY

SEQ ID NO: 43 - protein, 17 aa, synthetic construct
KSSQSLLNSRTRKNYLA

SEQ ID NO: 44 - protein, 7 aa, synthetic construct
WASTRES

SEQ ID NO: 45 - protein, 8 aa, synthetic construct
TQSFILRT

SEQ ID NO: 46 - protein, 119 aa, synthetic construct
EVQLVQSGAEVKKPGASVKVSCKASGFTFTSYYIHWVRKAPGQGLEWIGWIYPENDNTKYNEKFKDRVTITADTSTSTAYLELSSLRSEDTAVYYCARDGYSRYYFDYWGQGTLVTVSS

SEQ ID NO: 47 - protein, 112 aa, synthetic construct
DIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIK

SEQ ID NO: 48 - protein, 5 aa, synthetic construct
NYYIH

SEQ ID NO: 49 - protein, 17 aa, synthetic construct
WIYPGDGNTKYNEKFKG

SEQ ID NO: 50 - protein, 10 aa, synthetic construct
DSYSNYYFDY

SEQ ID NO: 51 - protein, 17 aa, synthetic construct
KSSQSLLNSRTRKNYLA

SEQ ID NO: 52 - protein, 7 aa, synthetic construct
WASTRES

SEQ ID NO: 53 - protein, 8 aa, synthetic construct
TQSFILRT

SEQ ID NO: 54 - protein, 119 aa, synthetic construct
EVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSS

SEQ ID NO: 55 - protein, 112 aa, synthetic construct; identical to SEQ ID NO: 47

SEQ ID NO: 56 - protein, 7 aa, synthetic construct
GFTFSNY

SEQ ID NO: 57 - protein, 6 aa, synthetic construct
DYDGGS

SEQ ID NO: 58 - protein, 7 aa, synthetic construct
GFTFSNF

SEQ ID NO: 59 - protein, 7 aa, synthetic construct
GFTFSDH

SEQ ID NO: 60 - protein, 6 aa, synthetic construct
SYDGDS

SEQ ID NO: 61 - protein, 6 aa, synthetic construct
SYDGLD

SEQ ID NO: 62 - protein, 6 aa, synthetic construct
SYDGDN

SEQ ID NO: 63 - protein, 7 aa, synthetic construct
  VARIANT 6: note = asparagine or aspartic acid
  VARIANT 7: note = tyrosine, phenylalanine, or histidine
GFTFSXX

SEQ ID NO: 64 - skipped

SEQ ID NO: 65 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 66 - protein, 339 aa, Macaca fascicularis
MESRKDITNEEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLATIHALIFAWNKWIDIKQFVWYTPPTFMIAVFLPVVVLIFKSILFLPCLRKKILKIRHGWEDVTKINKMEISSQL

SEQ ID NO: 67 - protein, 339 aa, Pongo abelii
MESRKDITNQEELWKMKPRRNLEEDDYLHKDTGETSMLKRPVLLHLHQTAHADEFDCPSELQQTRELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVILPIVVLIFKSILFLPCLRKKILKIRHGWEDVTKINKTEISSQL

SEQ ID NO: 68 - protein, 122 aa, synthetic construct
EVQLVESGGGLVQPGGSLRLSCAASGFTFSDHYMAWVREAPGKGLVWVSYIDYDGGSTSYGDSVKGRFTISRDNAKSTLYLQMNSLRAEDTAVYYCARRSGFYHVGYAMNAWGQGTTVTVSS

SEQ ID NO: 69 - protein, 112 aa, synthetic construct
DIVLTQTPLSLSVTPGQPASISCRSSQSLEYSDGYTYLEWYLKKPGQSPQLLIYGVSNRFSGVPDRFSGSGSGTDFTLKISRVEAEDVGVYYCFQATHDPLTFGGGTKVEIK

SEQ ID NO: 70 - protein, 219 aa, synthetic construct
DIVLTQTPLSLSVTPGQPASISCRSSQSLEYSDGYTYLEWYLQKPGQSPQLLIYGVSNRFSGVPDRFSGSGSGTDFTLKISRVEAEDVGVYYCFQATHDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 452 aa, synthetic construct
EVQLVESGGGLVQPGGSLRLSCAASGFTFSNYYMAWVRQAPGKGLVWVSYIDYDGGSTSYGDSVKGRFTISRDNAKSTLYLQMNSLRAEDTAVYYCARRSGYYHVGYAMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 219 aa, synthetic construct
DIVLTQTPLSLSVTPGQPASISCRSSQSLEYSDGYTYLEWYLKKPGQSPQLLIYGVSNRFSGVPDRFSGSGSGTDFTLKISRVEAEDVGVYYCFQATHDPLTFGGGTKVEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 452 aa, synthetic construct
EVQLVESGGGLVQPGGSLRLSCAASGFTFSDHYMAWVREAPGKGLVWVSYIDYDGGSTSYGDSVKGRFTISRDNAKSTLYLQMNSLRAEDTAVYYCARRSGFYHVGYAMNAWGQGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 219 aa, synthetic construct; identical to SEQ ID NO: 72

SEQ ID NO: 75 - protein, 452 aa, synthetic construct; identical to SEQ ID NO: 73

SEQ ID NO: 76 - protein, 219 aa, synthetic construct; identical to SEQ ID NO: 72

SEQ ID NO: 77 - protein, 452 aa, synthetic construct; identical to SEQ ID NO: 73

SEQ ID NO: 78 - protein, 219 aa, synthetic construct
DIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 449 aa, synthetic construct
EVQLVQSGAEVKKPGASVKVSCKASGFTFTSYYIHWVRKAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 80 - protein, 219 aa, synthetic construct; identical to SEQ ID NO: 78

SEQ ID NO: 81 - protein, 449 aa, synthetic construct
EVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 107 aa, synthetic construct
RTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 330 aa, synthetic construct
ASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 107 aa, synthetic construct
RTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 330 aa, synthetic construct
ASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 7 aa, synthetic construct
GFTFTSY

SEQ ID NO: 87 - protein, 6 aa, synthetic construct
YPENDN

SEQ ID NO: 88 - protein, 7 aa, synthetic construct
GYTFTNY

SEQ ID NO: 89 - protein, 6 aa, synthetic construct
YPGDGN

SEQ ID NO: 90 - protein, 6 aa, synthetic construct
TRYYIS

SEQ ID NO: 91 - protein, 18 aa, synthetic construct
CIYAGSRGITYYASWAKG

SEQ ID NO: 92 - protein, 22 aa, synthetic construct
ADLRPSDITDDYGDYVMDSFDP

SEQ ID NO: 93 - protein, 11 aa, synthetic construct
QASQNVGSYLA

SEQ ID NO: 94 - protein, 7 aa, synthetic construct
RASILSS

SEQ ID NO: 95 - protein, 14 aa, synthetic construct
QSFYYITSTTYAYS

SEQ ID NO: 96 - protein, 131 aa, synthetic construct
QSLEESGGDLVKPGASLTLTCNASGFDFSTRYYISWVRQAPGKGLEWIACIYAGSRGITYYASWAKGRFTISKTSSTTVTLQATSLTAADTATYFCARADLRPSDITDDYGDYVMDSFDPWGPGTLVTVSS

SEQ ID NO: 97 - protein, 112 aa, synthetic construct
DIVMTQTPASVEAAVGGTVTIKCQASQNVGSYLAWYQQKPGQPPKRLIYRASILSSGVPSRFKGSGSGTQFTLTINDLEAADAATYYCQSFYYITSTTYAYSFGGGTEVVVK

SEQ ID NO: 98 - protein, 6 aa, synthetic construct
RRYYIS

SEQ ID NO: 99 - protein, 18 aa, synthetic construct
CIYAGSRGITYYATWAKG

SEQ ID NO: 100 - protein, 22 aa, synthetic construct
ADLRPGDIADDYGDYVMDALHP

SEQ ID NO: 101 - protein, 11 aa, synthetic construct
QASQNIGSYLA

SEQ ID NO: 102 - protein, 7 aa, synthetic construct
RTSILSS

SEQ ID NO: 103 - protein, 14 aa, synthetic construct
QSFYYLTSTTYGYA

SEQ ID NO: 104 - protein, 132 aa, synthetic construct
QEQLEESGGGLVKPGASLTLTCPASGVSLSRRYYISWVRQAPGKGLEWIACIYAGSRGITYYATWAKGRFTVSKTSSTTVTLQMTSLTAADTATYFCARADLRPGDIADDYGDYVMDALHPWGPGTLVTVSS

SEQ ID NO: 105 - protein, 112 aa, synthetic construct
DIVMTQSPASVEAVVGGTVTIKCQASQNIGSYLAWYQQKSGQPPRLLIYRTSILSSGVPSRFKGSGSGTQFTLTISDLEAADAATYYCQSFYYLTSTTYGYAFGGGTEVVVR

SEQ ID NO: 106 - protein, 8 aa, synthetic construct
GFDFSTRY

SEQ ID NO: 107 - protein, 7 aa, synthetic construct
YAGSRGI

SEQ ID NO: 108 - protein, 8 aa, synthetic construct
GVSLSRRY

SEQ ID NO: 109 - protein, 7 aa, synthetic construct
YAGSRGI

SEQ ID NO: 110 - protein, 30 aa, synthetic construct
EVQLVESGGGLVQPGGSLRLSCAASGFTFS

SEQ ID NO: 111 - protein, 14 aa, synthetic construct
WVRQAPGKGLVWVS

SEQ ID NO: 112 - protein, 32 aa, synthetic construct
RFTISRDNAKSTLYLQMNSLRAEDTAVYYCAR

SEQ ID NO: 113 - protein, 11 aa, synthetic construct
WGQGTTVTVSS

SEQ ID NO: 114 - protein, 23 aa, synthetic construct
DIVLTQTPLSLSVTPGQPASISC

SEQ ID NO: 115 - protein, 15 aa, synthetic construct
WYLQKPGQSPQLLIY

SEQ ID NO: 116 - protein, 32 aa, synthetic construct
GVPDRFSGSGSGTDFTLKISRVEAEDVGVYYC

SEQ ID NO: 117 - protein, 10 aa, synthetic construct
FGGGTKVEIK

SEQ ID NO: 118 - protein, 14 aa, synthetic construct
WVREAPGKGLVWVS

SEQ ID NO: 119 - protein, 15 aa, synthetic construct
WYLKKPGQSPQLLIY

SEQ ID NO: 120 - protein, 14 aa, synthetic construct
WVRKAPGKGLVWVS

SEQ ID NO: 121 - protein, 15 aa, synthetic construct
WYLEKPGQSPQLLIY

SEQ ID NO: 122 - protein, 452 aa, synthetic construct
EVQLVESGGGLVQPGGSLRLSCAASGFTFSDHYMAWVREAPGKGLVWVSYIDYDGGSTSYGDSVKGRFTISRDNAKSTLYLQMNSLRAEDTAVYYCARRSGFYHVGYAMNAWGQGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 123 - protein, 219 aa, synthetic construct; identical to SEQ ID NO: 72

SEQ ID NO: 124 - protein, 449 aa, synthetic construct
EVQLVQSGAEVKKPGASVKVSCKASGFTFTSYYIHWVRKAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 219 aa, synthetic construct; identical to SEQ ID NO: 78

SEQ ID NO: 126 - protein, 452 aa, synthetic construct; identical to SEQ ID NO: 122

SEQ ID NO: 127 - protein, 219 aa, synthetic construct; identical to SEQ ID NO: 72

SEQ ID NO: 128 - protein, 449 aa, synthetic construct
EVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 219 aa, synthetic construct; identical to SEQ ID NO: 78

SEQ ID NO: 130 - protein, 452 aa, synthetic construct
EVQLVESGGGLVQPGGSLRLSCAASGFTFSDHYMAWVRKAPGKGLVWVSYIDYDGGSTSYGDSVKGRFTISRDNAKSTLYLQMNSLRAEDTAVYYCARRSGFYHVGYAMNAWGQGTTVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 219 aa, synthetic construct
DIVLTQTPLSLSVTPGQPASISCRSSQSLEYSDGYTYLEWYLEKPGQSPQLLIYGVSNRFSGVPDRFSGSGSGTDFTLKISRVEAEDVGVYYCFQATHDPLTFGGGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 449 aa, synthetic construct
EVQLVQSGAEVKKPGASVKVSCKASGFTFTSYYIHWVREAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 133 - protein, 219 aa, synthetic construct
DIVMTQSPDSLAVSLGERATINCKSSQSLLNSRTRKNYLAWYQKKPGQPPKLLIYWASTRESGVPDRFSGSGSGTDFTLTISSLQAEDVAVYYCTQSFILRTFGQGTKVEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 452 aa, synthetic construct; identical to SEQ ID NO: 130

SEQ ID NO: 135 - protein, 219 aa, synthetic construct; identical to SEQ ID NO: 131

SEQ ID NO: 136 - protein, 449 aa, synthetic construct
EVQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 137 - protein, 219 aa, synthetic construct; identical to SEQ ID NO: 133

SEQ ID NO: 138 - protein, 452 aa, synthetic construct
EVQLVESGGGLVQPGGSLRLSCAASGFTFSDHYMAWVRKAPGKGLVWVSYIDYDGGSTSYGDSVKGRFTISRDNAKSTLYLQMNSLRAEDTAVYYCARRSGFYHVGYAMNAWGQGTTVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 139 - protein, 219 aa, synthetic construct; identical to SEQ ID NO: 131

SEQ ID NO: 140 - protein, 449 aa, synthetic construct
EVQLVQSGAEVKKPGASVKVSCKASGFTFTSYYIHWVREAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 219 aa, synthetic construct; identical to SEQ ID NO: 133

SEQ ID NO: 142 - protein, 452 aa, synthetic construct; identical to SEQ ID NO: 138

SEQ ID NO: 143 - protein, 219 aa, synthetic construct; identical to SEQ ID NO: 131

SEQ ID NO: 144 - protein, 449 aa, synthetic construct
EVQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 219 aa, synthetic construct; identical to SEQ ID NO: 133

SEQ ID NO: 146 - protein, 112 aa, synthetic construct
DVVLTQTPVSLSVTLGDQASISCRSSQSLEYSDGYTYLEWYLQKPGQSPQLLIYGVSNRFSGVPDRFIGSGSGTDFTLKISRVEPEDLGVYYCFQATHDPLTFGSGTKLEIK

SEQ ID NO: 147 - protein, 116 aa, synthetic construct
EVQLVESGGGLVQPGRSMKLSCAASGFTFSDYYMAWVRQAPKKGLEWVASISYEGSSTYYGDSVKGRFTISRDNAKSTLYLQMNSLRSEDTATYYCARGYYHMDAWGQGASVTVSS

SEQ ID NO: 148 - protein, 112 aa, synthetic construct
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLPLTFGGGTKVEIK

SEQ ID NO: 149 - protein, 112 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARMDVWGQGTTVTVSS

SEQ ID NO: 150 - protein, 107 aa, synthetic construct [sequence omitted]

SEQ ID NO: 151 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 5 aa, synthetic construct
DKTHT

SEQ ID NO: 153 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 154 - protein, 7 aa, synthetic construct
GGGGSGG

SEQ ID NO: 155 - protein, 376 aa, synthetic construct
MGSMESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQLGNSGLNDIFEAQKIEWHEGENLYFQSDYKDDDDK

SEQ ID NO: 156 - DNA, 21 bp, synthetic construct
  source 1..21: mol_type = other DNA
tccaggggccagtggatagac

SEQ ID NO: 157 - DNA, 23 bp, synthetic construct
  source 1..23: mol_type = other DNA
cacacmggggccagtggatagac

SEQ ID NO: 158 - DNA, 24 bp, synthetic construct
  source 1..24: mol_type = other DNA
gcagccagggaccaagggatagac

SEQ ID NO: 159 - DNA, 25 bp, synthetic construct
  source 1..25: mol_type = other DNA
ggaagtaatcrtgaatcaggcagcc

SEQ ID NO: 160 - DNA, 21 bp, synthetic construct
  source 1..21: mol_type = other DNA
agatgggggtgtcgttttggc

SEQ ID NO: 161 - DNA, 21 bp, synthetic construct
  source 1..21: mol_type = other DNA
cgatggggstgttgttttggc

SEQ ID NO: 162 - DNA, 24 bp, synthetic construct
  source 1..24: mol_type = other DNA
gacagatggggctgttgttgtagc

SEQ ID NO: 163 - DNA, 21 bp, synthetic construct
  source 1..21: mol_type = other DNA
tgtcagtgggtagatggtggg

SEQ ID NO: 164 - DNA, 28 bp, synthetic construct
  source 1..28: mol_type = other DNA
tgtccttttcataaaaggtgtccagtgt